SEQUENCE LISTING (137 sequences)

SEQ ID NO: 1 - protein, 345 aa, Homo sapiens
MAQHHLWILLLCLQTWPEAAGKDSEIFTVNGILGESVTFPVNIQEPRQVKIIAWTSKTSVAYVTPGDSETAPVVTVTHRNYYERIHALGPNYNLVISDLRMEDAGDYKADINTQADPYTTTKRYNLQIYRRLGKPKITQSLMASVNSTCNVTLTCSVEKEEKNVTYNWSPLGEEGNVLQIFQTPEDQELTYTCTAQNPVSNNSDSISARQLCADIAMGFRTHHTGLLSVLAMFFLLVLILSSVFLFRLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVI

SEQ ID NO: 2 - DNA, 75 bp, Artificial Sequence
  Synthetic polynucleotide
ttgctgagcgtgctggctatgttctttctgcttgttctcattctgtcttcagtgtttttgttccgtttgttcaag

SEQ ID NO: 3 - protein, 583 aa, Homo sapiens
MESKGASSCRLLFCLLISATVFRPGLGWYTVNSAYGDTIIIPCRLDVPQNLMFGKWKYEKPDGSPVFIAFRSSTKKSVQYDDVPEYKDRLNLSENYTLSISNARISDEKRFVCMLVTEDNVFEAPTIVKVFKQPSKPEIVSKALFLETEQLKKLGDCISEDSYPDGNITWYRNGKVLHPLEGAVVIIFKKEMDPVTQLYTMTSTLEYKTTKADIQMPFTCSVTYYGPSGQKTIHSEQAVFDIYYPTEQVTIQVLPPKNAIKEGDNITLKCLGNGNPPPEEFLFYLPGQPEGIRSSNTYTLTDVRRNATGDYKCSLIDKKSMIASTAITVHYLDLSLNPSGEVTRQIGDALPVSCTISASRNATVVWMKDNIRLRSSPSFSSLHYQDAGNYVCETALQEVEGLKKRESLTLIVEGKPQIKMTKKTDPSGLSKTIICHVEGFPKPAIQWTITGSGSVINQTEESPYINGRYYSKIIISPEENVTLTCTAENQLERTVNSLNVSAISIPEHDEADEISDENREKVNDQAKLIVGIVVGLLLAALVAGVVYWLYMKKSKTASKHVNKDLGNMEENKKLEENNHKTEA

SEQ ID NO: 4 - DNA, 78 bp, Artificial Sequence
  Synthetic polynucleotide
ctaattgtgggaatcgttgttggtctcctccttgctgcccttgttgctggtgtcgtctactggctgtacatgaagaag

SEQ ID NO: 5 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 6 - DNA, 75 bp, Artificial Sequence
  Synthetic polynucleotide
atctacatctgggcgcccttggccgggacttgtggggtccttctcctgtcactggttatcaccctttactgcaac

SEQ ID NO: 7 - protein, 221 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQLRLHPLEKCSRMDY

SEQ ID NO: 8 - DNA, 75 bp, Artificial Sequence
  Synthetic polynucleotide
atcacccttggcctgctggtggctggcgtcctggttctgctggtttccctgggagtggccatccacctgtgctgc

SEQ ID NO: 9 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 10 - DNA, 75 bp, Artificial Sequence
  Synthetic polynucleotide
ttctggttacccataggatgtgcagcctttgttgtagtctgcattttgggatgcatacttatttgttggcttaca

SEQ ID NO: 11 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 12 - DNA, 75 bp, Artificial Sequence
  Synthetic polynucleotide
ttcctcctctggatccttgcagcagttagttcggggttgtttttttatagctttctcctcacagctgtttctttg

SEQ ID NO: 13 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP

SEQ ID NO: 14 - DNA, 81 bp, Artificial Sequence
  Synthetic polynucleotide
attgtcatcatcactgtggtagcagccgcagtcataatgggcactgcaggcctcagcacgtacctctataaccgccagcgg

SEQ ID NO: 15 - protein, 39 aa, Artificial Sequence
  Synthetic polypeptide
IEVMYPPPYLDNEKSNGTIIHVKGKHLCPSPLFPGPSKP

SEQ ID NO: 16 - DNA, 117 bp, Artificial Sequence
  Synthetic polynucleotide
caagagctgacttacacgtgtacagcccagaaccctgtcagcaacaattctgactccatctctgcccggcagctctgtgcagacatcgcaatgggcttccgtactcaccacaccggg

SEQ ID NO: 17 - DNA, 116 bp, Artificial Sequence
  Synthetic polynucleotide
accaactggagagaacagtaaactccttgaatgtctctgctataagtattccagaacacgatgaggcagacgagataagtgatgaaaacagagaaaaggtgaatgaccaggcaaaa

SEQ ID NO: 18 - DNA, 138 bp, Artificial Sequence
  Synthetic polynucleotide
cccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 19 - DNA, 117 bp, Artificial Sequence
  Synthetic polynucleotide
ctgagtgtggttgatttccttcccaccactgcccagcccaccaagaagtccaccctcaagaagagagtgtgccggttacccaggccagagacccagaagggcccactttgtagcccc

SEQ ID NO: 20 - DNA, 117 bp, Artificial Sequence
  Synthetic polynucleotide
tctcatgccaactattacttctgcaacctatcaatttttgatcctcctccttttaaagtaactcttacaggaggatatttgcatatttatgaatcacaactttgttgccagctgaag

SEQ ID NO: 21 - DNA, 117 bp, Artificial Sequence
  Synthetic polynucleotide
gacacgggactctacatctgcaaggtggagctcatgtacccaccgccatactacctgggcataggcaacggaacccagatttatgtaattgatccagaaccgtgcccagattctgac

SEQ ID NO: 22 - DNA, 117 bp, Artificial Sequence
  Synthetic polynucleotide
ggggaatcagtgactgtcactcgagatcttgagggcacctacctctgtcgggccaggagcactcaaggggaggtcacccgcaaggtgaccgtgaatgtgctctccccccggtatgag

SEQ ID NO: 23 - protein, 22 aa, Artificial Sequence
  Synthetic polypeptide
QNQLYNELNLGRREEYDVLDKR

SEQ ID NO: 24 - DNA, 66 bp, Artificial Sequence
  Synthetic polynucleotide
cagaaccagctctataacgagctcaatctaggacgaagagaggagtacgatgttttggacaagaga

SEQ ID NO: 25 - protein, 22 aa, Artificial Sequence
  Synthetic polypeptide
QNQLFNELNLGRREEFDVLDKR

SEQ ID NO: 26 - DNA, 66 bp, Artificial Sequence
  Synthetic polynucleotide
cagaaccagctctttaacgagctcaatctaggacgaagagaggagttcgatgttttggacaagaga

SEQ ID NO: 27 - protein, 23 aa, Artificial Sequence
  Synthetic polypeptide
QEGLYNELQKDKMAEAYSEIGMK

SEQ ID NO: 28 - DNA, 69 bp, Artificial Sequence
  Synthetic polynucleotide
caggaaggcctgtacaatgaactgcagaaagataagatggcggaggcctacagtgagattgggatgaaa

SEQ ID NO: 29 - protein, 23 aa, Artificial Sequence
  Synthetic polypeptide
QEGLFNELQKDKMAEAFSEIGMK

SEQ ID NO: 30 - DNA, 69 bp, Artificial Sequence
  Synthetic polynucleotide
caggaaggcctgttcaatgaactgcagaaagataagatggcggaggccttcagtgagattgggatgaaa

SEQ ID NO: 31 - protein, 22 aa, Artificial Sequence
  Synthetic polypeptide
HDGLYQGLSTATKDTYDALHMQ

SEQ ID NO: 32 - DNA, 66 bp, Artificial Sequence
  Synthetic polynucleotide
cacgatggcctttaccagggtctcagtacagccaccaaggacacctacgacgcccttcacatgcag

SEQ ID NO: 33 - protein, 22 aa, Artificial Sequence
  Synthetic polypeptide
HDGLFQGLSTATKDTFDALHMQ

SEQ ID NO: 34 - DNA, 66 bp, Artificial Sequence
  Synthetic polynucleotide
cacgatggccttttccaggggctcagtacagccaccaaggacaccttcgacgcccttcacatgcag

SEQ ID NO: 35 - protein, 112 aa, Artificial Sequence
  Synthetic polypeptide
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 36 - protein, 230 aa, Artificial Sequence
  Synthetic polypeptide
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSGQAG

SEQ ID NO: 37 - DNA, 693 bp, Artificial Sequence
  Synthetic polynucleotide
caggtgcagctgcaggagtccggcccaggactggtgaagccttcggagaccctgtccctcacctgcactgtctctggtggctccgtcagcagtggtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagagaggggaagaatggggcttttgatatctggggccaagggacaatggtcaccgtctcttcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactagtggccaggccggccac

SEQ ID NO: 38 - protein, 214 aa, Artificial Sequence
  Synthetic polypeptide
DIQMTQSPSSLSASVGDRVTITCRASQSISSYLNWYQQKPGKAPKLLIYAASSLQSGVPSGFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - DNA, 640 bp, Artificial Sequence
  Synthetic polynucleotide
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcagggttcagtggcagtggatctgggacagatttcactctcaccatcagcagtctgcaacctgaagattttgcaacttactactgtcaacagagttacagtaccccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctactgcctcagcagcaccctgacgctgagcaaagcagactacgagaaacacaaactctacgcctgcgaagtcacccatcagggcctgagctcgcccgtcacaaagagcttcaacaggggagagt

SEQ ID NO: 40 - protein, 214 aa, Homo sapiens
RHQMTQSPSSLSASVGDRVTITCRASQSISSYLNWYQQKPGKAPKLLIYAASSLQSGVPSGFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 120 aa, Artificial Sequence
  Synthetic polypeptide
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSS

SEQ ID NO: 42 - protein, 262 aa, Artificial Sequence
  Synthetic polynucleotide
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSSGGGGSGGGGSGGGGSRHQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGQAGHHHHHHGDYKDDDDKG

SEQ ID NO: 43 - protein, 486 aa, Artificial Sequence
  Synthetic polypeptide
MALPVTALLLPLALLLHAARPQVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSGGGGSGGGGSGGGGSRHQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 44 - DNA, 1458 bp, Artificial Sequence
  Synthetic polynucleotide
atggccctgccagtaacggctctgctgctgccacttgctctgctcctccatgcagccaggcctcaggttcagcttcaggagagtggcccaggcctggtgaagccaagtgagactctcagcttgacttgcacagtttctggaggcagtgtctcctcaggcagctattattggtcctggattcggcagccccctgggaaaggcctggagtggattgggtacatatattacagtggcagcacaaattacaatccatccctgaagtctcgagtaactatcagtgtggacacaagcaagaatcagttttcactcaaactgtcttctgtgactgctgctgacactgctgtttattattgtgccagggaggggaaaaatggggcatttgatatttggggtcagggcacaatggtgacagtcagctctggaggtggaggctcaggaggaggaggcagtggaggtggtgggtcacgccatcagatgactcagtccccctccagtctttctgcctcagttggggatagagtgaccatcacatgcagagcaagtcagagcatatcatcctatctgaactggtaccagcagaagccagggaaagcccccaaattgctgatttatgcagcctcaagtctccagagtggggtgccaagcaggttctcaggcagtggcagtgggacagatttcacattgacaatcagctccctccaacctgaagattttgccacctactattgccagcaatcctacagcacgcccctgacttttggaggtggcacaaaggtagagatcaagaggactgcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccaggggctcagtacagccaccaaggacaccttcgacgcccttcacatgcaggccctgccccctcgc

SEQ ID NO: 45 - DNA, 801 bp, Artificial Sequence
  Synthetic polynucleotide
atggccttaccagtgaccgccttgctcctgccgctggccttgctgctccacgccgccaggccgcaggtgcagctgcaggagtccggcccaggactggtgaagccttcggagaccctgtccctcacctgcactgtctctggtggctccgtcagcagtggtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagagaggggaagaatggggcttttgatatctggggccaagggacaatggtcaccgtctcttcaggtggaggcggttcaggcggaggtggctctggcggtggcggatcacgacatcagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgctcactttcggcggagggaccaaggtggagatcaaacggactgcggccgca

SEQ ID NO: 46 - DNA, 801 bp, Artificial Sequence
  Synthetic polynucleotide
atggcgctgccggtgaccgcgctgctgctgccgctggcgctgctgctgcatgcggcgcgcccgcaggtgcagctgcaggaaagcggcccgggcctggtgaaaccgagcgaaaccctgagcctgacctgcaccgtgagcggcggcagcgtgagcagcggcagctattattggagctggattcgccagccgccgggcaaaggcctggaatggattggctatatttattatagcggcagcaccaactataacccgagcctgaaaagccgcgtgaccattagcgtggataccagcaaaaaccagtttagcctgaaactgagcagcgtgaccgcggcggataccgcggtgtattattgcgcgcgcgaaggcaaaaacggcgcgtttgatatttggggccagggcaccatggtgaccgtgagcagcggcggcggcggcagcggcggcggcggcagcggcggcggcggcagccgccatcagatgacccagagcccgagcagcctgagcgcgagcgtgggcgatcgcgtgaccattacctgccgcgcgagccagagcattagcagctatctgaactggtatcagcagaaaccgggcaaagcgccgaaactgctgatttatgcggcgagcagcctgcagagcggcgtgccgagccgctttagcggcagcggcagcggcaccgattttaccctgaccattagcagcctgcagccggaagattttgcgacctattattgccagcagagctatagcaccccgctgacctttggcggcggcaccaaagtggaaattaaacgcaccgcggcggcg

SEQ ID NO: 47 - DNA, 801 bp, Artificial Sequence
  Synthetic polynucleotide
atggccctcccggtaacggctctgctgcttccactcgcactgctcttgcatgctgccagaccacaggtccagctgcaggagagtgggcctggactggttaagccgagtgagacactttccttgacgtgcactgtgagcgggggaagtgtgtcctcaggtagttattactggtcctggattcgccagccaccaggaaagggactggagtggataggttatatctattattctggcagcactaattacaatccttctctcaaaagtagggtgacaatttcagtggatacttccaaaaatcagtttagtctgaagctcagctctgtgacagctgctgatactgcagtttactactgcgccagggaggggaagaatggcgccttcgatatttggggacagggcactatggtgactgtatcaagcggaggcggtggcagcggcgggggagggagtggaggcggcgggtctcgacatcagatgacacagagcccatcatcacttagcgccagcgttggcgaccgggttacgataacatgcagggcttcccaatctatcagttcttatctgaactggtatcagcagaaaccaggtaaggcccccaagctgctcatctacgcagcctcatccctgcagagcggcgtccctagtcgattttccggtagtgggtcagggacagattttaccctgactatcagttcactgcagcccgaggacttcgcgacatactattgccaacagtcctatagtacacccttgacatttggcggcgggactaaagtagaaattaaacgcaccgcggccgca

SEQ ID NO: 48 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 49 - protein, 239 aa, Artificial Sequence
  Synthetic polypeptide
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQAAAPTTTPAPRPPTPAPTIASQPLSLRPEACRPAAGGAVHTRGLDFACDIYIWAPLAGTCGVLLLSLVITLYCN

SEQ ID NO: 50 - DNA, 45 bp, Artificial Sequence
  Synthetic polynucleotide
ggaggtggaggctcaggaggaggaggcagtggaggtggtgggtca

SEQ ID NO: 51 - DNA, 45 bp, Artificial Sequence
  Synthetic polynucleotide
ggtggaggcggttcaggcggaggtggctctggcggtggcggatca

SEQ ID NO: 52 - protein, 357 aa, Artificial Sequence
  Synthetic polypeptide
CAGGTTCAGCTTCAGGAGAGTGGCCCAGGCCTGGTGAAGCCAAGTGAGACTCTCAGCTTGACTTGCACAGTTTCTGGAGGCAGTGTCTCCTCAGGCAGCTATTATTGGTCCTGGATTCGGCAGCCCCCTGGGAAAGGCCTGGAGTGGATTGGGTACATATATTACAGTGGCAGCACAAATTACAATCCATCCCTGAAGTCTCGAGTAACTATCAGTGTGGACACAAGCAAGAATCAGTTTTCACTCAAACTGTCTTCTGTGACTGCTGCTGACACTGCTGTTTATTATTGTGCCAGGGAGGGGAAAAATGGGGCATTTGATATTTGGGGTCAGGGCACAATGGTGACAGTCAGCTCT

SEQ ID NO: 53 - DNA, 327 bp, Artificial Sequence
  Synthetic polynucleotide
cgccatcagatgactcagtccccctccagtctttctgcctcagttggggatagagtgaccatcacatgcagagcaagtcagagcatatcatcctatctgaactggtaccagcagaagccagggaaagcccccaaattgctgatttatgcagcctcaagtctccagagtggggtgccaagcaggttctcaggcagtggcagtgggacagatttcacattgacaatcagctccctccaacctgaagattttgccacctactattgccagcaatcctacagcacgcccctgacttttggaggtggcacaaaggtagagatcaagaggact

SEQ ID NO: 54 - DNA, 117 bp, Artificial Sequence
  Synthetic polynucleotide
attgaagttatgtatcctcctccttacctagacaatgagaagagcaatggaaccattatccatgtgaaagggaaacacctttgtccaagtcccctatttcccggaccttctaagccc

SEQ ID NO: 55 - DNA, 336 bp, Artificial Sequence
  Synthetic polynucleotide
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccaggggctcagtacagccaccaaggacaccttcgacgcccttcacatgcaggccctgccccctcgc

SEQ ID NO: 56 - protein, 465 aa, Artificial Sequence
  Synthetic polypeptide
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSGGGGSGGGGSGGGGSRHQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 57 - DNA, 1395 bp, Artificial Sequence
  Synthetic polynucleotide
caggttcagcttcaggagagtggcccaggcctggtgaagccaagtgagactctcagcttgacttgcacagtttctggaggcagtgtctcctcaggcagctattattggtcctggattcggcagccccctgggaaaggcctggagtggattgggtacatatattacagtggcagcacaaattacaatccatccctgaagtctcgagtaactatcagtgtggacacaagcaagaatcagttttcactcaaactgtcttctgtgactgctgctgacactgctgtttattattgtgccagggaggggaaaaatggggcatttgatatttggggtcagggcacaatggtgacagtcagctctggaggtggaggctcaggaggaggaggcagtggaggtggtgggtcacgccatcagatgactcagtccccctccagtctttctgcctcagttggggatagagtgaccatcacatgcagagcaagtcagagcatatcatcctatctgaactggtaccagcagaagccagggaaagcccccaaattgctgatttatgcagcctcaagtctccagagtggggtgccaagcaggttctcaggcagtggcagtgggacagatttcacattgacaatcagctccctccaacctgaagattttgccacctactattgccagcaatcctacagcacgcccctgacttttggaggtggcacaaaggtagagatcaagaggactgcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccaggggctcagtacagccaccaaggacaccttcgacgcccttcacatgcaggccctgccccctcgc

SEQ ID NO: 58 - protein, 145 aa, Artificial Sequence
  Synthetic polypeptide
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQ

SEQ ID NO: 59 - DNA, 435 bp, Artificial Sequence
  Synthetic polynucleotide
ccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgctttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccag

SEQ ID NO: 60 - DNA, 60 bp, Artificial Sequence
  Synthetic polynucleotide
atgcagatcccacaggcgccctggccagtcgtctgggcggtgctacaactgggctggcgg

SEQ ID NO: 61 - DNA, 495 bp, Artificial Sequence
  Synthetic polynucleotide
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgctttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccag

SEQ ID NO: 62 - DNA, 213 bp, Artificial Sequence
  Synthetic polynucleotide
cccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaac

SEQ ID NO: 63 - DNA, 219 bp, Artificial Sequence
  Synthetic polynucleotde
cccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaaccacagg

SEQ ID NO: 64 - DNA, 717 bp, Artificial Sequence
  Synthetic polynucleotide
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgctttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccaggcggccgcacccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaac

SEQ ID NO: 65 - protein, 199 aa, Homo sapiens; identical to SEQ ID NO: 9

SEQ ID NO: 66 - protein, 15 aa, Artificial Sequence
  Synthetic polypeptide
GGGGSGGGGSGGGGS

SEQ ID NO: 67 - protein, 20 aa, Artificial Sequence
  Synthetic polypeptide
MYRMQLLSCIALSLALVTNS

SEQ ID NO: 68 - protein, 20 aa, Artificial Sequence
  Synthetic polypeptide
MYSMQLASCVTLTLVLLVNS

SEQ ID NO: 69 - protein, 20 aa, Artificial Sequence
  Synthetic polypeptide
METPAQLLFLLLLWLPDTTG

SEQ ID NO: 70 - protein, 20 aa, Artificial Sequence
  Synthetic polypeptide
METDTLLLWVLLLWVPGSTG

SEQ ID NO: 71 - protein, 21 aa, Artificial Sequence
  Synthetic polypeptide
MALPVTALLLPLALLLHAARP

SEQ ID NO: 72 - protein, 18 aa, Artificial Sequence
  Synthetic polypeptide
MALPVTALLLPLALLLHA

SEQ ID NO: 73 - protein, 16 aa, Artificial Sequence
  Synthetic polypeptide
MKWVTFISLLFSSAYS

SEQ ID NO: 74 - protein, 30 aa, Artificial Sequence
  Synthetic polypeptide
MDSKGSSQKGSRLLLLLVVSNLLLCQGVVS

SEQ ID NO: 75 - protein, 622 aa, Homo sapiens [sequence omitted]

SEQ ID NO: 76 - protein, 10 aa, Artificial Sequence
  Synthetic polypeptide
GGSVSSGSYY

SEQ ID NO: 77 - protein, 7 aa, Artificial Sequence
  Synthetic polypeptide
IYYSGST

SEQ ID NO: 78 - protein, 12 aa, Artificial Sequence
  Synthetic polypeptide
AREGKNGAFDIW

SEQ ID NO: 79 - protein, 6 aa, Artificial Sequence
  Synthetic polypeptide
QSISSY

SEQ ID NO: 80 - protein, 4 aa, Artificial Sequence
  Synthetic polypeptide
AASS

SEQ ID NO: 81 - protein, 10 aa, Artificial Sequence
  Synthetic polypeptide
QQSYSTPLTF

SEQ ID NO: 82 - protein, 119 aa, Artificial Sequence
  Synthetic polypeptide
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSS

SEQ ID NO: 83 - protein, 109 aa, Artificial Sequence
  Synthetic polypeptide
RHQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRT

SEQ ID NO: 84 - protein, 243 aa, Artificial Sequence
  Synthetic polypeptide
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSGGGGSGGGGSGGGGSRHQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRT

SEQ ID NO: 85 - DNA, 729 bp, Artificial Sequence
  Synthetic polynucleotide
caggttcagcttcaggagagtggcccaggcctggtgaagccaagtgagactctcagcttgacttgcacagtttctggaggcagtgtctcctcaggcagctattattggtcctggattcggcagccccctgggaaaggcctggagtggattgggtacatatattacagtggcagcacaaattacaatccatccctgaagtctcgagtaactatcagtgtggacacaagcaagaatcagttttcactcaaactgtcttctgtgactgctgctgacactgctgtttattattgtgccagggaggggaaaaatggggcatttgatatttggggtcagggcacaatggtgacagtcagctctggaggtggaggctcaggaggaggaggcagtggaggtggtgggtcacgccatcagatgactcagtccccctccagtctttctgcctcagttggggatagagtgaccatcacatgcagagcaagtcagagcatatcatcctatctgaactggtaccagcagaagccagggaaagcccccaaattgctgatttatgcagcctcaagtctccagagtggggtgccaagcaggttctcaggcagtggcagtgggacagatttcacattgacaatcagctccctccaacctgaagattttgccacctactattgccagcaatcctacagcacgcccctgacttttggaggtggcacaaaggtagagatcaagaggact

SEQ ID NO: 86 - protein, 235 aa, Homo sapiens; identical to SEQ ID NO: 5

SEQ ID NO: 87 - protein, 247 aa, Mus musculus
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 88 - protein, 69 aa, Artificial Sequence
  Synthetic polypeptide
STTTKPVLRTPSPVHPTGTSQPQRPEDCRPRGSVKGTGLDFACDIYIWAPLAGICVALLLSLIITLICY

SEQ ID NO: 89 - DNA, 207 bp, Artificial Sequence
  Synthetic polynucleotide
tctactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctac

SEQ ID NO: 90 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 91 - DNA, 81 bp, Artificial Sequence
  Synthetic polynucleotide
ttttgggtgctggtggtggttggtggagtcctggcttgctatagcttgctagtaacagtggcctttattattttctgggtg

SEQ ID NO: 92 - protein, 27 aa, Artificial Sequence
  Synthetic polypeptide
FWVLVVVGGVLACYSLLVTVAFIIFWV

SEQ ID NO: 93 - DNA, 81 bp, Artificial Sequence; identical to SEQ ID NO: 91
  Synthetic polynucleotide

SEQ ID NO: 94 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 95 - protein, 112 aa, Artificial Sequence
  Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 96 - DNA, 336 bp, Artificial Sequence
  Synthetic polynucleotide
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 97 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 98 - DNA, 123 bp, Artificial Sequence
  Synthetic polynucleotide
aatagtagaaggaacagactccttcaaagtgactacatgaacatgactccccggaggcctgggctcactcgaaagccttaccagccctacgcccctgccagagactttgcagcgtaccgcccc

SEQ ID NO: 99 - protein, 41 aa, Artificial Sequence
  Synthetic polypeptide
NSRRNRLLQSDYMNMTPRRPGLTRKPYQPYAPARDFAAYRP

SEQ ID NO: 100 - DNA, 123 bp, Artificial Sequence; identical to SEQ ID NO: 98
  Synthetic polynucleotide

SEQ ID NO: 101 - protein, 41 aa, Artificial Sequence
  Synthetic polypeptide
RSKRSRLLHSDYMNMTPRRPGPTRKHYQPYAPPRDFAAYRS

SEQ ID NO: 102 - DNA, 123 bp, Artificial Sequence
  Synthetic polynucleotide
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 103 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 104 - protein, 42 aa, Artificial Sequence
  Synthetic polypeptide
KRGRKKLLYIFKQPFMRPVQTTQEEDGCSCRFPEEEEGGCEL

SEQ ID NO: 105 - DNA, 126 bp, Artificial Sequence
  Synthetic polynucleotide
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 106 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 107 - protein, 23 aa, Artificial Sequence
  Synthetic polypeptide
GSGATNFSLLKQAGDVEENPGPM

SEQ ID NO: 108 - protein, 41 aa, Artificial Sequence
  Synthetic polypeptide
RSKRSRLLHSDYMNMTPRRPGPTRKHYQPYAPPRDFAAYRK

SEQ ID NO: 109 - protein, 39 aa, Artificial Sequence
  Synthetic polypeptide
NQLERTVNSLNVPAISIPEHDEADEISDENREKVNDQAK

SEQ ID NO: 110 - protein, 42 aa, Artificial Sequence
  Synthetic polypeptide
AAANQLERTVNSLNVSAISIPEHDEADEISDENREKVNDQAK

SEQ ID NO: 111 - protein, 48 aa, Artificial Sequence
  Synthetic polypeptide
PEHDEADEISDENREKVNDQAKLIVGIVVGLLLAALVAGVVYWLYMKK

SEQ ID NO: 112 - protein, 37 aa, Artificial Sequence
  Synthetic polypeptide
ENREKVNDQAKLIVGIVVGLLLAALVAGVVYWLYMKK

SEQ ID NO: 113 - protein, 65 aa, Artificial Sequence
  Synthetic polypeptide
NQLERTVNSLNVPAISIPEHDEADEISDENREKVNDQAKLIVGIVVGLLLAALVAGVVYWLYMKK

SEQ ID NO: 114 - protein, 66 aa, Artificial Sequence
  Synthetic polypeptide
TCTAENQLERTVNSLNVSAISIPEHDEADEISDENREKVNDQAKLIVGIVVGLLLAALVAGVVYWL

SEQ ID NO: 115 - protein, 44 aa, Artificial Sequence
  Synthetic polypeptide
PEHDEADEISDENREKVNDQAKLIVGIVVGLLLAALVAGVVYWL

SEQ ID NO: 116 - protein, 61 aa, Artificial Sequence
  Synthetic polypeptide
NQLERTVNSLNVSAISIPEHDEADEISDENREKVNDQAKLIVGIVVGLLLAALVAGVVYWL

SEQ ID NO: 117 - protein, 68 aa, Artificial Sequence
  Synthetic polypeptide
AAANQLERTVNSLNVSAISIPEHDEADEISDENREKVNDQAKLIVGIVVGLLLAALVAGVVYWLYMKK

SEQ ID NO: 118 - protein, 241 aa, Artificial Sequence
  Synthetic polypeptide
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQAAAPTTTPAPRPPTPAPTIASQPLSLRPEACRPAAGGAVHTRGLDFACDIYIWAPLAGTCGVLLLSLVITLYCNHR

SEQ ID NO: 119 - DNA, 723 bp, Artificial Sequence
  Synthetic polynucleotide
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgctttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccaggcggccgcacccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaaccacagg

SEQ ID NO: 120 - DNA, 63 bp, Artificial Sequence
  Synthetic polynucleotide
atggccctgccagtaacggctctgctgctgccacttgctctgctcctccatgcagccaggcct

SEQ ID NO: 121 - protein, 19 aa, Artificial Sequence
  Synthetic polynucleotide
ATNFSLLKQAGDVEENPGP

SEQ ID NO: 122 - DNA, 57 bp, Artificial Sequence
  Synthetic polynucleotide
gcaacaaacttctcactactcaaacaagcaggtgacgtggaggagaatcccggccca

SEQ ID NO: 123 - DNA, 8613 bp, Artificial Sequence
  Synthetic polynucleotide
atggccctgccagtaacggctctgctgctgccacttgctctgctcctccatgcagccaggcctcaggttcagcttcaggagagtggcccaggcctggtgaagccaagtgagactctcagcttgacttgcacagtttctggaggcagtgtctcctcaggcagctattattggtcctggattcggcagccccctgggaaaggcctggagtggattgggtacatatattacagtggcagcacaaattacaatccatccctgaagtctcgagtaactatcagtgtggacacaagcaagaatcagttttcactcaaactgtcttctgtgactgctgctgacactgctgtttattattgtgccagggaggggaaaaatggggcatttgatatttggggtcagggcacaatggtgacagtcagctctggaggtggaggctcaggaggaggaggcagtggaggtggtgggtcacgccatcagatgactcagtccccctccagtctttctgcctcagttggggatagagtgaccatcacatgcagagcaagtcagagcatatcatcctatctgaactggtaccagcagaagccagggaaagcccccaaattgctgatttatgcagcctcaagtctccagagtggggtgccaagcaggttctcaggcagtggcagtgggacagatttcacattgacaatcagctccctccaacctgaagattttgccacctactattgccagcaatcctacagcacgcccctgacttttggaggtggcacaaaggtagagatcaagaggactgcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccaggggctcagtacagccaccaaggacaccttcgacgcccttcacatgcaggccctgccccctcgcggatctggagcaacaaacttctcactactcaaacaagcaggtgacgtggaggagaatcccggcccaatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgctttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccaggcggccgcacccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaaccacaggcggatccaataacagccactcgaggatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgaatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgcc

SEQ ID NO: 124 - DNA, 8604 bp, Artificial Sequence
  Synthetic polynucleotide
atggccctgccagtaacggctctgctgctgccacttgctctgctcctccatgcagccaggcctcaggttcagcttcaggagagtggcccaggcctggtgaagccaagtgagactctcagcttgacttgcacagtttctggaggcagtgtctcctcaggcagctattattggtcctggattcggcagccccctgggaaaggcctggagtggattgggtacatatattacagtggcagcacaaattacaatccatccctgaagtctcgagtaactatcagtgtggacacaagcaagaatcagttttcactcaaactgtcttctgtgactgctgctgacactgctgtttattattgtgccagggaggggaaaaatggggcatttgatatttggggtcagggcacaatggtgacagtcagctctggaggtggaggctcaggaggaggaggcagtggaggtggtgggtcacgccatcagatgactcagtccccctccagtctttctgcctcagttggggatagagtgaccatcacatgcagagcaagtcagagcatatcatcctatctgaactggtaccagcagaagccagggaaagcccccaaattgctgatttatgcagcctcaagtctccagagtggggtgccaagcaggttctcaggcagtggcagtgggacagatttcacattgacaatcagctccctccaacctgaagattttgccacctactattgccagcaatcctacagcacgcccctgacttttggaggtggcacaaaggtagagatcaagaggactgcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccaggggctcagtacagccaccaaggacaccttcgacgcccttcacatgcaggccctgccccctcgcggatctggagcaacaaacttctcactactcaaacaagcaggtgacgtggaggagaatcccggcccaatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgctttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccaggcggccgcacccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaaccacaggcggatccaataacagccactcgaggatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagaagccagtttgcatctgtcaggatcaatttcccattatgccagtcatattaattactagtcaattagttgatttttatttttgacatatacatgtgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgcc

SEQ ID NO: 125 - DNA, 63 bp, Artificial Sequence; identical to SEQ ID NO: 120
  Synthetic polynucleotide

SEQ ID NO: 126 - DNA, 24 bp, Artificial Sequence
  Synthetic polynucleotide
aggaccttacacagtcctgctgac

SEQ ID NO: 127 - DNA, 21 bp, Artificial Sequence
  Synthetic polynucleotide
agaacctagaacctcgctgga

SEQ ID NO: 128 - DNA, 20 bp, Artificial Sequence
  Synthetic polynucleotide
ctgcgatgccgtctactttg

SEQ ID NO: 129 - DNA, 27 bp, Artificial Sequence
  Synthetic polynucleotide
tgctgaaacattcaccttccatgcaga

SEQ ID NO: 130 - DNA, 25 bp, Artificial Sequence
  Synthetic polynucleotide
tgaaacatacgttcccaaagagttt

SEQ ID NO: 131 - DNA, 26 bp, Artificial Sequence
  Synthetic polynucleotide
ctctccttctcagaaagtgtgcatat

SEQ ID NO: 132 - DNA, 18 bp, Artificial Sequence
  Synthetic polynucleotide
gaaggtgaaggtcggagt

SEQ ID NO: 133 - DNA, 22 bp, Artificial Sequence
  Synthetic polynucleotide
catgggtggaatcatattggaa

SEQ ID NO: 134 - DNA, 22 bp, Artificial Sequence
  Synthetic polynucleotide
ccaggatggttcttagactccc

SEQ ID NO: 135 - DNA, 21 bp, Artificial Sequence
  Synthetic polynucleotide
tttagcacgaagctctccgat

SEQ ID NO: 136 - DNA, 20 bp, Artificial Sequence
  Synthetic polynucleotide
acgagggacaataggagcca

SEQ ID NO: 137 - DNA, 20 bp, Artificial Sequence
  Synthetic polynucleotide
ggcatactccgtctgctcag

(19) United States
(12) Patent Application Publication
(10) Pub. No.: US 2022/0125905 A1
(43) Pub. Date: Apr. 28, 2022

(54) MESOTHELIN CARS AND USES THEREOF

(71) Applicant: MEMORIAL SLOAN-KETTERING CANCER CENTER, New York, NY (US)

(72) Inventors: Prasad S. Adusumilli, New York, NY (US); Michel Sadelain, New York, NY (US)

(73) Assignee: MEMORIAL SLOAN-KETTERING CANCER CENTER, New York, NY (US)

(21) Appl. No.: 17/526,812

(22) Filed: Nov. 15, 2021

Related U.S. Application Data
(63) Continuation of application No. PCT/US2020/033382, filed on May 18, 2020.
(60) Provisional application No. 62/975,966, filed on Feb. 13, 2020.
(60) Provisional application No. 62/848,983, filed on May 16, 2019.

(51) Int. Cl. A61K 39/00; A61P 35/00; C07K 14/47; C07K 14/725; C07K 14/705; C07K 16/30

(52) U.S. Cl. CPC A61K 39/001168; A61P 35/00; C07K 14/4747; C07K 14/7051; A61K 2039/505; C07K 14/70521; C07K 14/70596; C07K 16/30; A61K 2039/5156; C07K 14/70517

(57) ABSTRACT

The presently disclosed subject matter provides polypeptide compositions comprising a chimeric antigen receptor (CAR) that targets mesothelin; and a dominant negative form of programmed death 1 (PD-1 DN). Also provided are immunoresponsive cells comprising such polypeptide compositions and uses of the polypeptide compositions and immunoresponsive cells for treatment, e.g., for treating solid tumors.

### CROSS-REFERENCE TO RELATED APPLICATION

This application is a Continuation of International Patent Application No. PCT/US2020/033382, filed May 18, 2020, which claims priority to U.S. Provisional Application No. 62/848,983 filed on May 16, 2019, and U.S. Provisional Application No. 62/975,966 filed on Feb. 13, 2020, the contents of each of which are incorporated by reference in their entireties, and to each of which priority is claimed.

### SEQUENCE LISTING

The specification further incorporates by reference the Sequence Listing submitted herewith via EFS on Nov. 15, 2021. Pursuant to 37 C.F.R. § 1.52(e) (5), the Sequence Listing text file, identified as 0866280118 SL.txt, is 144,365 bytes and was created on Nov. 15, 2021. The Sequence Listing electronically filed herewith, does not extend beyond the scope of the specification and thus does not contain new matter.

### 1. INTRODUCTION

The presently disclosed subject matter provides methods and compositions for enhancing the immune response toward cancers and pathogens. It relates to chimeric antigen receptors (CARs) that specifically target human mesothelin, and immunoresponsive cells comprising such CARs. The presently disclosed mesothelin-targeted CARs have enhanced immune-activating properties, including anti-tumor activity, while possessing features to minimize CAR-induced toxicity and immunogenicity.

### 2. BACKGROUND OF THE INVENTION

Cell-based immunotherapy is a therapy with curative potential for the treatment of cancer. T cells and other immune cells may be modified to target tumor antigens through the introduction of genetic material coding for artificial or synthetic receptors for antigen, termed Chimeric Antigen Receptors (CARs), specific to selected antigens. Targeted T cell therapy using CARs has shown recent clinical success in treating some hematologic malignancies. However, translating CAR-expressing T cell therapy to solid tumors poses several obstacles that must be overcome to achieve clinical benefit. Malignant cells adapt to generate an immunosuppressive microenvironment to protect themselves from immune recognition and elimination. This tumor microenvironment poses a challenge to methods of treatment involving stimulation of an immune response, such as targeted T cell therapies. Solid tumors may also be restricted within anatomical compartments that impede efficient T cell trafficking, lack expression of agonistic costimulatory ligands and/or express negative regulators of T cell function. The successful elimination of solid tumors thus requires effective tumor infiltration and overcoming tumor-induced immunosuppression. In addition, solid tumors pose a challenge for selecting optimal immune targets—antigens whose targeting would enable tumor eradication by potent T cells, with minimal or tolerable toxicity to non-tumor tissues.

Accordingly, there are needs for novel therapeutic strategies to design CARs for treating cancers, particularly, solid tumors, which strategies capable of inducing potent tumor eradication with minimal toxicity and immunogenicity.

### 3. SUMMARY OF THE INVENTION

The presently disclosed subject matter provides polypeptide compositions comprising (a) a chimeric antigen receptor (CAR) that specifically targets mesothelin (e.g., human mesothelin); and (b) a dominant negative form of programmed death 1 (PD-1 DN); immunoresponsive cells comprising such polypeptide compositions, and uses of these polypeptide compositions and immunoresponsive cells, e.g., for treating cancers.

The presently disclosed subject matter provides polypeptide compositions. In certain embodiments, the polypeptide composition comprises: i) a chimeric antigen receptor (CAR) and ii) a dominant negative form of programmed death 1 (PD-1 DN), wherein the CAR comprises (a) an extracellular antigen-binding domain and (b) an intracellular signaling domain comprising a modified CD3ζ polypeptide comprising an ITAM2 variant and an ITAM3 variant, wherein each of the ITAM2 variant and an ITAM3 variant comprises two loss-of-function mutations.

In certain embodiments, the extracellular antigen-binding domain comprises: a heavy chain variable region comprising a CDR1 comprising the amino acid sequence set forth in SEQ ID NO:76, a CDR2 comprising the amino acid sequence set forth in SEQ ID NO:77, and a CDR3 comprising the amino acid sequence set forth in SEQ ID NO:78; and a light chain variable region comprising a CDR1 comprising the amino acid sequence set forth in SEQ ID NO:79, a CDR2 comprising the amino acid sequence set forth in SEQ ID NO:80, and a CDR3 comprising the amino acid sequence set forth in SEQ ID NO:81.

In certain embodiments, the PD-1 DN comprises: (a) at least a portion of an extracellular domain of programmed death 1 (PD-1) comprising a ligand binding region, and (b) a first transmembrane domain.

In certain embodiments, the first transmembrane domain of the PD-1 DN comprises a CD8 polypeptide, a CD28 polypeptide, a CD3ζ polypeptide, a CD4 polypeptide, a 4-1BB polypeptide, an OX40 polypeptide, a CD166 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40/My88 peptide, a NKGD2 peptide, or a combination thereof. In certain embodiments, the first transmembrane domain of the PD-1 DN comprises a CD8 polypeptide. In certain embodiments, the CD8 polypeptide comprised in the first transmembrane domain of the PD-1 DN comprises amino acids 137 to 207 of SEQ ID NO: 86. In certain embodiments, the PD-1 DN lacks an intracellular domain. In certain embodiments, the PD-1 DN comprises amino acids 21 to 165 of SEQ ID NO: 48 and amino acids 137 to 207 of SEQ ID NO: 86.

In certain embodiments, the extracellular antigen-binding domain of the CAR specifically binds to human mesothelin with an EC50 value of from about 1 nM to about 25 nM. In certain embodiments, the extracellular antigen-binding domain of the CAR specifically binds to human mesothelin with an EC50 value of about 20 nM.

In certain embodiments, the extracellular antigen-binding domain of the CAR comprises a single-chain variable fragment (scFv), a Fab that is optionally crosslinked, or a F(ab)2. In certain embodiments, the extracellular antigen-binding domain of the CAR comprises a human scFv. In certain embodiments, the extracellular antigen-binding domain of the CAR recognizes human mesothelin with a mesothelin expression level of about 1,000 or more mesothelin binding sites/cell.

In certain embodiments, the heavy chain variable region comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous to identical to the amino acid sequence set forth in SEQ ID NO:82. In certain embodiments, the heavy chain variable region comprises the amino acid sequence set forth in SEQ ID NO:82.

In certain embodiments, the light chain variable region comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous to identical to the amino acid sequence set forth in SEQ ID NO: 83. In certain embodiments, the light chain variable region comprises the amino acid sequence set forth in SEQ ID NO: 83.

In certain embodiments, the heavy chain variable region comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous to identical to the amino acid sequence set forth in SEQ ID NO:82, and the light chain variable region comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous to identical to the amino acid sequence set forth in SEQ ID NO: 83. In certain embodiments, the heavy chain variable region comprises the amino acid sequence set forth in SEQ ID NO:82, and the light chain variable region comprises the amino acid sequence set forth in SEQ ID NO: 83.

In certain embodiments, the extracellular antigen-binding domain of the CAR comprises a linker between the heavy chain variable region and the light chain variable region.

In certain embodiments, a leader that is covalently joined to the N-terminus of the extracellular antigen-binding domain. In certain embodiments, the leader comprises a CD8 polypeptide. In certain embodiments, the CD8 polypeptide consists of the amino acid sequence set forth in SEQ ID NO: 71. In certain embodiments, the at least a portion of the extracellular domain of PD-1 comprises amino acids 21 to 165 of SEQ ID NO: 48.

In certain embodiments, each of the loss-of-function mutations in the modified CD3ζ polypeptide of the CAR is at a tyrosine amino acid residue. In certain embodiments, the ITAM2 variant comprises or consists of the amino acid sequence set forth in SEQ ID NO: 29. In certain embodiments, the ITAM3 variant comprises or consists of the amino acid sequence set forth in SEQ ID NO: 33. In certain embodiments, the modified CD3ζ polypeptide comprises a native ITAM1. In certain embodiments, the native ITAM1 comprises or consists of the amino acid sequence set forth in SEQ ID NO: 23. In certain embodiments, the modified CD3ζ polypeptide comprises or consists of the amino acid sequence set forth in SEQ ID NO: 35.

In certain embodiments, the CAR comprises or consists of the amino acid sequence set forth in SEQ ID NO: 56.

In certain embodiments, the CAR further comprises a second transmembrane domain. In certain embodiments, the second transmembrane domain of the CAR comprises a CD8 polypeptide, a CD28 polypeptide, a CD3ζ polypeptide, a CD4 polypeptide, a 4-1BB polypeptide, an OX40 polypeptide, a CD166 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40/My88 peptide, a NKGD2 peptide, or a combination thereof. In certain embodiments, the second transmembrane domain of the CAR comprises a CD28 polypeptide

In certain embodiments, the intracellular signaling domain of the CAR further comprises a co-stimulatory signaling domain. In certain embodiments, the co-stimulatory signaling region comprises a CD28 polypeptide, a 4-1BB polypeptide, an OX40 polypeptide, an ICOS polypeptide, a DAP-10 polypeptide, a CD27 polypeptide, a CD40/My88 polypeptide, a NKGD2 polypeptide, or a combinations thereof. In certain embodiments, the co-stimulatory signaling region comprises a CD28 polypeptide.

The presently disclosed subject matter provides immunoresponsive cells comprising a polypeptide composition disclosed herein. In certain embodiments, the PD-1 DN and/or the CAR is recombinantly expressed. In certain embodiments, the PD-1 DN and/or the CAR is expressed from a vector. In certain embodiments, the immunoresponsive cell is selected from the group consisting of a T cell, a Natural Killer (NK) cell, a pluripotent stem cell from which lymphoid cells may be differentiated. In certain embodiments, the pluripotent stem cell is an embryonic stem cell or an induced pluripotent stem cells. In certain embodiments, the immunoresponsive cell is a T cell. In certain embodiments, the T cell is selected from the group consisting of a cytotoxic T lymphocyte (CTL), a regulatory T cell, and a Natural Killer T (NKT) cell. In certain embodiments, the immunoresponsive cell is autologous. In certain embodiments, the immunoresponsive cell is allogenic.

The presently disclosed subject matter further provides compositions comprising an immunoresponsive cell disclosed herein. In certain embodiments, the composition is a pharmaceutical composition that further comprises a pharmaceutically acceptable excipient. In certain embodiments, the pharmaceutical composition comprises between about 10⁴ and 10⁶ of the immunoresponsive cells. In certain embodiments, the pharmaceutical composition comprises at least about 10⁵ of the immunoresponsive cells. In certain embodiments, the pharmaceutical composition comprises about 10⁵ of the immunoresponsive cells. In certain embodiments, the pharmaceutical composition is for preventing and/or treating a neoplasm in a subject, treating a subject having a relapse of a neoplasm, reducing tumor burden in a subject, increasing or lengthening survival of a subject having a neoplasm, preventing and/or treating an inflammatory disease in a subject, and/or preventing graft rejection in a subject who is a recipient of an organ transplant.

In addition, the presently disclosed subject matter provides nucleic acid compositions comprising a polynucleotide encoding a polypeptide composition disclosed herein. In certain embodiments, the polynucleotide comprises the nucleotide sequence set forth in SEQ ID NO: 123. In certain embodiments, the polynucleotide comprises the nucleotide sequence set forth in SEQ ID NO: 124. The presently disclosed subject matter further provides vectors comprising the presently disclosed nucleic acid compositions. In certain embodiments, the vector is a retroviral vector. In certain embodiments, the retroviral vector is a y-retroviral vector or a lentiviral vector.

The presently disclosed subject matter provides methods for producing an immunoresponsive cell disclosed herein. In certain embodiments, the method comprises introducing into an immunoresponsive cell a presently disclosed polypeptide composition, a presently disclosed nucleic acid composition, or a presently disclosed vector.

The presently disclosed subject matter provides kits comprising a presently disclosed polypeptide composition, a presently disclosed nucleic acid composition, a presently disclosed vector, an presently disclosed immunoresponsive cell, or a presently disclosed pharmaceutical composition. In certain embodiments, the kit further comprises written instructions for treating and/or preventing a neoplasm.

Furthermore, the presently disclosed subject matter provides various methods of using the above-described immunoresponsive cell. For example, the presently disclosed subject matter provides methods of reducing tumor burden in a subject, wherein the method comprises administering to the subject an effective amount of the immunoresponsive cells or the pharmaceutical composition disclosed herein. In certain embodiments, the method reduces the number of tumor cells, reduces tumor size, and/or eradicates the tumor in the subject.

The presently disclosed subject matter also provides methods of increasing or lengthening survival of a subject having a neoplasm, where the method comprises administering to the subject an effective amount of the presently disclosed immunoresponsive cell or a presently disclosed pharmaceutical composition.

In certain embodiments, the tumor or neoplasm is a solid tumor. In certain embodiments, the solid tumor is selected from the group consisting of mesothelioma, lung cancer, pancreatic cancer, ovarian cancer, breast cancer, colon cancer, pleural tumor, glioblastoma, esophageal cancer, gastric cancer, synovial sarcoma, thymic carcinoma, endometrial carcinoma, stomach cancer, cholangiocarcinoma, cervical cancer, salivary gland cancer, and a combination thereof.

The presently disclosed subject matter provides methods of treating a subject having a relapse of a neoplasm, the method comprising administering to the subject an effective amount of the immunoresponsive cells or the pharmaceutical composition disclosed herein. In certain embodiments, the subject received an immunotherapy prior to said administration of the immunoresponsive cells or the composition.

Additionally, the presently disclosed subject matter provides methods of increasing immune-activating cytokine production in response to a cancer cell or a pathogen in a subject. In certain embodiments, the method comprises administering to the subject an effective amount of the immunoresponsive cells or the pharmaceutical composition disclosed herein. In certain embodiments, the immune-activating cytokine is selected from the group consisting of granulocyte macrophage colony stimulating factor (GM-CSF), IFN-α, IFN-γ, TNF-α, IL-2, IL-3, IL-6, IL-11, IL-7, IL-12, IL-15, IL-21, interferon regulatory factor 7 (IRF7), and combinations thereof.

In accordance with the presently disclosed subject matter, the above-described various methods can comprise administering at least one immunomodulatory agent. In certain embodiments, the at least one immunomodulatory agent is selected from the group consisting of immunostimulatory agents, checkpoint immune blockade agents, radiation therapy agents, chemotherapy agents, and combinations thereof. In some embodiments, the immunostimulatory agents are selected from the group consisting of IL-12, an agonist costimulatory monoclonal antibody, and combinations thereof. In certain embodiments, the immunostimulatory agent is IL-12. In some embodiments, the agonist costimulatory monoclonal antibody is selected from the group consisting of an anti-4-1BB antibody, an anti-OX40 antibody, an anti-ICOS antibody, and combinations thereof. In certain embodiments, the agonist costimulatory monoclonal antibody is an anti-4-1BB antibody. In certain embodiments, the checkpoint immune blockade agents are selected from the group consisting of anti-PD-L1 antibodies, anti-CTLA-4 antibodies, anti-PD-1 antibodies, anti-LAG3 antibodies, anti-B7-H3 antibodies, anti-TIM3 antibodies, and combinations thereof. In certain embodiments, the checkpoint immune blockade agent is an anti-PD-L1 antibody or an anti-PD-1 antibody. In certain embodiments, the subject is a human.

In certain embodiments, the immunoresponsive cell is pleurally or intrapleurally administered to the subject.

The presently disclosed subject matter further provides a method of preventing and/or treating an inflammatory disease in a subject. In certain embodiments, the method comprises administering the presently disclosed immunoresponsive cell or the pharmaceutical composition to the subject. In certain embodiments, the immunoresponsive cell is an immunoinhibitory cell. In certain embodiments, the immunoinhibitory cell is a regulatory T cell. In certain embodiments, the inflammatory disease is pancreatitis. In certain embodiments, the subject is a human. In certain embodiments, the subject is a recipient of an organ transplant. In certain embodiments, the subject is a recipient of a pancreas transplant.

The presently disclosed subject matter further provides a method of preventing graft rejection in a subject who is a recipient of an organ transplant. In certain embodiments, the method comprises administering the presently disclosed immunoresponsive cell or the pharmaceutical composition to the subject. In certain embodiments, the immunoresponsive cell is an immunoinhibitory cell. In certain embodiments, the immunoinhibitory cell is a regulatory T cell. In certain embodiments, the subject is a human. In certain embodiments, the subject is a recipient of an pancreas transplant.

### 4. BRIEF DESCRIPTION OF THE FIGURES

The following Detailed Description, given by way of example, but not intended to limit the presently disclosed subject matter to specific embodiments described, may be understood in conjunction with the accompanying drawings.

FIG. 1 depicts a polypeptide composition in accordance with certain embodiments of the presently disclosed subject matter. The polypeptide composition comprises a CAR comprising an anti-mesothelin (MSLN) scFv, a CD28 transmembrane domain, a CD28 cytoplasmic signaling domain, a CD3zeta signaling domain (e.g., comprising an ITAM2 variant and an ITAM3 variant). The CAR is fused to the PD1DNR (and PD1 signaling domain) via a cleavable P2A peptide. SP: signaling peptide; scFv: single-chain variable fragment; TM: transmembrane domain; cyt: cytosolic domain; DNR: dominant negative receptor; LTR: long terminal repeat.

FIG. 2 depicts various constructs disclosed in Example 2.

FIGS. 3A-3D depict virus production in producer cell line RD114. RD114 cells were transduced with different dilutions of H29 viral supernatant (undiluted, 1:2, and 1:4) and stained for CAR expression by flow cytometry using an anti-Fab antibody. RD114 empty served as a negative control. FIG. 3A shows RD114 empty (as a negative control). FIG. 3B shows undiluted; FIG. 3C shows sup 1:2 diluted; and FIG. 3D shows sup 1:4 diluted.

FIGS. 4A-4E depict transduction of human T cells with M28z1XX-P2A-PD1DNR—donor H116-2. PHA-activated T cells were transduced with different concentrations of RD114 viral supernatant (FIG. 4A shows 1:2, FIG. 4B shows 1:5, FIG. 4C shows 1:7, FIG. 4D shows 1:15, and FIG. 4E shows un-transduced (“UT”)), and stained for CAR expression by anti-Fab staining and PD1DNR by anti-PD1 staining using flow cytometry.

FIG. 5A-5E depict transduction of human T cells with M28z1XX-P2A-PD1DNR—donor H18. PHA-activated T cells were transduced with different concentrations of RD114 viral supernatant (FIG. 5A shows 1:2, FIG. 5B shows 1:5, FIG. 5C shows 1:10, FIG. 5D shows 1:15, and FIG. 5E shows un-transduced (“UT”)) and stained for CAR expression by anti-Fab staining and PD1DNR by anti-PD1 staining using flow cytometry.

FIGS. 6A-6F depict transduction of human T cells with M28z1XX-P2A-PD1DNR—donor H19. PHA-activated T cells were transduced with different concentrations of RD114 viral supernatant (FIG. 6A shows 1:2, FIG. 6B shows 1:5, FIG. 6C shows 1:7; FIG. 6D shows 1:10, FIG. 6E shows 1:15, and FIG. 6F shows un-transduced (“UT”)) and stained for CAR expression by anti-Fab staining and PD1DNR by anti-PD1 staining using flow cytometry.

FIGS. 7A-7C depict the correlation of vector copy number (VCN) with median fluorescence intensity (MFI). PHA-activated T cells were transduced with different concentrations of RD114 viral supernatant and stained for CAR expression by anti-Fab staining and flow cytometry analysis. Genomic DNA of transduced T cells was isolated and vector copy number was determined as VCN/μg DNA using qPCR. The MFI of CAR-positive cells was correlated with the VCN/μg DNA for three different donors. FIG. 7A shows donor H19; FIG. 7B shows donor H18, and FIG. 7C shows donor H116-2.

FIG. 8 depicts that cytotoxicity for transduced T cells from 3 different donors. MSLN high target cells (MGM) were co-cultured with M28z1xx-PD1DNR CAR T cells from different donors at different E:T ratios using an impedance-based assay. The M28z1xx-PD1DNR CAR T-cell mediated cytolysis of MGM cells at E:T ratio 1:1. M28z1xx-PD1DNR CART cells killed high MSLN target cells.

FIG. 9 depicts an example of impedance-based cytotoxicity measurement (eCTL).

FIG. 10 depicts parameters of comparative analysis of various constructs using eCTL.

FIGS. 11A-11E depict MSLN and PD-L1 expressions of target cell lines. Mesothelioma (MGM (shown in FIG. 11A), MGM-PDL1 (shown in FIG. 11B) and MSTOG (shown in FIG. 11C)) and lung cancer (A549GM (shown in FIG. 11D) and A549G (shown in FIG. 11e)) cell lines were assessed for MSLN and PD-L1 expressions by flow cytometry. MGM, MGM-PDL1 and A549GM overexpressed MSLN; MGM-PDL1 cells additionally overexpressed PD-L1.

FIGS. 12A-12E depict CAR and PD1 expression of transduced T cells. Human T cells transduced with M28z (as shown in FIG. 12A), M28z1xx (as shown in FIG. 12B), M28z-PD1DNR (as shown in FIG. 12C) and M28z1xx-PD1DNR (as shown in FIG. 12D) were analyzed for CAR expression by anti-myc staining and PD1/PD1DNR expression by anti-PD1 staining using flow cytometry. FIG. 12E shows the un-transduced (“UT”) T cells.

FIGS. 13A-13C depict comparative analysis of anti-tumor efficacy of CART cells bearing the 1XX domain and PD1DNR for MSLN high tumor cells (MGM). MSLN high target cells (MGM) were co-cultured with either M28z, M28z1xx, M28z-PD1DNR, M28z1XX-PD1DNR or untransduced T cells at the indicated E:T ratios. Anti-tumor efficacy was assessed using an impedance-based assay. FIG. 13A shows the E:T ratio of about 3:1. FIG. 13B shows the E:T ratio of about 1:1. FIG. 13C shows the E:T ratio of about 0.33:1.

FIG. 14 depicts comparative analysis of cytotoxicity of CAR T cells bearing the 1xx domain and PD1DNR for MSLN high tumor cells (MGM). MSLN high target cells (MGM) labeled with chromium-51 were co-cultured with either M28z, M28z1xx, M28z-PD1DNR, M28z1xx-PD1DNR or untransduced T cells at the indicated E:T ratio for 18 hours. Cytotoxicity was determined by chromium-51 CTL.

FIGS. 15A-15C depict comparative analysis of anti-tumor efficacy of CART cells bearing the 1xx domain and PD1DNR for MSLN negative tumor cells (MSTOG). MSLN negative target cells (MSTOG) were co-cultured with either M28z, M28z1xx, M28z-PD1DNR, M28z1xx-PD1DNR or untransduced T cells at the indicated E:T ratios. Anti-tumor efficacy was assessed using an impedance-based assay. FIG. 15A shows the E:T ratio of about 3:1. FIG. 15B shows the E:T ratio of about 1:1. FIG. 15C shows the E:T ratio of about 0.33:1.

FIG. 16 depicts comparative analysis of cytotoxicity of CAR T cells bearing the 1XX domain and PD1DNR for MSLN negative tumor cells (MSTOG). MSLN negative target cells (MSTOG) labeled with chromium-51 were co-cultured with either M28z, M28z1XX, M28z-PD1DNR, M28z1XX-PD1DNR or untransduced T cells at the indicated E:T ratio for 18 hours. Cytotoxicity was determined by chromium-51 CTL.

FIGS. 17A-17C depict comparative analysis of anti-tumor efficacy of CAR T cells bearing the 1XX domain and PD1DNR for MSLN high tumor cells overexpressing PDL1. MSLN high target cells overexpressing PDL1 (MGM-PDL1) were co-cultured with either M28z, M28z1XX, M28z-PD1DNR, M28z1XX-PD1DNR or untransduced T cells at the indicated E:T ratios. Anti-tumor efficacy was assessed using an impedance-based assay. FIG. 17A shows the E:T ratio of about 3:1. FIG. 17B shows the E:T ratio of about 1:1. FIG. 17C shows the E:T ratio of about 0.33:1.

FIGS. 18A-18C depict comparative analysis of anti-tumor efficacy of CART cells bearing the 1xx domain and PD1DNR for MSLN high tumor cells (A549GM). MSLN high target cells (A549GM) were co-cultured with either M28z, M28z1xx, M28z-PD1DNR, M28z1xx-PD1DNR or untransduced T cells at the indicated E:T ratios. Anti-tumor efficacy was assessed using an impedance-based assay. FIG. 18A shows the E:T ratio of about 10:1. FIG. 18B shows the E:T ratio of about 5:1. FIG. 18C shows the E:T ratio of about 2:1.

FIGS. 19A-19C depict comparative analysis of anti-tumor efficacy of CAR T cells bearing the 1xx domain and PD1DNR: MSLN low tumor cells (A549G). MSLN low target cells (A549G) were co-cultured with either M28z, M28z1xx, M28z-PD1DNR, M28z1xx-PD1DNR or untransduced T cells at the indicated E:T ratios. Anti-tumor efficacy was assessed using an impedance-based assay. FIG. 19A shows the E:T ratio of about 10:1. FIG. 19B shows the E:T ratio of about 5:1. FIG. 19C shows the E:T ratio of about 2:1.

FIGS. 20A-20D depict the in vivo study results of various treatments. FIG. 20A shows the comparative in vivo efficacy of CAR T cells—M28z, M28z with PD1 antibody, and M28z with PD1DNR. FIG. 20B shows comparative in vivo efficacy of M28z and M28z1XX+PD1DNR CAR T cells. FIG. 20C shows tumor burden imaging showing systemic anti-tumor immunity following tumor rechallenges. FIG. 20D shows ex vivo immunofluorescence staining of orthotopic MPM tumors showing CAR T cells infiltration.

FIG. 21 depicts structure and components of M28z1XXPD1DNR CAR. In contrast to M28z, M28z1XXPD1DNR CAR T cells bear a mutated CD3ζ signaling domain with a single functional ITAM and co-express a PD1DNR that consists of CD8 transmembrane and hinge domains and lacks the intracellular PD1 signaling domain present in endogenous PD1.

FIG. 22 depicts structures of CAR T cell vectors.

FIG. 23 depicts expression of mesothelin (MSLN), PD-L1, and GFP on tumor cell lines. MGM, MGM-PDL1, and MSTOG tumor cells were analyzed for their expression of mesothelin (left panels), PD-L1 (middle panels), and GFP (right panels) by flow cytometry. Shown are density plots depicting the relative expression intensity plotted against the side scatter area (Y-axis).

FIGS. 24A-24D depict orthotopic MPM mouse model. FIG. 24A shows the gross appearance of human MPM (left upper panel) is reproduced in the mouse model of MPM (right upper panel), with tumor encasing heart, lungs, and mediastinal structures and the tumor invading the chest wall (bottom panel). FIG. 24B shows extensive vascularity of the tumor is demonstrated by the CD34 immunofluorescences. FIG. 24C shows tumor burden progression monitored by BLI correlates with tumor volume measurements by MRI at respective time points. FIG. 24D shows tumor burden progression monitored by serial BLI and MRI.

FIGS. 25A-25C depict expression of mesothelin in human tissues by immunohistochemical analysis. FIG. 25A shows expression of mesothelin in MPM versus normal pleura and pericardium. FIG. 25B shows expression of mesothelin in lung adenocarcinoma versus normal lung tissue. FIG. 25C shows expression of mesothelin in triple-negative breast cancer versus normal breast tissue.

FIG. 26 depicts that M28z1XXPD1DNR expression can be titrated using different dilutions of viral supernatant. Human T cells were transduced with different dilutions of viral supernatant encoding for M28z1XXPD1DNR (left panels) or mycM28z1XXPD1DNR (middle panels). Expression of CAR (Y-axis) and PD1 (X-axis) of viable CD3-positive cells was assessed by flow cytometry. Depicted results are from 1 donor representative of 3 different donors.

FIG. 27 depicts CAR expression measured by MFI correlates with VCN. Human T cells derived from 3 different donors were transduced with different dilutions of retroviral supernatant encoding for either M28z1XXPD1DNR or mycM28z1XXPD1DNR. The MFI of CAR-positive T cells (as determined by flow cytometry) was plotted against the VCN (as determined by qPCR). The R2 value was derived from a linear regression analysis (black line).

FIGS. 28A-28D depict expression of PD1 and PD1DNR in mycM28z1XXPD1DNR and mycM28z CAR T cells. FIG. 28A shows percent CAR surface expression of mycM28z and mycM28z1XXPD1DNR CAR T cells. FIG. 28B shows percent CD3-positive cells positive for PD1 surface expression. FIG. 28C shows MFI of PD1 surface expression of CD3-positive cells. FIG. 28D shows relative mRNA expression of PD1 extracellular and PD1 intracellular domain shown as fold change, compared with that of untransduced T cells.

FIG. 29 depicts M28z1XXPD1DNR-expressing T cells with or without myc-tag exhibit identical antitumor efficacy in vitro. Human T cells derived from 3 different donors were transduced with either M28z1XXPD1DNR (red) or mycM28z1XXPD1DNR (green) (transduction range, 37%-63%) and cocultured with MGM cells (green; arrow indicates time of T-cell addition). The antitumor efficacy of both constructs was compared at the indicted E:T ratios using an impedance-based cytotoxicity assay.

FIG. 30 shows that mycM28z1XXPD1DNR CAR T cells mediate antigen-specific, HLA-independent tumor lysis. Human T cells transduced with mycM28z1XXPD1DNR (blue) or mycM28z (red) were co-cultured with either MGM, MGM-PDL1, or MSTOG tumor cells at the indicated E:T ratios. The cytotoxicity of CART cells was assessed by 51Cr-release assay after 18 h of coculture. Untransduced T cells (orange) served as control.

FIG. 31 depicts accumulation of mycM28z1XXPD1DNR CART cells upon stimulation with mesothelin-expressing tumor cells. Human T cells transduced with mycM28z1XXPD1DNR (blue) or mycM28z (red) were repeatedly exposed to MGM or MGM-PDL1 target cells for 48 h at an E:T ratio of 1:1. The accumulation of CAR T cells was quantified by absolute CAR T-cell count after each antigen stimulation.

FIG. 32 shows that mycM28z1XXPD1DNR CAR T cells exhibited similar cytotoxicity to mycM28z CAR T cells upon initial antigen stimulation. Human T cells transduced with mycM28z1XXPD1DNR (blue) or mycM28z (red) were cocultured with 51Cr-labeled MGM or MGM-PDL1 target cells at the indicated E:T ratios. Cytotoxicity was assessed after 18 h using a 51Cr-release assay. Untransduced T cells (orange) served as control.

FIG. 33 shows that mycM28z1XXPD1DNR CAR T cells retained antitumor efficacy upon repeated antigen stimulation. Human T cells transduced with mycM28z1XXPD1DNR (blue) or mycM28z (red) were repeatedly exposed to MGM (left panels) or MGM-PDL1 (right panels) target cells for 48 h at an E:T ratio of 3:1 for 4 stimulations followed by an E:T ratio of 1:1 for 2 additional stimulations. The cytotoxicity of CART cells was assessed by 51Cr-release assay upon the fourth and seventh antigen stimulations at the indicated E:T ratios after 18 h of coculture.

FIG. 34 shows that mycM28z1XXPD1DNR CAR T cells secreted effector cytokines upon antigen stimulation. Human T cells transduced with mycM28z1XXPD1DNR (blue) or mycM28z (red) were repeatedly exposed to MGM (top row) or MGM-PDL1 (bottom row) target cells for 48 h at an E:T ratio of 1:1. Cell-free supernatant was collected 24 h after the first, third, and sixth antigen exposures, and effector cytokine secretion was assessed by Luminex assay.

FIG. 35 depicts intrapleural administration of a single low dose of 3×10⁴ to mycM28z1XXPD1DNR CAR T cells demonstrates antitumor efficacy in vivo. Female NSG mice bearing orthotopic MGM tumor were treated with a single intrapleural dose of either P28z CAR T cells (n=6, red bar) or mycM28z1XXPD1DNR CAR T cells (n=10, blue bar). Tumor burden was measured by BLI. The indicated time point represents day 15 after CAR T-cell administration, when P28z CAR T cell-treated mice started to become moribund. Statistical significance was determined using an unpaired Student's t test (2-tailed). ***p<0.001.

FIGS. 36A-36D depict that intrapleurally administered mycM28z1XXPD1DNR CAR T cells exhibited antitumor efficacy in vivo and increase survival. FIG. 3A shows serial tumor BLI of MGM-PDL1 tumor-bearing female NSG mice treated with a single dose of mycM28z (1×10⁵) or mycM28z1XXPD1DNR (1×10⁵ or 5×10⁴) CART cells (n=7-8). Shown are 4 mice per treatment group in the ventral position. FIG. 36B shows corresponding serial tumor BLI (average of dorsal and ventral) indicating tumor burden of each treated mouse. FIG. 36C shows corresponding mice weights following treatment. FIG. 36D shows Kaplan-Meier survival analysis comparing the in vivo efficacy of mycM28z and mycM28z1XXPD1DNR CAR T cells. The survival curve was analyzed using the log-rank test. *p<0.05, **p<0.01.

FIG. 37 depicts detection of mycM28z1XXPD1DNR CAR T cells in the primary tumor of intrapleurally treated mice. Pleural MGM tumor from mice treated with 5×10⁵ untransduced T cells (left), mycM28z CAR T cells (middle), or mycM28z1XXPD1DNR CAR T cells (right). Tumor tissue was collected 3 days after intrapleural T-cell injection, fixed, and stained ex vivo for tumor mesothelin (green), human CD45-positive cells (red), and DAPI (cell nucleus, blue) by immunofluorescence.

FIGS. 38A and 38B show that mycM28z1XXPD1DNR CAR T cells resisted tumor reestablishment upon repeated tumor challenge in vivo. FIG. 38A shows scheme illustrating the tumor rechallenge experiment: 68 days after intrapleural administration of mycM28z or mycM28z1XXPD1DNR CAR T cells (single dose of 1×10⁵ CAR T cells) and after pleural eradication of MGM-PDL1 tumor cells (inoculation dose of 8×10⁵), mice were rechallenged 10 times intraperitoneally with escalating doses (2×10⁶ to 11×10⁶) of MGM tumor cells every 4-8 days. FIG. 38B shows serial BLI indicating tumor burden following a single intrapleural dose of mycM28z (2 mice, red lines) or mycM28z1XXPD1DNR (3 mice, black lines) CAR T cells and tumor rechallenge starting at treatment day 68. Black arrows indicate time points of intraperitoneal tumor rechallenge with escalating doses.

FIGS. 39A-39C depict M28z1XXPD1DNR CAR T cells manufactured using the vector stocks for the clinical trial possess antitumor efficacy in vivo and prolong survival. FIG. 39A shows serial tumor BLI of MGM tumor-bearing female NSG mice treated with of 6×10⁴ (n=8) or 2×10⁵ (n=10) M28z1XXPD1DNR CAR T cells manufactured by CTCEF using the viral supernatant for the clinical trial. FIG. 39B shows corresponding mice weights following treatment. FIG. 39C shows Kaplan-Meier survival analysis.

FIG. 40 depicts average body weights at the interim sacrifice for male mice. Groups 1 (nontumor control), 3 (control vehicle), and 5 (test article) are shown.

FIG. 41 depicts average body weights at the interim sacrifice for female mice. Groups 2 (nontumor control), 4 (control), and 6 (test article) are shown.

FIG. 42 depicts average body weights at the final sacrifice for male mice. Groups 7 (nontumor control), 9 (control vehicle), and 11 (test article) are shown.

FIG. 43 depicts average body weights at the final sacrifice for female mice. Groups 8 (nontumor control), 10 (control vehicle), and 12 (test article) are shown.

FIG. 44 depicts identification of human T cells in the tumors of CAR T cell-treated and vehicle-treated mice. Intrapleural tumor tissue cells derived from CAR T cell-treated and vehicle-treated mice were stained with DAPI, anti-human CD45 APC/CY7, and anti-human CD3 PE/CY7 antibodies to detect viable human T cells by flow cytometry. Shown are density plots of human CD3 expression (X-axis) and human CD45 expression (Y-axis) of DAPI-negative (alive) single cells. The gate indicates cells stained positive for human CD45 and human CD3, representing human T cells.

FIG. 45 depicts identification of human T cells in the spleens of CAR T cell-treated and vehicle-treated mice. Spleen tissue cells derived from CAR T cell-treated and vehicle-treated mice were stained with DAPI, anti-human CD45 APC/CY7, and anti-human CD3 PE/CY7 antibodies to detect viable human T cells by flow cytometry. Shown are density plots of human CD3 expression (X-axis) and human CD45 expression (Y-axis) of DAPI-negative (alive) single cells. The gate indicates cells stained positive for human CD45 and human CD3, representing human T cells.

FIG. 46 depicts BLI of male mice.

FIG. 47 depicts BLI of female mice.

### 5. DETAILED DESCRIPTION OF THE INVENTION

The presently disclosed subject matter provides polypeptide compositions comprising a chimeric antigen receptor (CAR) targeting mesothelin and a dominant negative form of programmed death 1 (PD-1 DN), and immunoresponsive cells (e.g., T cells or NK cells) comprising the polypeptide composition. The presently disclosed subject matter also provides methods of using such polypeptide composition for inducing and/or enhancing an immune response of an immunoresponsive cell to a target antigen, and/or treating and/or preventing neoplasms or other diseases/disorders where an decrease in immune cell exhaustion is desired.

Persistent antigen exposure of T cells, such as in cancer, leads to an altered T-cell differentiation state, termed exhaustion, that renders CAR T cells dysfunctional (Youngblood et al., Int Immunol. 2010; 22(10):797-803; Wherry et al., Nat Rev Immunol. 2015; 15(8):486-499). Previous studies have shown that CAR activation potential is associated with three ITAMs (1-2-3) present in the CD3ζ cytoplasmic domain (Acuto et al., Nat Rev Immunol. 2003; 3(12):939-951; Love et al., Cold Spring Harb Perspect Biol. 2010; 2(6):a002485). Recent studies demonstrated that this CAR activation potential could be calibrated by mutating ITAMs, thereby reducing their functionality. Importantly, it was shown that, by introducing point mutations in the second- and third-position ITAMs (1-X-X; herein designated as “1XX”) of the CD3 domain, the fate of CART cells was changed from an exhaustive state to a balanced effector and memory state in the presence of high antigen exposure (Feucht et al., Nat Med. 2019; 25(1):82-88).

Another hurdle CAR T cells encounter in the solid tumor microenvironment is inhibition of their cytolytic activity mediated through PD1, an inhibitory receptor that is expressed upon antigen-mediated T-cell activation. In addition, tumor cells augment the expression of coinhibitory ligands such as PD-L1 following exposure to T-cell-secreted proapoptotic cytokines (McGray et al., Mol Ther. 2014; 22(1):206-218; Spranger et al., Sci Transl Med. 2013; 5(200):200ra116; Moon et al., Clin Cancer Res. 2014; 20(16):4262-4273). To overcome this hurdle, our group has combined mesothelin-targeted CAR T cells with a PD1 blocking antibody to rescue exhausted CAR T cells, restoring the antitumor efficacy of CAR T cells in our orthotopic mouse model (Cherkassky et al., J Clin Invest. 2016; 126(8):3130-3144; Grosser et al., Cancer Cell. 2019; 36(5):471-482). To avoid repeated dosage of PD1 checkpoint blockade agents and associated clinical adverse effects, our group has demonstrated that the use of a cell-intrinsic PD1 checkpoint blockade strategy—wherein a PD1DNR is co-transduced into the T cell along with a second-generation CAR—ultimately renders the transduced cells resistant to tumor PD-L1-mediated inhibition in the solid tumor microenvironment Cherkassky et al., J Clin Invest. 2016; 126(8):3130-3144; Grosser et al., Cancer Cell. 2019; 36(5):471-482).

Hence, to develop CAR T cells with an enhanced therapeutic profile, functional persistence, and resistance to tumor-mediated inhibition, the inventors incorporated the 1XX and PD1DNR components into the second-generation CAR vector design, which allows these cells to perform efficiently in the highly immunosuppressive microenvironment of solid tumors.

For purposes of clarity of disclosure and not by way of limitation, the detailed description is divided into the following subsections:

5.1. Definitions;

5.2. Polypeptide Compositions;
5.2.1. PD-1 DN;

5.2.2. Mesothelin-targeted CARs; and
5.2.3. Exemplified Polypeptide Compositions;

5.3. Immunoresponsive Cells;

5.4. Nucleic Acid Compositions and Vectors;

5.5. Polypeptides and Analogs;

5.6. Pharmaceutical Compositions and Administration;

5.7. Formulations;

5.8. Methods of Treatments; and

5.9. Kits

### 5.1. Definitions

Unless defined otherwise, all technical and scientific terms used herein have the meaning commonly understood by a person skilled in the art.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold or within 2-fold, of a value.

By “immunoresponsive cell” is meant a cell that functions in an immune response or a progenitor, or progeny thereof.

By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs), a signal transduction cascade is produced. In certain embodiments, when a chimeric antigen receptor (CAR) binds to an antigen, a formation of an immunological synapse occurs that includes clustering of many molecules near the bound receptor (e.g. CD4 or CD8, CD3γ/δ/ε/ζ, etc.). This clustering of membrane bound signaling molecules allows for ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after the activation of an immunoresponsive cell (e.g., a T cell) or concomitantly mediates through receptors including, but not limited to, CD28, CD137 (4-1BB), OX40, CD40 and ICOS. Receiving multiple co-stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fe fragment of intact antibody, clear more rapidly from the circulation, and may have less non-specific tissue binding of an intact antibody (Wahl et al., J. Nucl. Med. 24:316-325 (1983). As used herein, antibodies include whole native antibodies, bispecific antibodies; chimeric antibodies; Fab, Fab′, single chain V region fragments (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U.S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat system (Kabat, E. A., et al. (1991) Sequences of Proteins of Immunological Interest, Fifth Edition, U.S. Department of Health and Human Services, NIH Publication No. 91-3242). In certain embodiments, the CDRs are identified according to the Kabat system.

As used herein, the term “single-chain variable fragment” or “scFv” is a fusion protein of the variable regions of the heavy (V_{H}) and light chains (V_{L}) of an immunoglobulin covalently linked to form a V_{H}::V_{L} heterodimer. The V_{H} and V_{L} are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. “Linker”, as used herein, shall mean a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). In certain embodiments, the linker comprises or consists of the amino acid sequence set forth in SEQ ID NO: 66, which is provided below:

GGGGSGGGGSGGGGS [SEQ ID NO: 66].

An exemplary nucleotide sequence encoding the amino acid sequence of SEQ ID NO: 66 is set forth in SEQ ID NO: 50, which is provided below:

GGAGGTGGAGGCTCAGGAGGAGGAGGCAGTGGAGGTGGTGGGTCA [SEQ ID NO:50].

An exemplary nucleotide sequence encoding the amino acid sequence of SEQ ID NO:66 is set forth in SEQ ID NO: 51, which is provided below.

GGTGGAGGCGGTTCAGGCGGAGGTGGCTCTGGCGGTGGCGGATCA [SEQ ID NO:51]

Despite removal of the constant regions and the introduction of a linker, scFv proteins retain the specificity of the original immunoglobulin. Single chain Fv polypeptide antibodies can be expressed from a nucleic acid including V_{H}- and V_{L}-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 August 12; Shieh et al., J Immunol 2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Biol Chem 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, “F(ab)” refers to a fragment of an antibody structure that binds to an antigen but is monovalent and does not have a Fc portion, for example, an antibody digested by the enzyme papain yields two F(ab) fragments and an Fc fragment (e.g., a heavy (H) chain constant region; Fc region that does not bind to an antigen).

As used herein, “F(ab′)₂” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S-S bond for binding an antigen and where the remaining H chain portions are linked together. A “F(ab′)2” fragment can be split into two individual Fab′ fragments.

As used herein, the term “vector” refers to any genetic element, such as a plasmid, phage, transposon, cosmid, chromosome, virus, virion, etc., which is capable of replication when associated with the proper control elements and which can transfer gene sequences into cells. Thus, the term includes cloning and expression vehicles, as well as viral vectors and plasmid vectors.

As used herein, the term “expression vector” refers to a recombinant nucleic acid sequence, i.e. recombinant DNA molecule, containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, the term “affinity” is meant a measure of binding strength. Affinity can depend on the closeness of stereochemical fit between antibody combining sites and antigen determinants, on the size of the area of contact between them, and/or on the distribution of charged and hydrophobic groups. Methods for calculating the affinity of an antibody for an antigen are known in the art, including, but not limited to, various antigen-binding experiments, e.g., functional assays (e.g., flow cytometry assay).

The term “chimeric antigen receptor” or “CAR” as used herein refers to a molecule comprising an extracellular antigen-binding domain that is fused to an intracellular signaling domain that is capable of activating or stimulating an immunoresponsive cell, and a transmembrane domain. In certain embodiments, the extracellular antigen-binding domain of a CAR comprises a scFv. The scFv can be derived from fusing the variable heavy and light regions of an antibody. Alternatively or additionally, the scFv may be derived from Fab′s (instead of from an antibody, e.g., obtained from Fab libraries). In certain embodiments, the scFv is fused to the transmembrane domain and then to the intracellular signaling domain. In certain embodiments, the CAR is selected to have high binding affinity for the antigen.

As used herein, the term “nucleic acid molecules” include any nucleic acid molecule that encodes a polypeptide of interest or a fragment thereof. Such nucleic acid molecules need not be 100% homologous or identical to an endogenous nucleic acid sequence, but may exhibit substantial identity.

By “substantially identical” or “substantially homologous” is meant an amino acid sequence or a nucleic acid molecule exhibiting at least about 50% homologous or identical to a reference amino acid sequence (for example, any one of the amino acid sequences described herein) or a reference nucleic acid sequence (for example, any one of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% homologous or identical to the sequence of the reference amino acid or the reference nucleic acid used for comparison. Sequence identity can be measured by using sequence analysis software (for example,

Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences of scFv m903, m904, m905, m906, and m900) disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. The term “constitutive expression” or “constitutively expressed” as used herein refers to expression or expressed under all physiological conditions.

By “disease” is meant any condition, disease or disorder that damages or interferes with the normal function of a cell, tissue, or organ, e.g., neoplasm, and pathogen infection of cell.

By “effective amount” is meant an amount sufficient to have a therapeutic effect. In certain embodiments, an “effective amount” is an amount sufficient to arrest, ameliorate, or inhibit the continued proliferation, growth, or metastasis (e.g., invasion, or migration) of a neoplasm.

By “modulate” is meant positively or negatively alter. Exemplary modulations include a about 1%, about 2%, about 5%, about 10%, about 25%, about 50%, about 75%, or about 100% change.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

By “isolated cell” is meant a cell that is separated from the molecular and/or cellular components that naturally accompany the cell.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “neoplasm” is meant a disease characterized by the pathological proliferation of a cell or tissue and its subsequent migration to or invasion of other tissues or organs. Neoplastic growth is typically uncontrolled and progressive, and occurs under conditions that would not elicit, or would cause cessation of, multiplication of normal cells. Neoplasm can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasm include cancers, such as sarcomas, carcinomas, or plasmacytomas (malignant tumor of the plasma cells). In certain embodiments, the neoplasm is a solid tumor. The neoplasm can a primary tumor or primary cancer. In addition, the neoplasm can be in metastatic status.

As used herein, the term “a conservative sequence modification” refers to an amino acid modification that does not significantly affect or alter the binding characteristics of the presently disclosed mesothelin-targeted CAR (e.g., the extracellular antigen-binding domain of the CAR) comprising the amino acid sequence. Conservative modifications can include amino acid substitutions, additions and deletions. Modifications can be introduced into the extracellular antigen-binding domain of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

By “signal sequence” or “leader sequence” is meant a peptide sequence (e.g., 5, 10, 15, 20, 25 or 30 amino acids) present at the N-terminus of newly synthesized proteins that directs their entry to the secretory pathway. Exemplary leader sequences include, but is not limited to, a human IL-2 signal sequence (e.g. MYRMQLLSCIALSLALVTNS [SEQ ID NO: 67]), a mouse IL-2 signal sequence (e.g., MYSMQLASCVTLTLVLLVNS [SEQ ID NO: 68]); a human kappa leader sequence (e.g., METPAQLLFLLLLWLPDTTG [SEQ ID NO: 69]), a mouse kappa leader sequence (e.g., METDTLLLWVLLLWVPGSTG [SEQ ID NO: 70]); a human CD8 leader sequence (e.g., MALPVTALLLPLALLLHAARP [SEQ ID NO: 71]); a truncated human CD8 signal peptide (e.g., MALPVTALLLPLALLLHA [SEQ ID NO: 72]); a human albumin signal sequence (e.g., MKWVTFISLLFSSAYS [SEQ ID NO: 73]); and a human prolactin signal sequence (e.g., MDSKGSSQKGSRLLLLLVVSNLLLCQGVVS [SEQ ID NO: 74]).

In certain embodiments, the CAR comprises a CD8 signal peptide at the N-terminus, e.g., the signal peptide is connected to the extracellular antigen-binding domain of the CAR. In certain embodiments, the CD8 signal peptide comprises or consists of the amino acid sequence set forth in SEQ ID NO: 71.

An exemplary nucleotide encoding the amino acid sequence of SEQ ID NO: 71 is set forth in SEQ ID NO: 125. SEQ ID NO: 125 is provided below.
ATGGCCCTGCCAGTAACGGCTCTGCTGCTGCCACTTGCTCTGCTCCTCCATGCAGCCAGGCCT [SEQ ID NO: 125]

The terms “comprises”, “comprising”, and are intended to have the broad meaning ascribed to them in U.S. Patent Law and can mean “includes”, “including” and the like.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system, but can affect people with a poorly functioning or suppressed immune system.

Other aspects of the presently disclosed subject matter are described in the following disclosure and are within the ambit of the presently disclosed subject matter.

### 5.2. Polypeptide Compositions

The presently disclosed subject matter provides polypeptide compositions comprising a mesothelin-targeted chimeric antigen receptor (CAR) and a dominant negative form of programmed death 1 (PD-1 DN).

### 5.2.1. Dominant Negative Form of Programmed Death 1 (PD-1 DN)

The dominant negative form of programmed death 1 (referred to as “PD-1 DN”) can enhance the therapeutic efficacy of an immunoresponsive cell comprising a CAR. In certain embodiments, the PD-1 DN comprises (a) at least a portion of an extracellular domain of programmed death 1 (PD-1) comprising a ligand binding region, and (b) a transmembrane domain.

In certain embodiments, an immunoresponsive cell, such as a T cell, or a precursor cell thereof, is engineered to express a dominant negative form (DN form) of PD-1.

Malignant cells adapt to generate an immunosuppressive microenvironment that protects the cells from immune recognition and elimination (Sharpe et al., Dis. Model Mech. 2015; 8:337-350). The immunosuppressive microenvironment puts limitations on immunotherapy methods. The presently disclosed subject matter addresses this limitation by expressing in an immunoresponsive cell, or precursor cell thereof, a DN form of an inhibitor of a cell-mediated immune response. Details of DN forms of inhibitors of a cell-mediated immune response are disclosed in WO2017/040945 and WO2017/100428, the contents of each of which are incorporated herein in their entireties.

Programmed cell death protein 1 (PD-1) is a negative immune regulator of activated T cells upon engagement with its corresponding ligands, PD-L1 and PD-L2, expressed on endogenous macrophages and dendritic cells. PD-1 is a type I membrane protein of 268 amino acids. PD-1 has two ligands, PD-L1 and PD-L2, which are members of the B7 family. The protein's structure comprises an extracellular IgV domain followed by a transmembrane region and an intracellular tail. The intracellular tail contains two phosphorylation sites located in an immunoreceptor tyrosine-based inhibitory motif and an immunoreceptor tyrosine-based switch motif. PD-1 negatively regulates TCR signals. SHP-1 and SHP-2 phosphatases bind to the cytoplasmic tail of PD-1 upon ligand binding. Upregulation of PD-L1 is one mechanism tumor cells use to evade the host immune system. In pre-clinical and clinical trials, PD-1 blockade by antagonistic antibodies induced anti-tumor responses mediated through the host endogenous immune system.

In certain embodiments, a PD-1 polypeptide consists of the amino acid with a GenBank No. NP_005009.2 (SEQ ID NO: 48), or a fragment thereof. In certain embodiments, amino acids 1 to 20 of SEQ ID NO: 48 is the signal peptide (or peptide signal) of PD-1. In certain embodiments, amino acids 21 to 170 of SEQ ID NO: 48 is the extracellular domain of PD-1. In certain embodiments, amino acids 171 to 191 of SEQ ID NO: 48 is the transmembrane domain of PD-1. In certain embodiments, amino acids 192 to 288 of SEQ ID NO: 48 is the intracellular domain of PD-1. SEQ ID NO:48 is provided below:

[SEQ ID NO: 48]
MQIPQAPWPV VWAVLQLGWR PGWFLDSPDR PWNPPTFSPA LLVVTEGDNA TFTCSFSNTS ESFVLNWYRM
SPSNQTDKLA AFPEDRSQPG QDCRFRVTQL PNGRDFHMSV VRARRNDSGT YLCGAISLAP KAQIKESLRA
ELRVTERRAE VPTAHPSPSP RPAGQFQTLV VGVVGGLLGS LVLLVWVLAV ICSRAARGTI GARRTGQPLK
EDPSAVPVFS VDYGELDFQW REKTPEPPVP CVPEQTEYAT IVFPSGMGTS SPARRGSADG PRSAQPLRPE
DGHCSWPL

In certain embodiments, the extracellular domain of PD-1 comprises a ligand binding domain (referred to as “extracellular ligand binding domain”). In certain embodiments, the extracellular ligand binding domain of PD-1 is fused to one or more heterologous polypeptide sequences, that is, the PD-1 DN is a chimeric sequence. For example, the extracellular ligand binding domain of PD-1 can be fused at its N-terminus to a signal peptide that is optionally a heterologous signal peptide, including various signal peptides described herein. In addition, the PD-1 DN can comprise a transmembrane domain that is optionally a heterologous transmembrane domain, including any of various transmembrane domains described herein.

In certain embodiments, the PD-1 DN comprises the extracellular domain of a PD-1 polypeptide (e.g., amino acids 21 to 170 of SEQ ID NO:48) or a ligand binding portion thereof (e.g., amino acids 21 to 165 of SEQ ID NO:48). A cell expressing such a PD-1 DN may lack the ability or have reduced ability to signal in a PD-1 immune checkpoint pathway. In certain embodiments, the PD-1 DN is a deletion mutant consisting of a deletion of the intracellular domain (e.g., the PD-1 DN lacks amino acids 192 to 288 of SEQ ID NO:48) or a portion thereof. A PD-1 consisting of a deletion of the intracellular domain may have reduced or inhibited immune checkpoint pathway mediated by PD-1.

In certain embodiments, the PD-1 DN comprises the extracellular ligand binding domain of PD-1. In certain embodiments, the PD-1 DN comprises the extracellular ligand binding domain of a PD-1 polypeptide, and the transmembrane domain of a PD-1 polypeptide. In certain embodiments, the PD-1 DN comprises or consists of the amino acid sequence set forth in SEQ ID NO: 58 (or amino acids 21 to 165 of SEQ ID NO: 48). SEQ ID NO: 58 is provided below.

[SEQ ID NO: 58]
PGWFLDSPDR PWNPPTFSPA LLVVTEGDNA TFTCSFSNTS ESFVLNWYRM SPSNQTDKLA AFPEDRSQPG
QDCRFRVTQL PNGRDFHMSV VRARRNDSGT YLCGAISLAP KAQIKESLRA ELRVTERRAE VPTAHPSPSP
RPAGQ

An exemplary nucleotide sequence encoding SEQ ID NO: 58 (or amino acids 21 to 165 of SEQ ID NO: 48) is set forth in SEQ ID NO: 59, which is provided below.

[SEQ ID NO: 59]
CCAGGATGGTTCTTAGACTCCCCAGACAGGCCCTGGAACCCCCCCACCT
TCTCCCCAGCCCTGCTCGTGGTGACCGAAGGGGACAACGCCACCTTCAC
CTGCAGCTTCTCCAACACATCGGAGAGCTTCGTGCTAAACTGGTACCGC
ATGAGCCCCAGCAACCAGACGGACAAGCTGGCCGCTTTCCCCGAGGACC
GCAGCCAGCCCGGCCAGGACTGCCGCTTCCGTGTCACACAACTGCCCAA
CGGGCGTGACTTCCACATGAGCGTGGTCAGGGCCCGGCGCAATGACAGC
GGCACCTACCTCTGTGGGGCCATCTCCCTGGCCCCCAAGGCGCAGATCA
AAGAGAGCCTGCGGGCAGAGCTCAGGGTGACAGAGAGAAGGGCAGAAGT
GCCCACAGCCCACCCCAGCCCCTCACCCAGGCCAGCCGGCCAG

In certain embodiments, the PD-1 DN further comprises a signal peptide, e.g., the PD-1 DN comprises the extracellular ligand binding domain of a PD-1 polypeptide, the transmembrane domain of a PD-1 polypeptide, and the signal peptide of a PD-1 polypeptide. In certain embodiments, the signal peptide comprises or consists of amino acids 1-20 of SEQ ID NO: 48. An exemplary nucleotide sequence encoding amino acids 1-20 of SEQ ID NO: 48 is set forth in SEQ ID NO: 60, which is provided below.

[SEQ ID NO: 60]
ATGCAGATCCCACAGGCGCCCTGGCCAGTCGTCTGGGCGGTGCTACAAC
TGGGCTGGCGG

In certain embodiments, the PD-1 DN comprises or consists of amino acids 1 to 165 of SEQ ID NO: 48.

An exemplary nucleotide sequence encoding amino acids 1-165 of SEQ ID NO: 48 is set forth in SEQ ID NO: 61, which is provided below.

[SEQ ID NO: 61]
ATGCAGATCCCACAGGCGCCCTGGCCAGTCGTCTGGGCGGTGCTACAACT
GGGCTGGCGGCCAGGATGGTTCTTAGACTCCCCAGACAGGCCCTGGAACC
CCCCCACCTTCTCCCCAGCCCTGCTCGTGGTGACCGAAGGGGACAACGCC
ACCTTCACCTGCAGCTTCTCCAACACATCGGAGAGCTTCGTGCTAAACTG
GTACCGCATGAGCCCCAGCAACCAGACGGACAAGCTGGCCGCTTTCCCCG
AGGACCGCAGCCAGCCCGGCCAGGACTGCCGCTTCCGTGTCACACAACTG
CCCAACGGGCGTGACTTCCACATGAGCGTGGTCAGGGCCCGGCGCAATGA
CAGCGGCACCTACCTCTGTGGGGCCATCTCCCTGGCCCCCAAGGCGCAGA
TCAAAGAGAGCCTGCGGGCAGAGCTCAGGGTGACAGAGAGAAGGGCAGAA
GTGCCCACAGCCCACCCCAGCCCCTCACCCAGGCCAGCCGGCCAG

In certain embodiments, the PD-1 DN comprises or consists of amino acids 21 to 151 of SEQ ID NO:48. In certain embodiments, a PD-1 DN comprises or consists of amino acids 1 to 151 of SEQ ID NO:48. In certain embodiments, a PD-1 DN comprises or consists of amino acids 21 to 151 of SEQ ID NO:48. In certain embodiments, the PD-1 DN comprises or consists of an amino acid sequence starting at amino acid 21 of SEQ ID NO:48 through an amino acid between amino acids 151 to 165 of SEQ ID NO:48.

In certain embodiments, the PD-1 DN further comprises a CD8 polypeptide. In certain embodiments, the PD-1 DN comprises the extracellular domain of PD-1 or a portion thereof (e.g., the extracellular ligand binding domain) fused to the transmembrane domain and/or the hinge domain of CD8. In certain embodiments, the PD-1 DN comprises the transmembrane domain of CD8 (e.g., amino acids 183 to 203 of SEQ ID NO:86). Such embodiments are representative of a chimeric DN form comprising a transmembrane domain from a different (heterologous) polypeptide. As described above, a PD-1 DN comprising a heterologous domain such as a transmembrane domain can optionally include additional sequence from the heterologous polypeptide. In certain embodiments, the PD-1 DN comprises an additional sequence from the heterologous polypeptide N-terminal of the transmembrane domain. In certain embodiments, the PD-1 DN comprises the hinge domain of CD8. In certain embodiments, the heterologous sequence comprises an additional N-terminal sequence of a CD8 polypeptide (e.g., amino acids 137 to 182 (or optionally starting at amino acids 138 or 139) of SEQ ID NO:86). In certain embodiments, the PD-1 DN comprises an additional sequence from the heterologous polypeptide C-terminal of the transmembrane domain of CD8. In certain embodiments, the additional C-terminal sequence is amino acids 204 to 209 of SEQ ID NO:86.

In certain embodiments, the PD-1 DN comprises the transmembrane domain of a CD8 polypeptide (e.g., amino acids 183 to 203 of SEQ ID NO: 86), a hinge domain of a CD8 polypeptide (e.g., amino acids 137 to 182 of SEQ ID NO: 86), and an additional C-terminal sequence of a CD8 polypeptide (e.g., amino acids 204 to 207 of SEQ ID NO: 86. In certain embodiments, the PD-1 DN comprises a CD8 polypeptide consisting of amino acids 137 to 207 of SEQ ID NO:86.

An exemplary nucleotide sequence encoding amino acids 137 to 207 of SEQ ID NO: 86 is set forth in SEQ ID NO: 62, which is provided below:

[SEQ ID NO: 62]
CCCACCACGACGCCAGCGCCGCGACCACCAACCCCGGCGCCCACGATCGC
GTCGCAGCCCCTGTCCCTGCGCCCAGAGGCGTGCCGGCCAGCGGCGGGGG
GCGCAGTGCACACGAGGGGGCTGGACTTCGCCTGTGATATCTACATCTGG
GCGCCCCTGGCCGGGACTTGTGGGGTCCTTCTCCTGTCACTGGTTATCAC
CCTTTACTGCAAC

In certain embodiments, the PD-1 DN comprises the transmembrane domain of a CD8 polypeptide (e.g., amino acids 183 to 203 of SEQ ID NO: 86), a hinge domain of a CD8 polypeptide (e.g., amino acids 137 to 182 of SEQ ID NO: 86), and an additional C-terminal sequence of a CD8 polypeptide (e.g., amino acids 204 to 209 of SEQ ID NO: 86. In certain embodiments, the PD-1 DN comprises a CD8 polypeptide consisting of amino acids 137 to 209 of SEQ ID NO:86.

An exemplary nucleotide sequence encoding amino acids 137 to 209 of SEQ ID NO: 86 is set forth in SEQ ID NO: 63, which is provided below:

[SEQ ID NO: 63]
CCCACCACGACGCCAGCGCCGCGACCACCAACCCCGGCGCCCACGATCGC
GTCGCAGCCCCTGTCCCTGCGCCCAGAGGCGTGCCGGCCAGCGGCGGGGG
GCGCAGTGCACACGAGGGGGCTGGACTTCGCCTGTGATATCTACATCTGG
GCGCCCCTGGCCGGGACTTGTGGGGTCCTTCTCCTGTCACTGGTTATCAC
CCTTTACTGCAACCACAGG

In certain embodiments, the PD-1 DN comprises the amino acid sequence set forth in SEQ ID NO: 49, which is provided below.

[SEQ ID NO: 49]
MQIPQAPWPVVWAVLQLGWRPGWFLDSPDRPWNPPTFSPALLVVTEGDNA
TFTCSFSNTSESEVLNWYRMSPSNQTDKLAAFPEDRSQPGQDCRFRVTQL
PNGRDFHMSVVRARRNDSGTYLCGAISLAPKAQIKESLRAELRVTERRAE
VPTAHPSPSPRPAGQAAAPTTTPAPRPPTPAPTIASQPLSLRPEACRPAA
GGAVHTRGLDFACDIYIWAPLAGTCGVLLLSLVITLYCN

An exemplary nucleotide sequence encoding the amino acid sequence set forth in SEQ ID NO: 49 is set forth in SEQ ID NO: 64, which is provided below:

[SEQ ID NO: 64]
ATGCAGATCCCACAGGCGCCCTGGCCAGTCGTCTGGGCGGTGCTACAACT
GGGCTGGCGGCCAGGATGGTTCTTAGACTCCCCAGACAGGCCCTGGAACC
CCCCCACCTTCTCCCCAGCCCTGCTCGTGGTGACCGAAGGGGACAACGCC
ACCTTCACCTGCAGCTTCTCCAACACATCGGAGAGCTTCGTGCTAAACTG
GTACCGCATGAGCCCCAGCAACCAGACGGACAAGCTGGCCGCTTTCCCCG
AGGACCGCAGCCAGCCCGGCCAGGACTGCCGCTTCCGTGTCACACAACTG
CCCAACGGGCGTGACTTCCACATGAGCGTGGTCAGGGCCCGGCGCAATGA
CAGCGGCACCTACCTCTGTGGGGCCATCTCCCTGGCCCCCAAGGCGCAGA
TCAAAGAGAGCCTGCGGGCAGAGCTCAGGGTGACAGAGAGAAGGGCAGAA
GTGCCCACAGCCCACCCCAGCCCCTCACCCAGGCCAGCCGGCCAGGCGGC
CGCACCCACCACGACGCCAGCGCCGCGACCACCAACCCCGGCGCCCACGA
TCGCGTCGCAGCCCCTGTCCCTGCGCCCAGAGGCGTGCCGGCCAGCGGCG
GGGGGCGCAGTGCACACGAGGGGGCTGGACTTCGCCTGTGATATCTACAT
CTGGGCGCCCCTGGCCGGGACTTGTGGGGTCCTTCTCCTGTCACTGGTTA
TCACCCTTTACTGCAAC

In certain embodiments, the PD-1 DN comprises the amino acid sequence set forth in SEQ ID NO:118, which is provided below.

[SEQ ID NO: 118]
MQIPQAPWPVVWAVLQLGWRPGWFLDSPDRPWNPPTFSPALLVVTEGDNA
TFTCSFSNTSESEVLNWYRMSPSNQTDKLAAFPEDRSQPGQDCRFRVTQL
PNGRDFHMSVVRARRNDSGTYLCGAISLAPKAQIKESLRAELRVTERRAE
VPTAHPSPSPRPAGQAAAPTTTPAPRPPTPAPTIASQPLSLRPEACRPAA
GGAVHTRGLDFACDIYIWAPLAGTCGVLLLSLVITLYCNHR

An exemplary nucleotide sequence encoding the amino acid sequence set forth in SEQ ID NO: 118 is set forth in SEQ ID NO: 119, which is provided below:

[SEQ ID NO: 119]
ATGCAGATCCCACAGGCGCCCTGGCCAGTCGTCTGGGCGGTGCTACAACT
GGGCTGGCGGCCAGGATGGTTCTTAGACTCCCCAGACAGGCCCTGGAACC
CCCCCACCTTCTCCCCAGCCCTGCTCGTGGTGACCGAAGGGGACAACGCC
ACCTTCACCTGCAGCTTCTCCAACACATCGGAGAGCTTCGTGCTAAACTG
GTACCGCATGAGCCCCAGCAACCAGACGGACAAGCTGGCCGCTTTCCCCG
AGGACCGCAGCCAGCCCGGCCAGGACTGCCGCTTCCGTGTCACACAACTG
CCCAACGGGCGTGACTTCCACATGAGCGTGGTCAGGGCCCGGCGCAATGA
CAGCGGCACCTACCTCTGTGGGGCCATCTCCCTGGCCCCCAAGGCGCAGA
TCAAAGAGAGCCTGCGGGCAGAGCTCAGGGTGACAGAGAGAAGGGCAGAA
GTGCCCACAGCCCACCCCAGCCCCTCACCCAGGCCAGCCGGCCAGGCGGC
CGCACCCACCACGACGCCAGCGCCGCGACCACCAACCCCGGCGCCCACGA
TCGCGTCGCAGCCCCTGTCCCTGCGCCCAGAGGCGTGCCGGCCAGCGGCG
GGGGGCGCAGTGCACACGAGGGGGCTGGACTTCGCCTGTGATATCTACAT
CTGGGCGCCCCTGGCCGGGACTTGTGGGGTCCTTCTCCTGTCACTGGTTA
TCACCCTTTACTGCAACCACAGG

In certain embodiments, the transmembrane domain of the PD-1 DN comprises a hydrophobic alpha helix that spans at least a portion of the membrane. Different transmembrane domains result in different receptor stability. In accordance with the presently disclosed subject matter, the transmembrane domain of the PD-1 DN can comprise a native or modified transmembrane domain of any polypeptide disclose herein, e.g., any transmembrane domain that can be comprised in a chimeric antigen receptor. In certain embodiments, the transmembrane domain is a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40/My88 peptide, a NKGD2 peptide, a synthetic polypeptide (not based on a protein associated with the immune response), or a combination thereof. In certain embodiments, the transmembrane domain is a CD8 polypeptide. Detail of the these transmembrane domains will be described in the section below.

### 5.2.2. Mesothelin-Targeted Chimeric Antigen Receptor (CAR)

The presently disclosed polypeptide composition comprises a CAR that specifically targets mesothelin, e.g., human mesothelin.

CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen-binding domain (e.g., a scFv), which is fused to a transmembrane domain, which is fused to cytoplasmic/intracellular signaling domain. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add intracellular signaling domains from various co-stimulatory molecules (e.g., CD28, 4-1BB, ICOS, OX40, CD27, CD40/My88 and NKGD2) to the cytoplasmic tail of the CAR to provide additional signals to the T cell. “Second generation” CARs comprise those that provide both co-stimulation (e.g., CD28 or 4-1BB) and activation (CD3). “Third generation” CARs comprise those that provide multiple co-stimulation (e.g., CD28 and 4-1BB) and activation (CD3). In certain embodiments, the CAR is a second-generation CAR. In certain embodiments, the CAR comprises an extracellular antigen-binding domain that binds to an antigen, a transmembrane domain, and an intracellular signaling domain, wherein the intracellular signaling domain comprises a co-stimulatory signaling domain. In certain embodiments, the CAR further comprises a hinger/spacer region.

5.2.2.1. Extracellular Antigen Binding Domain of the CAR

The extracellular antigen-binding domain of the CAR specifically binds to mesothelin, e.g., human mesothelin. In certain embodiments, the extracellular antigen-binding domain is an scFv. In certain embodiments, the scFv is a human scFv. In certain embodiments, the scFv is a humanized scFv. In certain embodiments, the scFv is a murine scFv. In certain embodiments, the extracellular antigen-binding domain of the CAR is a Fab, which is optionally crosslinked. In certain embodiments, the extracellular antigen-binding domain of the CAR is a F(ab)₂. In certain embodiments, any of the foregoing molecules may be comprised in a fusion protein with a heterologous sequence to form the extracellular antigen-binding domain. In certain embodiments, the scFv is identified by screening scFv phage library with an antigen-Fc fusion protein. The scFv can be derived from a mouse bearing human V_{L} and/or V_{H} genes. The scFv can also be substituted with a camelid Heavy chain (e.g., VHH, from camel, lama, etc.) or a partial natural ligand for a cell surface receptor.

Mesothelin is an immunogenic cell surface antigen that is highly expressed in solid cancers. Mesothelin is involved in cell proliferation, adhesion, invasion, cell signaling, and metastasis. Studies have demonstrated that serum soluble mesothelin-related peptide secreted by mesothelin-expressing tumors can be measured in both humans and mice, and has been shown to correlate with therapy response and prognosis. In normal tissues, mesothelin is expressed only in the pleura, pericardium, and peritoneum, at low levels. The anti-mesothelin recombinant immunotoxin SS1P has shown in vivo specificity and significant antitumor activity in patients. In a pancreatic cancer vaccine trial, patients with survival advantage had consistent CD8⁺ T cell responses to mesothelin associated with vaccine-induced delayed-type hypersensitivity response. Specific T cell epitopes derived from mesothelin were shown to activate human T cells to efficiently lyse human tumors expressing mesothelin. Thus, there is strong supportive evidence that adoptive immunotherapy targeting mesothelin can target mesothelin-expressing tumors.

In certain embodiments, the CAR binds to human mesothelin. In certain embodiments, the human mesothelin comprises or consists of the amino acid sequence with a NCBI Reference No: AAV87530.1 (SEQ ID NO: 75) or a fragment thereof.

SEQ ID NO:75 is provided below:

[SEQ ID NO: 75]
MALPTARPLL GSCGTPALGS LLFLLFSLGW VQPSRTLAGE
TGQEAAPLDG VLANPPNISS LSPRQLLGFP CAEVSGLSTE
RVRELAVALA QKNVKLSTEQ LRCLAHRLSE PPEDLDALPL
DLLLFLNPDA FSGPQACTHF FSRITKANVD LLPRGAPERQ
RLLPAALACW GVRGSLLSEA DVRALGGLAC DLPGRFVAES
AEVLLPRLVS CPGPLDQDQQ EAARAALQGG GPPYGPPSTW
SVSTMDALRG LLPVLGQPII RSIPQGIVAA WRQRSSRDPS
WRQPERTILR PRFRREVEKT ACPSGKKARE IDESLIFYKK
WELEACVDAA LLATQMDRVN AIPFTYEQLD VLKHKLDELY
PQGYPESVIQ HLGYLFLKMS PEDIRKWNVT SLETLKALLE
VNKGHEMSPQ VATLIDRFVK GRGQLDKDTL DTLTAFYPGY
LCSLSPEELS SVPPSSIWAV RPQDLDTCDP RQLDVLYPKA
RLAFQNMNGS EYFVKIQSFL GGAPTEDLKA LSQQNVSMDL
ATFMKLRTDA VLPLTVAEVQ KLLGPHVEGL KAEERHRPVR
DWILRQRQDD LDTLGLGLQG GIPNGYLVLD LSVQEALSGT
PCLLGPGPVL TVLALLLAST LA

In certain embodiments, the extracellular antigen-binding domain of the CAR (embodied, for example, in a scFv or an analog thereof) binds to human mesothelin with an EC50 value of from about 1 nM to about 25 nM as measured by enzyme-linked immunosorbent assay (ELISA). In certain embodiments, the extracellular antigen-binding domain of the CAR has an EC50 value of about 20 nM as measured by ELISA. In certain embodiments, the extracellular antigen-binding domain of the CAR comprises an anti-mesothelin antibody or an antigen-binding portion thereof described in U.S. Pat. No. 8,357,783, which is herein incorporated by reference in its entirety. In certain embodiments, the extracellular antigen-binding domain of the CAR is derived from a heavy chain variable region and a light chain variable region of an antibody that binds to human mesothelin, e.g., antibody m912 as disclosed in Feng et al., Mol. Cancer Therapy (2009);8(5):1113-1118, which is herein incorporated by reference in its entirety. Antibody m912 was isolated from a human Fab library by panning against recombinant mesothelin. In certain embodiments, the extracellular antigen-binding domain of the CAR is derived from Fab's (e.g., from human or mouse Fab libraries).

Binding of the extracellular antigen-binding domain (embodiment, for example, in a scFv or an analog thereof) of the CAR can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or a scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a y counter or a scintillation counter or by autoradiography. In certain embodiments, the mesothelin targeted extracellular antigen-binding domain is labeled with a fluorescent marker. Non-limiting examples of fluorescent markers include green fluorescent protein (GFP), blue fluorescent protein (e.g., EBFP, EBFP2, Azurite, and mKalama1), cyan fluorescent protein (e.g., ECFP, Cerulean, and CyPet), and yellow fluorescent protein (e.g., YFP, Citrine, Venus, and YPet). In certain embodiments, the mesothelin-targeted human scFv is labeled with GFP.

In certain embodiments, the extracellular antigen-binding domain of the CAR binds to human mesothelin with a mesothelin level of about 1,000 or more mesothelin binding sites/cell. In certain embodiments, the extracellular antigen-binding domain of the CAR binds to human mesothelin with a mesothelin level of from about 1,000 to about 50,000 mesothelin binding sites/cell. In certain embodiments, the extracellular antigen-binding domain of the CAR does not bind to human mesothelin with a mesothelin expression level of less than 1,000 mesothelin binding sites/cell, e.g., the human mesothelin expressed normal tissues, e.g., normal pleura, pericardium, and peritoneum tissues. In certain embodiments, the extracellular antigen-binding domain of the CAR does not bind to human mesothelin with a mesothelin expression level of more than 50,000 mesothelin binding sites/cell. In certain embodiments, a human scFV comprised in the CAR binds to human mesothelin with a mesothelin expression level of from about 1,000 to about 50,000 mesothelin binding sites/cell. In certain embodiments, a human scFV comprised in the CAR does not bind to human mesothelin with a mesothelin expression level of more than 50,000 or less than 1,000 mesothelin binding sites/cell.

In certain embodiments, the extracellular antigen-binding domain of the CAR (e.g., a scFv) comprises a heavy chain variable region (V_{H}) comprising a CDR1 comprising or consisting of the amino acid sequence set forth in SEQ ID NO: 76, or a conservative modification thereof, a CDR2 comprising or consisting of the amino acid sequence set forth in SEQ ID NO: 77 or a conservative modification thereof, and a CDR3 comprising or consisting of the amino acid sequence set forth in SEQ ID NO: 78, a conservative modification thereof. In certain embodiments, the V_{H} comprises a CDR1 comprising or consisting of the amino acid sequence set forth in SEQ ID NO: 76, a CDR2 comprising or consisting of the amino acid sequence set forth in SEQ ID NO: 77, and a CDR3 comprising or consisting of the amino acid sequence set forth in SEQ ID NO: 78.

In certain embodiments, the extracellular antigen-binding domain of the CAR (e.g., a scFv) comprises a light chain variable region (V_{L}) comprising a CDR1 comprising or consisting of the amino acid sequence set forth in SEQ ID NO: 79 or a conservative modification thereof, a CDR2 comprising or consisting of the amino acid sequence set forth in SEQ ID NO: 80 or a conservative modification thereof, and a CDR3 comprising or consisting of the amino acid sequence set forth in SEQ ID NO: 81 or a conservative modification thereof. In certain embodiments, the V_{L} comprises a CDR1 comprising or consisting of the amino acid sequence set forth in SEQ ID NO: 79, a CDR2 comprising or consisting of the amino acid sequence set forth in SEQ ID NO: 80, and a CDR3 comprising or consisting of the amino acid sequence set forth in SEQ ID NO: 81.

In certain embodiments, the V_{H} comprises a CDR1 comprising or consisting of the amino acid sequence set forth in SEQ ID NO: 76 or a conservative modification thereof, a CDR2 comprising or consisting of the amino acid sequence set forth in SEQ ID NO: 77 or a conservative modification thereof, and a CDR3 comprising or consisting of the amino acid sequence set forth in SEQ ID NO: 78, a conservative modification thereof; and the V_{L} comprises a CDR1 comprising or consisting of the amino acid sequence set forth in SEQ ID NO: 79 or a conservative modification thereof, a CDR2 comprising or consisting of the amino acid sequence set forth in SEQ ID NO: 80 or a conservative modification thereof, and a CDR3 comprising or consisting of the amino acid sequence set forth in SEQ ID NO: 81 or a conservative modification thereof. In certain embodiments, the V_{H} comprises a CDR1 comprising or consisting of the amino acid sequence set forth in SEQ ID NO: 76, a CDR2 comprising or consisting of the amino acid sequence set forth in SEQ ID NO: 77, and a CDR3 comprising or consisting of the amino acid sequence set forth in SEQ ID NO: 78; and the V_{L} comprises a CDR1 comprising or consisting of the amino acid sequence set forth in SEQ ID NO: 79, a CDR2 comprising or consisting of the amino acid sequence set forth in SEQ ID NO: 80, and a CDR3 comprising or consisting of the amino acid sequence set forth in SEQ ID NO: 81. In certain embodiments, the CDRs are identified according to the Kabat numbering system.

In certain embodiments, the heavy chain variable region (V_{H}) comprises the amino acid sequence set forth in SEQ ID NO: 82. In certain embodiments, the light chain variable region (V_{L}) comprises the amino acid sequence set forth in SEQ ID NO: 83. In certain embodiments, the V_{H} comprises the amino acid sequence set forth in SEQ ID NO: 82 and the V_{L} comprises the amino acid sequence set forth in SEQ ID NO: 83 , optionally with (iii) a linker sequence, for example a linker peptide, between the V_{H} and the V_{L}. In certain embodiments, the linker comprises or consists of the amino acid sequence set forth in SEQ ID NO: 66. In certain embodiments, the V_{H} comprises an amino acid sequence that is at least about 80% (e.g., at least about 85%, at least about 90%, or at least about 95%) homologous or identical to the amino acid sequence set forth in SEQ ID NO: 82. For example, the V_{H} comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino acid sequence set forth in SEQ ID NO: 82. In certain embodiments, the V_{H} comprises the amino sequence set forth in SEQ ID NO: 82. In certain embodiments, the V_{L} comprises an amino acid sequence that is at least about 80% (e.g., at least about 85%, at least about 90%, or at least about 95%) homologous or identical to the amino acid sequence set forth in SEQ ID NO: 83. For example, the V_{L} comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino acid sequence set forth in SEQ ID NO: 83. In certain embodiments, the V_{L} comprises the amino acid sequence set forth in SEQ ID NO: 83. In certain embodiments, the V_{H} comprises an amino acid sequence that is at least about 80% (e.g., at least about 85%, at least about 90%, or at least about 95%) homologous or identical to the amino acid sequence set forth in SEQ ID NO: 82, and the V_{L} comprises an amino acid sequence that is at least about 80% (e.g., at least about 85%, at least about 90%, or at least about 95%) homologous or identical to the amino acid sequence set forth in SEQ ID NO: 83. In certain embodiments, the V_{H} comprises the amino acid sequence set forth in SEQ ID NO: 82 and the V_{L} comprises the amino acid sequence set forth in SEQ ID NO: 83.

An exemplary nucleic acid sequence encoding the amino acid sequence of SEQ ID NO:82 is set forth in SEQ ID NO:52.

An exemplary nucleic acid sequence encoding the amino acid sequence of SEQ ID NO:83 is set forth in SEQ ID NO:53.

In certain embodiments, the extracellular antigen-binding domain of the CAR (e.g., a scFv) comprises an amino acid sequence that is at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homologous or identical to the amino acid sequence set forth in SEQ ID NO:84. In certain embodiments, the extracellular antigen-binding domain of the CAR (e.g., a scFv) comprises or consists of the amino acid sequence set forth in SEQ ID NO: 84. In certain embodiments, the extracellular antigen-binding domain of the CAR (e.g., a scFv) specifically binds to a human mesothelin polypeptide (e.g., a human mesothelin polypeptide comprising the amino acid sequence set forth in SEQ ID NO: 75).

An exemplary nucleotide sequence encoding the amino acid sequence of SEQ ID NO: 84 is set forth in SEQ ID NO: 85.

In certain embodiments, the scFv is a human scFv.

SEQ ID Nos: 52, 53, and 76-85 are provided below:

[SEQ ID NO: 52]
CAGGTTCAGCTTCAGGAGAGTGGCCCAGGCCTGGTGAAGCCAAGTGAGAC
TCTCAGCTTGACTTGCACAGTTTCTGGAGGCAGTGTCTCCTCAGGCAGCT
ATTATTGGTCCTGGATTCGGCAGCCCCCTGGGAAAGGCCTGGAGTGGATT
GGGTACATATATTACAGTGGCAGCACAAATTACAATCCATCCCTGAAGTC
TCGAGTAACTATCAGTGTGGACACAAGCAAGAATCAGTTTTCACTCAAAC
TGTCTTCTGTGACTGCTGCTGACACTGCTGTTTATTATTGTGCCAGGGAG
GGGAAAAATGGGGCATTTGATATTTGGGGTCAGGGCACAATGGTGACAGT
CAGCTCT
[SEQ ID NO: 53]
CGCCATCAGATGACTCAGTCCCCCTCCAGTCTTTCTGCCTCAGTTGGGGA
TAGAGTGACCATCACATGCAGAGCAAGTCAGAGCATATCATCCTATCTGA
ACTGGTACCAGCAGAAGCCAGGGAAAGCCCCCAAATTGCTGATTTATGCA
GCCTCAAGTCTCCAGAGTGGGGTGCCAAGCAGGTTCTCAGGCAGTGGCAG
TGGGACAGATTTCACATTGACAATCAGCTCCCTCCAACCTGAAGATTTTG
CCACCTACTATTGCCAGCAATCCTACAGCACGCCCCTGACTTTTGGAGGT
GGCACAAAGGTAGAGATCAAGAGGACT
[SEQ ID NO: 76]
GGSVSSGSYY
[SEQ ID NO: 77]
IYYSGST
[SEQ ID NO: 78]
AREGKNGAFDIW
[SEQ ID NO: 79]
QSISSY
[SEQ ID NO: 80]
AASS
[SEQ ID NO: 81]
QQSYSTPLTF
[SEQ ID NO: 82]
QVQLQESGPGLVKPSETLSLTCTVSGGSVSSGSYYWSWIRQPPGKGLEWI
GYIYYSGSTNYNPSLKSRVTISVDTSKNQFSLKLSSVTAADTAVYYCARE
GKNGAFDIWGQGTMVTVSS
[SEQ ID NO: 83]
RHQMTQSPSSLSASVGDRVTITCRASQSISSYLNWYQQKPGKAPKLLIYA
ASSLQSGVPSRFSGSGSGTDFTLTISSLQPEDFATYYCQQSYSTPLTFGG
GTKVEIKRT
[SEQ ID NO: 84]
QVQLQESGPGLVKPSETLSLTCTVSGGSVSSGSYYWSWIRQPPGKGLEWI
GYIYYSGSTNYNPSLKSRVTISVDTSKNQFSLKLSSVTAADTAVYYCARE
GKNGAFDIWGQGTMVTVSSGGGGSGGGGSGGGGSRHQMTQSPSSLSASVG
DRVTITCRASQSISSYLNWYQQKPGKAPKLLIYAASSLQSGVPSRFSGSG
SGTDFTLTISSLQPEDFATYYCQQSYSTPLTFGGGTKVEIKRT
[SEQ ID NO: 85]
CAGGTTCAGCTTCAGGAGAGTGGCCCAGGCCTGGTGAAGCCAAGTGAGAC
TCTCAGCTTGACTTGCACAGTTTCTGGAGGCAGTGTCTCCTCAGGCAGCT
ATTATTGGTCCTGGATTCGGCAGCCCCCTGGGAAAGGCCTGGAGTGGATT
GGGTACATATATTACAGTGGCAGCACAAATTACAATCCATCCCTGAAGTC
TCGAGTAACTATCAGTGTGGACACAAGCAAGAATCAGTTTTCACTCAAAC
TGTCTTCTGTGACTGCTGCTGACACTGCTGTTTATTATTGTGCCAGGGAG
GGGAAAAATGGGGCATTTGATATTTGGGGTCAGGGCACAATGGTGACAGT
CAGCTCTGGAGGTGGAGGCTCAGGAGGAGGAGGCAGTGGAGGTGGTGGGT
CACGCCATCAGATGACTCAGTCCCCCTCCAGTCTTTCTGCCTCAGTTGGG
GATAGAGTGACCATCACATGCAGAGCAAGTCAGAGCATATCATCCTATCT
GAACTGGTACCAGCAGAAGCCAGGGAAAGCCCCCAAATTGCTGATTTATG
CAGCCTCAAGTCTCCAGAGTGGGGTGCCAAGCAGGTTCTCAGGCAGTGGC
AGTGGGACAGATTTCACATTGACAATCAGCTCCCTCCAACCTGAAGATTT
TGCCACCTACTATTGCCAGCAATCCTACAGCACGCCCCTGACTTTTGGAG
GTGGCACAAAGGTAGAGATCAAGAGGACT

In certain embodiments, the heavy chain variable region comprises an amino acid sequence that is at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homologous or identical to the amino acid sequence set forth in SEQ ID NO:36, which is provided below.

[SEQ ID NO: 36]
QVQLQESGPGLVKPSETLSLTCTVSGGSVSSGSYYWSWIRQPPGKGLE
WIGYIYYSGSTNYNPSLKSRVTISVDTSKNQFSLKLSSVTAADTAVYY
CAREGKNGAFDIWGQGTMVTVSSASTKGPSVFPLAPSSKSTSGGTAAL
GCLVKDYFPEPVTVSWNSGALTSGVHTFPAVLQSSGLYSLSSVVTVPS
SSLGTQTYICNVNHKPSNTKVDKKVEPKSCDKTSGQAG

An exemplary nucleic acid sequence encoding the amino acid sequence of SEQ ID NO:36 is set forth in SEQ ID NO:37, which is provided below.

| [SEQ ID NO: 37] | |
|---|---|
| caggtgcagctgcaggagtccggcccaggactggtgaagccttcggagaccctgtccctc | 60 |
| acctgcactgtctctggtggctccgtcagcagtggtagttactactggagctggatccgg | 120 |
| cagcccccagggaagggactggagtggattgggtatatctattacagtgggagcaccaac | 180 |
| tacaacccctccctcaagagtcgagtcaccatatcagtagacacgtccaagaaccagttc | 240 |
| tccctgaagctgagctctgtgaccgctgcggacacggccgtgtattactgtgcgagagag | 300 |
| gggaagaatggggcttttgatatctggggccaagggacaatggtcaccgtctcttcagcc | 360 |
| tccaccaagggcccatcggtcttccccctggcaccctcctccaagagcacctctgggggc | 420 |
| acagcggccctgggctgcctggtcaaggactacttccccgaaccggtgacggtgtcgtgg | 480 |
| aactcaggcgccctgaccagcggcgtgcacaccttcccggctgtcctacagtcctcagga | 540 |
| ctctactccctcagcagcgtggtgaccgtgccctccagcagcttgggcacccagacctac | 600 |
| atctgcaacgtgaatcacaagcccagcaacaccaaggtggacaagaaagttgagcccaaa | 660 |
| tcttgtgacaaaactagtggccaggccggccac | 693 |

In certain embodiments, the light chain variable region comprises an amino acid sequence that is at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homologous or identical to the amino acid sequence set forth in SEQ ID NO:38, which is provided below.

[SEQ ID NO: 38]
DIQMTQSPSSLSASVGDRVTITCRASQSISSYLNWYQQKPGKAPKLLI
YAASSLQSGVPSGFSGSGSGTDFTLTISSLQPEDFATYYCQQSYSTPL
TFGGGTKVEIKRTVAAPSVFIFPPSDEQLKSGTASVVCLLNNEYPREA
KVQWKVDNALQSGNSQESVTEQDSKDSTYSLSSTLTLSKADYEKHKVY
ACEVTHQGLSSPVTKSFNRGEC

An exemplary nucleic acid sequence encoding the amino acid sequence of SEQ ID NO:38 is set forth in SEQ ID NO:39, which is provided below.

| [SEQ ID NO: 39] | |
|---|---|
| gacatccagatgacccagtctccatcctccctgtctgcatctgtaggagacagagtcacc | 60 |
| atcacttgccgggcaagtcagagcattagcagctatttaaattggtatcagcagaaacca | 120 |
| gggaaagcccctaagctcctgatctatgctgcatccagtttgcaaagtggggtcccatca | 180 |
| gggttcagtggcagtggatctgggacagatttcactctcaccatcagcagtctgcaacct | 240 |
| gaagattttgcaacttactactgtcaacagagttacagtaccccgctcactttcggcgga | 300 |
| gggaccaaggtggagatcaaacgaactgtggctgcaccatctgtcttcatcttcccgcca | 360 |
| tctgatgagcagttgaaatctggaactgcctctgttgtgtgcctgctgaataacttctat | 420 |
| cccagagaggccaaagtacagtggaaggtggataacgccctccaatcgggtaactcccag | 480 |
| gagagtgtcacagagcaggacagcaaggacagcacctactgcctcagcagcaccctgacg | 540 |
| ctgagcaaagcagactacgagaaacacaaactctacgcctgcgaagtcacccatcagggc | 600 |
| ctgagctcgcccgtcacaaagagcttcaacaggggagagt | |

In certain embodiments, the light chain variable region comprises an amino acid sequence that is at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homologous or identical to the amino acid sequence set forth in SEQ ID NO:40, which is provided below.

[SEQ ID NO: 40]
RHQMTQSPSSLSASVGDRVTITCRASQSISSYLNWYQQKPGKAPKLLIYA
ASSLQSGVPSGFSGSGSGTDFTLTISSLQPEDFATYYCQQSYSTPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

In certain embodiments, the heavy chain variable region comprises an amino acid sequence that is at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homologous or identical to the amino acid sequence set forth in SEQ ID NO:41, which is provided below.

[SEQ ID NO: 41]
QVQLQESGPGLVKPSETLSLTCTVSGGSVSSGSYYWSWIRQPPGKGLEWI
GYIYYSGSTNYNPSLKSRVTISVDTSKNQFSLKLSSVTAADTAVYYCARE
GKNGAFDIWGQGTMVTVSSS

In certain embodiments, the light chain variable region comprises amino acids 1-107 of SEQ ID NO:38. In certain embodiments, the light chain variable region comprises amino acids 1-107 of SEQ ID NO:40.

In certain embodiments, the extracellular antigen-binding domain of the CAR (e.g., a scFv) comprises an amino acid sequence that is at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homologous or identical to the amino acid sequence set forth in SEQ ID NO:42, which is provided below.

[SEQ ID NO: 42]
Q V Q L Q E S G P G L V K P S E T L S L T C T V S G G S V S S G S Y Y W S W I R
Q P P G K G L E W I G Y I Y Y S G S T N Y N P S L K S R V T I S V D T S K N Q F
S L K L S S V T A A D T A V Y Y C A R E G K N G A F D I W G Q G T M V T V S S S
G G G G S G G G G S G G G G S R H Q M T Q S P S S L S A S V G D R V T I T C R A
S Q S I S S Y L N W Y Q Q K P G K A P K L L I Y A A S S L Q S G V P S R F S G S
G S G T D F T L T I S S L Q P E D F A T Y Y C Q Q S Y S T P L T F G G G T K V E
I K G Q A G H H H H H H G D Y K D D D D K G

An exemplary nucleic acid sequence encoding the amino acid sequence of SEQ ID NO:42 is set forth in SEQ ID NO:45, which is provided below.

[SEQ ID NO: 45]
atggccttaccagtgaccgccttgctcctgccgctggccttgctgctcc
acgccgccaggccgcaggtgcagctgcaggagtccggcccaggactggt
gaagccttcggagaccctgtccctcacctgcactgtctctggtggctcc
gtcagcagtggtagttactactggagctggatccggcagcccccaggga
agggactggagtggattgggtatatctattacagtgggagcaccaacta
caacccctccctcaagagtcgagtcaccatatcagtagacacgtccaag
aaccagttctccctgaagctgagctctgtgaccgctgcggacacggccg
tgtattactgtgcgagagaggggaagaatggggcttttgatatctgggg
ccaagggacaatggtcaccgtctcttcaggtggaggcggttcaggcgga
ggtggctctggcggtggcggatcacgacatcagatgacccagtctccat
cctccctgtctgcatctgtaggagacagagtcaccatcacttgccgggc
aagtcagagcattagcagctatttaaattggtatcagcagaaaccaggg
aaagcccctaagctcctgatctatgctgcatccagtttgcaaagtgggg
tcccatcaaggttcagtggcagtggatctgggacagatttcactctcac
catcagcagtctgcaacctgaagattttgcaacttactactgtcaacag
agttacagtaccccgctcactttcggcggagggaccaaggtggagatca
aacggactgcggccgca

An exemplary nucleic acid sequence encoding the amino acid sequence of SEQ ID NO:42 is set forth in SEQ ID NO:46, which is provided below. The nucleic acid sequence as set forth in SEQ ID NO:46 is synthetically optimized for codon usage, which can increase the expression of the CAR.

[SEQ ID NO: 46]
ATGGCGCTGCCGGTGACCGCGCTGCTGCTGCCGCTGGCGCTGCTGCTGC
ATGCGGCGCGCCCGCAGGTGCAGCTGCAGGAAAGCGGCCCGGGCCTGGT
GAAACCGAGCGAAACCCTGAGCCTGACCTGCACCGTGAGCGGCGGCAGC
GTGAGCAGCGGCAGCTATTATTGGAGCTGGATTCGCCAGCCGCCGGGCA
AAGGCCTGGAATGGATTGGCTATATTTATTATAGCGGCAGCACCAACTA
TAACCCGAGCCTGAAAAGCCGCGTGACCATTAGCGTGGATACCAGCAAA
AACCAGTTTAGCCTGAAACTGAGCAGCGTGACCGCGGCGGATACCGCGG
TGTATTATTGCGCGCGCGAAGGCAAAAACGGCGCGTTTGATATTTGGGG
CCAGGGCACCATGGTGACCGTGAGCAGCGGCGGCGGCGGCAGCGGCGGC
GGCGGCAGCGGCGGCGGCGGCAGCCGCCATCAGATGACCCAGAGCCCGA
GCAGCCTGAGCGCGAGCGTGGGCGATCGCGTGACCATTACCTGCCGCGC
GAGCCAGAGCATTAGCAGCTATCTGAACTGGTATCAGCAGAAACCGGGC
AAAGCGCCGAAACTGCTGATTTATGCGGCGAGCAGCCTGCAGAGCGGCG
TGCCGAGCCGCTTTAGCGGCAGCGGCAGCGGCACCGATTTTACCCTGAC
CATTAGCAGCCTGCAGCCGGAAGATTTTGCGACCTATTATTGCCAGCAG
AGCTATAGCACCCCGCTGACCTTTGGCGGCGGCACCAAAGTGGAAATTA
AACGCACCGCGGCGGCG

An exemplary nucleic acid sequence encoding the amino acid sequence of SEQ ID NO:42 is set forth in SEQ ID NO:47, which is provided below. The nucleic acid sequence as set forth in SEQ ID NO:47 is synthetically optimized for codon usage, which can increase the expression of the CAR.

[SEQ ID NO: 47]
atggccCTCCCGGTAACGGCTCTGCTGCTTCCACTCGCACTGCTCTTGC
ATGCTGCCAGACCACAGGTCCAGCTGCAGGAGAGTGGGCCTGGACTGGT
TAAGCCGAGTGAGACACTTTCCTTGACGTGCACTGTGAGCGGGGGAAGT
GTGTCCTCAGGTAGTTATTACTGGTCCTGGATTCGCCAGCCACCAGGAA
AGGGACTGGAGTGGATAGGTTATATCTATTATTCTGGCAGCACTAATTA
CAATCCTTCTCTCAAAAGTAGGGTGACAATTTCAGTGGATACTTCCAAA
AATCAGTTTAGTCTGAAGCTCAGCTCTGTGACAGCTGCTGATACTGCAG
TTTACTACTGCGCCAGGGAGGGGAAGAATGGCGCCTTCGATATTTGGGG
ACAGGGCACTATGGTGACTGTATCAAGCGGAGGCGGTGGCAGCGGCGGG
GGAGGGAGTGGAGGCGGCGGGTCTCGACATCAGATGACACAGAGCCCAT
CATCACTTAGCGCCAGCGTTGGCGACCGGGTTACGATAACATGCAGGGC
TTCCCAATCTATCAGTTCTTATCTGAACTGGTATCAGCAGAAACCAGGT
AAGGCCCCCAAGCTGCTCATCTACGCAGCCTCATCCCTGCAGAGCGGCG
TCCCTAGTCGATTTTCCGGTAGTGGGTCAGGGACAGATTTTACCCTGAC
TATCAGTTCACTGCAGCCCGAGGACTTCGCGACATACTATTGCCAACAG
TCCTATAGTACACCCTTGACATTTGGCGGCGGGACTAAAGTAGAAATTA
AACGCACCgcggccgca

The V_{H} and/or V_{L} amino acid sequences consisting of at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or sequence identity to a specific sequence (e.g., SEQ ID NO: 82, SEQ ID NO: 83, SEQ ID NO: 36, SEQ ID NO: 38, SEQ ID NO: 40, SEQ ID NO: 41, or SEQ ID NO: 42) may comprise substitutions (e.g., conservative substitutions), insertions, or deletions relative to the specified sequence(s), but retain the ability to bind to a target antigen (e.g., mesothelin). In certain embodiments, a total of 1 to 10 amino acids are substituted, inserted and/or deleted in a specific sequence (e.g., SEQ ID NO: 82, SEQ ID NO: 83, SEQ ID NO: 36, SEQ ID NO: 38, SEQ ID NO: 40, SEQ ID NO: 41, or SEQ ID NO: 42). In certain embodiments, substitutions, insertions, or deletions occur in regions outside the CDRs (e.g., in the FRs) of the extracellular antigen-binding domain. In certain embodiments, the extracellular antigen-binding domain comprises V_{H} and/or V_{L} sequence selected from the group consisting of SEQ ID NOs: 82, and 83, including post-translational modifications of that sequence (SEQ ID NO: 82 and 83).

5.2.2.2. Transmembrane Domain of the CAR

In certain embodiments, the CAR comprises a transmembrane domain. In certain embodiments, the transmembrane domain of the CAR comprises a hydrophobic alpha helix that spans at least a portion of the membrane. Different transmembrane domains result in different receptor stability. After antigen recognition, receptors cluster and a signal are transmitted to the cell. In accordance with the presently disclosed subject matter, the transmembrane domain of the CAR can comprise a native or modified transmembrane domain of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40/My88 peptide, a NKGD2 peptide, a synthetic polypeptide (not based on a protein associated with the immune response), or a combination thereof.

CD8

In certain embodiments, the transmembrane domain comprises a CD8 polypeptide (e.g., a transmembrane domain of CD8 or a portion thereof). In certain embodiments, the transmembrane domain comprises a transmembrane domain of human CD8 or a portion thereof. In certain embodiments, the CD8 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the sequence with a NCBI Reference No: NP_001139345.1 (SEQ ID NO: 86) or a fragment thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 86 which is at least about 20, or at least about 30, or at least about 40, or at least about 50, or at least about 60, or at least about 70, and up to about 235 amino acids in length. In certain embodiments, the CD8 polypeptide comprises or consists of an amino acid sequence of amino acids 1 to 235, 1 to 50, 50 to 100, 100 to 150, 150 to 200, 137 to 209, or 200 to 235 of SEQ ID NO: 86. In certain embodiments, the CAR of the presently disclosed subject matter comprises a transmembrane domain comprising a CD8 polypeptide that comprises or consists of an amino acid sequence of amino acids 137 to 209 of SEQ ID NO: 86. In certain embodiments, the transmembrane domain of the CAR comprises a CD8 polypeptide comprising or consisting of amino acids 137 to 207 of SEQ ID NO: 86.

[SEQ ID NO: 86]
MALPVTALLLPLALLLHAARPSQFRVSPLDRTWNLGETVELKCQVLLSN
PTSGCSWLFQPRGAAASPTFLLYLSQNKPKAAEGLDTQRFSGKRLGDTF
VLTLSDFRRENEGYYFCSALSNSIMYFSHFVPVFLPAKPTTTPAPRPPT
PAPTIASQPLSLRPEACRPAAGGAVHTRGLDFACDIYIWAPLAGTCGVL
LLSLVITLYCNHRNRRRVCKCPRPVVKSGDKPSLSARYV

In certain embodiments, the transmembrane domain comprises a transmembrane domain of mouse CD8 or a portion thereof. In certain embodiments, the CD8 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the sequence with a NCBI Reference No: AAA92533.1 (SEQ ID NO: 87) or a fragment thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 87 which is at least about 20, or at least about 30, or at least about 40, or at least about 50, or at least about 60, or at least about 70, or at least about 100, or at least about 200, and up to 247 amino acids in length. In certain embodiments, the CD8 polypeptide comprises or consists of an amino acid sequence of amino acids 1 to 247, 1 to 50, 50 to 100, 100 to 150, 150 to 200, 151 to 219, or 200 to 247 of SEQ ID NO: 87. In certain embodiments, the transmembrane domain of the CAR comprises a CD8 polypeptide that comprising or consisting of amino acids 151 to 219 of SEQ ID NO: 87.

```
[SEQ ID NO: 87]
    1 MASPLTRFLS LNLLLMGESI ILGSGEAKPQ APELRIFPKK MDAELGQKVD LVCEVLGSVS
  61 QGCSWLFQNS SSKLPQPTFV VYMASSHNKI TWDEKLNSSK LFSAVRDTNN KYVLTLNKFS
121 KENEGYYFCS VISNSVMYFS SVVPVLQKVN STTTKPVLRT PSPVHPTGTS QPQRPEDCRP
181 RGSVKGTGLD FACDIYIWAP LAGICVAPLL SLIITLICYH RSRKRVCKCP RPLVRQEGKP
241 RPSEKIV
```

In certain embodiments, the CD8 polypeptide comprises or consists of the amino acid sequence set forth in SEQ ID NO: 88, which is provided below:

[SEQ ID NO: 88]
STTTKPVLRTPSPVHPTGTSQPQRPEDCRPRGSVKGTGLDFACDIYIWA
PLAGICVALLLSLIITLICY

An exemplary nucleotide sequence encoding the the amino acid sequence of SEQ ID NO: 88 is set forth in SEQ ID NO: 89, which is provided below.

[SEQ ID NO: 89]
TCTACTACTACCAAGCCAGTGCTGCGAACTCCCTCACCTGTGCACCCTA
CCGGGACATCTCAGCCCCAGAGACCAGAAGATTGTCGGCCCCGTGGCTC
AGTGAAGGGGACCGGATTGGACTTCGCCTGTGATATTTACATCTGGGCA
CCCTTGGCCGGAATCTGCGTGGCCCTTCTGCTGTCCTTGATCATCACTC
TCATCTGCTAC

CD28

In certain embodiments, the transmembrane domain of the CAR comprises a CD28 polypeptide (e.g., a transmembrane domain of CD28 or a portion thereof). In certain embodiments, the transmembrane domain comprises a transmembrane domain of human CD28 or a portion thereof. In certain embodiments, the CD28 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the sequence with a NCBI Reference No: NP_006130 (SEQ ID NO: 90) or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 90 which is at least about 20, at least about 25, or at least about 30, or at least about 40, or at least about 50, and up to about 220 amino acids in length. In certain embodiments, the CD28 polypeptide comprises or consists of an amino acid sequence of amino acids 1 to 220, 1 to 50, 50 to 100, 100 to 150, 114 to 220, 153 to 179, 150 to 200, or 200 to 220 of SEQ ID NO: 90. In certain embodiments, the transmembrane domain of the CAR comprises a CD28 polypeptide comprising or consisting of SEQ ID NO: 92 (or amino acids 153 to 179 of SEQ ID NO: 90). An exemplary nucleic acid sequence encoding the amino acid sequence of SEQ ID NO: 92 or amino acids 153 to 179 of SEQ ID NO: 90 is set forth in SEQ ID NO: 93. In certain embodiments, the transmembrane domain of the CAR comprises a CD28 polypeptide comprising or consisting of an amino acid sequence of amino acids 114 to 220 of SEQ ID NO: 90. An exemplary nucleotide sequence encoding the amino acid sequence of SEQ ID NO: 92 (or amino acids 153 to 179 of SEQ ID NO: 90) is set forth in SEQ ID NO: 91. SEQ ID NOs: 90-93 are provided below:

```
[SEQ ID NO: 90]
    1 MLRLLLALNL FPSIQVTGNK ILVKQSPMLV AYDNAVNLSC KYSYNLFSRE FRASLHKGLD
  61 SAVEVCVVYG NYSQQLQVYS KTGFNCDGKL GNESVTFYLQ NLYVNQTDIY FCKIEVMYPP
121 PYLDNEKSNG TIIHVKGKHL CPSPLFPGPS KPFWVLVVVG GVLACYSLLV TVAFIIFWVR
181 SKRSRLLHSD YMNMTPRRPG PTRKHYQPYA PPRDFAAYRS
[SEQ ID NO: 91]
ttttgggtgctggtggtggttggtggagtcctggcttgctatagcttgctagtaacagtggcctttattattttctgggtg
[SEQ ID NO: 92]
FWVLVVVGGV LACYSLLVTV AFIIFWV.
[SEQ ID NO: 93]
TTTTGGGTGCTGGTGGTGGTTGGTGGAGTCCTGGCTTGCTATAGCTTGCTAGTAACAGTGGCCTTTATTATTTTCTGGGTG
```

In certain embodiments, the transmembrane domain comprises a transmembrane domain of mouse CD28 or a portion thereof. In certain embodiments, the CD28 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the sequence with a NCBI Reference No: NP_031668.3 (SEQ ID NO: 97) or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 97 which is at least about 20, or at least about 30, or at least about 40, or at least about 50, and up to 218 amino acids in length. In certain embodiments, the CD28 polypeptide comprises or consists of an amino acid sequence of amino acids 1 to 218, 1 to 50, 50 to 100, 100 to 150, 114 to 220, 150 to 200, 151 to 177, or 200 to 218 of SEQ ID NO: 97. In certain embodiments, the transmembrane domain of the CAR comprises a CD28 polypeptide comprising or consisting of amino acids 151 to 177 of SEQ ID NO: 97.

SEQ ID NO: 97 is provided below:

```
[SEQ ID NO: 97]
    1 MTLRLLFLAL NFFSVQVTEN KILVKQSPLL VVDSNEVSLS CRYSYNLLAK EFRASLYKGV
  61 NSDVEVCVGN GNFTYQPQFR SNAEFNCDGD FDNETVTFRL WNLHVNHTDI YFCKIEFMYP
121 PPYLDNERSN GTIIHIKEKH LCHTQSSPKL FWALVVVAGV LFCYGLLVTV ALCVIWTNSR
181 RNRLLQSDYM NMTPRRPGLT RKPYQPYAPA RDFAAYRP
```

CD84

In certain embodiments, the transmembrane domain of the CAR comprises a native or modified transmembrane domain of a CD84 polypeptide or a portion thereof. The CD84 polypeptide can have an amino acid sequence that is at least about 80%, at least about 85%, at least about 90%, at least about 95%, at least about 96%, at least about 97%, at least about 98%, at least about 99% or at least about 100% homologous or identical to the sequence with a NCBI Reference No: NP_001171808.1 (SEQ ID No: 1) or a fragment thereof, and/or may optionally comprise up to one or up to two or up to three conservative amino acid substitutions. In certain embodiments, the CD84 polypeptide comprises or consists of an amino acid sequence that is a consecutive portion of SEQ ID NO: 1 which is at least about 20, or at least about 30, or at least about 40, or at least about 50, and up to about 345 amino acids in length. In certain embodiments, the CD84 polypeptide comprises or consists of an amino acid sequence of amino acids 1 to 345, 1 to 50, 50 to 100, 100 to 150, 150 to 200, 226 to 250, 250 to 300, or 300 to 345 of SEQ ID NO: 1. In certain embodiments, the transmembrane domain of the CAR comprises or consists of a CD84 polypeptide comprising or consisting of amino acids 226 to 250 of SEQ ID NO: 1.

SEQ ID NO: 1 is provided below:

```
[SEQ ID NO: 1]
    1 maqhhlwill lclqtwpeaa gkdseiftvn gilgesvtfp vniqeprqvk iiawtsktsv
  61 ayvtpgdset apvvtvthrn yyerihalgp nynlvisdlr medagdykad intqadpytt
121 tkrynlqiyr rlgkpkitqs lmasvnstcn vtltcsveke eknvtynwsp lgeegnvlqi
181 fqtpedqelt ytctaqnpvs nnsdsisarq lcadiamgfr thhtgllsvl amffllvlil
241 ssvflfrlfk rrqgrifpeg sclntftknp yaaskktiyt yimasrntqp aesriydeil
301 qskvlpskee pvntvysevq fadkmgkast qdskppgtss yeivi
```

An exemplary nucleotide sequence encoding amino acids 226 to 250 of SEQ ID NO: 1 is set forth in SEQ ID NO: 2, which is provided below.

[SEQ ID NO: 2]
TTGCTGAGCGTGCTGGCTATGTTCTTTCTGCTTGTTCTCATTCTGTCTT
CAGTGTTTTTGTTCCGTTTGTTCAAG

CD166

In certain embodiments, the transmembrane domain of the CAR comprises a native or modified transmembrane domain of a CD166 polypeptide or a portion thereof. The CD166 polypeptide can have an amino acid sequence that is at least about 80%, at least about 85%, at least about 90%, at least about 95%, at least about 96%, at least about 97%, at least about 98%, at least about 99% or at least about 100% homologous or identical to the sequence with a NCBI Reference No: NP_001618.2 (SEQ ID NO: 3) or a fragment thereof, and/or may optionally comprise up to one or up to two or up to three conservative amino acid substitutions. In certain embodiments, the CD166 polypeptide comprises or consists of an amino acid sequence that is a consecutive portion of SEQ ID NO: 3 which is at least about 20, or at least about 30, or at least about 40, or at least about 50, at least about 100, and up to about 583 amino acids in length. In certain embodiments, the CD166 polypeptide comprises or consists of an amino acid sequence of amino acids 1 to 583, 1 to 50, 50 to 100, 100 to 150, 150 to 200, 200 to 300, 300 to 400, 400 to 500, 528 to 549, or 500 to 583 of SEQ ID NO: 3. In certain embodiments, the CD166 polypeptide comprised in the transmembrane domain of a presently disclosed CAR comprises or consists of an amino acid sequence of amino acids 528 to 553 of SEQ ID NO: 3. In certain embodiments, the CD166 polypeptide comprised in the transmembrane domain of the CAR comprises or consists of an amino acid sequence of amino acids 528 to 549 of SEQ ID NO: 3.

SEQ ID NO: 3 is provided below:

```
[SEQ ID NO: 3]
    1 MESKGASSCR LLFCLLISAT VFRPGLGWYT VNSAYGDTII IPCRLDVPQN LMFGKWKYEK
  61 PDGSPVFIAF RSSTKKSVQY DDVPEYKDRL NLSENYTLSI SNARISDEKR FVCMLVTEDN
121 VFEAPTIVKV FKQPSKPEIV SKALFLETEQ LKKLGDCISE DSYPDGNITW YRNGKVLHPL
181 EGAVVIIFKK EMDPVTQLYT MTSTLEYKTT KADIQMPFTC SVTYYGPSGQ KTIHSEQAVF
241 DIYYPTEQVT IQVLPPKNAI KEGDNITLKC LGNGNPPPEE FLFYLPGQPE GIRSSNTYTL
301 TDVRRNATGD YKCSLIDKKS MIASTAITVH YLDLSLNPSG EVTRQIGDAL PVSCTISASR
361 NATVVWMKDN IRLRSSPSFS SLHYQDAGNY VCETALQEVE GLKKRESLTL IVEGKPQIKM
421 TKKTDPSGLS KTIICHVEGF PKPAIQWTIT GSGSVINQTE ESPYINGRYY SKIIISPEEN
481 VTLTCTAENQ LERTVNSLNV SAISIPEHDE ADEISDENRE KVNDQAKLIV GIVVGLLLAA
541 LVAGVVYWLY MKKSKTASKH VNKDLGNMEE NKKLEENNHK TEA
```

An exemplary nucleotide sequence encoding amino acids 528 to 553 of SEQ ID NO: 3 is set forth in SEQ ID NO: 4, which is provided below.

[SEQ ID NO: 4]
CTAATTGTGGGAATCGTTGTTGGTCTCCTCCTTGCTGCCCTTGTTGCTG
GTGTCGTCTACTGGCTGTACATGAAGAAG

CD8a

In certain embodiments, the transmembrane domain of the CAR comprises a native or modified transmembrane domain of a CD8a polypeptide or a portion thereof. The CD8a polypeptide can have an amino acid sequence that is at least about 80%, at least about 85%, at least about 90%, at least about 95%, at least about 96%, at least about 97%, at least about 98%, at least about 99% or at least about 100% homologous or identical to the sequence consisting of a NCBI Reference No: NP_001139345.1 (SEQ ID No: 5), or fragments thereof, and/or may optionally comprise up to one or up to two or up to three conservative amino acid substitutions. In certain embodiments, the CD8a polypeptide comprises or consists of an amino acid sequence that is a consecutive portion of SEQ ID NO: 5 which is at least about 20, or at least about 30, or at least about 40, or at least about 50, and up to about 235 amino acids in length. In certain embodiments, the CD8a polypeptide comprises or consists of an amino acid sequence of amino acids 1 to 235, 1 to 50, 50 to 100, 100 to 150, 183 to 207, 150 to 200, or 200 to 235 of SEQ ID NO: 5. In certain embodiments, the transmembrane domain of the CAR comprises a CD8a polypeptide comprising or consisting of amino acids 183 to 207 of SEQ ID NO: 5. SEQ ID NO: 5 is provided below:

```
[SEQ ID NO: 5]
    1 MALPVTALLL PLALLLHAAR PSQFRVSPLD RTWNLGETVE LKCQVLLSNP TSGCSWLFQP
  61 RGAAASPTFL LYLSQNKPKA AEGLDTQRFS GKRLGDTFVL TLSDFRRENE GYYFCSALSN
121 SIMYFSHFVP VFLPAKPTTT PAPRPPTPAP TIASQPLSLR PEACRPAAGG AVHTRGLDFA
181 CDIYIWAPLA GTCGVLLLSL VITLYCNHRN RRRVCKCPRP VVKSGDKPSL SARYV
```

An exemplary nucleotide sequence encoding amino acids 183 to 207 of SEQ ID NO: 5 is set forth in SEQ ID NO: 6, which is provided below.

[SEQ ID NO: 6]
atctacatctgggcgcccttggccgggacttgtggggtccttctcctgtca
ctggttatcaccctttactgcaac

CD8b

In certain embodiments, the transmembrane domain of the CAR comprises a native or modified transmembrane domain of a CD8b polypeptide or a portion thereof. The CD8b polypeptide can have an amino acid sequence that is at least about 80%, at least about 85%, at least about 90%, at least about 95%, at least about 96%, at least about 97%, at least about 98%, at least about 99% or at least about 100% homologous or identical to the sequence with a NCBI Reference No: NP_742099.1 (SEQ ID No: 7), or fragments thereof, and/or may optionally comprise up to one or up to two or up to three conservative amino acid substitutions. In certain embodiments, the CD8b polypeptide comprises or consists of an amino acid sequence that is a consecutive portion of SEQ ID NO: 7 which is at least about 20, or at least about 30, or at least about 40, or at least about 50, and up to about 221 amino acids in length. In certain embodiments, the CD8b polypeptide comprises or consists of an amino acid sequence of amino acids 1 to 221, 1 to 50, 50 to 100, 100 to 150, 171 to 195, 150 to 200, or 200 to 221 of SEQ ID NO: 7. In certain embodiments, the transmembrane domain of the CAR comprises a CD8b polypeptide comprising or consisting of amino acids 171 to 195 of SEQ ID NO: 7. SEQ ID NO: 7 is provided below:

[SEQ ID NO: 7]

| | |
|---|---|
| 1 | MRPRLWLLLA AQLTVLHGNS VLQQTPAYIK VQTNKMVMLS |
| | CEAKISLSNM RIYWLRQRQA |
| 61 | PSSDSHHEFL ALWDSAKGTI HGEEVEQEKI AVFRDASRFI |
| | LNLTSVKPED SGIYFCMIVG |
| 121 | SPELTFGKGT QLSVVDFLPT TAQPTKKSTL KKRVCRLPRP |
| | ETQKGPLCSP ITLGLLVAGV |
| 181 | LVLLVSLGVA IHLCCRRRRA RLRFMKQLRL HPLEKCSRMD Y |

An exemplary nucleotide sequence encoding amino acids 171 to 195 of SEQ ID NO: 7 is set forth in SEQ ID NO: 8, which is provided below.

[SEQ ID NO: 8]
ATCACCCTTGGCCTGCTGGTGGCTGGCGTCCTGGTTCTGCTGGTTTCCCTG
GGAGTGGCCATCCACCTGTGCTGC

ICOS

In certain embodiments, the transmembrane domain of the CAR comprises a native or modified transmembrane domain of an ICOS polypeptide or a portion thereof. The ICOS polypeptide can have an amino acid sequence that is at least about 80%, at least about 85%, at least about 90%, at least about 95%, at least about 96%, at least about 97%, at least about 98%, at least about 99% or at least about 100% homologous or identical to the sequence with a NCBI Reference No: NP_036224.1 (SEQ ID No: 9) or a fragment thereof, and/or may optionally comprise up to one or up to two or up to three conservative amino acid substitutions. In certain embodiments, the ICOS polypeptide comprises or consists of an amino acid sequence that is a consecutive portion of SEQ ID NO: 9 which is at least about 20, or at least about 30, or at least about 40, or at least about 50, and up to about 199 amino acids in length. In certain embodiments, the ICOS polypeptide comprises or consists of an amino acid sequence of amino acids 1 to 199, 1 to 50, 50 to 100, 100 to 150, 141 to 165, or 150 to 199 of SEQ ID NO: 9. In certain embodiments, the transmembrane domain of the CAR comprises a ICOS polypeptide comprising or consisting of amino acids 141 to 165 of SEQ ID NO: 9. SEQ ID NO: 9 is provided below:

[SEQ ID NO: 9]

| | |
|---|---|
| 1 | MKSGLWYFFL FCLRIKVLTG EINGSANYEM FIFHNGGVQI |
| | LCKYPDIVQQ FKMQLLKGGQ |
| 61 | ILCDLTKTKG SGNTVSIKSL KFCHSQLSNN SVSFFLYNLD |
| | HSHANYYFCN LSIFDPPPFK |
| 121 | VTLTGGYLHI YESQLCCQLK FWLPIGCAAF VVVCILGCIL |
| | ICWLTKKKYS SSVHDPNGEY |
| 181 | MFMRAVNTAK KSRLTDVTL |

An exemplary nucleotide sequence encoding amino acids 141 to 165 of SEQ ID NO: 9 is set forth in SEQ ID NO: 10, which is provided below.

[SEQ ID NO: 10]
TTCTGGTTACCCATAGGATGTGCAGCCTTTGTTGTAGTCTGCATTTTGGGA
TGCATACTTATTTGTTGGCTTACA

CTLA-4

In certain embodiments, the transmembrane domain of the CAR comprises a native or modified transmembrane domain of a CTLA-4 polypeptide or a portion thereof. The CTLA-4 polypeptide can have an amino acid sequence that is at least about 80%, at least about 85%, at least about 90%, at least about 95%, at least about 96%, at least about 97%, at least about 98%, at least about 99% or at least about 100% homologous or identical to the sequence with a NCBI Reference No: NP_005205.2 (SEQ ID No: 11) or a fragment thereof, and/or may optionally comprise up to one or up to two or up to three conservative amino acid substitutions. In certain embodiments, the CTLA-4 polypeptide comprises or consists of an amino acid sequence that is a consecutive portion of SEQ ID NO: 11 which is at least about 20, or at least about 30, or at least about 40, or at least about 50, and up to about 223 amino acids in length. In certain embodiments, the CTLA-4 polypeptide comprises or consists of an amino acid sequence of amino acids 1 to 223, 1 to 50, 50 to 100, 100 to 150, 162 to 186, 150 to 200, or 200 to 223 of SEQ ID NO: 11. In certain embodiments, the transmembrane domain of the CAR comprises a CTLA-4 polypeptide comprising or consisting of amino acids 162 to 186 of SEQ ID NO: 11. SEQ ID NO: 11 is provided below:

[SEQ ID NO: 11]

| | |
|---|---|
| 1 | MACLGFQRHK AQLNLATRTW PCTLLFFLLF IPVFCKAMHV |
| | AQPAVVLASS RGIASFVCEY |
| 61 | ASPGKATEVR VTVLRQADSQ VTEVCAATYM MGNELTFLDD |
| | SICTGTSSGN QVNLTIQGLR |
| 121 | AMDTGLYICK VELMYPPPYY LGIGNGTQIY VIDPEPCPDS |
| | DFLLWILAAV SSGLFFYSFL |
| 181 | LTAVSLSKML KKRSPLTTGV YVKMPPTEPE CEKQFQPYFI |
| | PIN |

An exemplary nucleotide sequence encoding amino acids 162 to 186 of SEQ ID NO: 11 is set forth in SEQ ID NO: 12, which is provided below.

[SEQ ID NO: 12]
TTCCTCCTCTGGATCCTTGCAGCAGTTAGTTCGGGGTTGTTTTTTTATAGC
TTTCTCCTCACAGCTGTTTCTTTG

ICAM-1

In certain embodiments, the transmembrane domain of the CAR comprises a native or modified transmembrane domain of an ICAM-1 polypeptide or a portion thereof. The ICAM-1 polypeptide can have an amino acid sequence that is at least about 80%, at least about 85%, at least about 90%, at least about 95%, at least about 96%, at least about 97%, at least about 98%, at least about 99% or at least about 100% homologous or identical to the sequence with a NCBI Reference No: NP_000192.2 (SEQ ID No: 13) or a fragment thereof, and/or may optionally comprise up to one or up to two or up to three conservative amino acid substitutions. In certain embodiments, the ICAM-1 polypeptide comprises or consists of an amino acid sequence that is a consecutive portion of SEQ ID NO: 13 which is at least about 20, or at least about 30, or at least about 40, or at least about 50, and up to about 220 amino acids in length. In certain embodiments, the ICAM-1 polypeptide comprises or consists of an amino acid sequence of amino acids 1 to 532, 1 to 50, 50 to 100, 100 to 150, 150 to 200, 200 to 300, 300 to 400, 481 to 507, 400 to 500, or 500 to 532 of SEQ ID NO: 13. In certain embodiments, the transmembrane domain of the CAR comprises a ICAM-1 polypeptide comprising or consisting of amino acids 481 to 507 of SEQ ID NO: 13. SEQ ID NO: 13 is provided below:

[SEQ ID NO: 13]

| | |
|---|---|
| 1 | MAPSSPRPAL PALLVLLGAL FPGPGNAQTS VSPSKVILPR |
| | GGSVLVTCST SCDQPKLLGI |
| 61 | ETPLPKKELL LPGNNRKVYE LSNVQEDSQP MCYSNCPDGQ |
| | STAKTFLTVY WTPERVELAP |
| 121 | LPSWQPVGKN LTLRCQVEGG APRANLTVVL LRGEKELKRE |
| | PAVGEPAEVT TTVLVRRDHH |
| 181 | GANFSCRTEL DLRPQGLELF ENTSAPYQLQ TFVLPATPPQ |
| | LVSPRVLEVD TQGTVVCSLD |
| 241 | GLFPVSEAQV HLALGDQRLN PTVTYGNDSF SAKASVSVTA |
| | EDEGTQRLTC AVILGNQSQE |
| 301 | TLQTVTIYSF PAPNVILTKP EVSEGTEVTV KCEAHPRAKV |
| | TLNGVPAQPL GPRAQLLLKA |
| 361 | TPEDNGRSFS CSATLEVAGQ LIHKNQTREL RVLYGPRLDE |
| | RDCPGNWTWP ENSQQTPMCQ |
| 421 | AWGNPLPELK CLKDGTFPLP IGESVTVTRD LEGTYLCRAR |
| | STQGEVTRKV TVNVLSPRYE |
| 481 | IVIITVVAAA VIMGTAGLST YLYNRQRKIK KYRLQQAQKG |
| | TPMKPNTQAT PP |

An exemplary nucleotide sequence encoding amino acids 481 to 507 of SEQ ID NO: 13 is set forth in SEQ ID NO: 14, which is provided below.

[SEQ ID NO: 14]
ATTGTCATCATCACTGTGGTAGCAGCCGCAGTCATAATGGGCACTGCAGGC
CTCAGCACGTACCTCTATAACCGCCAGCGG

5.2.2.3. Hinge/Spacer Region of the CAR

In certain embodiments, the CAR comprises a hinge/spacer region that links the extracellular antigen-binding domain to the transmembrane domain. The hinge/spacer region can be flexible enough to allow the antigen binding domain to orient in different directions to facilitate antigen recognition. In certain embodiments, the hinge/spacer region of the CAR can comprise a native or modified hinge region of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40/My88 peptide, a NKGD2 peptide, a synthetic polypeptide (not based on a protein associated with the immune response), or a combination thereof. The hinge/spacer region can be the hinge region from IgG1, or the CH2CH3 region of immunoglobulin and portions of CD3, a portion of a CD28 polypeptide (e.g., a portion of SEQ ID NO: 90), a portion of a CD8 polypeptide (e.g., a portion of SEQ ID NO: 86, or a portion of SEQ ID NO: 87), a variation of any of the foregoing which is at least about 80%, at least about 85%, at least about 90%, at least about 95%, or at least about 100% homologous or identical thereto, or a synthetic spacer sequence.

CD28

In certain embodiments, the hinge/spacer region of the CAR comprises a native or modified hinge region of a CD28 polypeptide or a portion thereof, as described herein. In certain embodiments, the hinge/spacer region of the CAR comprises a CD28 polypeptide comprising or consisting of the amino acid sequence set forth in SEQ ID NO: 15 (or amino acids 114 to 152 of SEQ ID NO: 90). SEQ ID NO: 15 is provided below.

[SEQ ID NO: 15]
IEVMYPPPYLDNEKSNGTIIHVKGKHLCPSPLFPGPSKP

An exemplary nucleotide sequence encoding the amino acid sequence of SEQ ID NO: 15 (or amino acids 114 to 152 of SEQ ID NO: 90) is set forth in SEQ ID NO: 54, which is provided below.

[SEQ ID NO: 54]
attgaagttatgtatcctcctccttacctagacaatgagaagagcaatgga
accattatccatgtgaaagggaaacacctttgtccaagtcccctatttccc
ggaccttctaagccc

CD84

In certain embodiments, the hinge/spacer region of the CAR comprises a native or modified hinge region of a CD84 polypeptide or a portion thereof, as described herein. In certain embodiments, the hinge/spacer region of the CAR comprises a CD84 polypeptide comprising or consisting of amino acids 187 to 225 of SEQ ID NO: 1. An exemplary nucleotide sequence encoding amino acids 187 to 225 of SEQ ID NO: 1 is set forth in SEQ ID NO: 16, which is provided below.

[SEQ ID NO: 16]
CAAGAGCTGACTTACACGTGTACAGCCCAGAACCCTGTCAGCAACAATTCT
GACTCCATCTCTGCCCGGCAGCTCTGTGCAGACATCGCAATGGGCTTCCGT
ACTCACCACACCGGG

CD166

In certain embodiments, the hinge/spacer region of the CAR comprises a native or modified hinge region of a CD166 polypeptide or a portion thereof, as described herein. In certain embodiments, the hinge/spacer region of the CAR comprises a CD166 polypeptide comprising or consisting of amino acids 489 to 527 of SEQ ID NO:3. An exemplary nucleotide sequence encoding amino acids 489 to 527 of SEQ ID NO: 3 is set forth in SEQ ID NO: 17, which is provided below.

[SEQ ID NO: 17]
ACCAACTGGAGAGAACAGTAAACTCCTTGAATGTCTCTGCTATAAGTATTC
CAGAACACGATGAGGCAGACGAGATAAGTGATGAAAACAGAGAAAAGGTGA
ATGACCAGGCAAAA

In certain embodiments, the hinge/spacer region of the CAR comprises a CD166 polypeptide comprising or consisting of amino acids 484 to 527 of SEQ ID NO:3. In certain embodiments, the hinge/spacer region of the CAR comprises a CD166 polypeptide comprising or consisting of amino acids 506 to 527 of SEQ ID NO:3. In certain embodiments, the hinge/spacer region of the CAR comprises a CD166 polypeptide comprising or consisting of amino acids 517 to 527 of SEQ ID NO:3. In certain embodiments, the hinge/spacer region of the CAR comprises a CD166 polypeptide comprising or consisting of the amino acid sequence set forth in SEQ ID NO: 109 or SEQ ID NO: 110. SEQ ID Nos: 109 and 110 are provided below.

[SEQ ID NO: 109]
NQLERTVNSLNVPAISIPEHDEADEISDENREKVNDQAK
[SEQ ID NO: 110]
AAANQLERTVNSLNVSAISIPEHDEADEISDENREKVNDQAK

In certain embodiments, the CD166 polypeptide comprised in the hinge/spacer region and the transmembrane domain of the CAR comprises or consists of the amino acid sequence set forth in SEQ ID NO: 111, SEQ ID NO: 112, SEQ ID NO: 113, SEQ ID NO: 114, SEQ ID NO: 115, SEQ ID NO: 116, or SEQ ID NO: 117. SEQ ID Nos: 111-117 are provided below.

[SEQ ID NO: 111]
PEHDEADEISDENREKVNDQAKLIVGIVVGLLLAALVAGVVYWLYMKK
[SEQ ID NO: 112]
ENREKVNDQAKLIVGIVVGLLLAALVAGVVYWLYMKK
[SEQ ID NO: 113]
NQLERTVNSLNVPAISIPEHDEADEISDENREKVNDQAKLIVGIVVGLLLA
ALVAGVVYWLYMKK
[SEQ ID NO: 114]
TCTAENQLERTVNSLNVSAISIPEHDEADEISDENREKVNDQAKLIVGIVV
GLLLAALVAGVVYWL
[SEQ ID NO: 115]
PEHDEADEISDENREKVNDQAKLIVGIVVGLLLAALVAGVVYWL
[SEQ ID NO: 116]
NQLERTVNSLNVSAISIPEHDEADEISDENREKVNDQAKLIVGIVVGLLLA
ALVAGVVYWL
[SEQ ID NO: 117]
AAANQLERTVNSLNVSAISIPEHDEADEISDENREKVNDQAKLIVGIVVGL
LLAALVAGVVYWLYMKK

CD8a

In certain embodiments, the hinge/spacer region of the CAR comprises a native or modified hinge region of a CD8a polypeptide or a portion thereof, as described herein. In certain embodiments, the hinge/spacer region of the CAR comprises a CD8a polypeptide comprising or consisting of amino acids 137 to 182 of SEQ ID NO: 5. An exemplary nucleotide sequence encoding amino acids 137 to 182 of SEQ ID NO: 5 is set forth in SEQ ID NO: 18, which is provided below.

[SEQ ID NO: 18]
cccaccacgacgccagcgccgcgaccaccaacaccggcgcccaccatc
gcgtcgcagcccctgtccctgcgcccagaggcgtgccggccagcggcg
gggggcgcagtgcacacgagggggctggacttcgcctgtgat

CD8b

In certain embodiments, the hinge/spacer region of the CAR comprises a native or modified hinge region of a CD8b polypeptide as described herein. In certain embodiments, the CD8b polypeptide comprised in the hinge/spacer region of the CAR comprises or consists of amino acids 132 to 170 of SEQ ID NO: 7. An exemplary nucleotide sequence encoding amino acids 132 to 170 of SEQ ID NO: 7 is set forth in SEQ ID NO: 19, which is provided below.

[SEQ ID NO: 19]
CTGAGTGTGGTTGATTTCCTTCCCACCACTGCCCAGCCCACCAAGAAG
TCCACCCTCAAGAAGAGAGTGTGCCGGTTACCCAGGCCAGAGACCCAG
AAGGGCCCACTTTGTAGCCCC

ICOS

In certain embodiments, the hinge/spacer region of the CAR comprises a native or modified hinge region of an ICOS polypeptide or portion thereof, as described herein. In certain embodiments, the hinge/spacer region of the CAR comprises an ICOS polypeptide comprising or consisting of amino acids 102 to 140 of SEQ ID NO: 9. An exemplary nucleotide sequence encoding amino acids 102 to 140 of SEQ ID NO: 9 is set forth in SEQ ID NO: 20, which is provided below.

[SEQ ID NO: 20]
tctcatgccaactattacttctgcaacctatcaatttttgatcctcct
ccttttaaagtaactcttacaggaggatatttgcatatttatgaatca
caactttgttgccagctgaag

CTLA-4

In certain embodiments, the hinge/spacer region of the CAR comprises a native or modified hinge region of a CTLA-4 polypeptide or a portion thereof, as described herein. In certain embodiments, the hinge/spacer region of the CAR comprises a CTLA-4 polypeptide comprising or consisting of amino acids 123 to 161 of SEQ ID NO: 11. An exemplary nucleotide sequence encoding amino acids 123 to 161 of SEQ ID NO: 11 is set forth in SEQ ID NO: 21, which is provided below.

[SEQ ID NO: 21]
GACACGGGACTCTACATCTGCAAGGTGGAGCTCATGTACCCACCGCCA
TACTACCTGGGCATAGGCAACGGAACCCAGATTTATGTAATTGATCCA
GAACCGTGCCCAGATTCTGAC

ICAM-1

In certain embodiments, the hinge/spacer region of the CAR comprises a native or modified hinge region of a ICAM-1 polypeptide or a portion thereof, as described herein. In certain embodiments, the hinge/spacer region of the CAR comprises an ICAM-1 polypeptide comprising or consisting of amino acids 442 to 480 of SEQ ID NO: 13. An exemplary nucleotide sequence encoding amino acids 442 to 480 of SEQ ID NO: 13 is set forth in SEQ ID NO: 22, which is provided below.

[SEQ ID NO: 22]
GGGGAATCAGTGACTGTCACTCGAGATCTTGAGGGCACCTACCTCTGT
CGGGCCAGGAGCACTCAAGGGGAGGTCACCCGCAAGGTGACCGTGAAT
GTGCTCTCCCCCCGGTATGAG

In certain embodiments, the mesothelin-targeted CAR comprises a hinge/spacer region. In certain embodiments, the hinge/spacer region is positioned between the extracellular antigen-binding domain and the transmembrane domain. In certain embodiments, the hinge/spacer region comprises a CD8 polypeptide, a CD28 polypeptide, a CD3ζ polypeptide, a CD4 polypeptide, a 4-1BB polypeptide, an OX40 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40/My88 peptide, a NKGD2 peptide, a synthetic polypeptide (not based on a protein associated with the immune response), or a combination thereof. In certain embodiments, the transmembrane domain comprises a CD8 polypeptide, a CD28 polypeptide, a CD3ζ polypeptide, a CD4 polypeptide, a 4-1BB polypeptide, an OX40 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40/My88 peptide, a NKGD2 peptide, a synthetic polypeptide (not based on a protein associated with the immune response), or a combination thereof.

In certain embodiments, the transmembrane domain and the hinge/spacer region are derived from the same molecule. In certain embodiments, the transmembrane domain and the hinge/spacer region are derived from different molecules. In certain embodiments, the hinge/spacer region of the CAR comprises a CD28 polypeptide and the transmembrane domain of the CAR comprises a CD28 polypeptide. In certain embodiments, the hinge/spacer region of the CAR comprises a CD28 polypeptide and the transmembrane domain of the CAR comprises a CD28 polypeptide. In certain embodiments, the hinge/spacer region of the CAR comprises a CD84 polypeptide and the transmembrane domain of the CAR comprises a CD84 polypeptide. In certain embodiments, the hinge/spacer region of the CAR comprises a CD166 polypeptide and the transmembrane domain of the CAR comprises a CD166 polypeptide. In certain embodiments, the hinge/spacer region of the CAR comprises a CD8a polypeptide and the transmembrane domain of the CAR comprises a CD8a polypeptide. In certain embodiments, the hinge/spacer region of the CAR comprises a CD8b polypeptide and the transmembrane domain of the CAR comprises a CD8b polypeptide. In certain embodiments, the hinge/spacer region of the CAR comprises a CD28 polypeptide and the transmembrane domain of the CAR comprises an ICOS polypeptide.

5.2.2.4. Intracellular Signaling Domain of the CAR

A. CD3ζ

In certain embodiments, the CAR comprises an intracellular signaling domain. In certain embodiments, the intracellular signaling domain of the CAR comprises a CD3ζ polypeptide, which can activate or stimulate a cell (e.g., a cell of the lymphoid lineage, e.g., a T cell). Wild type (“native”) CD3 comprises three immunoreceptor tyrosine-based activation motifs (“ITAMs”) (e.g., ITAM1, ITAM2 and ITAM3), three basic-rich stretch (BRS) regions (BRS1, BRS2 and BRS3), and transmits an activation signal to the cell (e.g., a cell of the lymphoid lineage, e.g., a T cell) after antigen is bound. The intracellular signaling domain of the native CD3ζ-chain is the primary transmitter of signals from endogenous TCRs.

In certain embodiments, the intracellular signaling domain of the CAR comprises a native CD3ζ polypeptide. In certain embodiments, the native CD3ζ polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the sequence with a NCBI Reference No: NP_932170 (SEQ ID No: 94) or a fragment thereof. In certain embodiments, the native CD3ζ polypeptide comprises or consists of an amino acid sequence that is a consecutive portion of SEQ ID NO: 94, which is at least about 20, or at least about 30, or at least about 40, or at least about 50, or at least about 100, or at least about 110, and up to about 164 amino acids in length. In certain embodiments, a native CD3ζ polypeptide comprises or consists of an amino acid sequence of amino acids 1 to 50, 50 to 100, 100 to 150, 50 to 164, 55 to 164, or 150 to 164 of SEQ ID NO: 94. In certain embodiments, a native CD3ζ polypeptide comprises or consists of an amino acid sequence of amino acids 52 to 164 of SEQ ID NO: 94.

SEQ ID NO: 94 is provided below:

| [SEQ ID NO: 94] | |
|---|---|
| 1 | MKWKALFTAA ILQAQLPITE AQSFGLLDPK LCYLLDGILF IYGVILTALF LRVKFSRSAD |
| 61 | APAYQQGQNQ LYNELNLGRR EEYDVLDKRR GRDPEMGGKP QRRKNPQEGL YNELQKDKMA |
| 121 | EAYSEIGMKG ERRRGKGHDG LYQGLSTATK DTYDALHMQA LPPR |

In certain embodiments, a CD3ζ polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 95 or a fragment thereof, and/or may optionally comprise up to one or up to two or up to three conservative amino acid substitutions. SEQ ID NO: 95 is provided below:

[SEQ ID NO: 95]
RVKFSRSADA PAYQQGQNQL YNELNLGRRE EYDVLDKRRG
RDPEMGGKPR RKNPQEGLYN ELQKDKMAEA YSEIGMKGER
RRGKGHDGLY QGLSTATKDT YDALHMQALP PR

An exemplary nucleotide sequence encoding the amino acid sequence of SEQ ID NO: 95 is set forth in SEQ ID NO: 96, which is provided below.

[SEQ ID NO: 96]
AGAGTGAAGTTCAGCAGGAGCGCAGACGCCCCCGCGTACCAGCAGGGC
CAGAACCAGCTCTATAACGAGCTCAATCTAGGACGAAGAGAGGAGTAC
GATGTTTTGGACAAGAGACGTGGCCGGGACCCTGAGATGGGGGGAAAG
CCGAGAAGGAAGAACCCTCAGGAAGGCCTGTACAATGAACTGCAGAAA
GATAAGATGGCGGAGGCCTACAGTGAGATTGGGATGAAAGGCGAGCGC
CGGAGGGGCAAGGGGCACGATGGCCTTTACCAGGGTCTCAGTACAGCC
ACCAAGGACACCTACGACGCCCTTCACATGCAGGCCCTGCCCCCTCGC

In certain embodiments, the intracellular signaling domain of the CAR comprises a modified CD3ζ polypeptide. In certain embodiments, the intracellular signaling domain of the CAR comprises a modified human CD3ζ polypeptide. In certain embodiments, the modified CD3ζ polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 35 or a fragment thereof, and/or may optionally comprise up to one or up to two or up to three conservative amino acid substitutions. SEQ ID NO: 35 is provided below:

[SEQ ID NO: 35]
RVKFSRSADA PAYQQGQNQL YNELNLGRRE EYDVLDKRRG
RDPEMGGKPR RKNPQEGLFN ELQKDKMAEA FSEIGMKGER
RRGKGHDGLF QGLSTATKDT FDALHMQALP PR

An exemplary nucleotide sequence encoding the amino acid sequence of SEQ ID NO: 35 is set forth in SEQ ID NO: 55, which is provided below.

[SEQ ID NO: 55]
agagtgaagttcagcaggagcgcagacgcccccgcgtaccagcagggc
cagaaccagctctataacgagctcaatctaggacgaagagaggagtac
gatgttttggacaagagacgtggccgggaccctgagatggggggaaag
ccgagaaggaagaaccctcaggaaggcctgtTcaatgaactgcagaaa
gataagatggcggaggcctTcagtgagattgggatgaaaggcgagcgc
cggaggggcaaggggcacgatggcctttTccaggggctcagtacagcc
accaaggacacctTcgacgcccttcacatgcaggccctgccccctcgc

In certain embodiments, the modified CD3ζ polypeptide comprises one, two or three ITAM variants. In certain embodiments, the modified CD3ζ polypeptide comprises a native ITAM1. In certain embodiments, the native ITAM1 comprises or consist of the amino acid sequence set forth in SEQ ID NO: 23.

[SEQ ID NO: 23]
QNQLYNELNLGRREEYDVLDKR

An exemplary nucleic acid sequence encoding the amino acid sequence of SEQ ID NO: 23 is set forth in SEQ ID NO: 24, which is provided below.

[SEQ ID NO: 24]
cagaaccagctctataacgagctcaatctagga cgaagagaggagta
cgatgttttggacaagaga

In certain embodiments, the modified CD3ζ polypeptide comprises an ITAM1 variant comprising one or more loss-of-function mutations. In certain embodiments, the ITAM1 variant comprises or consists of two loss-of-function mutations. In certain embodiments, each of the one or more (e.g., two) loss of function mutations comprises or consists of a mutation of a tyrosine residue in ITAM1. In certain embodiments, the ITAM1 variant (e.g., the variant consisting of two loss-of-function mutations) comprises or consists of the amino acid sequence set forth in SEQ ID NO: 25, which is provided below.

[SEQ ID NO: 25]
QNQLFNELNLGRREEFDVLDKR

An exemplary nucleic acid sequence encoding the amino acid sequence of SEQ ID NO: 25 is set forth in SEQ ID NO: 26, which is provided below.

[SEQ ID NO: 26]
cagaaccagctctTtaacgagctcaatctagga cgaagagaggagtT
cgatgttttggacaagaga

In certain embodiments, the modified CD3ζ polypeptide comprises a native ITAM2. In certain embodiments, the native ITAM2 comprises or consists of the amino acid sequence set forth in SEQ ID NO: 27, which is provided below.

[SEQ ID NO: 27]
QEGLYNELQKDKMAEAYSEIGMK

An exemplary nucleic acid sequence encoding the amino acid sequence of SEQ ID NO: 27 is set forth in SEQ ID NO: 28, which is provided below.

[SEQ ID NO: 28]
caggaaggcctgtacaatgaactgcagaaagataagatggcggaggcctac
agtgagattgggatgaaa

In certain embodiments, the modified CD3ζ polypeptide comprises an ITAM2 variant comprising one or more loss-of-function mutations. In certain embodiments, the ITAM2 variant comprises or consists of two loss-of-function mutations. In certain embodiments, each of the one or more (e.g., two) the loss of function mutations comprises or consists of a mutation of a tyrosine residue in ITAM2. In certain embodiments, the ITAM2 variant (e.g., a variant consisting of two loss-of-function mutations) comprises or consists of the amino acid sequence set forth in SEQ ID NO: 29, which is provided below.

[SEQ ID NO: 29]
QEGLFNELQKDKMAEAFSEIGMK

An exemplary nucleic acid sequence encoding the amino acid sequence of SEQ ID NO: 29 is set forth in SEQ ID NO: 30, which is provided below.

[SEQ ID NO: 30]
caggaaggcctgtTcaatgaactgcagaaagataagatggcggaggcctTc
agtgagattgggatgaaa

In certain embodiments, the modified CD3ζ polypeptide comprises a native ITAM3. In certain embodiments, the native ITAM3 comprises or consists of the amino acid sequence set forth in SEQ ID NO: 31, which is provided below.

[SEQ ID NO: 31]
HDGLYQGLSTATKDTYDALHMQ

An exemplary nucleic acid sequence encoding the amino acid sequence of SEQ ID NO: 31 is set forth in SEQ ID NO: 32, which is provided below.

[SEQ ID NO: 32]
cacgatggcctttaccagggtctcagtacagccaccaaggacacctacgac
gcccttcacatgcag

In certain embodiments, the modified CD3ζ polypeptide comprises an ITAM3 variant comprising one or more loss-of-function mutations. In certain embodiments, the ITAM3 variant comprises or consists of two loss-of-function mutations. In certain embodiments, each of the one or more (e.g., two) the loss of function mutations comprises or consists of a mutation of a tyrosine residue in ITAM3. In certain embodiments, the ITAM3 variant (e.g., a variant consisting of two loss-of-function mutations) comprises or consists of the amino acid sequence set forth in SEQ ID

NO: 33, which is provided below.

[SEQ ID NO: 33]
HDGLFQGLSTATKDTFDALHMQ

An exemplary nucleic acid sequence encoding the amino acid sequence of SEQ ID NO: 33 is set forth in SEQ ID NO: 34, which is provided below.

[SEQ ID NO: 34]
cacgatggcctttTccaggggctcagtacagccaccaaggacacctTcgac
gcccttcacatgcag

In certain embodiments, the intracellular signaling domain of the CAR comprises a modified CD3ζ polypeptide comprising an ITAM1 variant comprising or consisting of one or more loss-of-function mutations, an ITAM2 variant comprising or consisting of one or more loss-of-function mutations, and/or an ITAM3 variant comprising or consisting of one or more loss-of-function mutations, or a combination thereof.

In certain embodiments, the intracellular signaling domain of the CAR comprises a modified CD3ζ polypeptide comprising an ITAM2 variant comprising or consisting of one or more (e.g., two) loss-of-function mutations and an ITAM3 variant comprising or consisting of one or more (e.g., two) loss-of-function mutations. In certain embodiments, the intracellular signaling domain of the CAR comprises a modified CD3ζ polypeptide comprising a native ITAM1, an ITAM2 variant comprising or consisting of two loss-of-function mutations and an ITAM3 variant comprising or consisting of two loss-of-function mutations. In certain embodiments, the intracellular signaling domain of the CAR comprises a modified CD3ζ polypeptide comprising a native ITAM1 consisting of the amino acid sequence set forth in SEQ ID NO: 23, an ITAM2 variant consisting of the amino acid sequence set forth in SEQ ID NO: 29, and an ITAM3 variant consisting of the amino acid sequence set forth in SEQ ID NO: 33 (e.g., a construct designated as “1XX”). In certain embodiments, the modified CD3ζ polypeptide comprises or consists of the amino acid sequence set forth in SEQ ID NO: 35.

In certain embodiments, the intracellular signaling domain of the CAR comprises a modified CD3ζ polypeptide comprising an ITAM1 variant comprising or consisting of one or more (e.g., two) loss-of-function mutations and an ITAM3 variant comprising or consisting of one or more (e.g., two) loss-of-function mutations. In certain embodiments, the intracellular signaling domain of the CAR comprises a modified CD3ζ polypeptide comprising an ITAM1 variant comprising or consisting of two loss-of-function mutations, a native ITAM2, and an ITAM3 variant comprising or consisting of two loss-of-function mutations. In certain embodiments, the intracellular signaling domain of the CAR comprises a modified CD3ζ polypeptide comprising an ITAM1 variant consisting of the amino acid sequence set forth in SEQ ID NO: 25, a native ITAM2 consisting of the amino acid sequence set forth in SEQ ID NO: 27, and an ITAM3 variant consisting of the amino acid sequence set forth in SEQ ID NO: 33 (e.g., a construct designated as “X2X”).

In certain embodiments, the intracellular signaling domain of the CAR comprises a modified CD3ζ polypeptide comprising an ITAM1 variant comprising or consisting of one or more (e.g., two) loss-of-function mutations and an ITAM2 variant comprising or consisting of one or more (e.g., two) loss-of-function mutations. In certain embodiments, the intracellular signaling domain of the CAR comprises a modified CD3ζ polypeptide comprising or consisting of an ITAM1 variant comprising two loss-of-function mutations, an ITAM2 variant comprising or consisting of two loss-of-function mutations, and a native ITAM3. In certain embodiments, the intracellular signaling domain of the CAR comprises a modified CD3ζ polypeptide comprising an ITAM1 variant consisting of the amino acid sequence set forth in SEQ ID NO: 25, an ITAM2 variant consisting of the amino acid sequence set forth in SEQ ID NO: 29, and a native ITAM3 consisting of the amino acid sequence set forth in SEQ ID NO: 31 (e.g., a construct designated as “XX3”).

In certain embodiments, the intracellular signaling domain of the CAR comprises a modified CD3ζ polypeptide comprising an ITAM1 variant comprising one or more (e.g., two) loss-of-function mutations. In certain embodiments, the intracellular signaling domain of the CAR comprises a modified CD3ζ polypeptide comprising an ITAM1 variant comprising or consisting of two loss-of-function mutations, a native ITAM2, and a native ITAM3. In certain embodiments, the intracellular signaling domain of the CAR comprises a modified CD3ζ polypeptide comprising an ITAM1 variant consisting of the amino acid sequence set forth in SEQ ID NO: 25, a native ITAM2 consisting of the amino acid sequence set forth in SEQ ID NO: 27 and a native ITAM3 consisting of the amino acid sequence set forth in SEQ ID NO: 31 (e.g., a construct designated as “X23”).

In certain embodiments, the intracellular signaling domain of the CAR comprises a modified CD3ζ polypeptide comprising a native ITAM1, a native ITAM2, and an ITAM3 variant comprising one or more (e.g., two) loss-of-function mutations. In certain embodiments, the intracellular signaling domain of the CAR comprises a modified CD3ζ polypeptide comprising a native ITAM1, a native ITAM2, and an ITAM1 variant comprising or consisting of two loss-of-function mutations. In certain embodiments, the intracellular signaling domain of the CAR comprises a modified CD3ζ polypeptide comprising a native ITAM1 consisting of the amino acid sequence set forth in SEQ ID NO: 23, a native ITAM2 consisting of the amino acid sequence set forth in SEQ ID NO: 27 and an ITAM3 variant consisting of the amino acid sequence set forth in SEQ ID NO: 33 (e.g., a construct designated as “12X”).

In certain embodiments, the intracellular signaling domain of the CAR comprises a modified CD3ζ polypeptide comprising a native ITAM1, an ITAM2 variant comprising one or more (e.g., two) loss-of-function mutations, and a native ITAM3. In certain embodiments, the intracellular signaling domain of the CAR comprises a modified CD3ζ polypeptide comprising a native ITAM1, an ITAM2 variant comprising or consisting of two loss-of-function mutations, and a native ITAM3. In certain embodiments, the intracellular signaling domain of the CAR comprises a modified CD3ζ polypeptide comprising a native ITAM1 consisting of the amino acid sequence set forth in SEQ ID NO: 23, an ITAM2 variant consisting of the amino acid sequence set forth in SEQ ID NO: 29 and a native ITAM3 variant consisting of the amino acid sequence set forth in SEQ ID NO: 31 (e.g., a construct designated as “1X3”).

In certain embodiments, the intracellular signaling domain of the CAR comprises a modified CD3ζ polypeptide comprising a deletion of one or two ITAMs. In certain embodiments, the modified CD3ζ polypeptide comprises or consists of a deletion of ITAM1 and ITAM2, e.g., the modified CD3ζ polypeptide comprises a native ITAM3 or an ITAM3 variant, and does not comprise an ITAM1 or an ITAM2. In certain embodiments, the modified CD3ζ polypeptide comprises a native ITAM3 consisting of the amino acid sequence set forth in SEQ ID NO: 31, and does not comprise an ITAM1 (native or modified), or an ITAM2 (native or modified) (e.g., a construct designated as “D12”).

In certain embodiments, the modified CD3ζ polypeptide comprises or consists of a deletion of ITAM2 and ITAM3, e.g., the modified CD3ζ polypeptide comprises a native ITAM1 or an ITAM1 variant, and does not comprise an ITAM2 or an ITAM3. In certain embodiments, the modified CD3 polypeptide comprises a native ITAM1 consisting of the amino acid sequence set forth in SEQ ID NO: 23, and does not comprise an ITAM2 (native or modified), or an ITAM3 (native or modified) (e.g., a construct designated as “D23”).

In certain embodiments, the modified CD3ζ polypeptide comprises or consists of a deletion of ITAM1 and ITAM3, e.g., the modified CD3ζ polypeptide comprises a native ITAM2 or an ITAM2 variant, and does not comprise an ITAM1 or an ITAM3. In certain embodiments, the modified CD3ζ polypeptide comprises a native ITAM2 consisting of the amino acid sequence set forth in SEQ ID NO: 27, and does not comprise an ITAM1 (native or modified), or an ITAM3 (native or modified) (e.g., a construct designated as “D13”).

In certain embodiments, the modified CD3ζ polypeptide comprises or consists of a deletion of ITAM1, e.g., the modified CD3ζ polypeptide comprises a native ITAM2 or an ITAM2 variant, and a native ITAM3 or an ITAM3 variant, and does not comprise an ITAM1 (native or modified).

In certain embodiments, the modified CD3ζ polypeptide comprises or consists of a deletion of ITAM2, e.g., the modified CD3ζ polypeptide comprises a native ITAM1 or an ITAM1 variant, and a native ITAM3 or an ITAM3 variant, and does not comprise an ITAM2 (native or modified).

In certain embodiments, the modified CD3ζ polypeptide comprises or consists of a deletion of ITAM3, e.g., the modified CD3ζ polypeptide comprises a native ITAM1 or an ITAM1 variant, and a native ITAM2 or an ITAM2 variant, and does not comprise an ITAM3 (native or modified).

B. Co-Stimulatory Signaling Region

In certain embodiments, the intracellular signaling domain of the CAR further comprises at least a co-stimulatory signaling region. In certain embodiments, the co-stimulatory signaling region comprises at least a portion of a co-stimulatory molecule, which can provide optimal lymphocyte activation.

As used herein, “co-stimulatory molecules” refer to cell surface molecules other than antigen receptors or their ligands that are required for an efficient response of lymphocytes to antigen. Non-limiting examples of co-stimulatory molecules include CD28, 4-1BB, OX40, ICOS, DAP-10, CD27, CD40, and NKGD2. The co-stimulatory molecule can bind to a co-stimulatory ligand, which is a protein expressed on cell surface that upon binding to its receptor produces a co-stimulatory response, i.e., an intracellular response that effects the stimulation provided when an antigen binds to its CAR molecule. Co-stimulatory ligands include, but are not limited to CD80, CD86, CD70, OX40L, and 4-1BBL. As one example, a 4-1BB ligand (i.e., 4-1BBL) may bind to 4-1BB (also known as “CD137”) for providing an intracellular signal that in combination with a CAR signal induces an effector cell function of the CAR⁺ T cell. CARs comprising an intracellular signaling domain that comprises a co-stimulatory signaling region comprising 4-1BB, ICOS or DAP-10 are disclosed in U.S. Pat. No. 7,446,190, which is herein incorporated by reference in its entirety.

In certain embodiments, the intracellular signaling domain of the CAR comprises a co-stimulatory signaling region that comprises a CD28 polypeptide (e.g., an intracellular domain of CD28 or a portion thereof). In certain embodiments, the co-stimulatory signaling region comprises an intracellular domain of human CD28 or a portion thereof. In certain embodiments, the co-stimulatory signaling region comprises a CD28 polypeptide comprising or consisting of amino acids 180 to 220 of SEQ ID NO: 90.

In certain embodiments, the co-stimulatory signaling region comprises a CD28 polypeptide comprising or consisting of the amino acid sequence set forth in SEQ ID NO: 101 (or amino acids 180 to 220 of SEQ ID NO: 90). SEQ ID NO: 101 is provided below.

[SEQ ID NO: 101]
RSKRSRLLHS DYMNMTPRRP GPTRKHYQPY APPRDFAAYR S

An exemplary nucleotide sequence encoding the amino acid sequence of SEQ ID NO: 101 (or amino acids 180 to 220 of SEQ ID NO: 90) is set forth in SEQ ID NO: 102, which is provided below.

[SEQ ID NO: 102]
AGGAGTAAGAGGAGCAGGCTCCTGCACAGTGACTACATGAACATGACTCCC
CGCCGCCCCGGGCCCACCCGCAAGCATTACCAGCCCTATGCCCCACCACGC
GACTTCGCAGCCTATCGCTCC

In certain embodiments, the co-stimulatory signaling region comprises a CD28 polypeptide comprising or consisting of the amino acid sequence set forth in SEQ ID NO: 108 (or amino acids 180 to 219 of SEQ ID NO: 90). SEQ ID NO: 108 is provided below.

[SEQ ID NO: 108]
RSKRSRLLHS DYMNMTPRRP GPTRKHYQPY APPRDFAAYR K

In certain embodiments, the co-stimulatory signaling region comprises an intracellular domain of mouse CD28 or a portion thereof. In certain embodiments, the co-stimulatory signaling region comprises or consists of amino acids 178 to 218 of SEQ ID NO: 97.

An exemplary nucleotide sequence encoding amino acids 178 to 218 of SEQ ID NO: 97 is set forth in SEQ ID NO: 98, which is provided below.

[SEQ ID NO: 98]
aat agtagaagga acagactcct tcaaagtgac tacatgaaca
tgactccccg gaggcctggg ctcactcgaa agccttacca
gccctacgcc cctgccagag actttgcagc gtaccgcccc

In certain embodiments, the co-stimulatory signaling region comprises or consists of a CD28 polypeptide comprising or consisting of the amino acid sequence set forth in SEQ ID NO: 99. SEQ ID NO: 99 is provided below:

[SEQ ID NO: 99]
NSRRNRLLQS DYMNMTPRRP GLTRKPYQPY APARDFAAYR P.

An exemplary nucleic acid sequence encoding the amino acid sequence of SEQ ID NO: 99 is set forth in SEQ ID NO: 100, which is provided below.

[SEQ ID NO: 100]
AATAGTAGAAGGAACAGACTCCTTCAAAGTGACTACATGAACATGACTCCC
CGGAGGCCTGGGCTCACTCGAAAGCCTTACCAGCCCTACGCCCCTGCCAGA
GACTTTGCAGCGTACCGCCCC

In certain embodiments, the co-stimulatory signaling region comprises a portion of a first co-stimulatory molecule and a portion of a second co-stimulatory molecule, e.g., an intracellular domain of CD28 and an intracellular domain of 4-1BB or an intracellular domain of CD28 and an intracellular domain of OX40.

In certain embodiments, the co-stimulatory signaling region comprises a 4-1BB polypeptide (e.g., an intracellular domain of 4-1BB or a portion thereof). In certain embodiments, the co-stimulatory signaling region comprises an intracellular domain of human 4-1BB or a portion thereof. 4-1BB can act as a tumor necrosis factor (TNF) ligand and have stimulatory activity. In certain embodiments, the 4-1BB polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the sequence with a NCBI Reference No: NP_001552.2 (SEQ ID NO: 103) or a fragment thereof, and/or may optionally comprise up to one or up to two or up to three conservative amino acid substitutions. In certain embodiments, the 4-1BB polypeptide comprises or consists of an amino acid sequence that is a consecutive portion of SEQ ID NO: 103 which is at least about 20, at least about 25, or at least about 30, or at least about 40, or at least about 50, and up to about 255 amino acids in length. In certain embodiments, the 4-1BB polypeptide comprises or consists of an amino acid sequence of amino acids 1 to 255, 1 to 50, 50 to 100, 100 to 150, 150 to 200, 214 to 255, or 200 to 255 of SEQ ID NO: 103. In certain embodiments, the co-stimulatory signaling region comprises a 4-1BB polypeptide comprising or consisting of SEQ ID NO: 104 (or amino acids 214 to 255 of SEQ ID NO: 103). SEQ ID NOs: 103 and 104 are provided below:

| [SEQ ID NO: 103] | |
|---|---|
| 1 | MGNSCYNIVA TLLLVLNFER TRSLQDPCSN CPAGTFCDNN RNQICSPCPP NSFSSAGGQR |
| 61 | TCDICRQCKG VFRTRKECSS TSNAECDCTP GFHCLGAGCS MCEQDCKQGQ ELTKKGCKDC |
| 121 | CFGTFNDQKR GICRPWTNCS LDGKSVLVNG TKERDVVCGP SPADLSPGAS SVTPPAPARE |
| 181 | PGHSPQIISF FLALTSTALL FLLFFLTLRF SVVKRGRKKL LYIFKQPFMR PVQTTQEEDG |
| 241 | CSCRFPEEEE GGCEL |

[SEQ ID NO: 104]
KRGRKKLLYI FKQPFMRPVQ TTQEEDGCSC RFPEEEEGGC EL.

An exemplary nucleotide sequence encoding the amino acid sequence of SEQ ID NO: 104 (or amino acids 214 to 255 of SEQ ID NO: 103) is set forth in SEQ ID NO: 105, which is provided below.

[SEQ ID NO: 105]
AAACGGGGCAGAAAGAAACTCCTGTATATATTCAAACAACCATTTATGAGA
CCAGTACAAACTACTCAAGAGGAAGATGGCTGTAGCTGCCGATTTCCAGAA
GAAGAAGAAGGAGGATGTGAACTG

In certain embodiments, the co-stimulatory signaling region comprises an OX40 polypeptide (e.g., an intracellular domain of OX40 or a portion thereof). In certain embodiments, the co-stimulatory signaling region comprises an intracellular domain of human OX40 or a portion thereof. In certain embodiments, the OX40 polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the sequence with a NCBI Reference No: NP_003318.1 (SEQ ID NO: 106) or a fragment thereof, and/or may optionally comprise up to one or up to two or up to three conservative amino acid substitutions. In certain embodiments, the OX40 polypeptide comprises or consists of an amino acid sequence that is a consecutive portion of SEQ ID NO: 106 which is at least about 20, at least about 25, or at least about 30, or at least about 40, or at least about 50, and up to about 277 amino acids in length. In certain embodiments, the OX40 polypeptide comprises or consists of an amino acid sequence of amino acids 1 to 277, 1 to 50, 50 to 100, 100 to 150, 150 to 200, or 200 to 277 of SEQ ID NO: 106. SEQ ID NO: 106 is provided below.

```
[SEQ ID NO: 106]
    1 MCVGARRLGR GPCAALLLLG LGLSTVTGLH CVGDTYPSND RCCHECRPGN GMVSRCSRSQ
  61 NTVCRPCGPG FYNDVVSSKP CKPCTWCNLR SGSERKQLCT ATQDTVCRCR AGTQPLDSYK
121 PGVDCAPCPP GHFSPGDNQA CKPWTNCTLA GKHTLQPASN SSDAICEDRD PPATQPQETQ
181 GPPARPITVQ PTEAWPRTSQ GPSTRPVEVP GGRAVAAILG LGLVLGLLGP LAILLALYLL
241 RRDQRLPPDA HKPPGGGSFR TPIQEEQADA HSTLAKI
```

In certain embodiments, the co-stimulatory signaling region comprises an ICOS polypeptide (e.g., an intracellular domain of ICOS or a portion thereof). In certain embodiments, the co-stimulatory signaling region comprises an intracellular domain of human ICOS or a portion thereof. In certain embodiments, the ICOS polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical homologous to the sequence with a NCBI Reference No: NP_036224 (SEQ ID NO: 65) or a fragment thereof, and/or may optionally comprise up to one or up to two or up to three conservative amino acid substitutions. In certain embodiments, the ICOS polypeptide comprises or consists of an amino acid sequence that is a consecutive portion of SEQ ID NO: 65 which is at least about 20, at least about 25, or at least about 30, or at least about 40, or at least about 50, and up to about 199 amino acids in length. In certain embodiments, the ICOS polypeptide comprises or consists of an amino acid sequence of amino acids 1 to 199, 1 to 50, 50 to 100, 100 to 150, or 150 to 199 of SEQ ID NO: 65. SEQ ID NO: 65 is provided below.

```
[SEQ ID NO: 65]
    1 MKSGLWYFFL FCLRIKVLTG EINGSANYEM FIFHNGGVQI LCKYPDIVQQ FKMQLLKGGQ
  61 ILCDLIKTKG SGNTVSIKSL KFCHSQLSNN SVSFFLYNLD HSHANYYFCN LSIFDPPPFK
121 VTLIGGYLHI YESQLCCQLK FWLPIGCAAF VVVCILGCIL ICWLTKKKYS SSVHDPNGEY
181 MFMRAVNTAK KSRLTDVTL
```

In certain embodiments, a presently disclosed mesothelin-targeted CAR further comprises an inducible promoter, for expressing nucleic acid sequences in human cells. Promoters for use in expressing CAR genes can be a constitutive promoter, such as ubiquitin C (UbiC) promoter.

In certain embodiments, mutation sites and/or junction between domains/motifs/regions of the CAR derived from different proteins are de-immunized. Immunogenicity of junctions between different CAR moieties can be predicted using NetMHC 4.0 Server. For each peptide containing at least one amino acid from next moiety, binding affinity to HLA A, B and C, for all alleles, can be predicted. A score of immunogenicity of each peptide can be assigned for each peptide. Immunogenicity score can be calculated using the formula Immunogenicity score=[(50-binding affinity)*HLA frequency]ₙ. n is the number of prediction for each peptide.

5.2.2.5. Exemplified CARs

In certain embodiments, the mesothelin-targeted CAR comprises:

(a) an extracellular antigen-binding domain comprising a V_{H} comprising a CDR1 consisting of the amino acid sequence set forth in SEQ ID NO: 76, a CDR2 consisting of the amino acid sequence set forth in SEQ ID NO: 77, and a CDR3 consisting of the amino acid sequence set forth in SEQ ID NO: 78; and a V_{L} comprising a CDR1 consisting of the amino acid sequence set forth in SEQ ID NO: 79, a CDR2 consisting of the amino acid sequence set forth in SEQ ID NO: 80, and a CDR3 consisting of the amino acid sequence set forth in SEQ ID NO: 81;

(b) a transmembrane domain comprising a CD28 polypeptide (e.g., a transmembrane domain of human CD28 or a portion thereof);

(c) a CD28 hinge/spacer region (e.g., a hinge/spacer region of human CD28 or a portion thereof); and

(d) an intracellular signaling domain comprising (i) a modified CD3 polypeptide (e.g., a modified human CD3ζ polypeptide) comprising a native ITAM1, an ITAM2 variant consisting of two loss-of-function mutations, and an ITAM3 variant consisting of two loss-of-function mutations, and (ii) a co-stimulatory signaling region comprising a CD28 polypeptide (e.g., a human CD28 polypeptide, e.g., an intracellular domain of a human CD28 or a portion thereof).

In certain embodiments, the transmembrane domain comprises a CD28 polypeptide consisting of the amino acid sequence set forth in SEQ ID NO: 92 (or amino acids 153 to 179 of SEQ ID NO: 90).

In certain embodiments, the CD28 hinge/spacer region consists of the amino acid sequence set forth in SEQ ID NO: 15 (or amino acids 114 to 152 of SEQ ID NO: 90).

In certain embodiments, the modified CD3ζ polypeptide consists of the amino acid sequence set forth in SEQ ID NO: 35.

In certain embodiments, the co-stimulatory signaling region comprises a CD28 polypeptide consisting of the amino acid sequence set forth in SEQ ID NO: 101 (or amino acids 180 to 220 of SEQ ID NO: 90).

In certain embodiments, the CAR comprises an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 56. In certain embodiments, the CAR comprises an amino acid sequence set forth in SEQ ID NO: 56. SEQ ID NO: 56 is provided below.

[SEQ ID NO: 56]
QVQLQESGPGLVKPSETLSLTCTVSGGSVSSGSYYWSWIRQPPGKGLEWIGYIYYSGSTNYNPSLKSRVTISVDTSKNQF
SLKLSSVTAADTAVYYCAREGKNGAFDIWGQGTMVTVSSGGGGSGGGGSGGGGSRHQMTQSPSSLSASVGDRVTITCRAS
QSISSYLNWYQQKPGKAPKLLIYAASSLQSGVPSRFSGSGSGTDFTLTISSLQPEDFATYYCQQSYSTPLTFGGGTKVEI
KRTAAAIEVMYPPPYLDNEKSNGTIIHVKGKHLCPSPLFPGPSKPFWVLVVVGGVLACYSLLVTVAFIIFWVRSKRSRLL
HSDYMNMTPRRPGPTRKHYQPYAPPRDFAAYRSRVKFSRSADAPAYQQGQNQLYNELNLGRREEYDVLDKRRGRDPEMGG
KPRRKNPQEGLFNELQKDKMAEAFSEIGMKGERRRGKGHDGLFQGLSTATKDTFDALHMQALPPR

An exemplary nucleotide sequence encoding the amino acid sequence of SEQ ID NO: 56 is set forth in SEQ ID NO: 57, which is provided below.

[SEQ ID NO: 57]
CAGGTTCAGCTTCAGGAGAGTGGCCCAGGCCTGGTGAAGCCAAGTGAGACTCTCAGCTTGACTTGCACAGTTTCTGGAGG
CAGTGTCTCCTCAGGCAGCTATTATTGGTCCTGGATTCGGCAGCCCCCTGGGAAAGGCCTGGAGTGGATTGGGTACATAT
ATTACAGTGGCAGCACAAATTACAATCCATCCCTGAAGTCTCGAGTAACTATCAGTGTGGACACAAGCAAGAATCAGTTT
TCACTCAAACTGTCTTCTGTGACTGCTGCTGACACTGCTGTTTATTATTGTGCCAGGGAGGGGAAAAATGGGGCATTTGA
TATTTGGGGTCAGGGCACAATGGTGACAGTCAGCTCTGGAGGTGGAGGCTCAGGAGGAGGAGGCAGTGGAGGTGGTGGGT
CACGCCATCAGATGACTCAGTCCCCCTCCAGTCTTTCTGCCTCAGTTGGGGATAGAGTGACCATCACATGCAGAGCAAGT
CAGAGCATATCATCCTATCTGAACTGGTACCAGCAGAAGCCAGGGAAAGCCCCCAAATTGCTGATTTATGCAGCCTCAAG
TCTCCAGAGTGGGGTGCCAAGCAGGTTCTCAGGCAGTGGCAGTGGGACAGATTTCACATTGACAATCAGCTCCCTCCAAC
CTGAAGATTTTGCCACCTACTATTGCCAGCAATCCTACAGCACGCCCCTGACTTTTGGAGGTGGCACAAAGGTAGAGATC
AAGAGGACTGCGGCCGCAATTGAAGTTATGTATCCTCCTCCTTACCTAGACAATGAGAAGAGCAATGGAACCATTATCCA
TGTGAAAGGGAAACACCTTTGTCCAAGTCCCCTATTTCCCGGACCTTCTAAGCCCTTTTGGGTGCTGGTGGTGGTTGGTG
GAGTCCTGGCTTGCTATAGCTTGCTAGTAACAGTGGCCTTTATTATTTTCTGGGTGAGGAGTAAGAGGAGCAGGCTCCTG
CACAGTGACTACATGAACATGACTCCCCGCCGCCCCGGGCCCACCCGCAAGCATTACCAGCCCTATGCCCCACCACGCGA
CTTCGCAGCCTATCGCTCCAGAGTGAAGTTCAGCAGGAGCGCAGACGCCCCCGCGTACCAGCAGGGCCAGAACCAGCTCT
ATAACGAGCTCAATCTAGGACGAAGAGAGGAGTACGATGTTTTGGACAAGAGACGTGGCCGGGACCCTGAGATGGGGGGA
AAGCCGAGAAGGAAGAACCCTCAGGAAGGCCTGTTCAATGAACTGCAGAAAGATAAGATGGCGGAGGCCTTCAGTGAGAT
TGGGATGAAAGGCGAGCGCCGGAGGGGCAAGGGGCACGATGGCCTTTTCCAGGGGCTCAGTACAGCCACCAAGGACACCT
TCGACGCCCTTCACATGCAGGCCCTGCCCCCTCGC

In certain embodiments, the CAR further comprises a CD8 leader. In certain embodiments, the CD8 leader comprises or consists of the amino acid sequence set forth in SEQ ID NO: 71.

An exemplary nucleotide sequence encoding the amino acid sequence of SEQ ID NO: 71 is set forth in SEQ ID NO: 120, which is provided below.

[SEQ ID NO: 120]
ATGGCCCTGCCAGTAACGGCTCTGCTGCTGCCACTTGCTCTGCTCCTCC
ATGCAGCCAGGCCT

In certain embodiments, the CAR comprises an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 43, which is provided below. In certain embodiments, the CAR comprises or consists of the amino acid sequence set forth in SEQ ID NO: 43. SEQ ID NO: 43 includes a CD8 leader consists of the amino acid sequence set forth in SEQ ID NO: 71. SEQ ID NO: 43 is provided below:

[SEQ ID NO: 43]
MALPVTALLLPLALLLHAARPQVQLQESGPGLVKPSETLSLTCTVSGGSVSSGSYYWSWI
RQPPGKGLEWIGYIYYSGSTNYNPSLKSRVTISVDTSKNQFSLKLSSVTAADTAVYYCAR
EGKNGAFDIWGQGTMVTVSSGGGGSGGGGSGGGGSRHQMTQSPSSLSASVGDRVTITCRA
SQSISSYLNWYQQKPGKAPKLLIYAASSLQSGVPSRFSGSGSGTDFTLTISSLQPEDFAT
YYCQQSYSTPLTFGGGTKVEIKRTAAAIEVMYPPPYLDNEKSNGTIIHVKGKHLCPSPLF
PGPSKPFWVLVVVGGVLACYSLLVTVAFIIFWVRSKRSRLLHSDYMNMTPRRPGPTRKHY
QPYAPPRDFAAYRSRVKFSRSADAPAYQQGQNQLYNELNLGRREEYDVLDKRRGRDPEMG
GKPRRKNPQEGLFNELQKDKMAEAFSEIGMKGERRRGKGHDGLFQGLSTATKDTFDALHM
QALPPR

An exemplary nucleotide sequence encoding the amino acid sequence of SEQ ID NO: 43 is set forth in SEQ ID NO: 44, which is provided below.

[SEQ ID NO: 44]
ATGGCCCTGCCAGTAACGGCTCTGCTGCTGCCACTTGCTCTGCTCCTCCATGCAGCCAGGCCTCAGGTTCAGCTTCA
GGAGAGTGGCCCAGGCCTGGTGAAGCCAAGTGAGACTCTCAGCTTGACTTGCACAGTTTCTGGAGGCAGTGTCTCCT
CAGGCAGCTATTATTGGTCCTGGATTCGGCAGCCCCCTGGGAAAGGCCTGGAGTGGATTGGGTACATATATTACAGT
GGCAGCACAAATTACAATCCATCCCTGAAGTCTCGAGTAACTATCAGTGTGGACACAAGCAAGAATCAGTTTTCACT
CAAACTGTCTTCTGTGACTGCTGCTGACACTGCTGTTTATTATTGTGCCAGGGAGGGGAAAAATGGGGCATTTGATA
TTTGGGGTCAGGGCACAATGGTGACAGTCAGCTCTGGAGGTGGAGGCTCAGGAGGAGGAGGCAGTGGAGGTGGTGGG
TCACGCCATCAGATGACTCAGTCCCCCTCCAGTCTTTCTGCCTCAGTTGGGGATAGAGTGACCATCACATGCAGAGC
AAGTCAGAGCATATCATCCTATCTGAACTGGTACCAGCAGAAGCCAGGGAAAGCCCCCAAATTGCTGATTTATGCAG
CCTCAAGTCTCCAGAGTGGGGTGCCAAGCAGGTTCTCAGGCAGTGGCAGTGGGACAGATTTCACATTGACAATCAGC
TCCCTCCAACCTGAAGATTTTGCCACCTACTATTGCCAGCAATCCTACAGCACGCCCCTGACTTTTGGAGGTGGCAC
AAAGGTAGAGATCAAGAGGACTGCGGCCGCAATTGAAGTTATGTATCCTCCTCCTTACCTAGACAATGAGAAGAGCA
ATGGAACCATTATCCATGTGAAAGGGAAACACCTTTGTCCAAGTCCCCTATTTCCCGGACCTTCTAAGCCCTTTTGG
GTGCTGGTGGTGGTTGGTGGAGTCCTGGCTTGCTATAGCTTGCTAGTAACAGTGGCCTTTATTATTTTCTGGGTGAG
GAGTAAGAGGAGCAGGCTCCTGCACAGTGACTACATGAACATGACTCCCCGCCGCCCCGGGCCCACCCGCAAGCATT
ACCAGCCCTATGCCCCACCACGCGACTTCGCAGCCTATCGCTCCAGAGTGAAGTTCAGCAGGAGCGCAGACGCCCCC
GCGTACCAGCAGGGCCAGAACCAGCTCTATAACGAGCTCAATCTAGGACGAAGAGAGGAGTACGATGTTTTGGACAA
GAGACGTGGCCGGGACCCTGAGATGGGGGGAAAGCCGAGAAGGAAGAACCCTCAGGAAGGCCTGTTCAATGAACTGC
AGAAAGATAAGATGGCGGAGGCCTTCAGTGAGATTGGGATGAAAGGCGAGCGCCGGAGGGGCAAGGGGCACGATGGC
CTTTTCCAGGGGCTCAGTACAGCCACCAAGGACACCTTCGACGCCCTTCACATGCAGGCCCTGCCCCCTCGC

### 5.2.3. Exemplified Polypeptide Compositions

In certain embodiments, the polypeptide composition comprises a mesothelin-targeted CAR comprising or consisting of the amino acid sequence set forth in SEQ ID NO: 56, and a PD-1 DN comprising or consisting of amino acids 21 to 165 of SEQ ID NO: 48.

In certain embodiments, the polypeptide composition comprises a mesothelin-targeted CAR comprising or consisting of the amino acid sequence set forth in SEQ ID NO: 56, and a PD-1 DN comprising or consisting of amino acids 1 to 165 of SEQ ID NO: 48.

In certain embodiments, the polypeptide composition comprises a mesothelin-targeted CAR comprising or consisting of the amino acid sequence set forth in SEQ ID NO: 56, and a PD-1 DN comprising or consisting of the amino acid sequence set forth in SEQ ID NO: 49.

In certain embodiments, the polypeptide composition comprises a mesothelin-targeted CAR comprising or consisting of the amino acid sequence set forth in SEQ ID NO: 56, and a PD-1 DN comprising or consisting of the amino acid sequence set forth in SEQ ID NO: 118.

In certain embodiments, the polypeptide composition comprises a mesothelin-targeted CAR comprising or consisting of the amino acid sequence set forth in SEQ ID NO: 56 and a CD8 leader comprising or consisting of the amino acid sequence set forth in SEQ ID NO: 71, and a PD-1 DN comprising or consisting of amino acids 21 to 165 of SEQ ID NO: 48.

In certain embodiments, the polypeptide composition comprises a mesothelin-targeted CAR comprising or consisting of the amino acid sequence set forth in SEQ ID NO: 56 and a CD8 leader comprising or consisting of the amino acid sequence set forth in SEQ ID NO: 71, and a PD-1 DN comprising or consisting of amino acids 1 to 165 of SEQ ID NO: 48.

In certain embodiments, the polypeptide composition comprises a mesothelin-targeted CAR comprising or consisting of the amino acid sequence set forth in SEQ ID NO: 56 and a CD8 leader comprising or consisting of the amino acid sequence set forth in SEQ ID NO: 71, and a PD-1 DN comprising or consisting of the amino acid sequence set forth in SEQ ID NO: 49.

In certain embodiments, the polypeptide composition comprises a mesothelin-targeted CAR comprising or consisting of the amino acid sequence set forth in SEQ ID NO: 56 and a CD8 leader comprising or consisting of the amino acid sequence set forth in SEQ ID NO: 71, and a PD-1 DN comprising or consisting of the amino acid sequence set forth in SEQ ID NO: 118.

### 5.3. Immunoresponsive Cells

The presently disclosed subject matter provides immunoresponsive cells comprising a polypeptide composition disclosed herein. In certain embodiments, the CAR is capable of activating the immunoresponsive cell. In certain embodiments, the polypeptide composition is capable of promoting an anti-tumor effect of the immunoresponsive cell. The immunoresponsive cells can be transduced with the polypeptide composition such that the cells co-express the CAR and the PD-1 DN.

The immunoresponsive cells of the presently disclosed subject matter can be cells of the lymphoid lineage. The lymphoid lineage, comprising B, T and natural killer (NK) cells, provides for the production of antibodies, regulation of the cellular immune system, detection of foreign agents in the blood, detection of cells foreign to the host, and the like. Non-limiting examples of immunoresponsive cells of the lymphoid lineage include T cells, Natural Killer (NK) cells, embryonic stem cells, and pluripotent stem cells (e.g., those from which lymphoid cells may be differentiated). T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., T_{EM} cells and T_{EMRA} cells, Regulatory T cells (also known as suppressor T cells), Natural killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR or a TCR. In certain embodiments, the immunoresponsive cell is a T cell. The T cell can be a CD4⁺ T cell or a CD8⁺ T cell. In certain embodiments, the T cell is a CD4⁺ T cell. In certain embodiments, the T cell is a CD8⁺ T cell.

Natural killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells). The immunoresponsive cells (e.g., T cells) can be autologous, non-autologous (e.g., allogeneic), or derived in vitro from engineered progenitor or stem cells.

In certain embodiments, the presently disclosed immunoresponsive cell comprises a mesothelin-targeted CAR comprising or consisting of the amino acid sequence set forth in SEQ ID NO: 56, and a PD-1 DN comprising or consisting of amino acids 1 to 165 of SEQ ID NO: 48.

In certain embodiments, the presently disclosed immunoresponsive cells that comprise one or more of the CAR and/or PD-1/DN polypeptides of the presently disclosed subject matter are allogeneic or autologous EBV-sensitized cytotoxic T lymphocytes (CTLs). For example, generation of EBV-sensitized cytotoxic T cells may involve isolating PBMCs from of an EBV-seropositive autologous or allogenic donor and enriching them for T cells by depletion of monocytes and NK cells. EBV-sensitized cytotoxic T cells may also be produced by contacting donor PBMCs or purified donor T cells with “stimulator” cells that express one or more EBV antigen(s) and present the EBV antigen(s) to unstimulated T cells, thereby causing stimulation and expansion of EBV-sensitized CTLs. Notably, in certain embodiments, such methods comprise obtaining a sample of cells (e.g., PBMC) from a subject comprising CD3⁺ cells and contacting said CD3⁺ cells with antigen and/or antigen-presenting stimulator cells. In certain embodiments, the CD3⁺ T cells are isolated from the sample prior to contacting the antigen by a method known in the art (e.g., positive selection of CD3⁺ cells from the sample and/or negative selection by depletion of undesired cells or components from the sample). In certain embodiments, such methods comprise selection using fluorescence activated cell sorting (FACS), with anti-CD3 beads (e.g., magnetic beads), plastic adherence, depletion of NK cells using anti-CD56, elutriation, and/or combinations thereof. EBV antigens include, for example, latent membrane protein (LMP) and EBV nuclear antigen (EBNA) proteins, such as LMP-1, LMP-2A, and LMP-2B and EBNA-1, EBNA-2, EBNA-3A, EBNA-3B, EBNA-3C and EBNA-LP. Cytotoxic T cells that comprise T cell receptor(s) which recognize one or more EBV-specific antigens are deemed to have been “sensitized” to those EBV antigen(s) and are therefore termed “EBV-sensitized cytotoxic T cells” herein. Known methods for generating allogeneic or autologous EBV-specific cytotoxic T cell populations that may comprise one or more of the presently disclosed CAR polypeptides are described, for example, in Barker et al., Blood 116(23):5045-49 (2010); Doubrovina et al., Blood 119(11):2644-56 (2012); Koehne et al., Blood 99(5):1730-40 (2002); and Smith et al., Cancer Res. 72(5):1116-25 (2012), which are incorporated by reference for these teachings. Similarly, cytotoxic T cells may be “sensitized” to other viral antigens, including cytomegalovirus (CMV), papillomavirus (e.g., HPV), adenovirus, polyomavirus (e.g., BKV, JCV, and Merkel cell virus), retrovirus (e.g., HTLV-I, also including lentivirus such as HIV), picomavirus (e.g., Hepatitis A virus), hepadnavirus (e.g., Hepatitis B virus), hepacivirus (e.g., Hepatitis C virus), deltavirus (e.g., Hepatitis D virus), hepevirus (e.g., Hepatitis E virus), and the like. In certain embodiments, the target antigen is from an oncovirus. In certain embodiments, the T cells used for generating the presently disclosed CAR-T cells are polyfunctional T-cells, e.g., those T cells that are capable of inducing multiple immune effector functions, that provide a more effective immune response to a pathogen than do cells that produce, for example, only a single immune effector (e.g. a single biomarker such as a cytokine or CD107a). Less-polyfunctional, monofunctional, or even “exhausted” T cells may dominate immune responses during chronic infections, thus negatively impacting protection against virus-associated complications. In certain embodiments, the presently disclosed CAR-T cells are polyfunctional. In certain embodiments, at least about 50% of the T cells used for generating the presently disclosed CAR-T cells are CD4⁺ T cells. In certain such embodiments, the T cells are less than about 50% CD4⁺ T cells. In certain embodiments, the T cells are predominantly CD4⁺ T cells.

In certain embodiments, at least about 50% of the T cells used for generating the presently disclosed CAR-T cells are CD8⁺ T cells. In certain such embodiments, the T cells are less than about 50% CD8⁺ T cells. In certain embodiments, the T cells are predominantly CD8⁺ T cells. In certain embodiments, the T cells (e.g., the sensitized T cells and/or CAR-T cells described herein) are stored in a cell library or bank before they are administered to the subject.

A presently disclosed immunoresponsive cell can further comprise at least one exogenous co-stimulatory ligand, such that the immunoresponsive cell co-expresses or is induced to co-express the mesothelin-specific CAR and the at least one exogenous co-stimulatory ligand. The interaction between the mesothelin-specific CAR and at least one co-stimulatory ligand provides a non-antigen-specific signal important for full activation of an immunoresponsive cell (e.g., T cell). Co-stimulatory ligands include, without limitation, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. TNF superfamily members include, without limitation, nerve growth factor (NGF), CD40L (CD40L)/CD154, CD137L/4-1BBL, TNF-α, CD134L/OX40L/CD252, CD27L/CD70, Fas ligand (FasL), CD30L/CD153, tumor necrosis factor beta (TNFβ)/lymphotoxin-alpha (LTα), lymphotoxin-beta (LTβ), CD257/B cell-activating factor (BAFF)/Blys/THANK/Tall-1, glucocorticoid-induced TNF Receptor ligand (GITRL), and TNF-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins—they possess an immunoglobulin domain (fold). Immunoglobulin superfamily ligands include, without limitation, CD80 and CD86, both ligands for CD28, PD-L1/(B7-H1) that ligands for PD-1.

In certain embodiments, the at least one co-stimulatory ligand is selected from the group consisting of 4-1BBL, CD80, CD86, CD70, OX40L, CD48, TNFRSF14, PD-L1, and combinations thereof. In certain embodiments, the co-stimulatory ligand is 4-1BBL. 4-1BBL can be covalently joined to the 5′ terminus of the extracellular antigen-binding domain of the mesothelin-targeted CAR. Alternatively, 4-1BBL can be covalently joined to the 3′ terminus of the intracellular signaling domain of the mesothelin-targeted CAR.

Furthermore, a presently disclosed immunoresponsive cell can further comprise at least one exogenous cytokine, such that the immunoresponsive cell co-expresses or is induced to co-express the mesothelin-specific CAR and the at least one exogenous cytokine. In certain embodiments, the at least one exogenous cytokine is selected from the group consisting of IL-2, IL-3, IL-6, IL-7, IL-11, IL-12, IL-15, IL-17, and IL-21. In certain embodiments, the at least one exogenous cytokine comprises IL-12. In certain embodiments, the immunoresponsive cell co-expresses the mesothelin-targeted CAR and an exogenous IL-12. IL-12 can be covalently joined to the 3′ terminus of the intracellular signaling domain of the mesothelin-targeted CAR.

Additionally, the immunoresponsive cells can express a second CAR that binds to a second antigen that is mesothelin or an antigen other than mesothelin. CARs that can be used as a second CAR in combination with the mesothelin-specific CAR in the presently disclosed subject matter include those described in Sadelain, et al., “The Basic Principles of Chimeric Antigen Receptor Design.” Cancer Discovery, OF1-11, (2013), Chicaybam, et al., (2011), Brentjens et al. Nature Medicine 9:279-286 (2003), and U.S. Pat. No. 7,446,190, which are herein incorporated by reference in their entireties, e.g., CD19-targeted CARs (see U.S. Pat. No. 7,446,190; U.S. 2013/0071414,), HER2-targeted CARs (see Ahmed, et al., Clin Cancer Res., 2010), MUC16-targeted CARs (see Chekmasova, et al., 2011), prostate-specific membrane antigen (PSMA)-targeted CARs (for example, Zhong, et al., Molecular Therapy, 18(2):413-420 (2010), all of which are herein incorporated by reference in their entireties. Immunoresponsive cells expressing two or more antigen recognizing receptors (e.g., CARs) are described in WO 2014/055668, which is herein incorporated by reference in its entirety.

The second antigen can be a tumor antigen or a pathogen antigen. Any suitable tumor antigen (antigenic peptide) is suitable for use in the tumor-related embodiments described herein. Sources of tumor antigen include, but are not limited to cancer proteins. The second antigen can be expressed as a peptide or as an intact protein or portion thereof. The intact protein or a portion thereof can be native or mutagenized. Suitable second antigens include, but are not limited to, prostate specific membrane antigen (PSMA) and prostate stem cell antigen (PCSA). In some embodiments, the tumor antigen can be carbonic anhydrase IX (CAIX), carcinoembryonic antigen (CEA), CD5, CD7, CD10, CD19, CD20, CD22, CD30, CD33, CD34, CD38, CD41, CD44, CD49f, CD56, CD74, CD123, CD133, CD138, an antigen of a cytomegalovirus (CMV) infected cell (e.g., a cell surface antigen), epithelial glycoprotein2 (EGP 2), epithelial glycoprotein-40 (EGP-40), epithelial cell adhesion molecule (EpCAM), receptor tyrosine-protein kinases Erb-B2, Erb-B3, Erb-B4, folate-binding protein (FBP), fetal acetylcholine receptor (AChR), folate receptor-a, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Ra2), x-light chain, kinase insert domain receptor (KDR), Lewis A (CA19.9), Lewis Y (LeY), L1 cell adhesion molecule (L1CAM), melanoma antigen family A, 1 (MAGE-AI), Mucin 16 (Muc-16), Mucin 1 (Muc-1), NKG2D ligands, cancer-testis antigen NY-ESO-1, oncofetal antigen (h5T4), prostate stem cell antigen (PSCA), prostate-specific membrane antigen (PSMA), tumor-associated glycoprotein 72 (TAG-72), vascular endothelial growth factor R2 (VEGF-R2), Wilms tumor protein (WT-1), type 1 tyrosine-protein kinase transmembrane receptor (ROR1), or a combination thereof.

Suitable pathogenic antigens for use in the treatment of pathogen infection or other infectious disease, for example, in an immunocompromised subject include, without limitation, viral antigens present in Cytomegalovirus (CMV), Epstein Barr Virus (EBV), Human Immunodeficiency Virus (HIV), and influenza virus. The immunoresponsive cells that include a second CAR targeting a viral antigen can be used for treating viral diseases. In certain embodiments, the mesothelin-targeted CAR and a second CAR that binds to a CMV antigen are co-expressed in the immunoresponsive cells (e.g., cytotoxic T lymphocytes) can be used for treating CMV.

The mesothelin-specific or mesothelin-targeted human lymphocytes that can be used in the methods of the presently disclosed subject matter include, without limitation, peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the a and f3 heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells). The immunoresponsive cells (e.g., T cells) can be autologous, non-autologous (e.g., allogeneic), or derived in vitro from engineered progenitor or stem cells.

Assays may be used to compare the influence of co-stimulatory signaling on enhancing mesothelin-targeted CAR-transduced T cell proliferation, effector function, and accumulation upon repeated (weekly) antigen stimulation. Peripheral blood lymphocytes (PBL) may be harvested from healthy volunteers under an IRB-approved protocol and transduced. Gene transfer efficiency may be monitored by FACS analysis to quantify the fraction of GFP⁺ (transduced) T cells and/or by quantitative PCR. Using a well-established cocultivation system (Gade, T. P., et al. Cancer Res. 65, 9080-9088 (2005); Gong, M. C., et al. Neoplasia. 1, 123-127 (1999); Latouche, J. B. & Sadelain, M. Nat.Biotechnol. 18, 405-409 (2000)), it may be determined whether fibroblast AAPCs expressing mesothelin (vs mesothelin-controls) direct cytokine release from transduced T cells (cell supernatant LUMINEX assay for IL-2, IL-4, IL-10, IFN-γ, TNF-α, and GM-CSF), T cell proliferation (by CFSE labeling), and T cell survival (by Annexin V staining). The influence of CD80 and/or 4-1BBL on T cell survival, proliferation, and efficacy may be evaluated. T cells may be exposed to repeated stimulation by mesothelin⁺ (MSLN⁺) target cells and determine whether T cell proliferation and cytokine response remained similar or diminished with repeated stimulation. Cytotoxicity assays with multiple E:T ratios may be conducted using chromium-release assays. Statistical analysis may be optionally performed with 2-way ANNOVA, followed by pairwise multiple comparison procedures, where data may be expressed as mean±SEM. The CD4 and CD8 T cell subtypes (activated effector, central memory, effector memory) may be identified to determine what conditions favor maintenance or expansion of the central memory phenotype.

In certain embodiments, a presently disclosed immunoresponsive cell (e.g., T cell) expresses from about 1 to about 4, from about 2 to about 4, from about 3 to about 4, from about 1 to about 2, from about 1 to about 3, or from about 2 to about 3 vector copy numbers/cell of the mesothelin-targeted CAR. For example, a presently disclosed immunoresponsive cell (e.g., T cell) expresses about 1, about 2, about 3, or about 4 vector copy numbers/cell of the mesothelin-targeted CAR. In certain embodiments, a presently disclosed immunoresponsive cell (e.g., T cell) expresses from about 3 to about 4 vector copy numbers/cell of the mesothelin-targeted CAR. In certain embodiments, the cytotoxicity and cytokine production of the immunoresponsive cell (e.g., T cell) are proportional to the expression level of the mesothelin-specific CAR in the cell. For example, the higher the CAR expression level in an immunoresponsive cell, the greater cytotoxicity and cytokine production the immunoresponsive cell exhibits. An immunoresponsive cell (e.g., T cell) having a high mesothelin-CAR expression level can induce antigen-specific cytokine production or secretion and/or exhibit cytotoxicity to a tissue or a cell having a low level of mesothelin expression, e.g., about 2,000 or less, about 1,000 or less, about 900 or less, about 800 or less, about 700 or less, about 600 or less, about 500 or less, about 400 or less, about 300 or less, about 200 or less, about 100 or less of mesothelin binding sites/cell. Additionally or alternatively, the cytotoxicity and cytokine production of a presently disclosed immunoresponsive cell (e.g., T cell) are proportional to the expression level of human mesothelin in a target tissue or a target cell. For example, the higher the expression level of human mesothelin in the target, the greater cytotoxicity and cytokine production the immunoresponsive cell exhibits.

In certain embodiments, the target cells are heterogeneous MSLN-expressing cells, which are a population of cells comprising low MSLN-expressing cells and high MSLN-expressing cells. The presently disclosed immunoresponsive cell can exhibit increased cytotoxicity and antitumor activity to low MSLN-expressing cells (e.g., about 2,000 or less, about 1,000 or less, about 900 or less, about 800 or less, about 700 or less, about 600 or less, about 500 or less, about 400 or less, about 300 or less, about 200 or less, or about 100 or less MSLN binding sites/cell) in the presence of high MSLN-expressing cells. In certain embodiments, even in the presence of high MSLN-expressing cells, the immunoresponsive cell does not exhibit increased cytotoxicity or nonspecific kill to MSLN-negative cells. Thus, the immunoresponsive cell can exhibit increased cytotoxicity and antitumor activity to low MSLN-expressing cells in the presence of high MSLN-expressing cells while retain safety to MSLN-negative cells.

In certain embodiments, the immunoresponsive cell can express one or more adhesion molecules, which can increase the avidity of the MSLN-specific CAR, especially when the CAR is a low affinity CAR. Non-limiting examples of adhesion molecules include CD2 and VLA-4. CD2 expressed on the immunoresponsive cell can bind to CD58 expressed on a target cell (e.g., a cancerous cell). VLA-4 expressed on the immunoresponsive cell can bind to VCAM-1 on a target cell (e.g., a cancerous cell).

The unpurified source of CTLs may be any known in the art, such as the bone marrow, fetal, neonate or adult or other hematopoietic cell source, e.g., fetal liver, peripheral blood or umbilical cord blood. Various techniques can be employed to separate the cells. For instance, negative selection methods can remove non-CTLs initially. mAbs are particularly useful for identifying markers associated with particular cell lineages and/or stages of differentiation for both positive and negative selections.

A large proportion of terminally differentiated cells can be initially removed by a relatively crude separation. For example, magnetic bead separations can be used initially to remove large numbers of irrelevant cells. In certain embodiments, at least about 80%, usually at least 70% of the total hematopoietic cells will be removed prior to cell isolation.

Procedures for separation include, but are not limited to, density gradient centrifugation; resetting; coupling to particles that modify cell density; magnetic separation with antibody-coated magnetic beads; affinity chromatography; cytotoxic agents joined to or used in conjunction with a mAb, including, but not limited to, complement and cytotoxins; and panning with antibody attached to a solid matrix, e.g. plate, chip, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels.

The cells can be selected against dead cells, by employing dyes associated with dead cells such as propidium iodide (PI). In certain embodiments, the cells are collected in a medium comprising 2% fetal calf serum (FCS) or 0.2% bovine serum albumin (BSA) or any other suitable, e.g., sterile, isotonic medium.

### 5.4. Nucleic Acid Compositions and Vectors

The present discloses subject matter provides nucleic acid compositions encoding the polypeptide compositions disclosed herein (e.g., disclosed in Section 5.2). In certain embodiments, the nucleic acid composition comprises a polynucleotide encoding the a polypeptide composition disclosed herein (e.g., one disclosed in Section 5.2). Also provided are vectors comprising such nucleic acid compositions, and cells comprising such nucleic acid compositions or vectors.

In certain embodiments, the nucleic acid composition further comprises a promoter that is operably linked to the polypeptide composition. In certain embodiments, the promoter is endogenous or exogenous. In certain embodiments, the exogenous promoter is selected from an elongation factor (EF)-1 promoter, a CMV promoter, a SV40 promoter, a PGK promoter, and a metallothionein promoter. In certain embodiments, the promoter is an inducible promoter. In certain embodiment, the inducible promoter is selected from a NFAT transcriptional response element (TRE) promoter, a CD69 promoter, a CD25 promoter, and an IL-2 promoter.

The nucleic acid compositions can be administered to subjects or and/delivered into cells by art-known methods or as described herein.

Genetic modification of an immunoresponsive cell (e.g., a T cell or a NK cell) can be accomplished by transducing a substantially homogeneous cell composition with a recombinant DNA construct. In certain embodiments, a retroviral vector (either gamma-retroviral or lentiviral) is employed for the introduction of the DNA construct into the cell. For example, a polynucleotide encoding an antigen-recognizing receptor can be cloned into a retroviral vector and expression can be driven from its endogenous promoter, from the retroviral long terminal repeat, or from a promoter specific for a target cell type of interest. Non-viral vectors may be used as well.

For initial genetic modification of an immunoresponsive cell to include an antigen recognizing receptor (e.g., a CAR or TCR), a retroviral vector is generally employed for transduction, however any other suitable viral vector or non-viral delivery system can be used. The CAR and the PD-1 DN can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides , e.g., P2A, T2A, E2A and F2A peptides). In certain embodiments, the P2A peptide comprises or consists of the amino acid sequence set forth in SEQ ID NO: 107, which is provided below:

[SEQ ID NO: 107]
GSGATNFSLLKQAGDVEENPGPM

In certain embodiments, the P2A peptide comprises or consists of the amino acid sequence set forth in SEQ ID NO: 121, which is provided below:

[SEQ ID NO: 121]
ATNFSLLKQAGDVEENPGP

An exemplary nucleotide sequence encoding the amino acid sequence of SEQ ID NO: 121 is set forth in 122, which is provided below:

[SEQ ID NO: 122]
GCAACAAACTTCTCACTACTCAAACAAGCAGGTGACGTGGAGGAGAA
TCCCGGCCCA

Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify an immunoresponsive cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

In certain embodiments, a vector encoding a presently disclosed polypeptide composition is a retroviral vector, e.g., a SGF y-retroviral vector, which can be Moloney murine leukemia-based retroviral vector. In certain embodiments, the vector comprises or consists of the nucleotide sequence set forth in SEQ ID NO: 123, which is provided below:

[SEQ ID NO: 123]
ATGGCCCTGCCAGTAACGGCTCTGCTGCTGCCACTTGCTCTGCTCCTCCATGCAGCCAGGCCTCAGGTTCAGCTTCAGGA
GAGTGGCCCAGGCCTGGTGAAGCCAAGTGAGACTCTCAGCTTGACTTGCACAGTTTCTGGAGGCAGTGTCTCCTCAGGCA
GCTATTATTGGTCCTGGATTCGGCAGCCCCCTGGGAAAGGCCTGGAGTGGATTGGGTACATATATTACAGTGGCAGCACA
AATTACAATCCATCCCTGAAGTCTCGAGTAACTATCAGTGTGGACACAAGCAAGAATCAGTTTTCACTCAAACTGTCTTC
TGTGACTGCTGCTGACACTGCTGTTTATTATTGTGCCAGGGAGGGGAAAAATGGGGCATTTGATATTTGGGGTCAGGGCA
CAATGGTGACAGTCAGCTCTGGAGGTGGAGGCTCAGGAGGAGGAGGCAGTGGAGGTGGTGGGTCACGCCATCAGATGACT
CAGTCCCCCTCCAGTCTTTCTGCCTCAGTTGGGGATAGAGTGACCATCACATGCAGAGCAAGTCAGAGCATATCATCCTA
TCTGAACTGGTACCAGCAGAAGCCAGGGAAAGCCCCCAAATTGCTGATTTATGCAGCCTCAAGTCTCCAGAGTGGGGTGC
CAAGCAGGTTCTCAGGCAGTGGCAGTGGGACAGATTTCACATTGACAATCAGCTCCCTCCAACCTGAAGATTTTGCCACC
TACTATTGCCAGCAATCCTACAGCACGCCCCTGACTTTTGGAGGTGGCACAAAGGTAGAGATCAAGAGGACTgcggccgc
aattgaagttatgtatcctcctccttacctagacaatgagaagagcaatggaaccattatccatgtgaaagggaaacacc
tttgtccaagtcccctatttcccggaccttctaagcccttttgggtgctggtggtggttggtggagtcctggcttgctat
agcttgctagtaacagtggcctttattattttctgggtgaggagtaagaggagcaggctcctgcacagtgactacatgaa
catgactccccgccgccccgggcccacccgcaagcattaccagccctatgccccaccacgcgacttcgcagcctatcgct
ccagagtgaagttcagcaggagcgcagacgcccccgcgtaccagcagggccagaaccagctctataacgagctcaatcta
ggacgaagagaggagtacgatgttttggacaagagacgtggccgggaccctgagatggggggaaagccgagaaggaagaa
ccctcaggaaggcctgtTcaatgaactgcagaaagataagatggcggaggcctTcagtgagattgggatgaaaggcgagc
gccggaggggcaaggggcacgatggcctttTccaggggctcagtacagccaccaaggacacctTcgacgcccttcacatg
caggccctgccccctcgcGGATCTGGAGCAACAAACTTCTCACTACTCAAACAAGCAGGTGACGTGGAGGAGAATCCCGG
CCCAATGCAGATCCCACAGGCGCCCTGGCCAGTCGTCTGGGCGGTGCTACAACTGGGCTGGCGGCCAGGATGGTTCTTAG
ACTCCCCAGACAGGCCCTGGAACCCCCCCACCTTCTCCCCAGCCCTGCTCGTGGTGACCGAAGGGGACAACGCCACCTTC
ACCTGCAGCTTCTCCAACACATCGGAGAGCTTCGTGCTAAACTGGTACCGCATGAGCCCCAGCAACCAGACGGACAAGCT
GGCCGCTTTCCCCGAGGACCGCAGCCAGCCCGGCCAGGACTGCCGCTTCCGTGTCACACAACTGCCCAACGGGCGTGACT
TCCACATGAGCGTGGTCAGGGCCCGGCGCAATGACAGCGGCACCTACCTCTGTGGGGCCATCTCCCTGGCCCCCAAGGCG
CAGATCAAAGAGAGCCTGCGGGCAGAGCTCAGGGTGACAGAGAGAAGGGCAGAAGTGCCCACAGCCCACCCCAGCCCCTC
ACCCAGGCCAGCCGGCCAGGCGGCCGCACCCACCACGACGCCAGCGCCGCGACCACCAACCCCGGCGCCCACGATCGCGT
CGCAGCCCCTGTCCCTGCGCCCAGAGGCGTGCCGGCCAGCGGCGGGGGGCGCAGTGCACACGAGGGGGCTGGACTTCGCC
TGTGATATCTACATCTGGGCGCCCCTGGCCGGGACTTGTGGGGTCCTTCTCCTGTCACTGGTTATCACCCTTTACTGCAA
CCACAGGCGGATCCAATAAcagccactcgaggatccGGATTAGTCCAATTTGTTAAAGACAGGATATCAGTGGTCCAGGC
TCTAGTTTTGACTCAACAATATCACCAGCTGAAGCCTATAGAGTACGAGCCATAGATAAAATAAAAGATTTTATTTAGTC
TCCAGAAAAAGGGGGGAATGAAAGACCCCACCTGTAGGTTTGGCAAGCTAGCTTAAGTAACGCCATTTTGCAAGGCATGG
AAAAATACATAACTGAGAATAGAGAAGTTCAGATCAAGGTCAGGAACAGATGGAACAGCTGAATATGGGCCAAACAGGAT
ATCTGTGGTAAGCAGTTCCTGCCCCGGCTCAGGGCCAAGAACAGATGGAACAGCTGAATATGGGCCAAACAGGATATCTG
TGGTAAGCAGTTCCTGCCCCGGCTCAGGGCCAAGAACAGATGGTCCCCAGATGCGGTCCAGCCCTCAGCAGTTTCTAGAG
AACCATCAGATGTTTCCAGGGTGCCCCAAGGACCTGAAATGACCCTGTGCCTTATTTGAACTAACCAATCAGTTCGCTTC
TCGCTTCTGTTCGCGCGCTTCTGCTCCCCGAGCTCAATAAAAGAGCCCACAACCCCTCACTCGGGGCGCCAGTCCTCCGA
TTGACTGAGTCGCCCGGGTACCCGTGTATCCAATAAACCCTCTTGCAGTTGCATCCGACTTGTGGTCTCGCTGTTCCTTG
GGAGGGTCTCCTCTGAGTGATTGACTACCCGTCAGCGGGGGTCTTTCACACATGCAGCATGTATCAAAATTAATTTGGTT
TTTTTTCTTAAGTATTTACATTAAATGGCCATAGTACTTAAAGTTACATTGGCTTCCTTGAAATAAACATGGAGTATTCA
GAATGTGTCATAAATATTTCTAATTTTAAGATAGTATCTCCATTGGCTTTCTACTTTTTCTTTTATTTTTTTTTGTCCTC
TGTCTTCCATTTGTTGTTGTTGTTGTTTGTTTGTTTGTTTGTTGGTTGGTTGGTTAATTTTTTTTTAAAGATCCTACACT
ATAGTTCAAGCTAGACTATTAGCTACTCTGTAACCCAGGGTGACCTTGAAGTCATGGGTAGCCTGCTGTTTTAGCCTTCC
CACATCTAAGATTACAGGTATGAGCTATCATTTTTGGTATATTGATTGATTGATTGATTGATGTGTGTGTGTGTGATTGT
GTTTGTGTGTGTGACTGTGAAAATGTGTGTATGGGTGTGTGTGAATGTGTGTATGTATGTGTGTGTGTGAGTGTGTGTGT
GTGTGTGTGCATGTGTGTGTGTGTGACTGTGTCTATGTGTATGACTGTGTGTGTGTGTGTGTGTGTGTGTGTGTGTGTGT
GTGTGTGTGTGTTGTGAAAAAATATTCTATGGTAGTGAGAGCCAACGCTCCGGCTCAGGTGTCAGGTTGGTTTTTGAGAC
AGAGTCTTTCACTTAGCTTGGAATTCactggccgtcgttttacaacgtcgtgactgggaaaaccctggcgttacccaact
taatcgccttgcagcacatccccctttcgccagctggcgtaatagcgaagaggcccgcaccgatcgcccttcccaacagt
tgcgcagcctgaatggcgaatggcgcctgatgcggtattttctccttacgcatctgtgcggtatttcacaccgcatatgg
tgcactctcagtacaatctgctctgatgccgcatagttaagccagccccgacacccgccaacacccgctgacgcgccctg
acgggcttgtctgctcccggcatccgcttacagacaagctgtgaccgtctccgggagctgcatgtgtcagaggttttcac
cgtcatcaccgaaacgcgcgatgacgaaagggcctcgtgatacgcctatttttataggttaatgtcatgataataatggt
ttcttagacgtcaggtggcacttttcggggaaatgtgcgcggaacccctatttgtttatttttctaaatacattcaaata
tgtatccgctcatgagacaataaccctgataaatgcttcaataatattgaaaaaggaagagtatgagtattcaacatttc
cgtgtcgcccttattcccttttttgcggcattttgccttcctgtttttgctcacccagaaacgctggtgaaagtaaaaga
tgctgaagatcagttgggtgcacgagtgggttacatcgaactggatctcaacagcggtaagatccttgagagttttcgcc
ccgaagaacgttttccaatgatgagcacttttaaagttctgctatgtggcgcggtattatcccgtattgacgccgggcaa
gagcaactcggtcgccgcatacactattctcagaatgacttggttgagtactcaccagtcacagaaaagcatcttacgga
tggcatgacagtaagagaattatgcagtgctgccataaccatgagtgataacactgcggccaacttacttctgacaacga
tcggaggaccgaaggagctaaccgcttttttgcacaacatgggggatcatgtaactcgccttgatcgttgggaaccggag
ctgaatgaagccataccaaacgacgagcgtgacaccacgatgcctgtagcaatggcaacaacgttgcgcaaactattaac
tggcgaactacttactctagcttcccggcaacaattaatagactggatggaggcggataaagttgcaggaccacttctgc
gctcggcccttccggctggctggtttattgctgataaatctggagccggtgagcgtgggtctcgcggtatcattgcagca
ctggggccagatggtaagccctcccgtatcgtagttatctacacgacggggagtcaggcaactatggatgaacgaaatag
acagatcgctgagataggtgcctcactgattaagcattggtaactgtcagaccaagtttactcatatatactttagattg
atttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaat
ctgctgcttgcaaacaaaaaaaccaccgctaccagcggtggtttgtttgccggatcaagagctaccaactctttttccga
aggtaactggcttcagcagagcgcagataccaaatactgtccttctagtgtagccgtagttaggccaccacttcaagaac
tctgtagcaccgcctacatacctcgctctgctaatcctgttaccagtggctgctgccagtggcgataagtcgtgtcttac
cgggttggactcaagacgatagttaccggataaggcgcagcggtcgggctgaacggggggttcgtgcacacagcccagct
tggagcgaacgacctacaccgaactgagatacctacagcgtgagcattgagaaagcgccacgcttcccgaagggagaaag
gcggacaggtatccggtaagcggcagggtcggaacaggagagcgcacgagggagcttccagggggaaacgcctggtatct
ttatagtcctgtcgggtttcgccacctctgacttgagcgtcgatttttgtgatgctcgtcaggggggcggagcctatgga
aaaacgccagcaacgcggcctttttacggttcctggccttttgctggccttttgctcacatgttctttcctgcgttatcc
cctgattctgtggataaccgtattaccgcctttgagtgagctgataccgctcgccgcagccgaacgaccgagcgcagcga
gtcagtgagcgaggaagcggaagagcgcccaatacgcaaaccgcctctccccgcgcgttggccgattcattaatgcagct
ggcacgacaggtttcccgactggaaagcgggcagtgagcgcaacgcaattaatgtgagttagctcactcattaggcaccc
caggctttacactttatgcttccggctcgtatgttgtgtggaattgtgagcggataacaatttcacacaggaaacagcta
tgaccatgattacgccAAGCTTTGCTCTTAGGAGTTTCCTAATACATCCCAAACTCAAATATATAAAGCATTTGACTTGT
TCTATGCCCTAGGGGGCGGGGGGAAGCTAAGCCAGCTTTTTTTAACATTTAAAATGTTAATTCCATTTTAAATGCACAGA
TGTTTTTATTTCATAAGGGTTTCAATGTGCATGAATGCTGCAATATTCCTGTTACCAAAGCTAGTATAAATAAAAATAGA
TAAACGTGGAAATTACTTAGAGTTTCTGTCATTAACGTTTCCTTCCTCAGTTGACAACATAAATGCGCTGCTGAGCAAGC
CAGTTTGCATCTGTCAGGATCAATTTCCCATTATGCCAGTCATATTAATTACTAGTCAATTAGTTGATTTTTATTTTTGA
CATATACATGTGAATGAAAGACCCCACCTGTAGGTTTGGCAAGCTAGCTTAAGTAACGCCATTTTGCAAGGCATGGAAAA
ATACATAACTGAGAATAGAAAAGTTCAGATCAAGGTCAGGAACAGATGGAACAGCTGAATATGGGCCAAACAGGATATCT
GTGGTAAGCAGTTCCTGCCCCGGCTCAGGGCCAAGAACAGATGGAACAGCTGAATATGGGCCAAACAGGATATCTGTGGT
AAGCAGTTCCTGCCCCGGCTCAGGGCCAAGAACAGATGGTCCCCAGATGCGGTCCAGCCCTCAGCAGTTTCTAGAGAACC
ATCAGATGTTTCCAGGGTGCCCCAAGGACCTGAAATGACCCTGTGCCTTATTTGAACTAACCAATCAGTTCGCTTCTCGC
TTCTGTTCGCGCGCTTATGCTCCCCGAGCTCAATAAAAGAGCCCACAACCCCTCACTCGGGGCGCCAGTCCTCCGATTGA
CTGAGTCGCCCGGGTACCCGTGTATCCAATAAACCCTCTTGCAGTTGCATCCGACTTGTGGTCTCGCTGTTCCTTGGGAG
GGTCTCCTCTGAGTGATTGACTACCCGTCAGCGGGGGTCTTTCATTTGGGGGCTCGTCCGGGATCGGGAGACCCCTGCCC
AGGGACCACCGACCCACCACCGGGAGGTAAGCTGGCCAGCAACTTATCTGTGTCTGTCCGATTGTCTAGTGTCTATGACT
GATTTTATGCGCCTGCGTCGGTACTAGTTAGCTAACTAGCTCTGTATCTGGCGGACCCGTGGTGGAACTGACGAGTTCGG
AACACCCGGCCGCAACCCTGGGAGACGTCCCAGGGACTTCGGGGGCCGTTTTTGTGGCCCGACCTGAGTCCTAAAATCCC
GATCGTTTAGGACTCTTTGGTGCACCCCCCTTAGAGGAGGGATATGTGGTTCTGGTAGGAGACGAGAACCTAAAACAGTT
CCCGCCTCCGTCTGAATTTTTGCTTTCGGTTTGGGACCGAAGCCGCGCCGCGCGTCTTGTCTGCTGCAGCATCGTTCTGT
GTTGTCTCTGTCTGACTGTGTTTCTGTATTTGTCTGAAAATATGGGCCCGGGCTAGcctgttaccactCCCTTAAGTTTG
ACCTTAGGTCACTGGAAAGATGTCGAGCGGATCGCTCACAACCAGTCGGTAGATGTCAAGAAGAGACGTTGGGTTACCTT
CTGCTCTGCAGAATGGCCAACCTTTAACGTCGGATGGCCGCGAGACGGCACCTTTAACCGAGACCTCATCACCCAGGTTA
AGATCAAGGTCTTTTCACCTGGCCCGCATGGACACCCAGACCAGGTccccTACATCGTGACCTGGGAAGCCTTGGCTTTT
GACCCCCCTCCCTGGGTCAAGCCCTTTGTACACCCTAAGCCTCCGCCTCCTCTTCCTCCATCCGCCCCGTCTCTCCCCCT
TGAACCTCCTCGTTCGACCCCGCCTCGATCCTCCCTTTATCCAGCCCTCACTCCTTCTCTAGGCGCCCCCATATGGCCAT
ATGAGATCTTATATGGGGCACCCCCGCCCCTTGTAAACTTCCCTGACCCTGACATGACAAGAGTTACTAACAGCCCCTCT
CTCCAAGCTCACTTACAGGCTCTCTACTTAGTCCAGCACGAAGTCTGGAGACCTCTGGCGGCAGCCTACCAAGAACAACT
GGACCGACCGGTGGTACCTCACCCTTACCGAGTCGGCGACACAGTGTGGGTCCGCCGACACCAGACTAAGAACCTAGAAC
CTCGCTGGAAAGGACCTTACACAGTCCTGCTGACCACCCCCACCGCCCTCAAAGTAGACGGCATCGCAGCTTGGATACAC
GCCGCCCACGTGAAGGCTGCCGACCCCGGGGGTGGACCATCCTCTAGACTGCC

In certain embodiments, the vector comprises or consists of the nucleotide sequence set forth in SEQ ID NO: 124, which is provided below:

[SEQ ID NO: 124]
ATGGCCCTGCCAGTAACGGCTCTGCTGCTGCCACTTGCTCTGCTCCTCCATGCAGCCAGGCCTCAGGTTCAGCT
TCAGGAGAGTGGCCCAGGCCTGGTGAAGCCAAGTGAGACTCTCAGCTTGACTTGCACAGTTTCTGGAGGCAGTGTCTCCT
CAGGCAGCTATTATTGGTCCTGGATTCGGCAGCCCCCTGGGAAAGGCCTGGAGTGGATTGGGTACATATATTACAGTGGC
AGCACAAATTACAATCCATCCCTGAAGTCTCGAGTAACTATCAGTGTGGACACAAGCAAGAATCAGTTTTCACTCAAACT
GTCTTCTGTGACTGCTGCTGACACTGCTGTTTATTATTGTGCCAGGGAGGGGAAAAATGGGGCATTTGATATTTGGGGTC
AGGGCACAATGGTGACAGTCAGCTCTGGAGGTGGAGGCTCAGGAGGAGGAGGCAGTGGAGGTGGTGGGTCACGCCATCAG
ATGACTCAGTCCCCCTCCAGTCTTTCTGCCTCAGTTGGGGATAGAGTGACCATCACATGCAGAGCAAGTCAGAGCATATC
ATCCTATCTGAACTGGTACCAGCAGAAGCCAGGGAAAGCCCCCAAATTGCTGATTTATGCAGCCTCAAGTCTCCAGAGTG
GGGTGCCAAGCAGGTTCTCAGGCAGTGGCAGTGGGACAGATTTCACATTGACAATCAGCTCCCTCCAACCTGAAGATTTT
GCCACCTACTATTGCCAGCAATCCTACAGCACGCCCCTGACTTTTGGAGGTGGCACAAAGGTAGAGATCAAGAGGACTgc
ggccgcaattgaagttatgtatcctcctccttacctagacaatgagaagagcaatggaaccattatccatgtgaaaggga
aacacctttgtccaagtcccctatttcccggaccttctaagcccttttgggtgctggtggtggttggtggagtcctggct
tgctatagcttgctagtaacagtggcctttattattttctgggtgaggagtaagaggagcaggctcctgcacagtgacta
catgaacatgactccccgccgccccgggcccacccgcaagcattaccagccctatgccccaccacgcgacttcgcagcct
atcgctccagagtgaagttcagcaggagcgcagacgcccccgcgtaccagcagggccagaaccagctctataacgagctc
aatctaggacgaagagaggagtacgatgttttggacaagagacgtggccgggaccctgagatggggggaaagccgagaag
gaagaaccctcaggaaggcctgtTcaatgaactgcagaaagataagatggcggaggcctTcagtgagattgggatgaaag
gcgagcgccggaggggcaaggggcacgatggcctttTccaggggctcagtacagccaccaaggacacctTcgacgccctt
cacatgcaggccctgccccctcgcGGATCTGGAGCAACAAACTTCTCACTACTCAAACAAGCAGGTGACGTGGAGGAGAA
TCCCGGCCCAATGCAGATCCCACAGGCGCCCTGGCCAGTCGTCTGGGCGGTGCTACAACTGGGCTGGCGGCCAGGATGGT
TCTTAGACTCCCCAGACAGGCCCTGGAACCCCCCCACCTTCTCCCCAGCCCTGCTCGTGGTGACCGAAGGGGACAACGCC
ACCTTCACCTGCAGCTTCTCCAACACATCGGAGAGCTTCGTGCTAAACTGGTACCGCATGAGCCCCAGCAACCAGACGGA
CAAGCTGGCCGCTTTCCCCGAGGACCGCAGCCAGCCCGGCCAGGACTGCCGCTTCCGTGTCACACAACTGCCCAACGGGC
GTGACTTCCACATGAGCGTGGTCAGGGCCCGGCGCAATGACAGCGGCACCTACCTCTGTGGGGCCATCTCCCTGGCCCCC
AAGGCGCAGATCAAAGAGAGCCTGCGGGCAGAGCTCAGGGTGACAGAGAGAAGGGCAGAAGTGCCCACAGCCCACCCCAG
CCCCTCACCCAGGCCAGCCGGCCAGGCGGCCGCACCCACCACGACGCCAGCGCCGCGACCACCAACCCCGGCGCCCACGA
TCGCGTCGCAGCCCCTGTCCCTGCGCCCAGAGGCGTGCCGGCCAGCGGCGGGGGGCGCAGTGCACACGAGGGGGCTGGAC
TTCGCCTGTGATATCTACATCTGGGCGCCCCTGGCCGGGACTTGTGGGGTCCTTCTCCTGTCACTGGTTATCACCCTTTA
CTGCAACCACAGGCGGATCCAATAAcagccactcgaggatccGGATTAGTCCAATTTGTTAAAGACAGGATATCAGTGGT
CCAGGCTCTAGTTTTGACTCAACAATATCACCAGCTGAAGCCTATAGAGTACGAGCCATAGATAAAATAAAAGATTTTAT
TTAGTCTCCAGAAAAAGGGGGGAATGAAAGACCCCACCTGTAGGTTTGGCAAGCTAGCTTAAGTAACGCCATTTTGCAAG
GCATGGAAAAATACATAACTGAGAATAGAGAAGTTCAGATCAAGGTCAGGAACAGATGGAACAGCTGAATATGGGCCAAA
CAGGATATCTGTGGTAAGCAGTTCCTGCCCCGGCTCAGGGCCAAGAACAGATGGAACAGCTGAATATGGGCCAAACAGGA
TATCTGTGGTAAGCAGTTCCTGCCCCGGCTCAGGGCCAAGAACAGATGGTCCCCAGATGCGGTCCAGCCCTCAGCAGTTT
CTAGAGAACCATCAGATGTTTCCAGGGTGCCCCAAGGACCTGAAATGACCCTGTGCCTTATTTGAACTAACCAATCAGTT
CGCTTCTCGCTTCTGTTCGCGCGCTTCTGCTCCCCGAGCTCAATAAAAGAGCCCACAACCCCTCACTCGGGGCGCCAGTC
CTCCGATTGACTGAGTCGCCCGGGTACCCGTGTATCCAATAAACCCTCTTGCAGTTGCATCCGACTTGTGGTCTCGCTGT
TCCTTGGGAGGGTCTCCTCTGAGTGATTGACTACCCGTCAGCGGGGGTCTTTCACATGCAGCATGTATCAAAATTAATTT
GGTTTTTTTTCTTAAGTATTTACATTAAATGGCCATAGTACTTAAAGTTACATTGGCTTCCTTGAAATAAACATGGAGTA
TTCAGAATGTGTCATAAATATTTCTAATTTTAAGATAGTATCTCCATTGGCTTTCTACTTTTTCTTTTATTTTTTTTTGT
CCTCTGTCTTCCATTTGTTGTTGTTGTTGTTTGTTTGTTTGTTTGTTGGTTGGTTGGTTAATTTTTTTTTAAAGATCCTA
CACTATAGTTCAAGCTAGACTATTAGCTACTCTGTAACCCAGGGTGACCTTGAAGTCATGGGTAGCCTGCTGTTTTAGCC
TTCCCACATCTAAGATTACAGGTATGAGCTATCATTTTTGGTATATTGATTGATTGATTGATTGATGTGTGTGTGTGTGA
TTGTGTTTGTGTGTGTGAtTGTGtAtATGTGTGTATGGtTGTGTGTGAtTGTGTGTATGTATGTtTGTGTGTGAtTGTGT
GTGTGTGatTGTGCATGTGTGTGTGTGTGAtTGTGTtTATGTGTATGAtTGTGTGTGTGTGTGTGTGTGTGTGTGTGTGT
GTGTGTGTGTGTGTGTTGTGtAtAtATATTTATGGTAGTGAGAGgCAACGCTCCGGCTCAGGTGTCAGGTTGGTTTTTGA
GACAGAGTCTTTCACTTAGCTTGGAATTCactggccgtcgttttacaacgtcgtgactgggaaaaccctggcgttaccca
acttaatcgccttgcagcacatccccctttcgccagctggcgtaatagcgaagaggcccgcaccgatcgcccttcccaac
agttgcgcagcctgaatggcgaatggcgcctgatgcggtattttctccttacgcatctgtgcggtatttcacaccgcata
tggtgcactctcagtacaatctgctctgatgccgcatagttaagccagccccgacacccgccaacacccgctgacgcgcc
ctgacgggcttgtctgctcccggcatccgcttacagacaagctgtgaccgtctccgggagctgcatgtgtcagaggtttt
caccgtcatcaccgaaacgcgcgagacgaaagggcctcgtgatacgcctatttttataggttaatgtcatgataataatg
gtttcttagacgtcaggtggcacttttcggggaaatgtgcgcggaacccctatttgtttatttttctaaatacattcaaa
tatgtatccgctcatgagacaataaccctgataaatgcttcaataatattgaaaaaggaagagtatgagtattcaacatt
tccgtgtcgcccttattcccttttttgcggcattttgccttcctgtttttgctcacccagaaacgctggtgaaagtaaaa
gatgctgaagatcagttgggtgcacgagtgggttacatcgaactggatctcaacagcggtaagatccttgagagttttcg
ccccgaagaacgttttccaatgatgagcacttttaaagttctgctatgtggcgcggtattatcccgtattgacgccgggc
aagagcaactcggtcgccgcatacactattctcagaatgacttggttgagtactcaccagtcacagaaaagcatcttacg
gatggcatgacagtaagagaattatgcagtgctgccataaccatgagtgataacactgcggccaacttacttctgacaac
gatcggaggaccgaaggagctaaccgcttttttgcacaacatgggggatcatgtaactcgccttgatcgttgggaaccgg
agctgaatgaagccataccaaacgacgagcgtgacaccacgatgcctgtagcaatggcaacaacgttgcgcaaactatta
actggcgaactacttactctagcttcccggcaacaattaatagactggatggaggcggataaagttgcaggaccacttct
gcgctcggcccttccggctggctggtttattgctgataaatctggagccggtgagcgtgggtctcgcggtatcattgcag
cactggggccagatggtaagccctcccgtatcgtagttatctacacgacggggagtcaggcaactatggatgaacgaaat
agacagatcgctgagataggtgcctcactgattaagcattggtaactgtcagaccaagtttactcatatatactttagat
tgatttaaaacttcatttttaatttaaaaggatctaggtgaagatcctttttgataatctcatgaccaaaatcccttaac
gtgagttttcgttccactgagcgtcagaccccgtagaaaagatcaaaggatcttcttgagatcctttttttctgcgcgta
atctgctgcttgcaaacaaaaaaaccaccgctaccagcggtggtttgtttgccggatcaagagctaccaactctttttcc
gaaggtaactggcttcagcagagcgcagataccaaatactgtTcttctagtgtagccgtagttaggccaccacttcaaga
actctgtagcaccgcctacatacctcgctctgctaatcctgttaccagtggctgctgccagtggcgataagtcgtgtctt
accgggttggactcaagacgatagttaccggataaggcgcagcggtcgggctgaacggggggttcgtgcacacagcccag
cttggagcgaacgacctacaccgaactgagatacctacagcgtgagcTAtgagaaagcgccacgcttcccgaagggagaa
aggcggacaggtatccggtaagcggcagggtcggaacaggagagcgcacgagggagcttccagggggaaacgcctggtat
ctttatagtcctgtcgggtttcgccacctctgacttgagcgtcgatttttgtgatgctcgtcaggggggcggagcctatg
gaaaaacgccagcaacgcggcctttttacggttcctggccttttgctggccttttgctcacatgttctttcctgcgttat
cccctgattctgtggataaccgtattaccgcctttgagtgagctgataccgctcgccgcagccgaacgaccgagcgcagc
gagtcagtgagcgaggaagcggaagagcgcccaatacgcaaaccgcctctccccgcgcgttggccgattcattaatgcag
ctggcacgacaggtttcccgactggaaagcgggcagtgagcgcaacgcaattaatgtgagttagctcactcattaggcac
cccaggctttacactttatgcttccggctcgtatgttgtgtggaattgtgagcggataacaatttcacacaggaaacagc
tatgaccatgattacgccAAGCTTTGCTCTTAGGAGTTTCCTAATACATCCCAAACTCAAATATATAAAGCATTTGACTT
GTTCTATGCCCTAGGGGGCGGGGGGAAGCTAAGCCAGCTTTTTTTAACATTTAAAATGTTAATTCCATTTTAAATGCACA
GATGTTTTTATTTCATAAGGGTTTCAATGTGCATGAATGCTGCAATATTCCTGTTACCAAAGCTAGTATAAATAAAAATA
GATAAACGTGGAAATTACTTAGAGTTTCTGTCATTAACGTTTCCTTCCTCAGTTGACAACATAAATGCGCTGCTGAGAAG
CCAGTTTGCATCTGTCAGGATCAATTTCCCATTATGCCAGTCATATTAATTACTAGTCAATTAGTTGATTTTTATTTTTG
ACATATACATGTGAAAGACCCCACCTGTAGGTTTGGCAAGCTAGCTTAAGTAACGCCATTTTGCAAGGCATGGAAAAATA
CATAACTGAGAATAGAAAAGTTCAGATCAAGGTCAGGAACAGATGGAACAGCTGAATATGGGCCAAACAGGATATCTGTG
GTAAGCAGTTCCTGCCCCGGCTCAGGGCCAAGAACAGATGGAACAGCTGAATATGGGCCAAACAGGATATCTGTGGTAAG
CAGTTCCTGCCCCGGCTCAGGGCCAAGAACAGATGGTCCCCAGATGCGGTCCAGCCCTCAGCAGTTTCTAGAGAACCATC
AGATGTTTCCAGGGTGCCCCAAGGACCTGAAATGACCCTGTGCCTTATTTGAACTAACCAATCAGTTCGCTTCTCGCTTC
TGTTCGCGCGCTTcTGCTCCCCGAGCTCAATAAAAGAGCCCACAACCCCTCACTCGGcGCGCCAGTCCTCCGATTGACTG
AGTCGCCCGGGTACCCGTGTATCCAATAAACCCTCTTGCAGTTGCATCCGACTTGTGGTCTCGCTGTTCCTTGGGAGGGT
CTCCTCTGAGTGATTGACTACCCGTCAGCGGGGGTCTTTCATTTGGGGGCTCGTCCGGGATCGGGAGACCCCTGCCCAGG
GACCACCGACCCACCACCGGGAGGTAAGCTGGCCAGCAACTTATCTGTGTCTGTCCGATTGTCTAGTGTCTATGACTGAT
TTTATGCGCCTGCGTCGGTACTAGTTAGCTAACTAGCTCTGTATCTGGCGGACCCGTGGTGGAACTGACGAGTTCGGAAC
ACCCGGCCGCAACCCTGGGAGACGTCCCAGGGACTTCGGGGGCCGTTTTTGTGGCCCGACCTGAGTCCTAAAATCCCGAT
CGTTTAGGACTCTTTGGTGCACCCCCCTTAGAGGAGGGATATGTGGTTCTGGTAGGAGACGAGAACCTAAAACAGTTCCC
GCCTCCGTCTGAATTTTTGCTTTCGGTTTGGGACCGAAGCCGCGCCGCGCGTCTTGTCTGCTGCAGCATCGTTCTGTGTT
GTCTCTGTCTGACTGTGTTTCTGTATTTGTCTGAAAATATGGGCCCGGGCTAGcctgttaccactCCCTTAAGTTTGACC
TTAGGTCACTGGAAAGATGTCGAGCGGATCGCTCACAACCAGTCGGTAGATGTCAAGAAGAGACGTTGGGTTACCTTCTG
CTCTGCAGAATGGCCAACCTTTAACGTCGGATGGCCGCGAGACGGCACCTTTAACCGAGACCTCATCACCCAGGTTAAGA
TCAAGGTCTTTTCACCTGGCCCGCATGGACACCCAGACCAGGTccccTACATCGTGACCTGGGAAGCCTTGGCTTTTGAC
CCCCCTCCCTGGGTCAAGCCCTTTGTACACCCTAAGCCTCCGCCTCCTCTTCCTCCATCCGCCCCGTCTCTCCCCCTTGA
ACCTCCTCGTTCGACCCCGCCTCGATCCTCCCTTTATCCAGCCCTCACTCCTTCTCTAGGCGCCCCCATATGGCCATATG
AGATCTTATATGGGGCACCCCCGCCCCTTGTAAACTTCCCTGACCCTGACATGACAAGAGTTACTAACAGCCCCTCTCTC
CAAGCTCACTTACAGGCTCTCTACTTAGTCCAGCACGAAGTCTGGAGACCTCTGGCGGCAGCCTACCAAGAACAACTGGA
CCGACCGGTGGTACCTCACCCTTACCGAGTCGGCGACACAGTGTGGGTCCGCCGACACCAGACTAAGAACCTAGAACCTC
GCTGGAAAGGACCTTACACAGTCCTGCTGACCACCCCCACCGCCCTCAAAGTAGACGGCATCGCAGCTTGGATACACGCC
GCCCACGTGAAGGCTGCCGACCCCGGGGGTGGACCATCCTCTAGACTGCC

Non-viral approaches can also be employed for genetic modification of an immunoresponsive cell. For example, a nucleic acid molecule can be introduced into an immunoresponsive cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

Any targeted genome editing methods can be used to express the polypeptide composition. In certain embodiments, a CRISPR system is used to express the polypeptide composition disclosed herein. In certain embodiments, zinc-finger nucleases are used to express the polypeptide composition disclosed herein. In certain embodiments, a TALEN system is used to express the polypeptide composition disclosed herein.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor la enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

### 5.5. Polypeptides and Analogs

Also included in the presently disclosed subject matter are polypeptide disclosed herein (e.g., mesothelin, CD28, CD8, CD3ζ, and PD-1 DN, etc.) or fragments thereof that are modified in ways that enhance their anti-neoplastic activity when expressed in an immunoresponsive cell. The presently disclosed subject matter provides methods for optimizing an amino acid sequence or nucleic acid sequence by producing an alteration in the sequence. Such alterations may include certain mutations, deletions, insertions, or post-translational modifications. The presently disclosed subject matter further includes analogs of any polypeptide disclosed herein (including, but not limited to, mesothelin, CD28, CD8, CD3ζ and PD-1 DN). Analogs can differ from a polypeptide disclosed herein by amino acid sequence differences, by post-translational modifications, or by both.

Analogs can exhibit at least about 85%, about 90%, about 91%, about 92%, about 93%, about 94%, about 95%, about 96%, about 97%, about 98%, about 99% or more homologous to all or part of an amino, acid sequence of the presently disclosed subject matter. The length of sequence comparison is at least 5, 10, 15 or 20 amino acid residues, e.g., at least 25, 50, or 75 amino acid residues, or more than 100 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the polypeptides by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., β or γ amino acids.

In addition to full-length polypeptides, the presently disclosed subject matter also provides fragments of any one of the polypeptides or peptide domains disclosed herein. As used herein, the term “a fragment” means at least 5, 10, 13, or 15 amino acids. In certain embodiments, a fragment comprises at least 20 contiguous amino acids, at least 30 contiguous amino acids, or at least 50 contiguous amino acids. In certain embodiments, a fragment comprises at least 60 to 80, 100, 200, 300 or more contiguous amino acids. Fragments can be generated by methods known to those skilled in the art or may result from normal protein processing (e.g., removal of amino acids from the nascent polypeptide that are not required for biological activity or removal of amino acids by alternative mRNA splicing or alternative protein processing events).

Non-protein analogs have a chemical structure designed to mimic the functional activity of a protein disclosed herein. Such analogs may exceed the physiological activity of the original polypeptide. Methods of analog design are well known in the art, and synthesis of analogs can be carried out according to such methods by modifying the chemical structures such that the resultant analogs increase the anti-neoplastic activity of the original polypeptide when expressed in an immunoresponsive cell. These chemical modifications include, but are not limited to, substituting alternative R groups and varying the degree of saturation at specific carbon atoms of a reference polypeptide. In certain embodiments, the protein analogs are relatively resistant to in vivo degradation, resulting in a more prolonged therapeutic effect upon administration. Assays for measuring functional activity include, but are not limited to, those described in the Examples below.

In accordance with the presently disclosed subject matter, the polynucleotides encoding an extracellular antigen-binding domain that specifically binds to human mesothelin (e.g., a scFv, a Fab, or a (Fab)₂), CD3ζ, CD8, CD28, 4-1BB, 4-1BBL, and IL-12, can be modified by codon optimization. Codon optimization can alter both naturally occurring and recombinant gene sequences to achieve the highest possible levels of productivity in any given expression system. Factors that are involved in different stages of protein expression include codon adaptability, mRNA structure, and various cis-elements in transcription and translation. Any suitable codon optimization methods or technologies that are known to ones skilled in the art can be used to modify the polynucleotides of the presently disclosed subject matter, including, but not limited to, OptimumGene™, Encor optimization, and Blue Heron.

Codon optimization can be performed based on four different algorithms (e.g., Blue Heron and Encore algorithms). The codon optimization sequences obtained from all four algorithms are blended, and all CPGs and BAM-H1 are removed for optimal cloning. In certain embodiments, the codon optimized nucleotide sequence is about 70% homologous to the original sequence prior to codon optimization. In order to obtain efficient expression in an immunoresponsive cell (e.g., human primary T cells), the codon optimized nucleotide sequence is ligated to a CD8 leader, e.g., a polynucleotide encoding SEQ ID NO:71. The CD8 leader provides optimal signal cleavage preceding scFv heavy chain (QVQL). Codon optimization optimize mesothelin CAR expression in an immunoresponsive cell, e.g., multiple human donor primary T cells, with good transduction efficiency. Multiple CAR vector copy numbers in multiple donors T cells are tested for functional efficiency, specificity and sensitivity against multiple hematological and solid cancer cells with varying mesothelin expression. The codon optimized mesothelin-targeted CAR with a vector copy number of 1-4 (more specifically, about 3-4) provides highly efficient cytotoxicity against high mesothelin expressing targets, yet minimal reactivity against low mesothelin expressing targets, i.e. normal tissue. The above-described genetic engineering in generating a specific mesothelin CAR that is reactive against cancer cells expressing high mesothelin while sparing normal tissue expressing low mesothelin is optimal for use as clinical vector for cancer therapy while assuring safety.

### 5.6. Pharmaceutical Compositions and Administration

The presently disclosed subject matte provides compositions comprising the presently disclosed cells (e.g., as disclosed in Section 5.3). The amount of cells comprised in the compositions can vary depending on the purpose of the uses for the composition, and/or the size, age, sex, weight, and condition of the subject who receives the compositions. In certain embodiments, the composition comprises between about 10⁴ and about 10¹⁰, between about 10⁴ and about 10⁶, between about 10⁵ and about 10⁶, between about 10⁵ and about 10⁷, between about 10⁵ and about 10⁹, or between about 10⁶ and about 10⁸ of the presently disclosed immunoresponsive cells. In certain embodiments, the composition comprises at least about 1×10⁵, at least about 5×10⁵, at least about 1×10⁶, at least about 1×10⁷, at least about 1×10⁸ of the presently disclosed immunoresponsive cells. In certain embodiments, the composition comprises about 1×10⁵ of the presently disclosed cells.

Compositions comprising the presently disclosed immunoresponsive cells can be provided systemically or directly to a subject for inducing and/or enhancing an immune response to an antigen and/or treating and/or preventing a neoplasm, pathogen infection, or infectious disease, inflammatory disease, or graft rejection. In certain embodiments, the presently disclosed immunoresponsive cells or compositions comprising thereof are directly injected into an organ of interest (e.g., an organ affected by a neoplasm). Alternatively, the presently disclosed immunoresponsive cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of T cells, NK cells, or CTL cells in vitro or in vivo.

The presently disclosed immunoresponsive cells can be administered in any physiologically acceptable vehicle, normally intravascularly, although they may also be introduced into bone or other convenient site where the cells may find an appropriate site for regeneration and differentiation (e.g., thymus). Usually, at least about 1×10⁵ cells will be administered, eventually reaching about 1×10¹⁰ or more. The presently disclosed immunoresponsive cells can comprise a purified population of cells. Those skilled in the art can readily determine the percentage of the presently disclosed immunoresponsive cells in a population using various well-known methods, such as fluorescence activated cell sorting (FACS). Suitable ranges of purity in populations comprising the presently disclosed immunoresponsive cells are about 50% to about 55%, about 5% to about 60%, and about 65% to about 70%. In certain embodiments, the purity is about 70% to about 75%, about 75% to about 80%, or about 80% to about 85%. In certain embodiments, the purity is about 85% to about 90%, about 90% to about 95%, and about 95% to about 100%. Dosages can be readily adjusted by those skilled in the art (e.g., a decrease in purity may require an increase in dosage). The cells can be introduced by injection, catheter, or the like.

The presently disclosed compositions can be pharmaceutical compositions comprising the presently disclosed immunoresponsive cells or their progenitors and a pharmaceutically acceptable carrier. Administration can be autologous or heterologous. For example, immunoresponsive cells, or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition of the presently disclosed subject matter (e.g., a pharmaceutical composition comprising a presently disclosed immunoresponsive cell), it can be formulated in a unit dosage injectable form (solution, suspension, emulsion).

### 5.7. Formulations

Compositions comprising the presently disclosed immunoresponsive cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the genetically modified immunoresponsive cells in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the genetically modified immunoresponsive cells or their progenitors.

The compositions can be isotonic, i.e., they can have the same osmotic pressure as blood and lacrimal fluid. The desired isotonicity of the compositions may be accomplished using sodium chloride, or other pharmaceutically acceptable agents such as dextrose, boric acid, sodium tartrate, propylene glycol or other inorganic or organic solutes. Sodium chloride can be particularly for buffers containing sodium ions.

Viscosity of the compositions, if desired, can be maintained at the selected level using a pharmaceutically acceptable thickening agent. For example, methylcellulose is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

The quantity of cells to be administered will vary for the subject being treated. In certain embodiments, between about 10⁴ and about 10¹⁰, between about 10⁵ and about 10⁹, between about 10⁴ and about 10⁶, between about 10⁵ and about 10⁶, between about 10⁵ and about 10⁷, or between about 10⁶ and about 10⁸ of the presently disclosed immunoresponsive cells are administered to a human subject. More effective cells may be administered in even smaller numbers. In certain embodiments, at least about 1×10⁵, at least about 1×10⁶, at least about 1×10⁷, 1×10⁸, at least about 2×10⁸, at least about 3×10⁸, at least about 4×10⁸, or at least about 5×10⁸ of the presently disclosed immunoresponsive cells are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art. In certain embodiments, about 1×10⁵ of the presently disclosed cells are administered to a subject.

The skilled artisan can readily determine the amount of cells and optional additives, vehicles, and/or carrier in compositions and to be administered in methods. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, or about 0.05 to about 5 wt %. For any composition to be administered to an animal or human, the followings can be determined: toxicity such as by determining the lethal dose (LD) and LD50 in a suitable animal model e.g., rodent such as mouse; the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### 5.8. Methods of Treatment

The immunoresponsive cells and compositions comprising thereof of the presently disclosed subject matter can be used for the treatment and/or prevention of a neoplasm, pathogen infection, infectious disease, inflammatory disease, or graft rejection. Such immunoresponsive cells can be administered to a subject (e.g., a human subject) in need thereof for the treatment or prevention of a solid tumor (e.g. mesothelioma, lung cancer, pancreatic cancer, ovarian cancer, breast cancer, colon cancer, pleural tumor, glioblastoma, esophageal cancer, gastric cancer, synovial sarcoma, thymic carcinoma, endometrial carcinoma, stomach cancer, and/or cholangiocarcinoma). In certain embodiments, the immunoresponsive cell is a T cell. The T cell can be a CD4⁺ T cell or a CD8⁺ T cell. In certain embodiments, the T cell is a CD4⁺ T cell.

The presently disclosed subject matter provides methods for inducing and/or increasing an immune response in a subject in need thereof. The presently disclosed immunoresponsive cells and compositions comprising thereof can be used for treating and/or preventing a neoplasm in a subject. The presently disclosed immunoresponsive cells and compositions comprising thereof can be used for prolonging the survival of a subject suffering from a neoplasm. The presently disclosed immunoresponsive cells and compositions comprising thereof can also be used for treating and/or preventing a pathogen infection or other infectious disease in a subject, such as an immunocompromised human subject. Such methods comprise administering the presently disclosed immunoresponsive cells in an amount effective or a composition (e.g., pharmaceutical composition) comprising thereof to achieve the desired effect, be it palliation of an existing condition or prevention of recurrence. For treatment, the amount administered is an amount effective in producing the desired effect. An effective amount can be provided in one or a series of administrations. An effective amount can be provided in a bolus or by continuous perfusion.

An “effective amount” (or, “therapeutically effective amount”) is an amount sufficient to effect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the immunoresponsive cells administered.

For adoptive immunotherapy using antigen-specific T cells, cell doses in the range of about 10⁶ to about 10¹⁰ (e.g., about 10⁹) are typically infused. However, due to the high efficiency of the presently disclosed polypeptide compositions, a lesser amount of the presently disclosed cells is required to achieve the desired effects. For example, about 1×10⁵ of the presently disclosed cells are sufficient to achieve the desired effects.

Upon administration of the immunoresponsive cells into the subject and subsequent differentiation, the immunoresponsive cells are induced that are specifically directed against one specific antigen (e.g., human mesothelin). “Induction” of T cells can include inactivation of antigen-specific T cells such as by deletion or anergy. Inactivation is particularly useful to establish or reestablish tolerance such as in autoimmune disorders. The immunoresponsive cells of the presently disclosed subject matter can be administered by any methods known in the art, including, but not limited to, pleural administration, intravenous administration, subcutaneous administration, intranodal administration, intratumoral administration, intrathecal administration, intrapleural administration, intraperitoneal administration, and direct administration to the thymus. In certain embodiments, the immunoresponsive cells and/or the compositions comprising thereof are pleurally administered to the subject in need. In certain embodiments, the immunoresponsive cells and/or the compositions comprising thereof are intrapleurally administered to the subject in need.

The presently disclosed subject matter provides various methods of using the immunoresponsive cells (e.g., T cells). For example, the presently disclosed subject matter provides methods of reducing tumor burden in a subject. In certain embodiments, the method of reducing tumor burden comprises administering an effective amount of the presently disclosed immunoresponsive cells or a composition comprising thereof to the subject. The presently disclosed immunoresponsive cell can reduce the number of tumor cells, reduce tumor size, and/or eradicate the tumor in the subject. The tumor can be a solid tumor. Non-limiting examples of solid tumor include mesothelioma, lung cancer, pancreatic cancer, ovarian cancer, breast cancer, colon cancer, pleural tumor, glioblastoma, esophageal cancer, gastric cancer, synovial sarcoma, thymic carcinoma, endometrial carcinoma, stomach cancer, and cholangiocarcinoma.

The presently disclosed subject matter also provides methods of increasing or lengthening survival of a subject having a neoplasm. In certain embodiments, the method of increasing or lengthening survival of a subject having neoplasia neoplasm comprises administering an effective amount of the presently disclosed immunoresponsive cells or a composition comprising thereof to the subject. The method can reduce or eradicate tumor burden in the subject. Additionally, the presently disclosed subject matter provides methods for increasing an immune response in a subject, comprising administering the presently disclosed immunoresponsive cell or a composition comprising thereof to the subject. The presently disclosed subject matter further provides methods for treating and/or preventing a neoplasm in a subject, comprising administering the presently disclosed immunoresponsive cell or a composition comprising thereof to the subject.

In certain embodiments, the neoplasm is a solid tumor. The neoplasm can a primary tumor or primary cancer. In addition, the neoplasm can be in metastatic status.

Cancers whose growth may be inhibited using the immunoresponsive cells of the presently disclosed subject matter comprise cancers typically responsive to immunotherapy. Non-limiting examples of cancers for treatment include mesothelioma, lung cancer (e.g. non-small cell lung cancer), pancreatic cancer, ovarian cancer, breast cancer (e.g., metastatic breast cancer, metastatic triple-negative breast cancer), colon cancer, pleural tumor, glioblastoma, esophageal cancer, gastric cancer, synovial sarcoma, thymic carcinoma, endometrial carcinoma, stomach cancer, cholangiocarcinoma, cervical cancer, and salivary gland cancer. Additionally, the presently disclosed subject matter comprises refractory or recurrent malignancies whose growth may be inhibited using the immunoresponsive cells of the presently disclosed subject matter.

Examples of other neoplasms or cancers that may be treated using the methods of the presently disclosed subject matter include bone cancer, intestinal cancer, liver cancer, skin cancer, cancer of the head or neck, melanoma (cutaneous or intraocular malignant melanoma), renal cancer (e.g. clear cell carcinoma), throat cancer, prostate cancer (e.g. hormone refractory prostate adenocarcinoma), blood cancers (e.g. leukemias, lymphomas, and myelomas), uterine cancer, rectal cancer, cancer of the anal region, bladder cancer, brain cancer, stomach cancer, testicular cancer, carcinoma of the fallopian tubes, carcinoma of the endometrium, carcinoma of the cervix, carcinoma of the vagina, carcinoma of the vulva, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, include Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, nile duct carcinoma, choriocarcinoma, seminoma, embryonal carcinoma, Wilm's tumor, cervical cancer, salivary gland cancer, uterine cancer, testicular cancer, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

Additionally, the presently disclosed subject matter provides methods of increasing immune-activating cytokine production in response to a cancer cell or a pathogen in a subject. In certain embodiments, the method comprises administering the presently disclosed immunoresponsive cell or composition comprising thereof to the subject. The immune-activating cytokine can be granulocyte macrophage colony stimulating factor (GM-C SF), IFN-α, IFN-β, IFN-γ, TNF-α, IL-2, IL-3, IL-6, IL-11, IL-7, IL-12, IL-15, IL-21, interferon regulatory factor 7 (IRF7), and combinations thereof. In certain embodiments, the immunoresponsive cells increase the production of GM-CSF, IFN-γ, and/or TNF-α.

The presently disclosed subject matter provides therapies that are particularly useful for treating solid tumors (e.g., mesothelioma, lung cancer, pancreatic cancer, ovarian cancer, breast cancer, colon cancer, pleural tumor, glioblastoma, esophageal cancer, gastric cancer, synovial sarcoma, thymic carcinoma, endometrial carcinoma, stomach cancer, and cholangiocarcinoma). Solid tumors can be primary tumors or tumors in metastatic state. Certain solid tumors are heterogeneous MSLN expressing tumors, e.g., breast cancer (e.g., TNBC), lung cancer, ovarian cancer, pancreatic cancer, esophagus cancer, colon cancer, gastric cancer, and malignant pleural mesothelioma (MPM). Heterogeneous MSLN expressing cells (e.g., tumor cells) are a population of cells comprising low MSLN-expressing cells and high MSLN-expressing cells. The presently disclosed immunoresponsive cell can exhibit increased cytotoxicity and antitumor activity to low MSLN-expressing cells (e.g., about 2,000 or less, about 1,000 or less, about 900 or less, about 800 or less, about 700 or less, about 600 or less, about 500 or less, about 400 or less, about 300 or less, about 200 or less, or about 100 or less MSLN binding sites/cell), in the presence of high MSLN-expressing cells. In certain embodiments, even in the presence of high MSLN-expressing cells, immunoresponsive cell does not exhibit increased cytotoxicity or nonspecific kill to MSLN-negative cells. Thus, the immunoresponsive cell can exhibit increased cytotoxicity and antitumor activity to low MSLN-expressing cells in the presence of high MSLN-expressing cells while retain safety to MSLN-negative cells.

Furthermore, the presently disclosed subject matter provides methods for treating subjects with a pathogen infection (e.g., viral infection, bacterial infection, fungal infection, parasite infection, or protozoal infection). The presently disclosed subject matter is particularly useful for enhancing an immune response in an immunocompromised subject. Exemplary viral infections susceptible to treatment using a method of the invention include, but are not limited to, Cytomegalovirus (CMV), Epstein Barr Virus (EBV), Human Immunodeficiency Virus (HIV), and influenza virus infections. Accordingly, the presently disclosed subject matter provides a method of treating or preventing a pathogen infection in a subject, the method comprising administering an effective amount of the presently disclosed immunoresponsive cells or composition comprising thereof.

In accordance with the presently disclosed subject matter, the above-described various methods can comprise administering at least one immunomodulatory agent. Non-limiting examples of immunomodulatory agents include immunostimulatory agents, checkpoint immune blockade agents, radiation therapy agents, and chemotherapy agents. In certain embodiments, the immunomodulatory agent is an immunostimulatory agent. Non-limiting examples of immunostimulatory agents include IL-12, and agonist costimulatory monoclonal antibodies. In certain embodiments, the immunostimulatory agent is IL-12. In certain embodiments, the presently disclosed immunoresponsive cells or composition comprising thereof in combination with anti-IL-12 antibody can be used to treat breast cancer (BC), e.g., metastatic triple-negative breast cancer (TNBC). Non-limiting examples of agonist costimulatory monoclonal antibodies include anti-4-1BB antibodies, anti-OX40 antibodies, and anti-ICOS antibodies. In certain embodiments, the agonist costimulatory monoclonal antibody is an anti-4-1BB antibody.

In certain embodiments, the presently disclosed immunoresponsive cells or composition comprising thereof not only can exhibit tumor-targeted adoptive T-cell therapy but can enhance T cell function through the design of improved antigen receptors and through intervention in the host microenvironment by immunomodulation using IL-12. Among all immunotherapeutic approaches, IL-12, a multifunctional cytokine, has been considered to be one of the most promising approaches to treat BC (Boggio, K., et al., Cancer Res 60, 359-364 (2000); Czerniecki, B. J., et al., Cancer Res 67, 1842-1852 (2007); Nanni, P., et al., J Exp Med 194, 1195-1205 (2001)). IL-12 is considered a master regulator of adaptive type 1 cell-mediated immunity, the critical pathway involved in antitumor responses (Del Vecchio, M., et al., Clin Cancer Res 13, 4677-4685 (2007)). IL-12 modulates antitumor responses at various levels, including polarization of CD4 T cells toward a Th1 phenotype (Wesa, et al., J Immunother 30, 75-82 (2007)), boosting of T cell and NK effector functions (Curtsinger et al., J Exp Med 197, 1141-1151 (2003).), remodeling the innate immune response (Chmielewski et al., Cancer Res 71, 5697-5706 (2011)), and regulating tumor angiogenesis (Voest et al., J Natl Cancer Inst 87, 581-586 (1995)). The immunomodulating and antiangiogenic functions of IL-12 have provided the rationale for using this cytokine in combination with the immunoresponsive cell of the presently disclosed subject matter for treating cancers, e.g., BC (e.g., TNBC). Among 148 clinical trials including administration of IL-12 to patients with cancer (36 of which were reported recently), successful phase II studies with intraperitoneal (Lenzi et al. Clin.Cancer Res. 8, 3686-3695 (2002)) or subcutaneous (Mahvi et al. Cancer Gene Ther. 14, 717-723 (2007); Kang et al. Hum.Gene Ther. 12, 671-684 (2001)). IL-12 have shown that paracrine secretion of IL-12, generated by gene transfer, can induce immunity against the tumor locally and at a distant site. Although several studies have documented the anticancer effectiveness of IL-12 in preclinical models of breast cancer (BC) (Boggio et al. Cancer Res 60, 359-364 (2000); Nanni et al. J Exp Med 194, 1195-1205 (2001)), the significant toxicity resulting from administration of recombinant human IL-12 observed in several clinical trials in advanced cancers precludes its clinical use. To overcome this limitation, a number of groups have demonstrated that intratumoral delivery of IL-12, using adenoviral vectors, induces tumor regression and T cell activation in preclinical models of BC (Gyorffy et al. J Immunol 166, 6212-6217 (2001); Bramson et al. Hum Gene Ther 7, 1995-2002 (1996)). More recently, Sabel et al. used polylactic acid microspheres to release IL-12 into the tumor and found that the antitumor response was mediated primarily by NK cells (Sabel et al. Breast Cancer Res Treat 122, 325-336 (2010)). Others have used mesenchymal stromal cells to locally deliver IL-12 to mouse BC (Eliopoulos et al. Cancer Res 68, 4810-4818 (2008)). A phase I trial of paclitaxel and trastuzumab, in combination with IL-12, in patients with HER2/neu-expressing malignancies showed an impressive synergy between IL-12 and trastuzumab for stimulation of NK-cell cytokine secretion (Bekaii-Saab et al. Molecular cancer therapeutics 8, 2983-2991 (2009)). Therefore, IL-12 can have considerable promise as an anticancer agent, and its use as a co-stimulant in an adoptive T cell therapy approach is well-justified.

In certain embodiments, the immunomodulatory agent is a checkpoint immune blockade agent. Non-limiting examples of checkpoint immune blockade agents include anti-PD-L1 antibodies, anti-CTLA-4 antibodies, anti-PD-1 antibodies, anti-LAG3 antibodies, anti-B7-H3 antibodies, and anti-TIM3 antibodies. In certain embodiments, the checkpoint immune blockade agent is an anti-PD-L1 antibody. In certain embodiments, the immunoresponsive cell of the presently disclosed subject matter or composition comprising thereof in combination with anti-PD-L1 antibody can be used to treat breast cancer (BC), e.g., TNBC.

Programmed cell death ligand 1 (PD-L1/B7-H4/CD274) is an inhibitory signal typically expressed in actively inflamed tissues, serving as a negative feedback loop to limit T cell activation. PD-L1 expression is typically absent from uninflamed normal tissues (including breast (Dong et al. Nature medicine 8, 793-800 (2002))) and is instead most prevalent in cancer tissues, particularly in those with an inflammatory infiltrate (Spranger et al. Science translational medicine 5, 200ra1 16 (2013)). This association with inflammation is likely due to PD-L1 upregulation upon tumor cell exposure to T cell-secreted cytokines generated upon T cell activation. This pattern of expression is exhibited by BCs, with 50%-75% of BC specimens staining positive for PD-L1 and with expression strongly associated with severe lymphocytic infiltrate (Brown et al. Journal of immunology 170, 1257-1266 (2003); Ghebeh et al. Neoplasia 8, 190-198 (2006); Ghebeh et al. BMC cancer 8, 57 (2008)). BC-infiltrating T cells also expressed PD-L1 in 54% of patients (Ghebeh et al. BMC cancer 8, 57 (2008)). BCs may also innately express PD-L1 secondary to oncogenic signaling. Activation of the PI(3)K pathway results in PD-L1 protein upregulation in BC cells, and PI(3)K activation in patient tumors significantly correlates with PD-L1 expression (Crane et al. Oncogene 28, 306-312 (2009)). The expression of PD-1 by activated T cells spatially and temporally links ligand with receptor expression within the immunosuppressive TME. Expression of PD-L1 in BC tissues suggests it as an immunotherapeutic target for these patients. Efficacy of PD-L1/PD-1 blockade in multiple preclinical cancer models (including breast (Ge et al. Cancer letters 336, 253-259 (2013))) paved the way for phase I trials using PD-L1- or PD-1-targeting antibodies for patients with advanced cancers. A phase I study (using a PD-1 antibody) demonstrated efficacy only in PD-L1+ patients (Topalian et al. The New England journal of medicine 366, 2443-2454 (2012)). Genetically engineered T cells offer unique advantages for overcoming co-inhibitory checkpoints and the typical lack of co-stimulation found within the TME. CAR-expressing T cells are indeed engineered to optimize their co-stimulatory requirements to support T cell expansion, survival, and function.

In some embodiments, the immunomodulatory agent is a radiation therapy agent. The localized, radiation-induced immunological milieu not only can provide the preconditions to enhance the engraftment of targeted T cells in the tumor (thereby eliminating the need for systemic lymphodepleting regimens), but that the immunological responses resulting from a combination of radiation therapy and adoptive T cell therapy also enhance abscopal antitumor efficacy. In radiation-resistant tumors, 4-1BB co-stimulatory signaling in CAR T cells can overcome immunoinhibition. In some embodiments, the immunomodulatory agent is a chemotherapy agents, including, but not limited to, cisplatin. Cisplatin-induced secretion of chemokines and cytokines can promote MSLN-targeted and endogenous T-cell responses. Studies have shown that patients with lung adenocarcinoma (LAC) and malignant pleural mesothelioma (MPM) who present with high levels of cytotoxic tumor infiltrating lymphocytes (cTILs) and low levels of regulatory T cells (Tregs) have a better prognosis and longer progression-free survival (Servais, et al., Clin Cancer Res (May 1, 2012);18:2478-2489; Kachala et al., Clin Cancer Res (2013);20(4); 1020-8). An adoptive T-cell therapy using a MSLN-targeted CAR can be used to promote cTILs in LAC and MPM. Servais (2012) and Kachala (2013) report that MSLN is over-expressed and promotes aggressiveness in LAC and MPM—justifying the choice of MSLN as a target for CAR T-cell therapy. The higher proportion of TILs following cisplatin and radiation therapy are associated with improved outcomes both in mouse models and in patients.

Tumor radiation- and cisplatin therapy-induced tumoral and abscopal immunomodulation can provide the preconditioning required for better engraftment of adoptively transferred T cells; T-cell co-stimulatory strategies to exploit the tumor and stromal immunomodulation can potentiate the antitumor efficacy of both endogenous and adoptively transferred T cells.

Additionally, the above-described various methods of using the immunoresponsive cells (e.g., T cells) expressing a mesothelin-specific CAR, e.g., for treating cancer in a subject, or for reducing tumor burden in a subject, can be combined with cancer cell antigen modulation. Immunoresponsive cells (e.g., T cells) expressing a mesothelin-specific CAR can target and kill the MSLN expressed on the membrane (referred to as “cell membrane MSLN”) of a tumor or cancerous cell but not cytoplasmic MSLN. Certain tumors or cancers (e.g., lung cancer, and mesothelioma) have low cell membrane MSLN, but high cytoplasmic MSLN. Cancer cell antigen modulation can increase the expression of cell membrane MSLN in a tumor or cancerous cell, which can make the tumor or cancerous cell more likely be targeted by the CAR-expressing immunoresponsive cell, and thus, more susceptible to the killing by the immunoresponsive cell. In certain embodiments, the cancer cell antigen modulation is radiation.

Further modification can be introduced to the immunoresponsive cells (e.g., T cells) to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the CAR-expressing T cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T cell elimination by administering anti-EGFR monoclonal antibody (e.g., cetuximab). EGFRt can be covalently joined to the 3′ terminus of the intracellular signaling domain of the mesothelin-targeted CAR. The suicide gene can be included within the vector comprising nucleic acids encoding the presently disclosed mesothelin-specific CARs. In this way, administration of a prodrug designed to activate the suicide gene (e.g., a prodrug (e.g., AP1903 that can activate iCasp-9) during malignant T-cell transformation (e.g., GVHD) triggers apoptosis in the suicide gene-activated CAR-expressing T cells.

In addition, the presently disclosed subject matter provides a method of preventing and/or treating an inflammatory disease in a subject. In certain embodiments, the method comprises administering the presently disclosed immunoresponsive cell or composition comprising thereof to the subject. In certain embodiments, the immunoresponsive cell is an immunoinhibitory cell. In certain embodiments, the immunoinhibitory cell is a regulatory T cell. In certain embodiments, the inflammatory disease is pancreatitis. In certain embodiments, the subject is a human. In certain embodiments, the subject is a recipient of an organ transplant, e.g., a recipient of a pancreas transplant.

Furthermore, the presently disclosed subject matter provides a method of preventing graft rejection in a subject who is a recipient of an organ transplant. In certain embodiments, the method comprises administering the presently disclosed immunoresponsive cell or composition comprising thereof to the subject. In certain embodiments, the immunoresponsive cell is an immunoinhibitory cell. In certain embodiments, the immunoinhibitory cell is a regulatory T cell. In certain embodiments the subject is a human. In a further embodiment, the subject is a recipient of a pancreas transplant.

A presently disclosed mesothelin-targeted CAR can be transduced into an immunoinhibitory cell, e.g., a regulatory T cell. The transduced immunoinhibitory cell can be administered to a subject (e.g., a human) having inflammatory conditions or an inflammatory disease. In some embodiments, the inflamed site or the site of the inflammatory disease has a high expression level of mesothelin, which is recognized by the presently disclosed MSLN-CAR. The inflammatory condition can be extreme, e.g., severe pancreatitis. In addition, the transduced immunoinhibitory cell can be administered to a subject who is a recipient of an organ transplant.

Additionally, a presently disclosed mesothelin-targeted CAR as well as a second CAR targeting an MHC antigen can be co-transduced into an immunoinhibitory cell (e.g., regulatory T cell) so that the immunoinhibitory cell can specifically collect at the site of the transplanted pancreas. In certain embodiments, a MHC class I subject receives a pancreas transplant from a MHC class II donor; the regulatory T cells of the recipient are transduced with the presently disclosed MSLN-specific CAR and a second CAR targeting a MHC class II antigen, and thus, the transduced regulatory T cells of the recipient collect/pool at the site of the transplanted pancreas and avoid graft or organ rejection.

Suitable human subjects for therapy typically comprise two treatment groups that can be distinguished by clinical criteria. Subjects with “advanced disease” or “high tumor burden” are those who bear a clinically measurable tumor. A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition is administered to these subjects to elicit an anti-tumor response, with the objective of palliating their condition. Ideally, reduction in tumor mass occurs as a result, but any clinical improvement constitutes a benefit. Clinical improvement includes decreased risk or rate of progression or reduction in pathological consequences of the tumor.

A second group of suitable subjects is known in the art as the “adjuvant group.” These are individuals who have had a history of neoplasm, but have been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for each different neoplasm. Features typical of high-risk subgroups are those in which the tumor has invaded neighboring tissues, or who show involvement of lymph nodes.

Another group have a genetic predisposition to neoplasm but have not yet evidenced clinical signs of neoplasm. For instance, women testing positive for a genetic mutation associated with breast cancer, but still of childbearing age, can wish to receive one or more of the immunoresponsive cells described herein in treatment prophylactically to prevent the occurrence of neoplasm until it is suitable to perform preventive surgery.

As a consequence of surface expression of an anti-mesothelin CAR and a PD-1 DN that enhances the anti-tumor effect of the immunoresponsive cell, adoptively transferred T or NK cells are endowed with augmented and selective cytolytic activity at the tumor site. Furthermore, subsequent to their localization to tumor or viral infection and their proliferation, the T cells turn the tumor or viral infection site into a highly conductive environment for a wide range of immune cells involved in the physiological anti-tumor or antiviral response (tumor infiltrating lymphocytes, NK-, NKT-cells, dendritic cells, and macrophages).

Additionally, the presently disclosed subject matter provides methods for treating and/or preventing a pathogen infection (e.g., viral infection, bacterial infection, fungal infection, parasite infection, or protozoal infection) in a subject, e.g., in an immunocompromised subject. The method can comprise administering an effective amount of the presently disclosed immunoresponsive cells or a composition comprising thereof to a subject having a pathogen infection. Exemplary viral infections susceptible to treatment include, but are not limited to, Cytomegalovirus (CMV), Epstein Barr Virus (EBV), Human Immunodeficiency Virus (HIV), and influenza virus infections.

Further modification can be introduced to the presently disclosed immunoresponsive cells (e.g., T cells) to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the presently disclosed immunoresponsive cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T cell elimination by administering anti-EGFR monoclonal antibody (e.g., cetuximab). EGFRt can be covalently joined to the upstream of the antigen-recognizing receptor of a presently disclosed CAR. The suicide gene can be included within the vector comprising nucleic acids encoding a presently disclosed CAR. In this way, administration of a prodrug designed to activate the suicide gene (e.g., a prodrug (e.g., AP1903 that can activate iCasp-9) during malignant T-cell transformation (e.g., GVHD) triggers apoptosis in the suicide gene-activated CAR-expressing T cells. The incorporation of a suicide gene into the a presently disclosed CAR gives an added level of safety with the ability to eliminate the majority of CART cells within a very short time period. A presently disclosed immunoresponsive cell (e.g., a T cell) incorporated with a suicide gene can be pre-emptively eliminated at a given timepoint post CAR T cell infusion, or eradicated at the earliest signs of toxicity.

### 5.9. Kits

The presently disclosed subject matter provides kits for inducing and/or enhancing an immune response and/or treating and/or preventing a neoplasm or a pathogen infection in a subject. In certain embodiments, the kit comprises an effective amount of presently disclosed immunoresponsive cells or a pharmaceutical composition comprising thereof. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain embodiments, the kit includes an isolated nucleic acid molecule encoding an anti-mesothelin CAR and an isolated nucleic acid molecule encoding a PD-1 DN in expressible form, which may optionally be comprised in the same or different vectors.

If desired, the immunoresponsive cells and/or nucleic acid molecules are provided together with instructions for administering the cells or nucleic acid molecules to a subject having or at risk of developing a neoplasm or pathogen or immune disorder. The instructions generally include information about the use of the composition for the treatment and/or prevention of a neoplasm or a pathogen infection. In certain embodiments, the instructions include at least one of the following: description of the therapeutic agent; dosage schedule and administration for treatment or prevention of a neoplasm, pathogen infection, or immune disorder or symptoms thereof; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### 6. EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the presently disclosed cells and compositions, and are not intended to limit the scope of what the inventors regard as their invention.

### Example 1

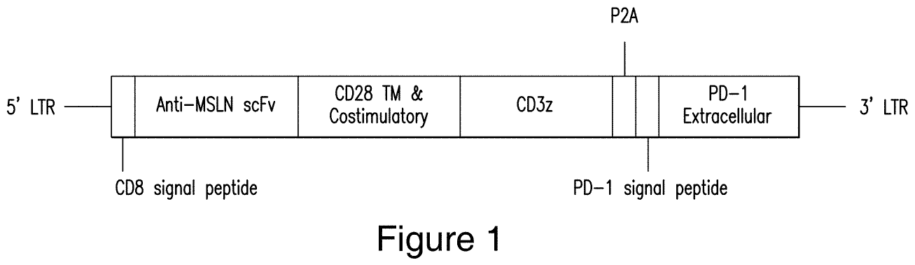

A presently disclosed polypeptide composition was generated. The polypeptide composition comprises: (i) a CAR that binds to human mesothelin and (ii) a dominant negative form of programmed death 1 (PD-1 DN), as shown in FIG. 1. The mesothelin-targeted CAR comprises (a) a CD8 signal peptide (e.g., a CD8 signal peptide consisting of the amino acid sequence set forth in SEQ ID NO: 71), (b) an extracellular antigen-binding domain that is a scFv comprising a VH comprising a CDR1 consisting of the amino acid sequence set forth in SEQ ID NO: 76, a CDR2 consisting of the amino acid sequence set forth in SEQ ID NO: 77, and a CDR3 having the amino acid sequence set forth in SEQ ID NO: 78; and a V_{L} comprising a CDR1 consisting of the amino acid sequence set forth in SEQ ID NO: 79, a CDR2 consisting of the amino acid sequence set forth in SEQ ID NO: 80, and a CDR3 consisting of the amino acid sequence set forth in SEQ ID NO: 81, (c) a transmembrane domain that comprises a CD28 polypeptide (e.g., a CD28 polypeptide consisting of the amino acid sequence set forth in SEQ ID NO: 92 (or amino acids 153 to 179 of SEQ ID NO: 90)), (d) a CD28 hinge/spacer region (e.g., a CD28 polypeptide consisting of the amino acid sequence set forth in SEQ ID NO: 15 (or amino acids 114 to 152 of SEQ ID NO: 90)), and (e) an intracellular signaling domain comprising a modified CD3ζ polypeptide consisting of the amino acid sequence set forth in SEQ ID NO: 35, and a co-stimulatory signaling region that comprises a CD28 polypeptide (e.g., a CD28 polypeptide consisting of the amino acid sequence set forth in SEQ ID NO: 101 (or amino acids 180 to 220 of SEQ ID NO: 90)). The PD-1 DN comprises a PD-1 signal peptide consisting of amino acids 1 to 20 of SEQ ID NO: 48 , a PD-1 extracellular domain consisting of amino acids 21 to 165 of SEQ ID NO: 48, and a CD8 polypeptide consisting of amino acids 137 to 207 of SEQ ID NO: 86. The polypeptide composition also comprises a P2A peptide having the amino acid sequence set forth in SEQ ID NO: 121, and is positioned between the CAR and the PD-1 DN, as shown in FIG. 1. The polypeptide composition is designed as “M28z1XXPD1DNR”.

The CAR comprised in the polypeptide construct has the amino acid sequence set forth in SEQ ID NO: 56. An exemplary nucleotide sequence encoding the polypeptide construct is set forth in SEQ ID NO: 123. Another exemplary nucleotide sequence encoding the polypeptide composition is set forth in SEQ ID NO: 124.

### Example 2

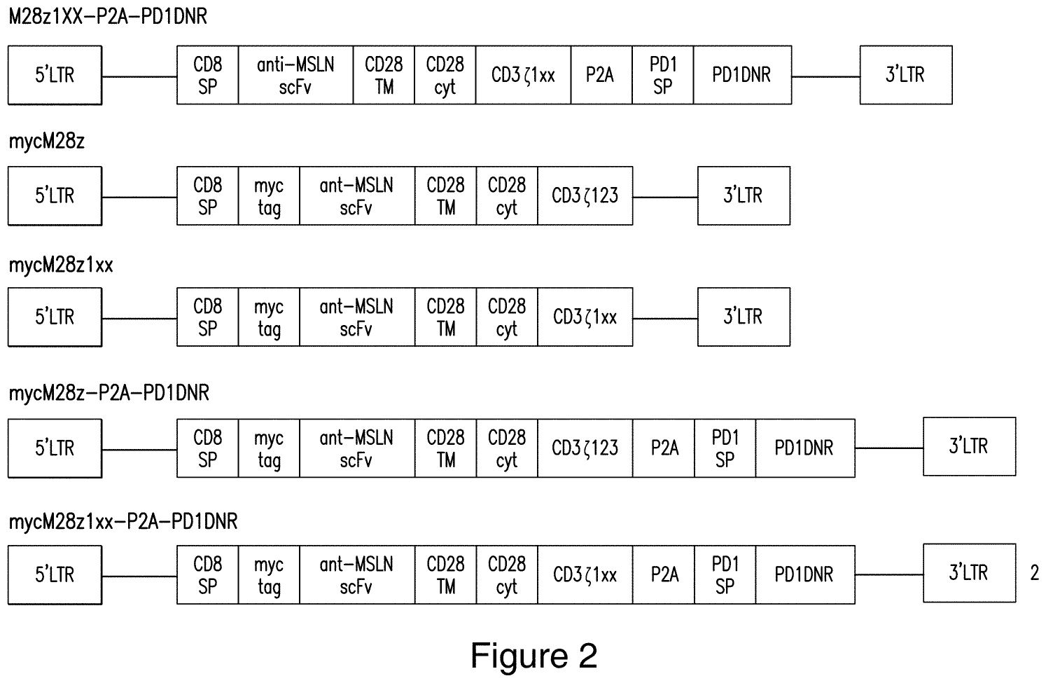

The activities of M28z1XX-P2A-PD1DNR having the structure of the polypeptide composition as described in Example 1 was studied. The structures of alternative and control constructs were compared to M28z1XX-P2A-PD1DNR as shown in FIG. 2.

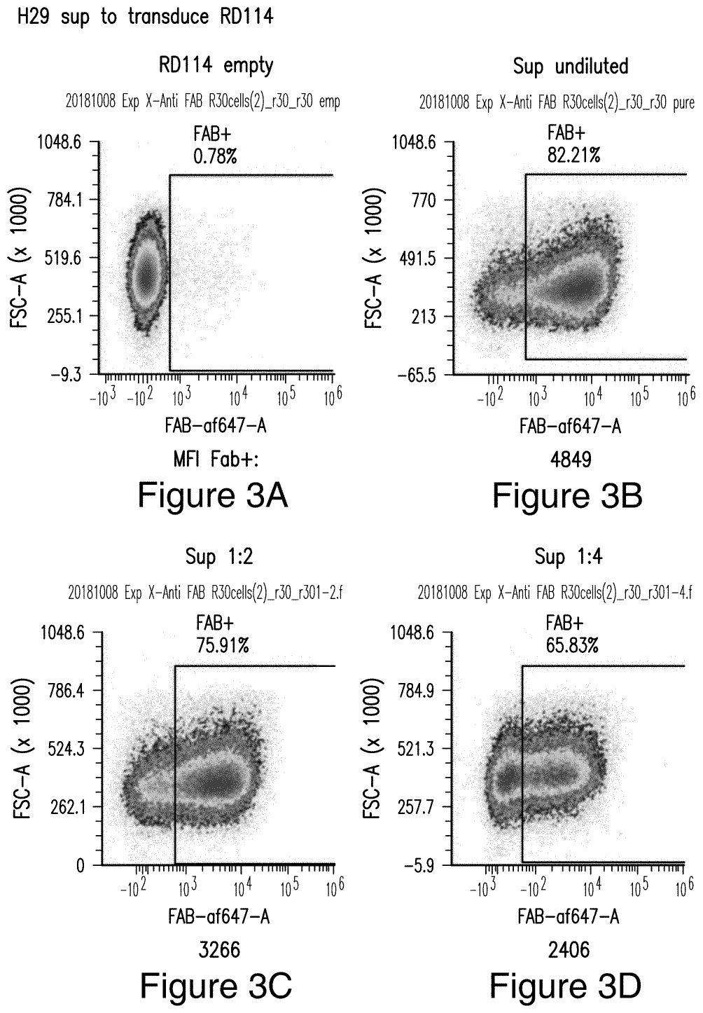

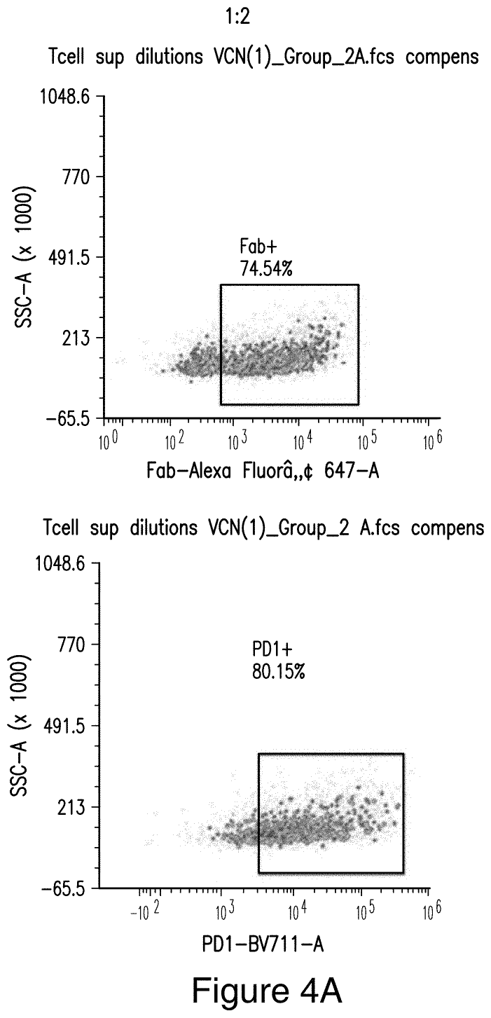

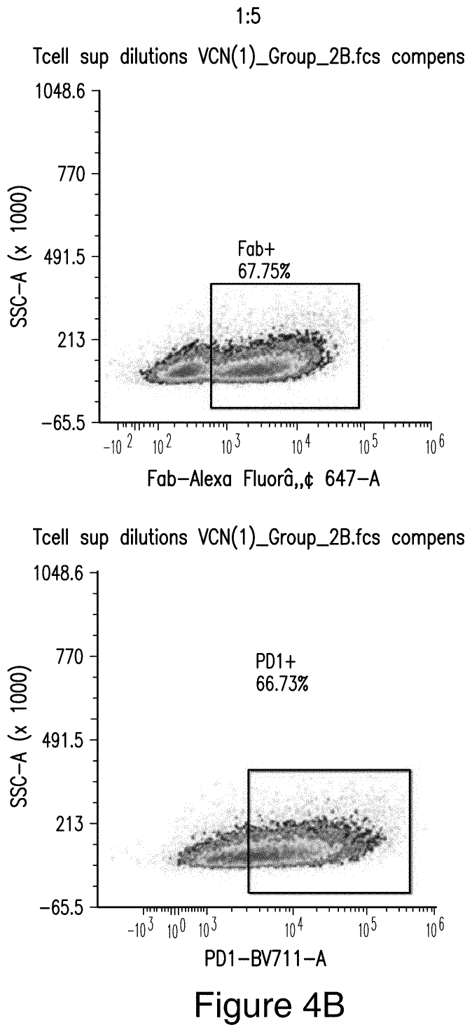

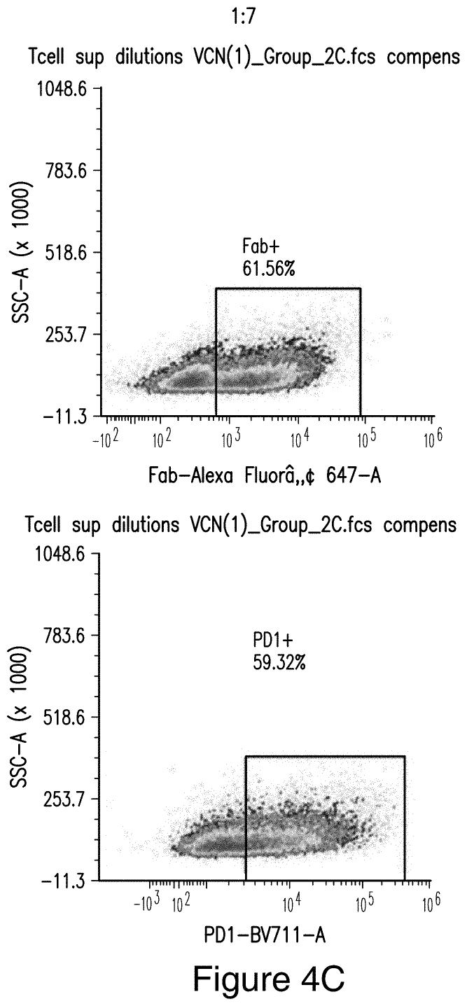

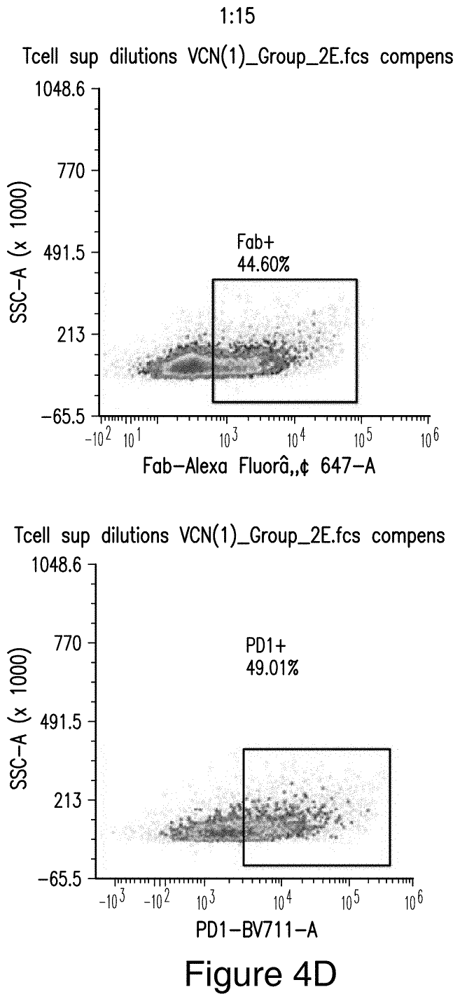

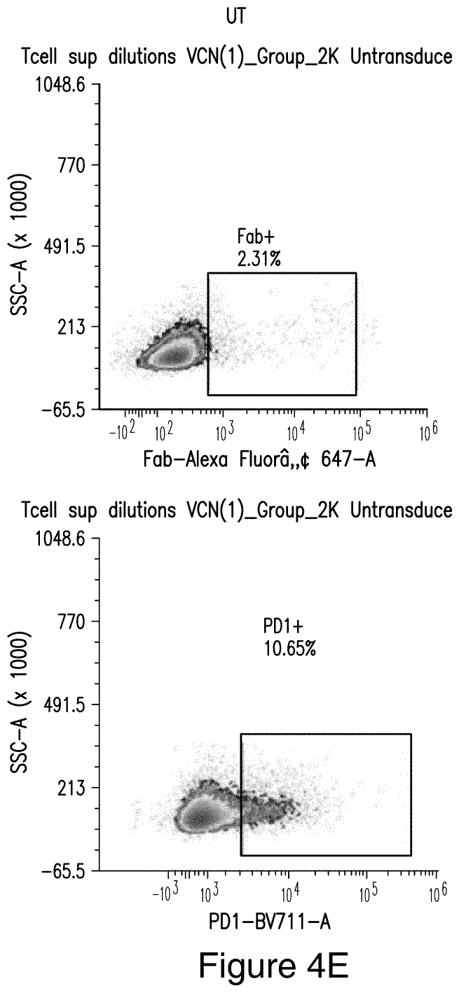

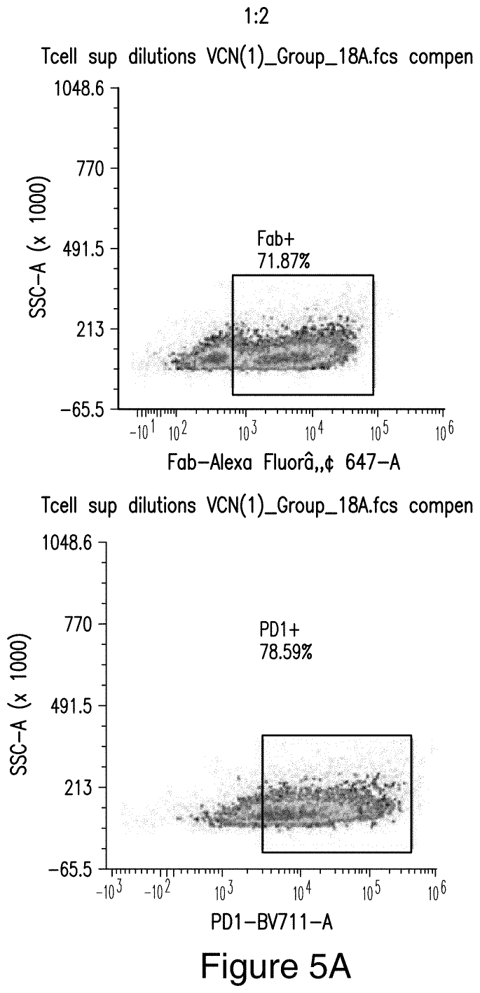

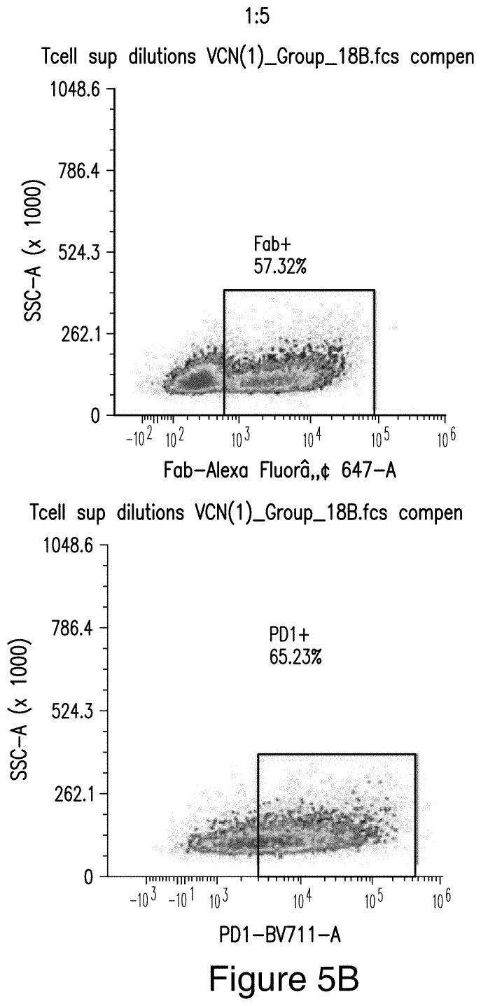

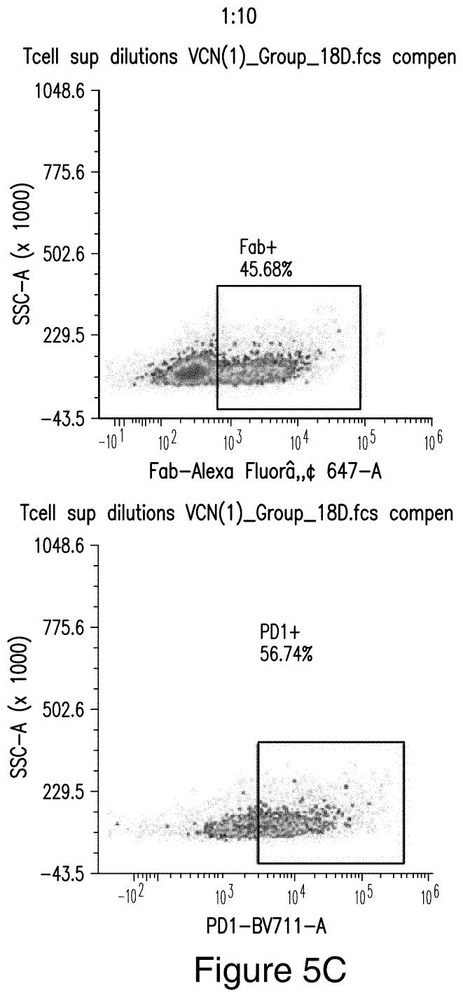

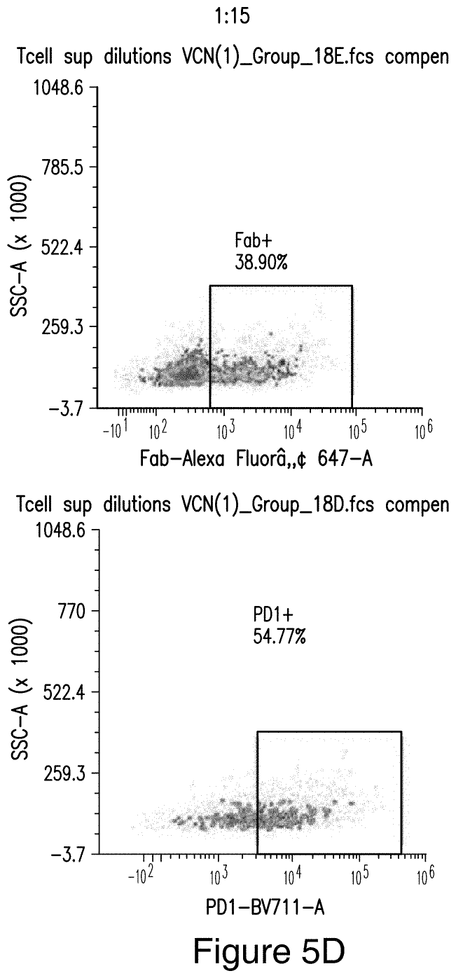

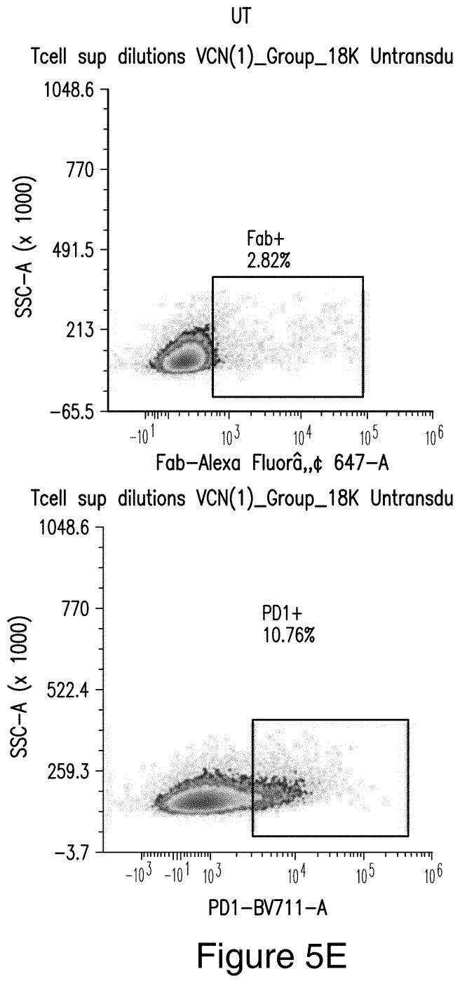

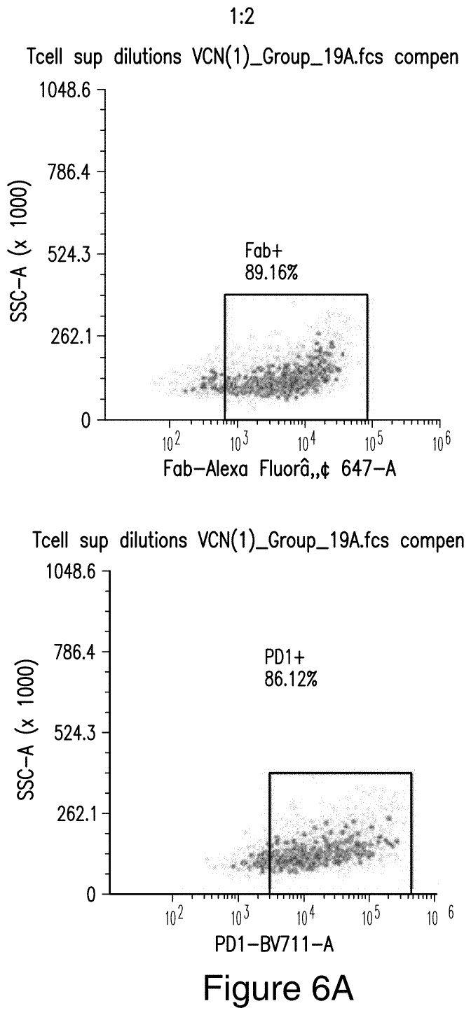

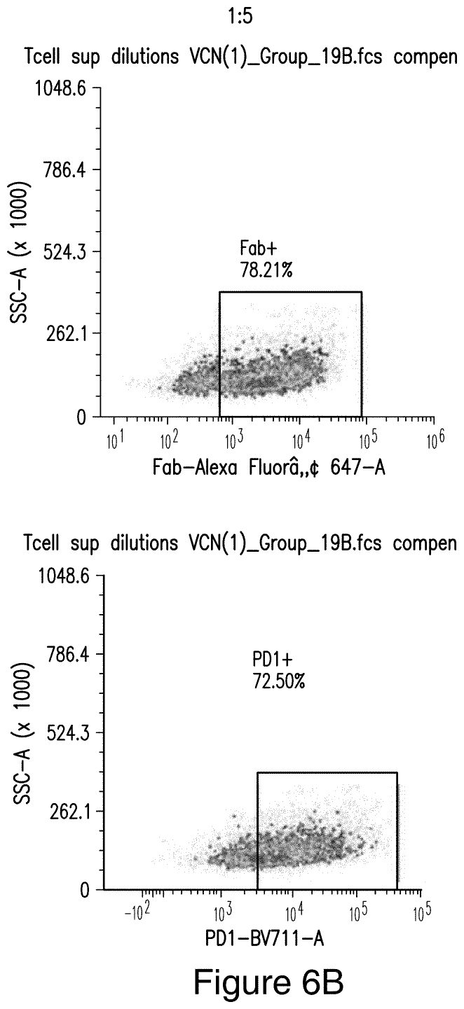

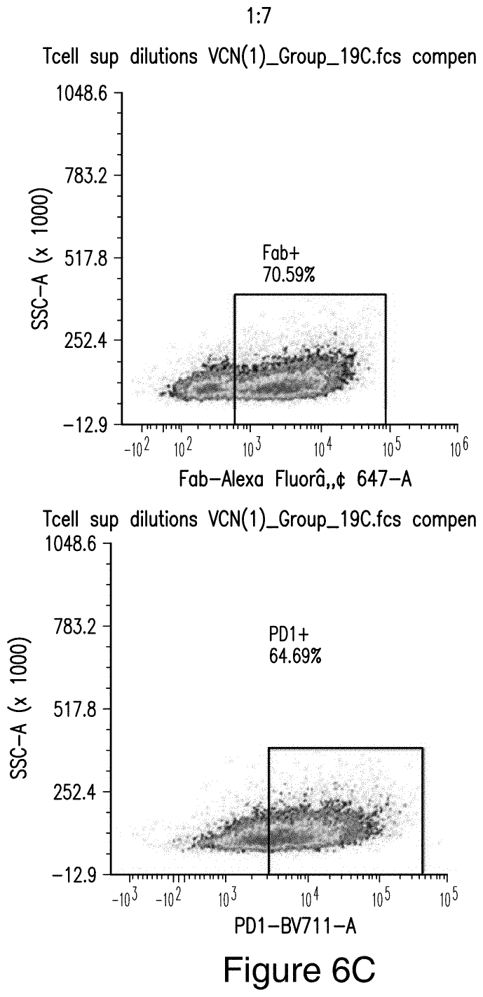

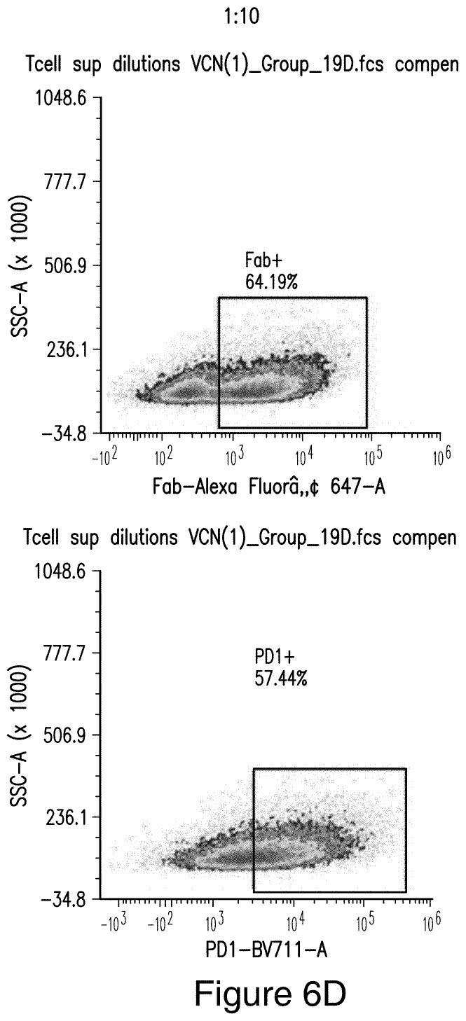

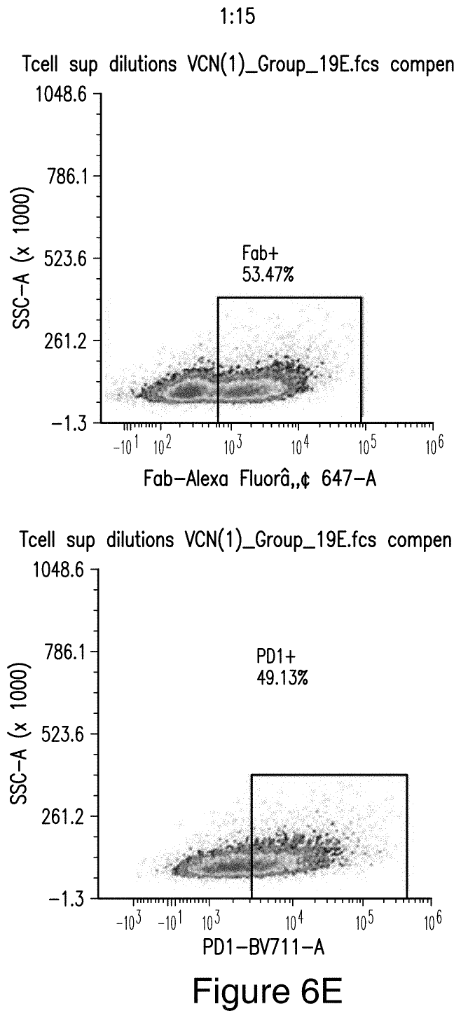

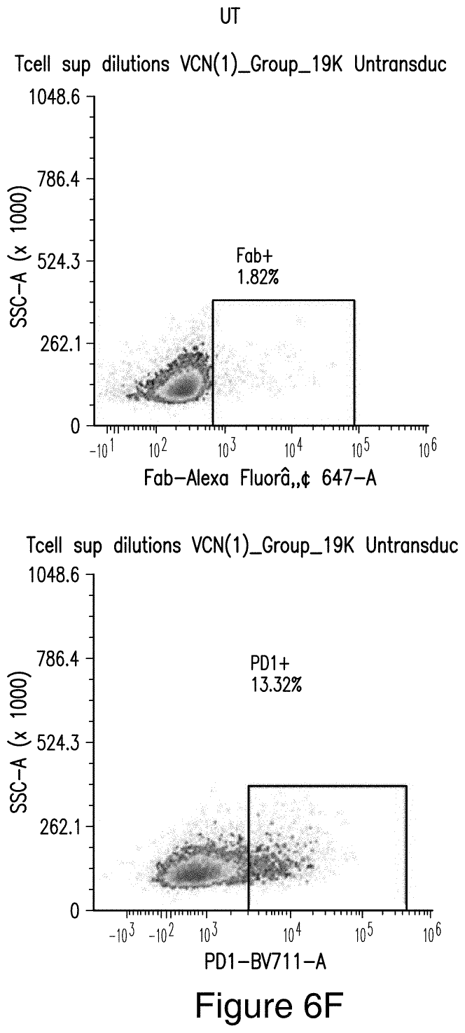

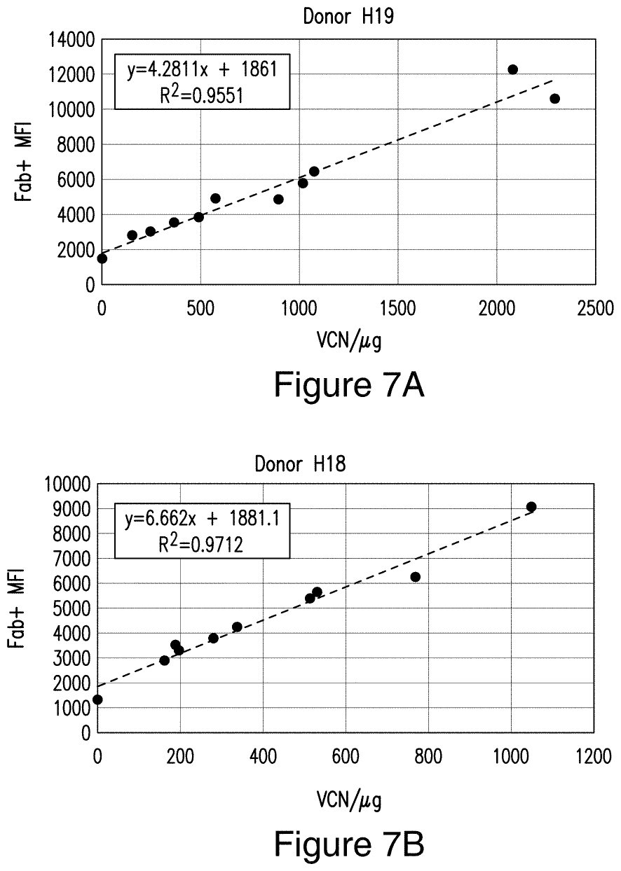

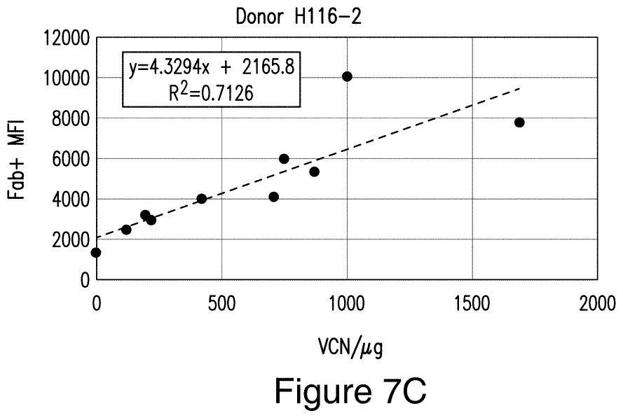

Viral vectors comprising the CAR constructs were generated in producer cell line RD114 as shown in FIGS. 3A-3D. RD114 cells were transduced with different dilutions of H29 viral supernatant (undiluted, 1:2, and 1:4) and stained for CAR expression by flow cytometry using an anti-Fab antibody. RD114 empty served as a negative control. Human T cells were successful transduced with M28z1XX-P2A-PD1DNR as shown in FIGS. 4A-4E, 5A-5E, and 6A-6F. PHA-activated T cells were transduced with different concentrations of RD114 viral supernatant and stained for CAR expression by anti-Fab staining and PD1DNR by anti-PD1 staining using flow cytometry. Whether the vector copy number (VCN) was correlated with median fluorescence intensity (MFI) was studied. PHA-activated T cells were transduced with different concentrations of RD114 viral supernatant and stained for CAR expression by anti-Fab staining and flow cytometry analysis. Genomic DNA of transduced T cells was isolated and vector copy number was determined as VCN/μg DNA using qPCR. As shown in FIGS. 7A-7C, the MFI of CAR-positive cells was correlated with the VCN/μg DNA for all three tested donors. Transduction ratios of human CD4⁺ and CD8⁺ T cells are shown in Table 1.

TABLE 1

| | | CD3+ Fab+ | | CD3+ Fab+ PD1+ | | CD3+ CD8+ | | CD3+ CD4+ | |
|---|---|---|---|---|---|---|---|---|---|
| | | | MFI of | | MFI of | % | % | % | % |
| Donor | Dilution | % | positive | % | positive | total | Fab+ | total | Fab+ |
| H116-2 | 1:7 | 62 | 4138 | 59 | 9142 | 33 | 69 | 64 | 58 |
| H18 | 1:5 | 57 | 5410 | 65 | 10555 | 46 | 63 | 50 | 52 |
| H19 | 1:10 | 64 | 3863 | 57 | 8107 | 50 | 69 | 45 | 60 |

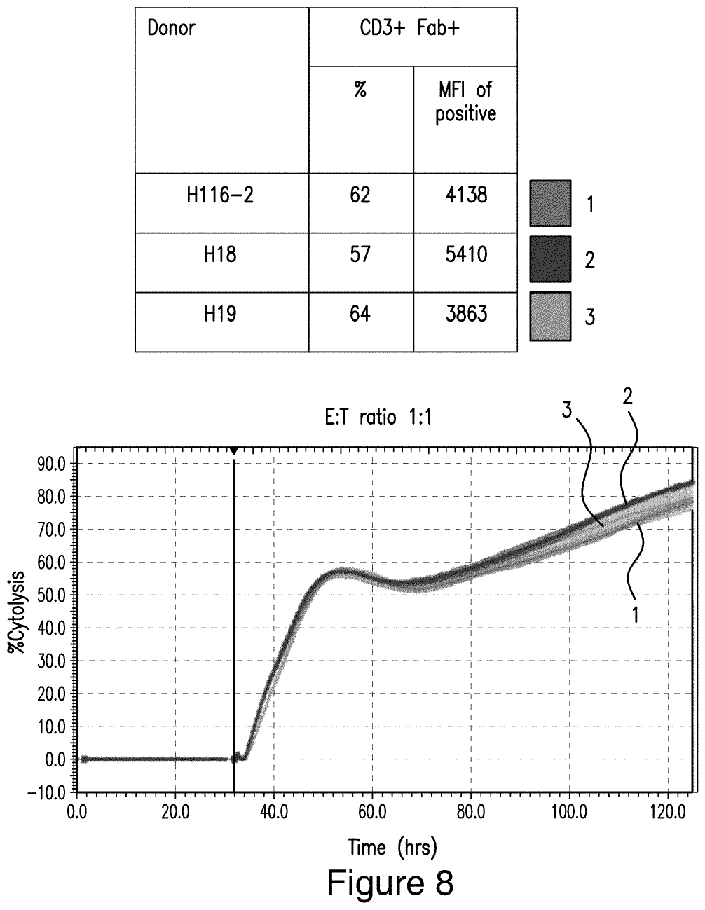

Next, the cytolytic effects of M28z1xx-PD1DNR CAR T cells were studied. MSLN high target cells (MGM) were co-cultured with M28z1XX-PD1DNR CAR T cells from different donors at different E:T ratios using an impedance-based assay. The results are shown in FIG. 8. As shown in FIG. 8, M28z1XX-PD1DNR CAR transduced T cells demonstrated effective cytotoxicity for all three tested different donors. Effector cytotoxicity was across multiple E:T ratios (data not shown).

### Conclusions:

M28zxx-PD1DNR vectors were successfully produced in RD114 cells. Stable producer cell lines were successfully established for all constructs. Viral vectors were titrated to yield transduction of ˜40-60% in multiple donor T cells. CD4 and CD8 T cells were successfully transduced to express CAR and PD1DNR. A correlation was observed between vector copy number and transduction.

### Example 3

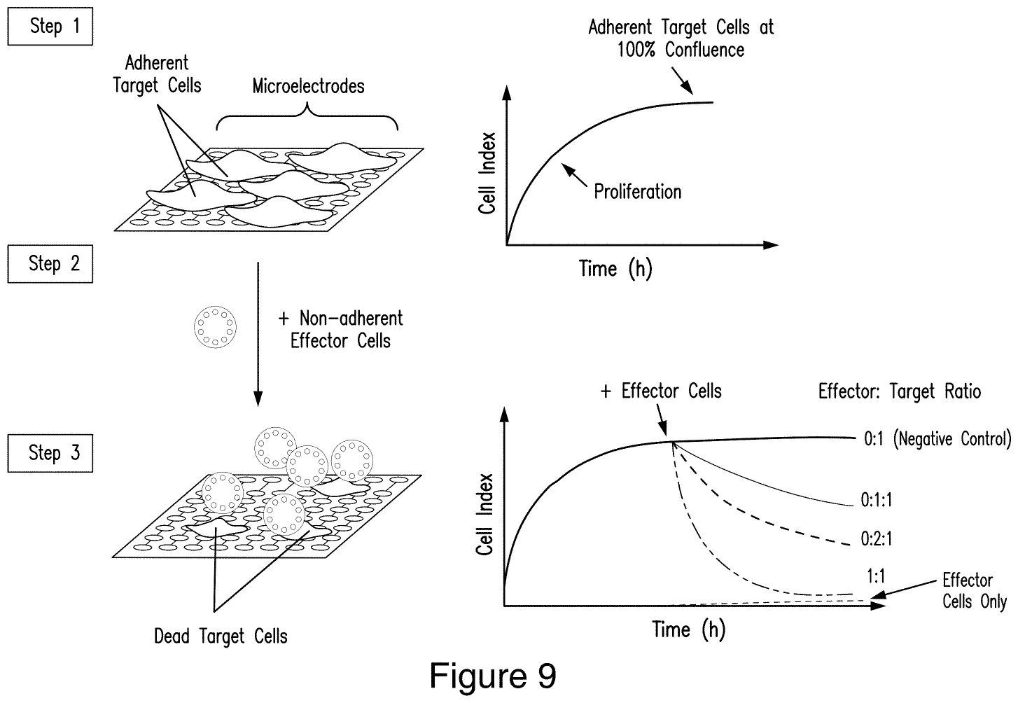

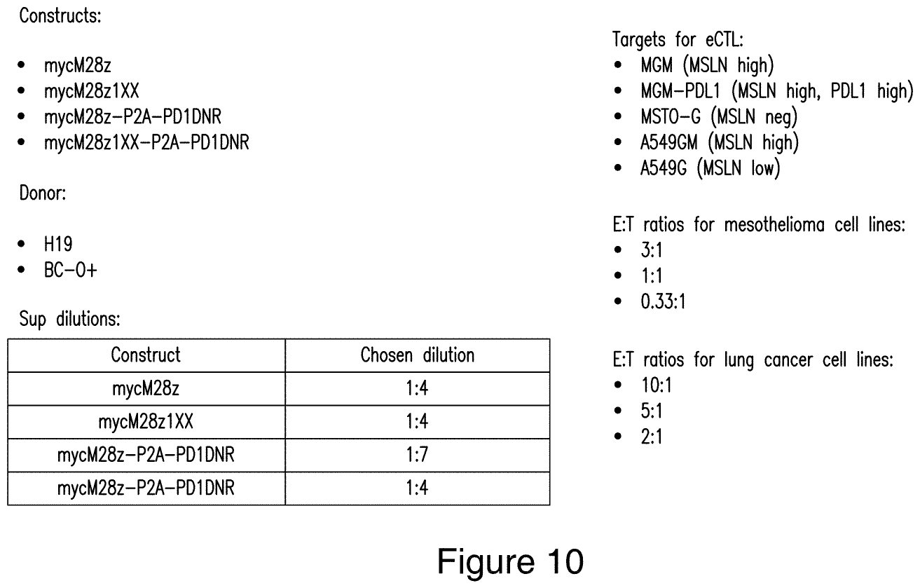

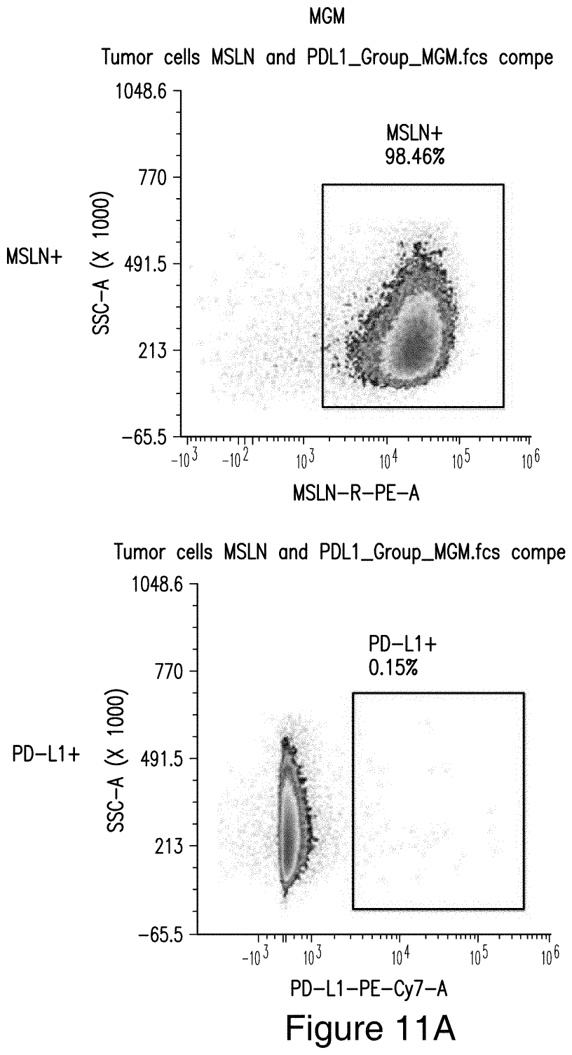

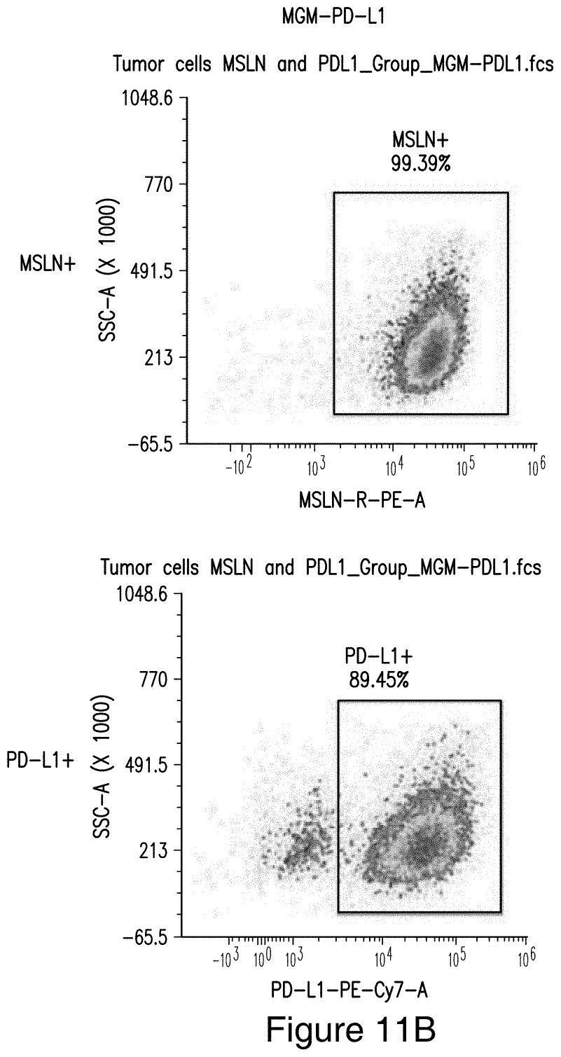

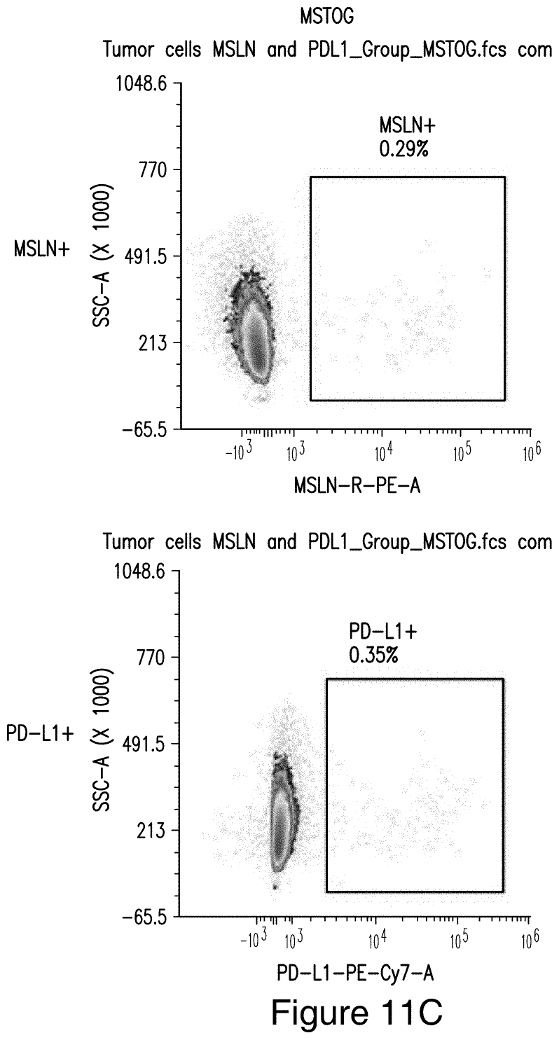

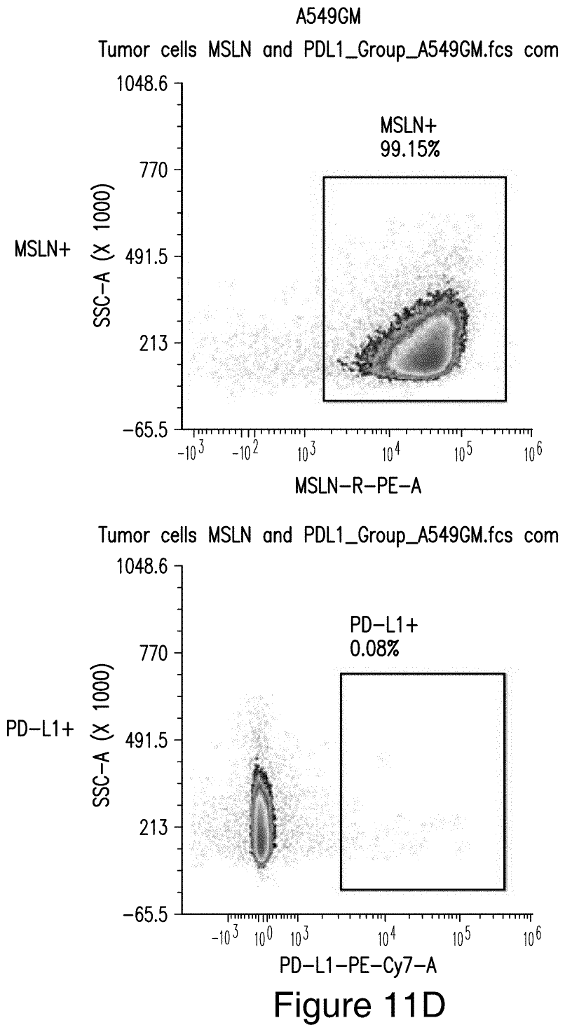

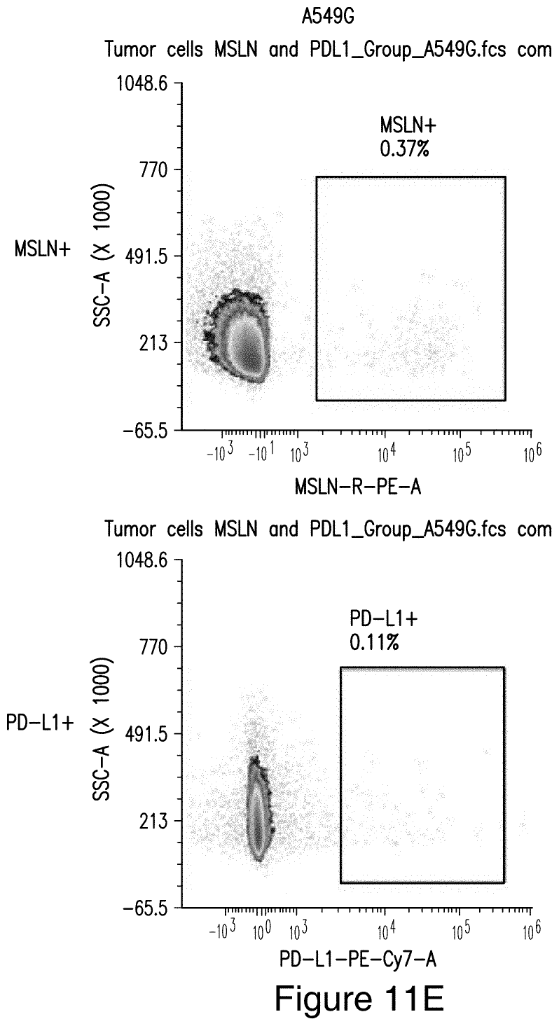

This example describes the comparative analysis of various constructs including M28z1XX-PD1DNR. The cytotoxicity was measured by using impedance assay. The principle of impedance-based cytotoxicity measurement (eCTL) is shown in FIG. 9. The parameters of the comparative analysis are shown in FIG. 10, including the CAR constructs, donors, CAR targets and E:T ratios. MSLN and PD-L1 expressions in target cell lines were measured. Mesothelioma (MGM, MGM-PDL1 and MSTOG) and lung cancer (A549GM and A549G) cell lines were assessed for MSLN and PD-L1 expressions by flow cytometry. The results are shown in FIGS. 11A-11E. MGM, MGM-PDL1 and A549GM overexpressed MSLN. MGM-PDL1 cells additionally overexpressed PD-L1.

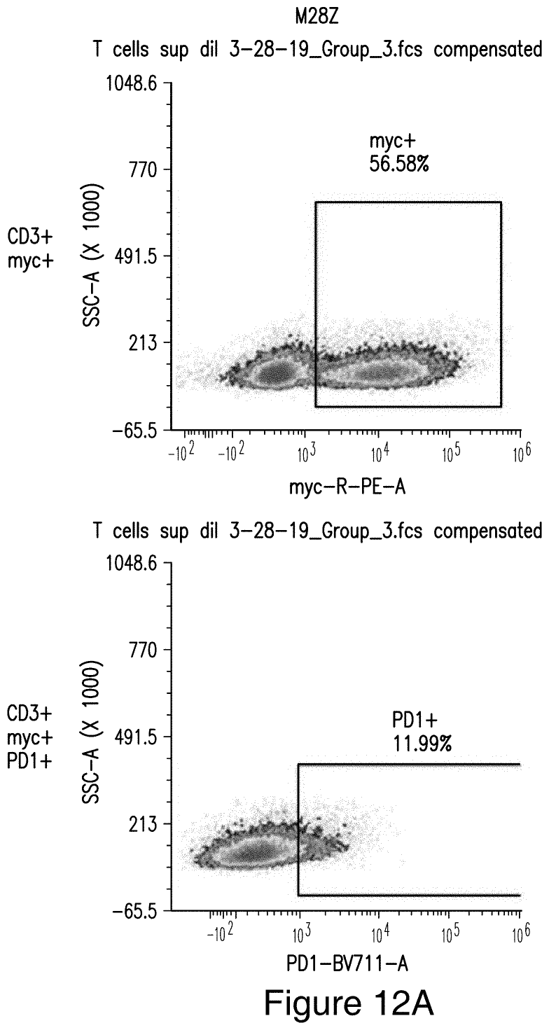

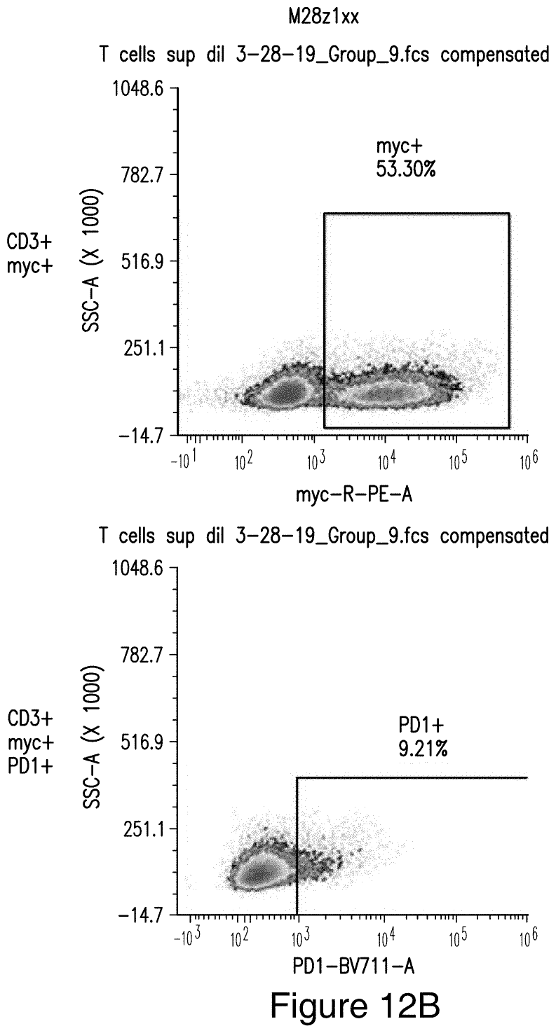

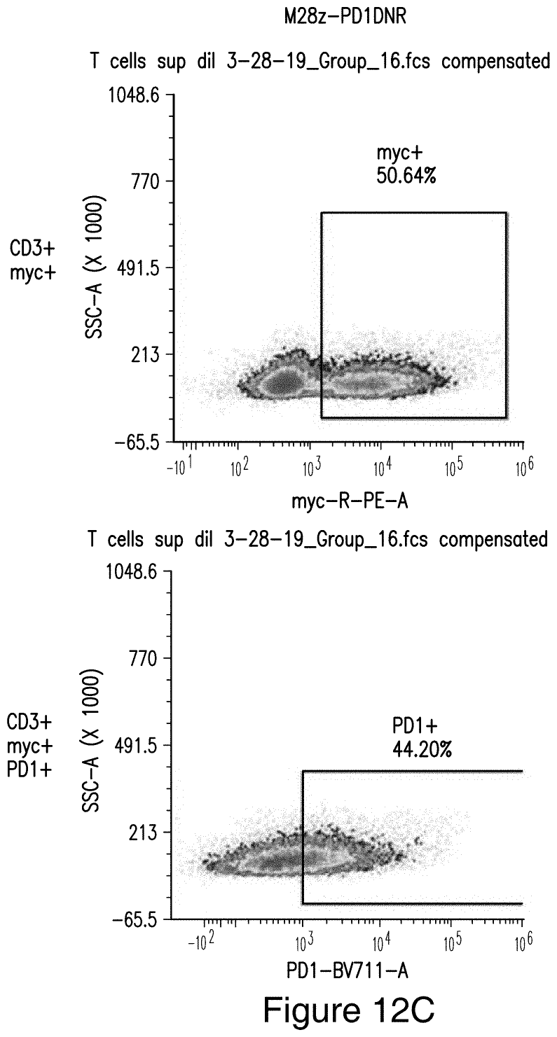

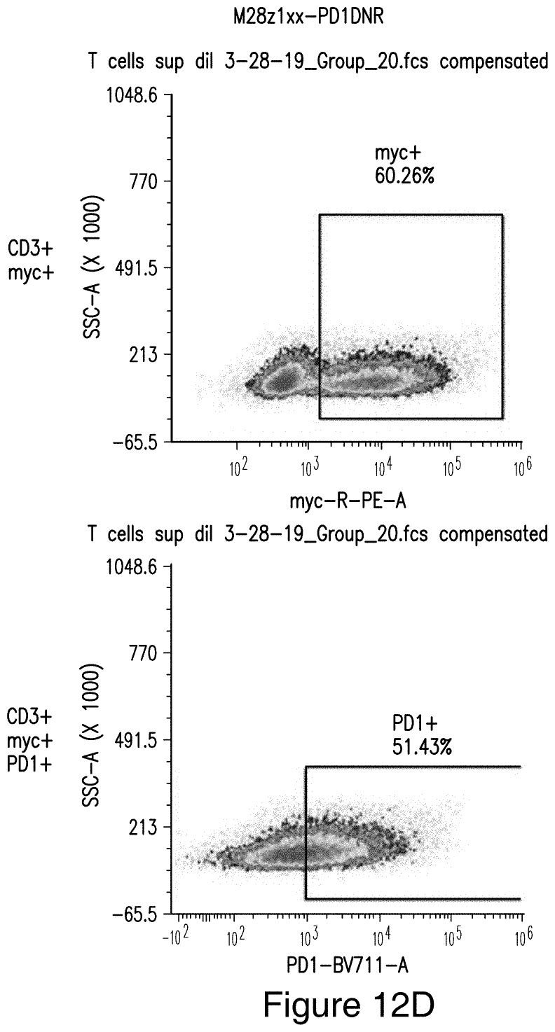

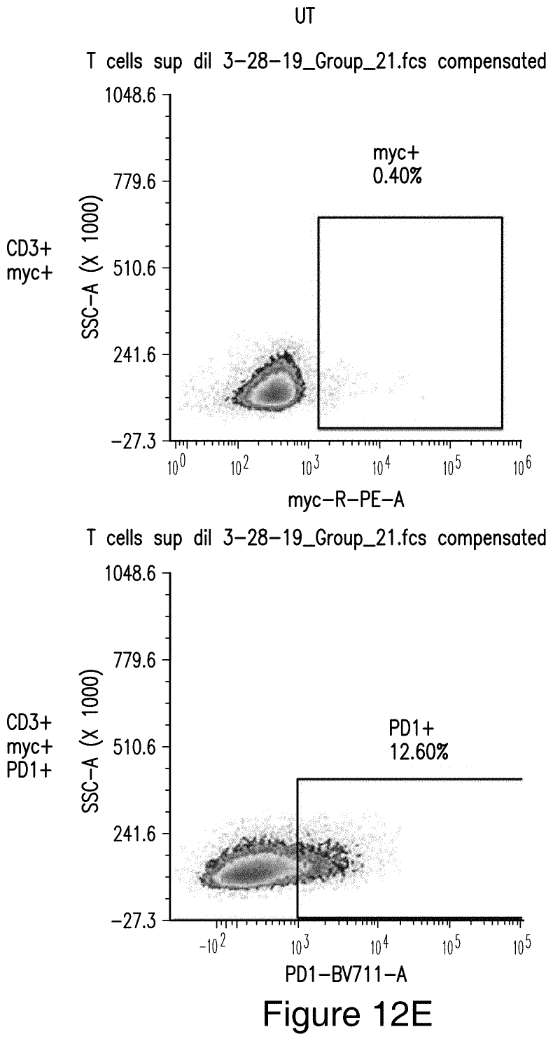

CAR and PD1 expression of transduced T cells were also measured. Human T cells transduced with M28z, M28z1xx, M28z-PD1DNR or M28z1xx-PD1DNR were analyzed for CAR expression by anti-myc staining and PD1/PD1DNR expression by anti-PD1 staining using flow cytometry. The results are shown in FIGS. 12A-12E.

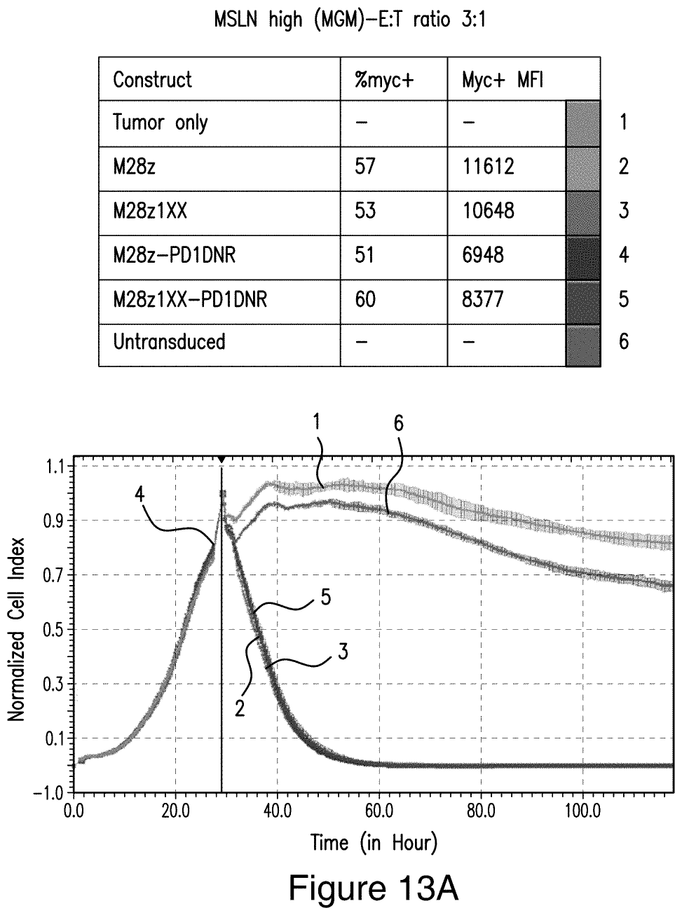

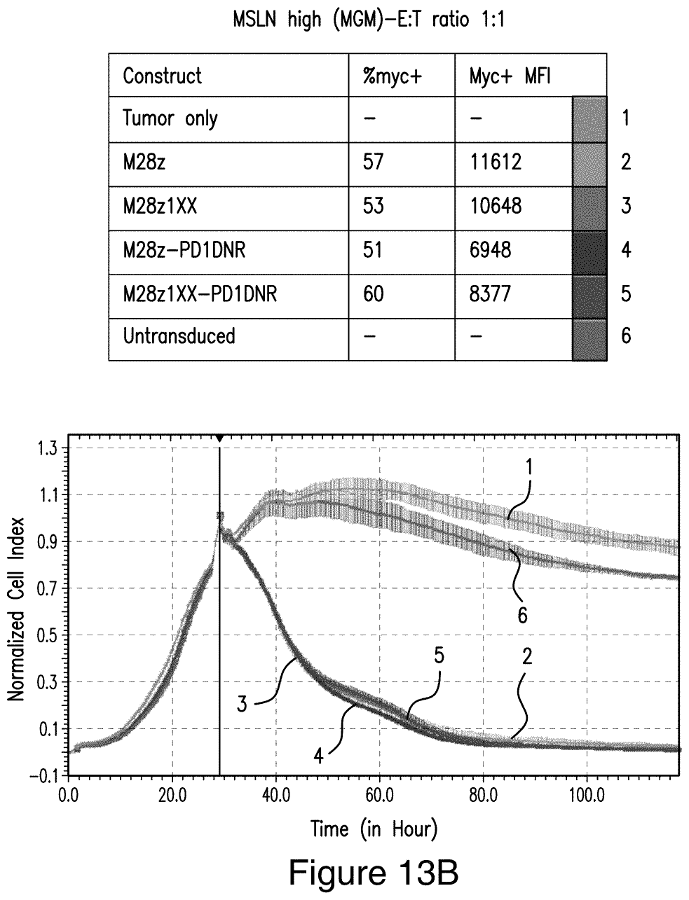

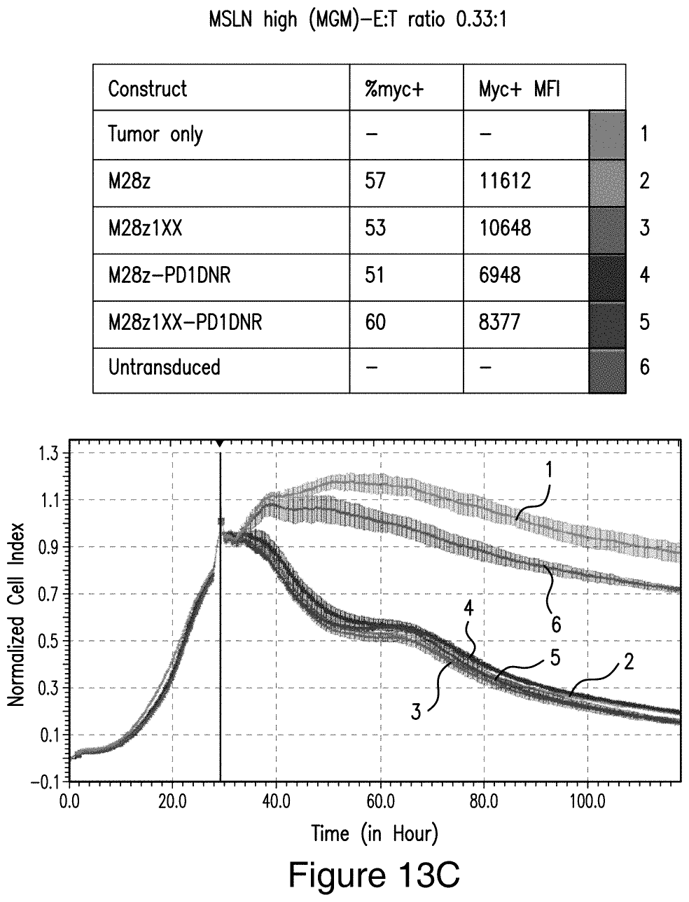

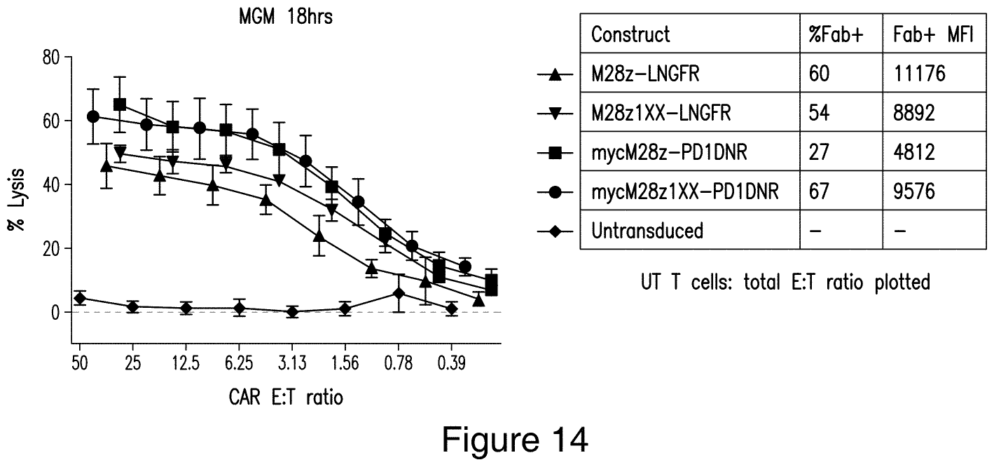

Comparative analysis of anti-tumor efficacy of CAR T cells expressing M28z, M28z1xx, M28z-PD1DNR or M28z1xx-PD1DNR against MSLN high tumor cells (MGM) was conducted. MSLN high target cells (MGM) were co-cultured with either M28z, M28z1xx, M28z-PD1DNR, M28z1XX-PD1DNR or untransduced T cells at various E:T ratios. Anti-tumor efficacy was assessed using an impedance-based assay. The results are shown in FIGS. 13A-13C. In addition, MSLN high target cells (MGM) labeled with chromium-51 were co-cultured with either M28z, M28z1xx, M28z-PD1DNR, M28z1xx-PD1DNR or untransduced T cells at various E:T ratio for 18 hours. Cytotoxicity was determined by chromium-51 CTL. The results are shown in FIG. 14.

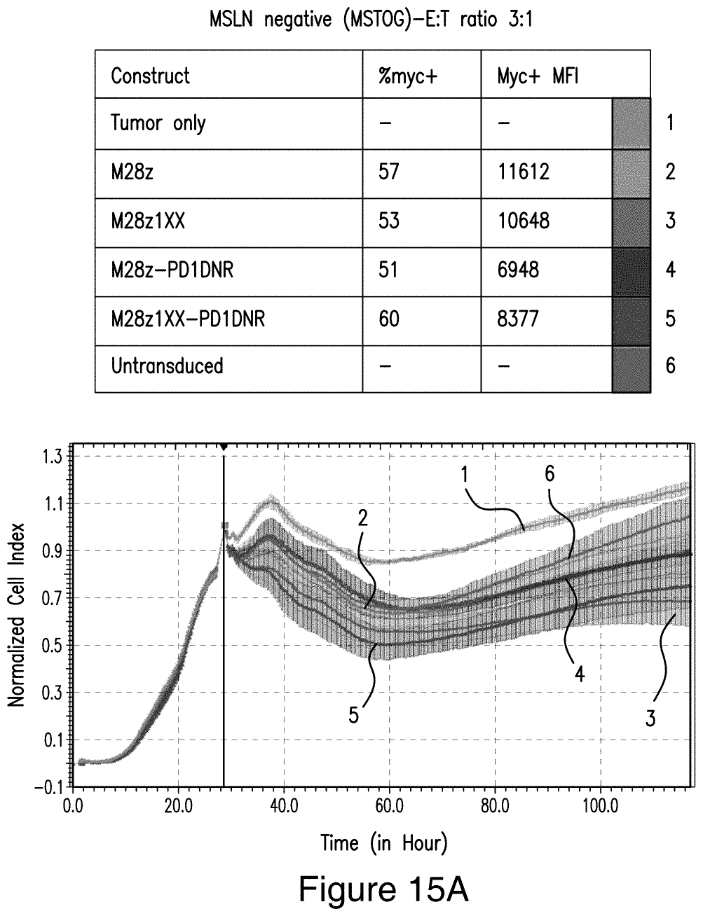

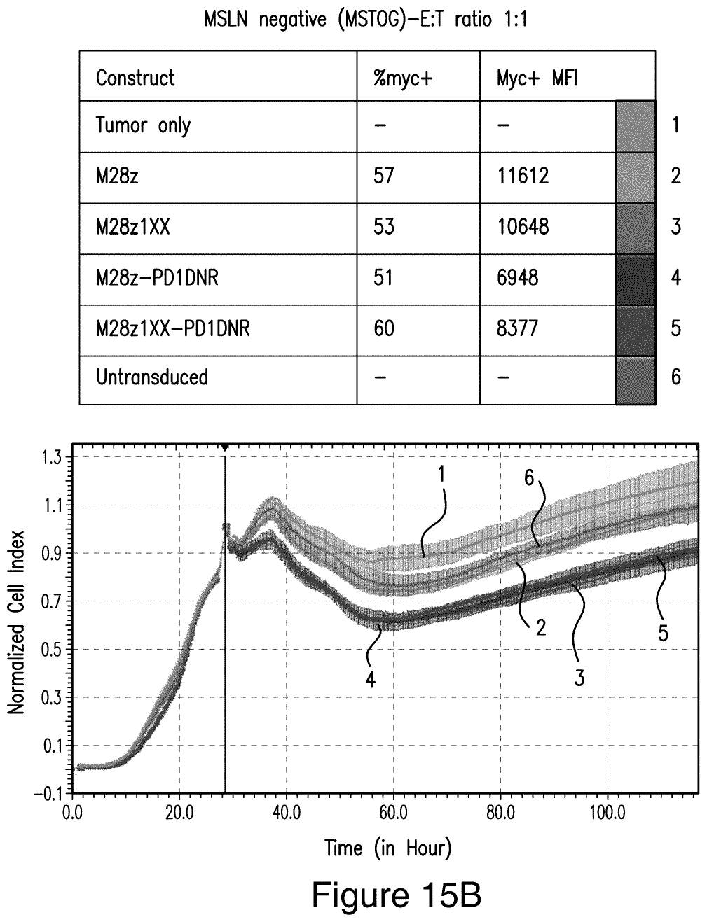

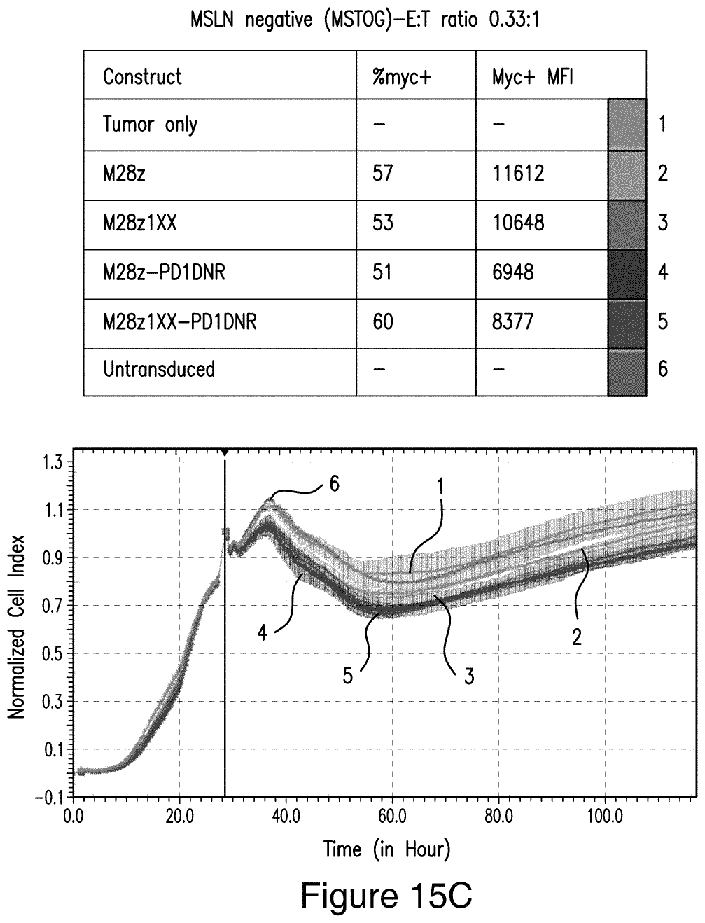

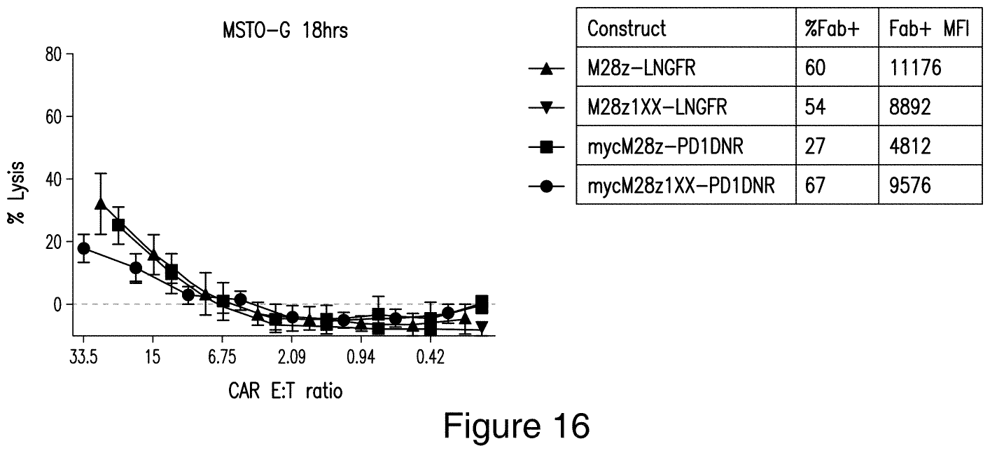

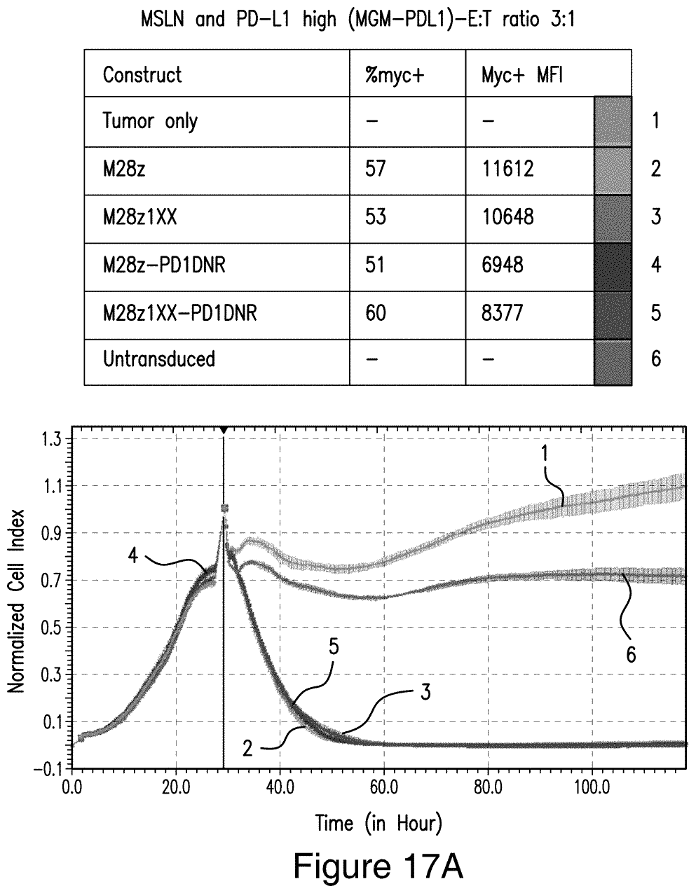

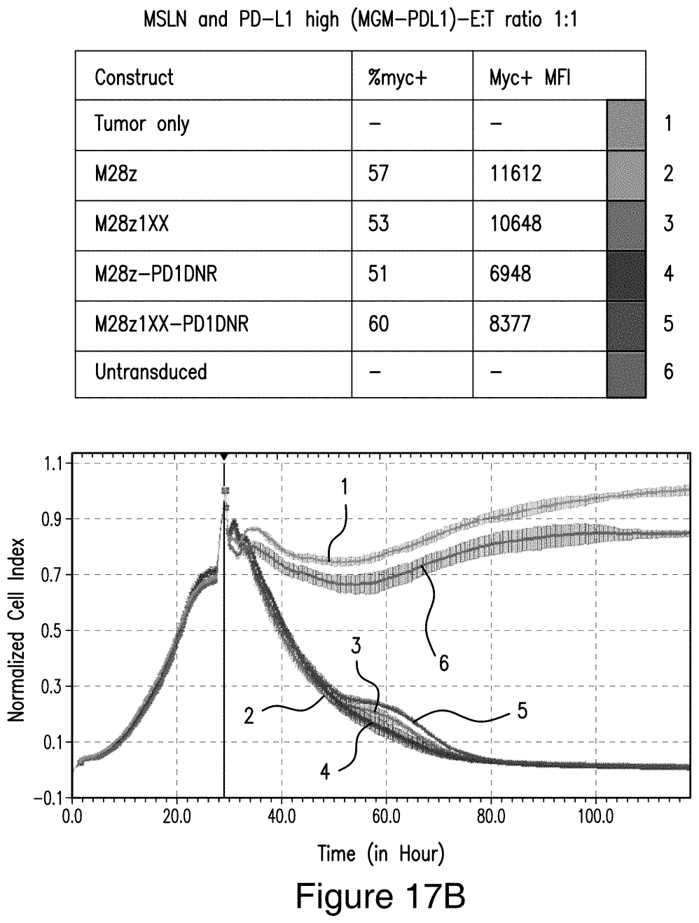

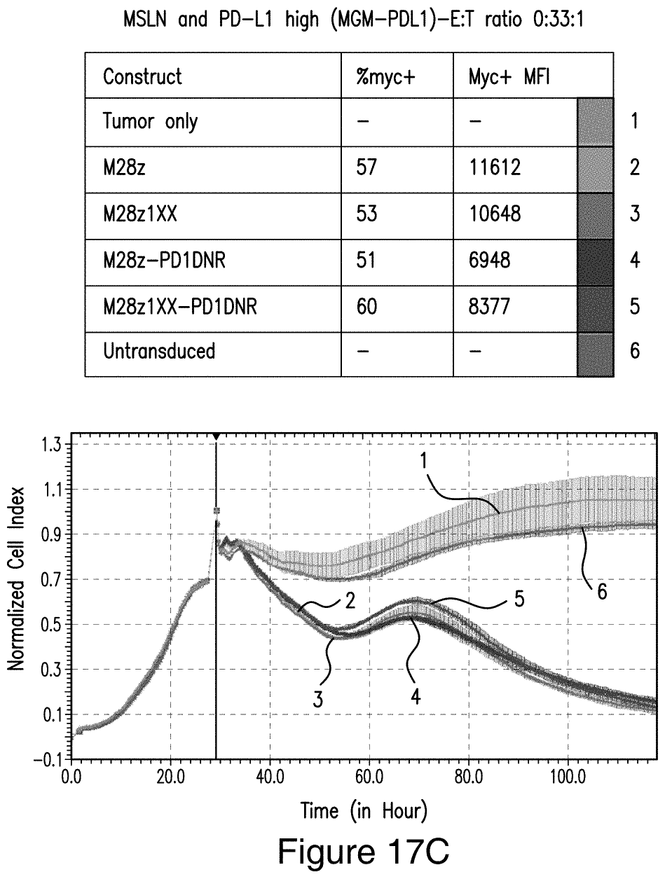

Next, comparative analysis of anti-tumor efficacy of CAR T cells expressing M28z, M28z1xx, M28z-PD1DNR or M28z1xx-PD1DNR against MSLN negative tumor cells (MSTOG) was measured. MSLN negative target cells (MSTOG) were co-cultured with either M28z, M28z1xx, M28z-PD1DNR, M28z1xx-PD1DNR or untransduced T cells at the indicated E:T ratios. Anti-tumor efficacy was assessed using an impedance-based assay. The results are shown in FIGS. 15A-15C. In addition, MSLN negative target cells (MSTOG) labeled with chromium-51 were co-cultured with either M28z, M28z1XX, M28z-PD1DNR, M28z1XX-PD1DNR or untransduced T cells at various E:T ratio for 18 hours. Cytotoxicity was determined by chromium-51 CTL. The results are shown in FIG. 16.

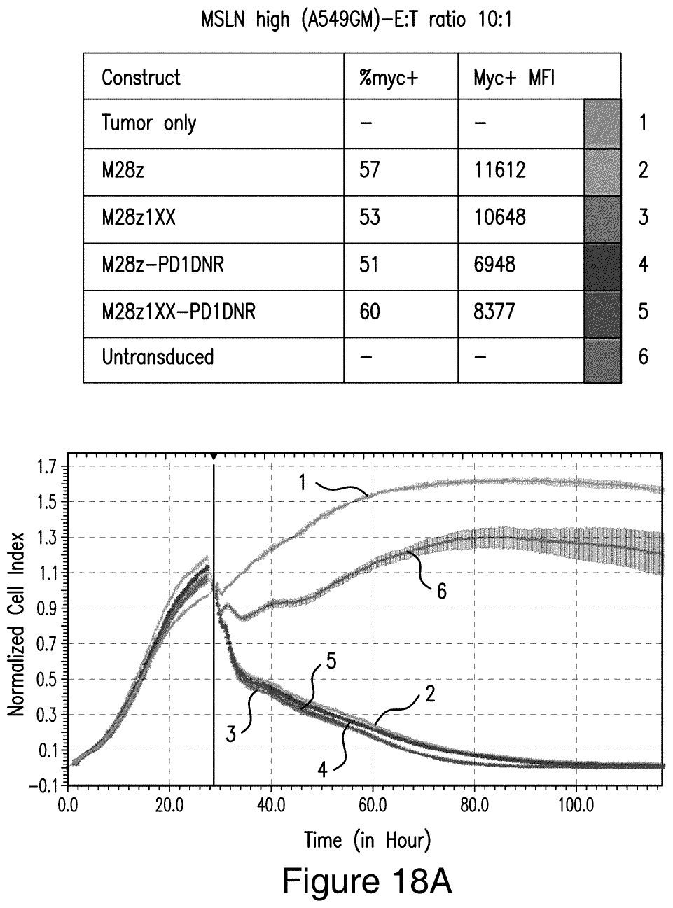

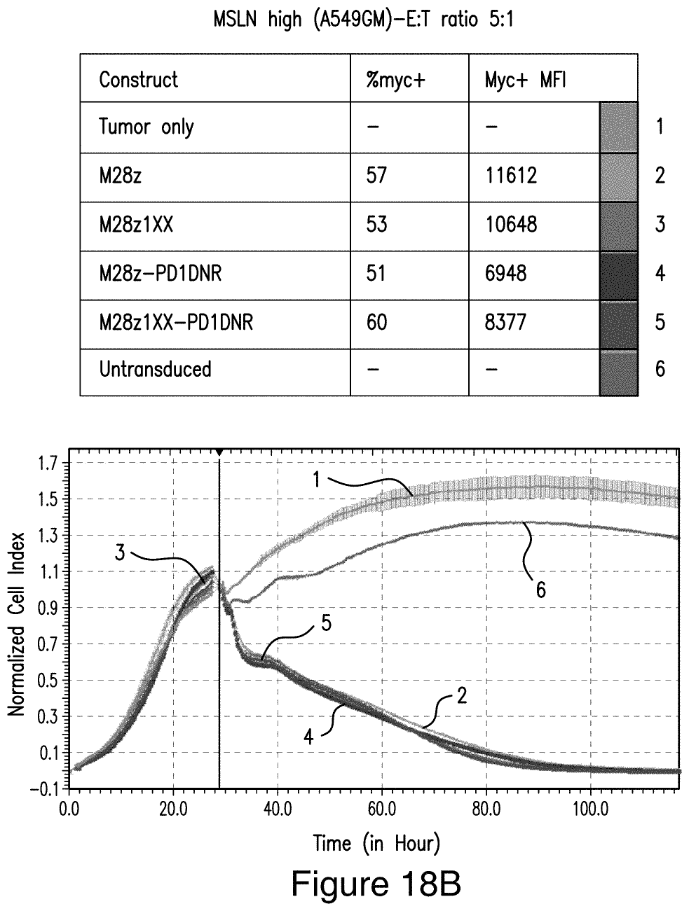

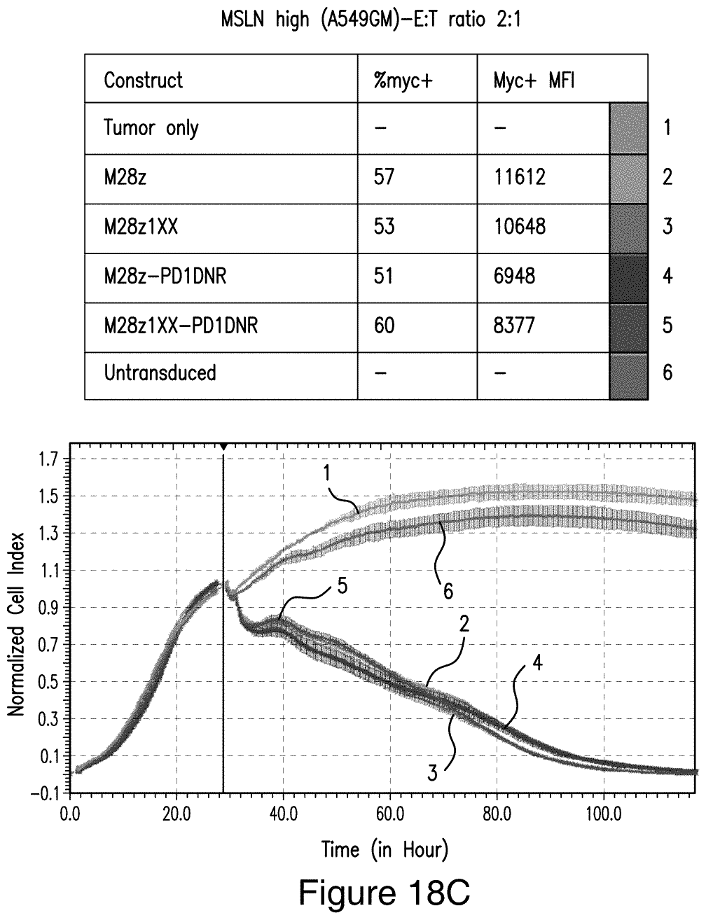

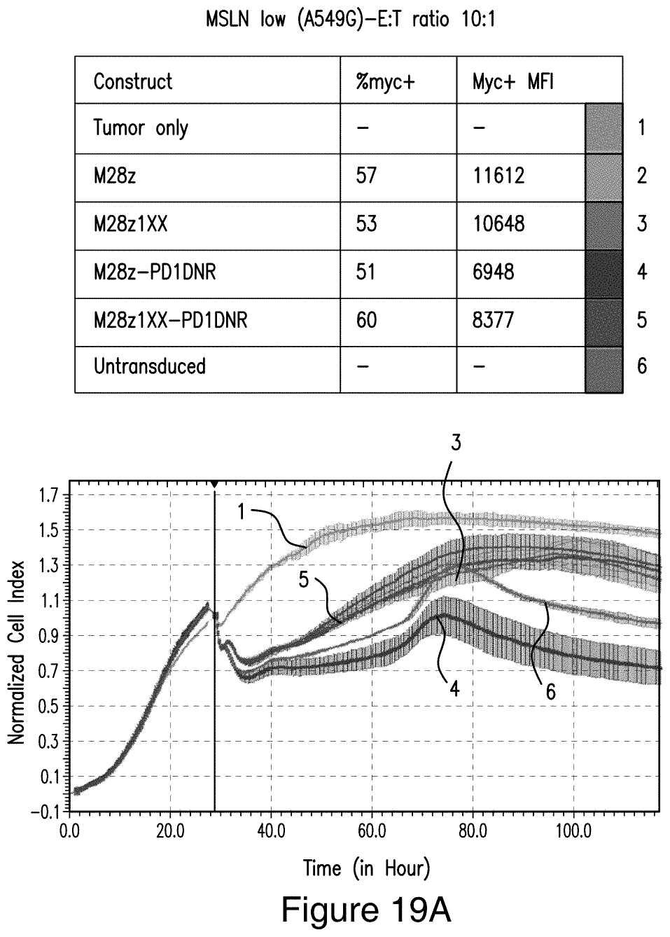

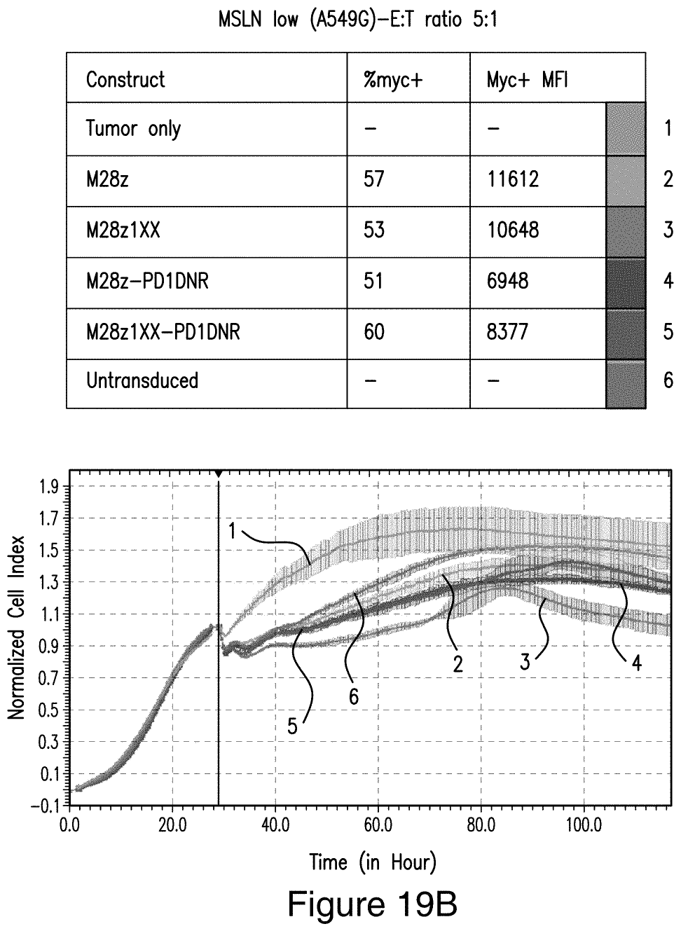

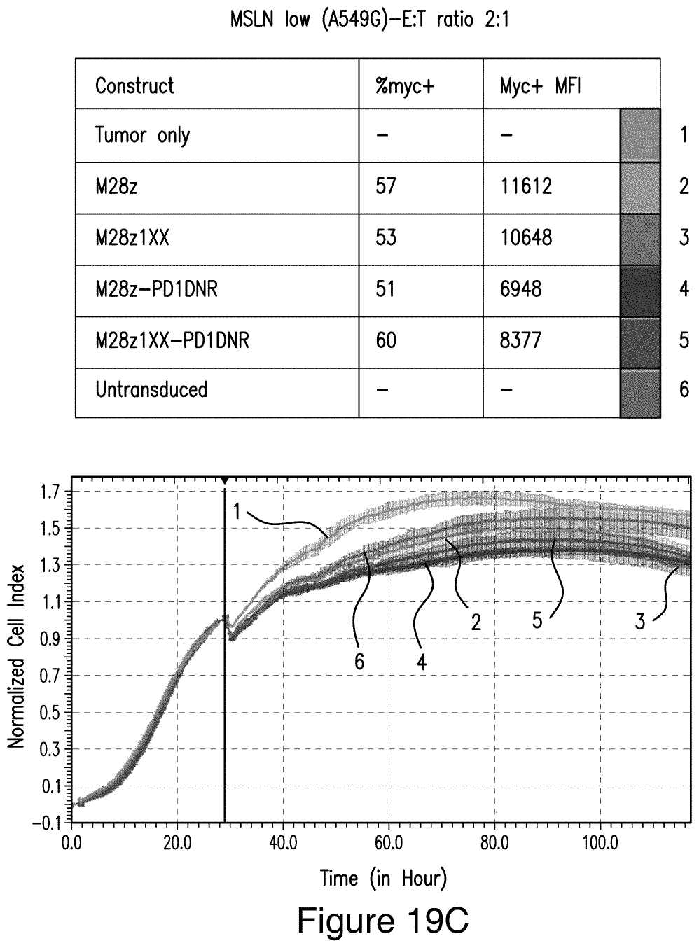

Furthermore, comparative analysis of anti-tumor efficacy of CAR T cells expressing M28z, M28z1xx, M28z-PD1DNR or M28z1xx-PD1DNR against MSLN high tumor cells overexpressing PDL1 was measured. MSLN high target cells overexpressing PDL1 (MGM-PDL1) were co-cultured with either M28z, M28z1XX, M28z-PD1DNR, M28z1XX-PD1DNR or untransduced T cells at various E:T ratios. Anti-tumor efficacy was assessed using an impedance-based assay. The results are shown in FIGS. 19A-19C. Similarly, comparative analysis of anti-tumor efficacy of CAR T cells expressing M28z, M28z1xx, M28z-PD1DNR or M28z1xx-PD1DNR against MSLN high tumor cells (A549GM) was measured, and the results are shown in FIGS. 18A-18C; and comparative analysis of anti-tumor efficacy of CART cells expressing M28z, M28z1xx, M28z-PD1DNR or M28z1xx-PD1DNR against MSLN low tumor cells (A549G) was measured, and the results are shown in FIGS. 19A-19C.

### Conclusions:

M28z1xx-PD1DNR constructs killed MSLN⁺ target cells in an E:T ratio-dependent manner, where the results were reproduced with different T cells donors in different cancers (lung cancer and mesothelioma cell line). The targeted killing was correlated with the levels of MSLN expression and was efficient against MSLN⁺ target cells with high expression of PD-L1.

### Example 4

### Regional Delivery of Clinical-Grade Mesothelin-Targeted CAR T Cells with Cell-Intrinsic PD-1 Checkpoint Blockade: Translation to a Phase I Trial

Summary: This example provides evidence of the preclinical safety and enhanced antitumor efficacy of clinical-grade M28z1XXPD1DNR CART cells.

Methods: Comparative cytotoxicity, proliferation, and cytokine secretion of human T cells engineered to express M28z or M28z1XXPD1DNR CAR were assessed by chromium-release, accumulation, and Luminex assays, respectively. The antitumor efficacy of a single dose (1×10⁵ CAR T cells; E:T 1:1000) of intrapleurally administered M28z or M28z1XXPD1DNR CAR T cells was investigated in NSG mice with orthotopic pleural mesothelioma by serial bioluminescence imaging and by comparing survival. Following tumor eradication, functional persistence of CAR T cells was tested by repeated tumor challenge (increasing doses of 2×10⁶ to 11×10⁶ tumor cells).

Results: In vitro, both M28z and M28z1XXPD1DNR CAR T cells exhibited antigen-specific cytotoxicity, accumulation, and effector cytokine secretion (see Table 2). In vivo, a single dose of M28z1XXPD1DNR CAR T cells led to tumor eradication, enhanced survival, and resistance to tumor reestablishment upon 10 tumor rechallenges (see Table 2) versus a single dose of M28z CAR T cells.

Conclusion: The data on the safety, tumor eradication, and functional persistence of CAR T cells without the use of anti-PD1 antibody support the initiation of a phase I clinical trial of intrapleural administration of M28z1XXPD1DNR CAR T cells in patients with pleural mesothelioma.

TABLE 2
Comparison of M28z and
M28z1XXPD1DNR CAR T-cell constructs

| | M28z | | M28z1XXPD1DNR | |
|---|---|---|---|---|
| Target | Mesothelin | | Mesothelin | |
| Costimulatory domain | CD28 | | CD28 | |
| CD3z | No mutations | | 2 ITAMs | |
| | | | mutated (1XX) | |
| T-cell intrinsic | No | | Yes | |
| checkpoint blockade | | | | |
| (PD1DNR) | | | | |
| In vitro results | | | | |
| Human T-cell | 25%-82% | | 30%-87% | |
| transduction, range | | | | |
| PD-1 extracellular | | | | |
| domain mRNA | | | | |
| expression | | | | |
| compared to untrans- | 4 | | 157 | |
| duced, fold | | | | |
| Cytotoxicity, range | | | | |
| E:T 10:1 | 35%-45% | | 25%-51% | |
| E:T 5:1 | 28%-44% | | 20%-38% | |
| E:T 2:1 | 17%-32% | | 14%-24% | |
| Accumulation, range, fold | 110-390 | | 53-622 | |
| Effector cytokines | | | | |
| (E:T 1:1, 24 h), range | | | | |
| IL-2 | 18-23 | ng/mL | 9-19 | ng/mL |
| TNF-α | 545-977 | pg/mL | 380-852 | pg/mL |
| IFN-γ | 8-11 | ng/mL | 6-15 | ng/mL |
| In vivo results | | | | |
| Tumor eradication | 26 | days | 19 | days |
| Median survival | 56 | days | Not reached | |
| Tumor progression as | | | | |
| measured by biolumin- | | | | |
| escence imaging following | | | | |
| rechallenge | | | | |
| Rechallenged 3 times | +1 | log | +0.2 | logs |
| over 15 days | | | | |
| Rechallenged 10 times | +1-2 | logs | +0.2 | logs |
| over 52 days | | | | |

| | | |
|---|---|---|
| In transition to phase | | |
| Current status | In transition | Clinical-grade |
| | to phase | vector |
| | II clinical trial | and viral |
| | | supernatant |
| | | produced |

### Example 5

### A Next-Gen CAR T-Cell with Cell-Intrinsic PD-1 Blockade: Clinical Rationale, Preclinical and Clinical Trial Protocol Development

Malignant pleural mesothelioma (MPM) is a low mutational burden and low-PDL1 expressing cancer with discouraging responses to anti-PD1 antibodies. In an ongoing phase I/II trial (NCT02414269, n=41), the safety and antitumor efficacy of intrapleurally administered mesothelin-targeted chimeric antigen receptor (M28z CAR) T cells followed by PD-1 antibody have been established. CAR T cells with cell-intrinsic anti-PD1 strategy are safe and can provide anti-tumor efficacy against both low- and high-PDL1 tumor without the need for repeated administration of anti-PD1 antibody.

Summary: This example provides evidence of the preclinical safety and enhanced antitumor efficacy of clinical-grade M28z1XXPD1DNR CART cells.

Methods: Clinical-grade M28z and M28z1XXPD1DNR (modified CD3z domain with PD-1 dominant negative receptor) CAR were transduced in multiple donor T cells as effectors, and MPM cells with low- and high-PDL1 were used as targets. At varying E:T ratios, comparative in vitro, and in vivo anti-tumor efficacy was assessed in mice with orthotopic MPM. Systemic anti-tumor immunity was tested by repeated tumor challenges at a distant site.

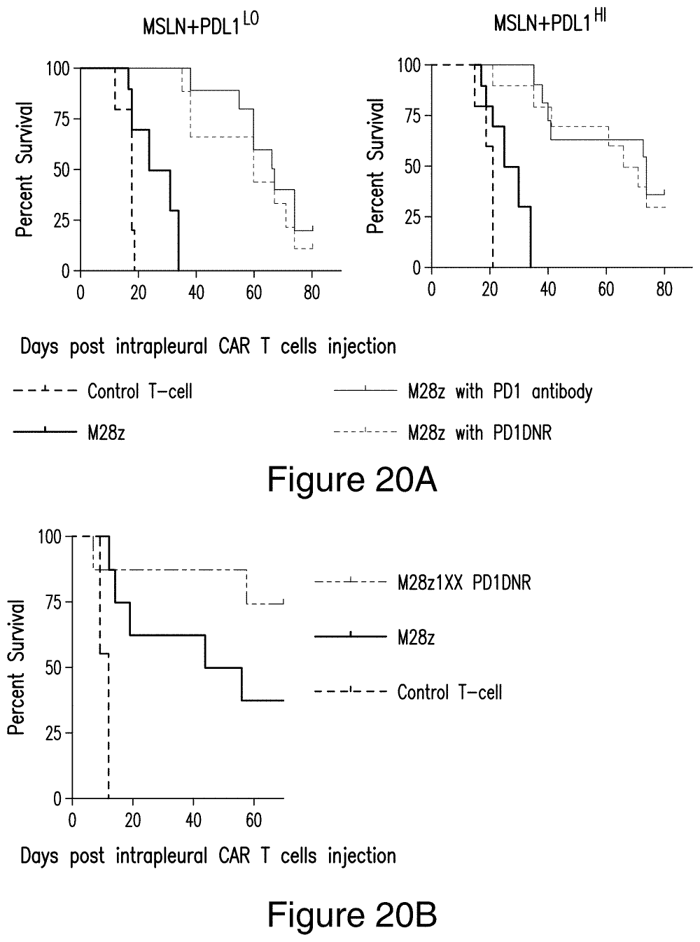

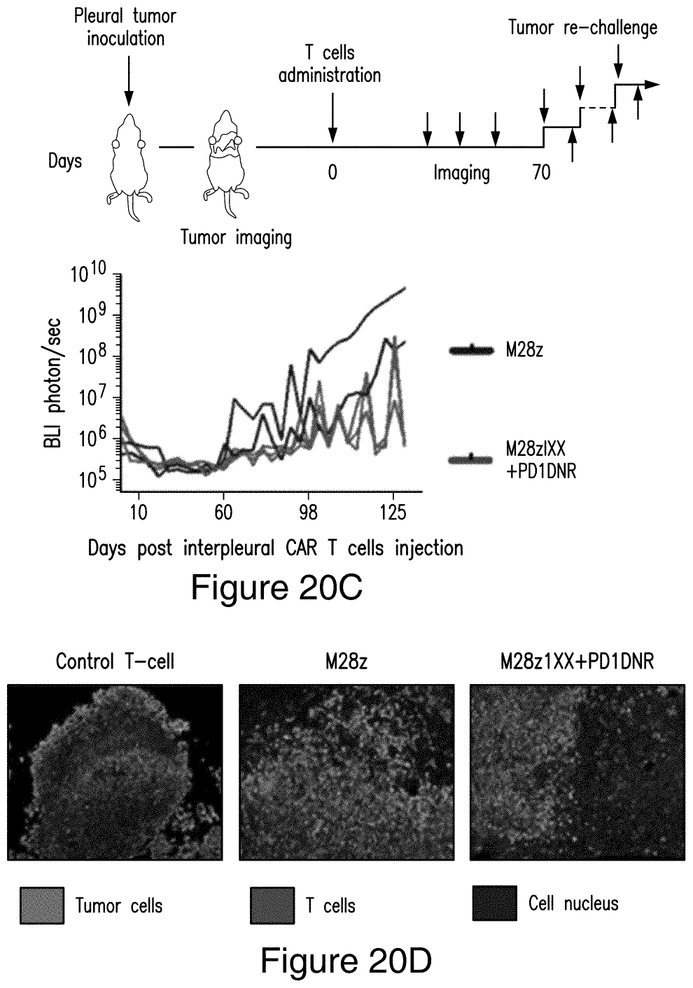

Results: In vitro, no significant differences were noted between M28z and M28z1XXPD1DNR CARs (antigen-specific cytotoxicity, accumulation, and effector cytokine secretion). In vivo, a single dose (1×10⁵ CAR T cells) of intrapleurally administered M28z CAR T cells either with repeated administration of anti-PD1 antibody or with cell-intrinsic PD1DNR led to comparable tumor eradication, enhanced survival with weight gain. See FIG. 20A and Table 3. In mice with orthotopic MPM, a single, low-dose (1×10⁵ CAR T cells) of M28z1XXPD1DNR CAR T cells eradicated pleural tumor, demonstrated enhanced systemic immunity compared to M28z CAR T cells by resisting tumor re-challenges at a distant peritoneal site without any toxicity (PD1DNR bind to mouse PDL1/2). See FIGS. 20B and 20C. Harvested tumors demonstrate higher and deeper infiltration of CAR T cells compared to un-transduced T cells. See FIG. 20D.

Conclusion: A single, low-dose of intrapleurally administered M28z1XXPD1DNR CAR T cells demonstrate feasibility, safety, tumor eradication, functional persistence and systemic anti-tumor immunity.

TABLE 3
Comparison of Characteristics of Therapy
with PD-1 DNR CAR T Cells vs.
Checkpoint Blockade Agents with CAR T cells

| | PD-1 DNR | Checkpoint Blockade |
|---|---|---|
| Characteristic | CAR T cells | Agents with CAR T cells |
| Mechanism | Cell intrinsic | Cell extrinsic |
| Potential doses | Single | Multiple |
| Targeted therapy | Yes | No |
| Toxicity | Localized | Systemic |
| | to tumor | |
| Limits to tumor | Unlikely | Limits of antibody |
| penetration | | penetration |
| Patient selection | Not required | Tumor PD-L1 high |
| Antigen | Multiple | Multiple |
| responsiveness | | |

### Example 6

### 1. Summary

Malignant pleural mesothelioma (MPM) is a rare and lethal malignancy associated with asbestos exposure. MPM is a regionally aggressive primary malignancy of the pleura with invasion into vital organs or the chest wall as a characteristic (Carbone et al., CA Cancer J Clin. 2019; 69(5):402-429). The majority of the patients (60%-70%) at presentation have locoregionally advanced disease and are unresectable (Nelson et al., J Clin Oncol. 2017; 35(29):3354-3362; Flores et al., J Thorac Oncol. 2007; 2(10):957-965). Even with successful completion of a combination of chemotherapy, aggressive surgical resection and radiation therapy, the median survival of treated patients is only 9-17 months (Flores et al., J Thorac Oncol. 2007; 2(10):957-965). There have been no new FDA-approved therapies for MPM since 2003 (Tsao et al., J Thorac Oncol. 2018; 13(11):1655-1667). The present standard of care for first-line systemic treatment of patients with MPM is cisplatin plus pemetrexed, on the basis of a median overall survival of 12.1 months compared, with 9.3 months for cisplatin alone (Vogelzang et al., J Clin Oncol. 2003; 21(14):2636-2644). As patients with MPM have a low tumor mutational burden and low programmed death-ligand 1 (PD-L1) expression, their response to immune checkpoint inhibitors is limited, and a tremendous unmet need persists (Yarchoan et al., JCI Insight. 2019; 4(6); Forde et al., Curr Treat

Options Oncol. 2019; 20(2):18). MPM's localized nature, potential accessibility, and relative lack of metastases at presentation make it a suitable candidate for regional targeted therapies (Nelson et al., J Clin Oncol. 2017; 35(29):3354-3362).

This Example describes the nonclinical studies conducted to support the clinical use of an intrapleural dose of M28z1XXPD1DNR chimeric antigen receptor (CAR) T cells, an investigational new drug for the treatment of patients with a diagnosis (histologically or cytologically documented) of MPM who have received at least one chemotherapeutic regimen and are documented to have tumor.

This is a single-center phase I study with a maximum of 36 participants designed to assess the safety, dose requirement, and targeting efficiency of genetically directed autologous M28z1XXPD1DNR CAR T cells following pretreatment with cyclophosphamide. There are 5 planned dose levels in this study: 1×10⁶, 3×10⁶, 6×10⁶, 1×10⁷, and 3×10⁷ M28z1XXPD1DNR CAR T cells/kg, provided there are no dose-limiting toxicities. M28z1XXPD1DNR CAR T cells are infused through an indwelling pleural catheter. Patients are screened for the expression of mesothelin by immunohistochemical analysis of biopsied tumor and/or blood levels of soluble mesothelin-related peptides before treatment.

M28z1XXPD1DNR CAR T cells are autologous T cells transduced ex vivo with a gamma retroviral vector stock supernatant generated from a vector-producing master cell bank, 293 VEC-GALV-SFG-M28z1XXPD1DNR. The main components of the CAR encoded in the vector are:

1) Human anti-mesothelin scFv for targeting tumors expressing mesothelin,

2) Human CD28 costimulatory domain for signaling T-cell survival and proliferation,

3) Point-mutated human CD3ζ with a single functional immunoreceptor tyrosine-based activation motif (ITAM) for calibrated T-cell activation, and

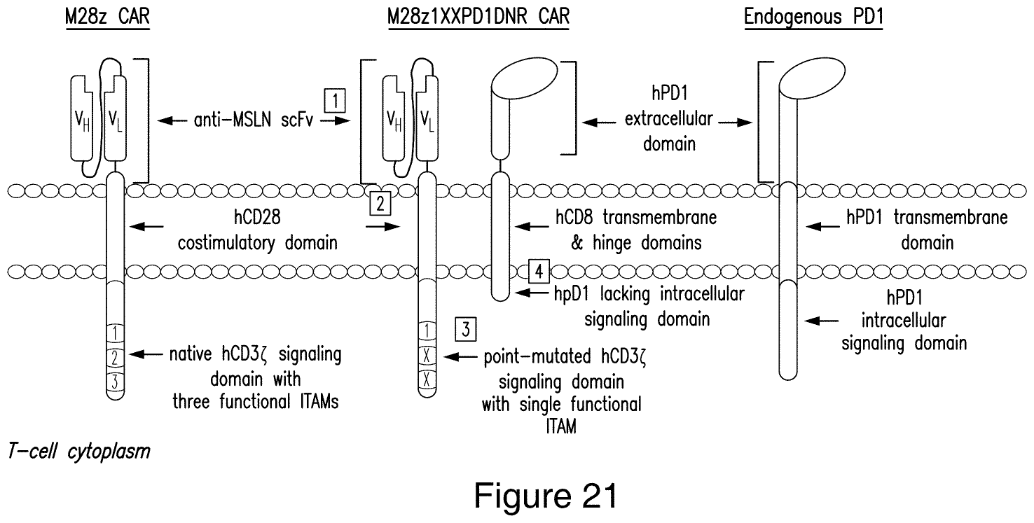

4) Programmed cell death protein 1 (PD1) dominant negative receptor (PD1DNR) for protecting T cells from going into a state of dysfunction or exhaustion upon antigen exposure (see FIG. 21).

Mesothelin is a cancer cell-surface antigen that is overexpressed in majority of MPM, lung cancers, triple-negative breast cancers, pancreatic cancers, and ovarian cancers and in some esophageal cancers (Pastan et al., Cancer Res. 2014; 74(11):2907-2912; Kachala et al., Clin Cancer Res. 2014; 20(4):1020-1028; Tang et al., Anticancer Agents Med Chem. 2013; 13(2):276-280; Servais et al., Clin Cancer Res. 2012; Kelly et al., Mol Cancer Ther. 2012; 11(3):517-525; Tchou et al., Breast Cancer Res Treat. 2012; 133(2):799-804). The inventors have previously demonstrated that mesothelin overexpression promotes aggressiveness in lung adenocarcinomas (n=1200) (Kachala et al., Clin Cancer Res. 2014; 20(4):1020-1028), MPMs (n=250) (Servais et al., Clin Cancer Res. 2012; Kelly et al., Mol Cancer Ther. 2012; 11(3):517-525), and triple-negative breast cancers (n=250) (Tozbikian et al., PLoS One. 2014; 9(12):e114900). In addition to mesothelin expression being relatively high in tumors, compared with normal tissues, it is also expressed at very low levels on normal peritoneal, pleural, and pericardial mesothelial surfaces (Villena-Vargas et al., Ann Cardiothorac Surg. 2012; 1(4):466-471, making it an ideal target for solid-tumor CAR T-cell therapy. The biologic function of mesothelin is not well-understood and is under investigation.

Previously, mesothelin-targeted CAR T cells were given intravenously to humans (3×10⁸ cells/m² or 4.8×10⁷ cells/dose) in a clinical study conducted at the University of Pennsylvania (NCT01355965), where the CAR comprises a murine scFv. The anaphylactic reaction noted in 1 patient was reportedly caused by anti-murine antibody responses that were developed against the humanized mouse scFv portion of the CAR construct used in that study (Beatty et al., Cancer Immunol Res. 2014). In contrast, in a recent phase I study (NCT02414269) conducted by the inventors' laboratory, mesothelin-targeted CAR T cells (up to 6×10⁷ CAR T cells/kg) were administered intrapleurally that are composed of a fully human scFv derived from a human Fab library (Feng et al., Mol Cancer Ther. 2009). To date, 40 patients have been treated without the observation of dose-limiting toxicities. Preliminary efficacy has been observed in this study in a cohort of patients (n=18) who received CAR T-cell therapy followed by a minimum of 3 doses of pembrolizumab (anti-PD1), with an additional 3-month period of follow-up. Importantly, 83% of patients in this cohort did not require new or additional treatment at 6 months, and half of patients did not receive additional treatments for 18 months. Furthermore, in a majority of patients, CAR T cells were detected in the peripheral blood >100 days after intrapleural administration, indicating persistence of these cells in the patient's body.

In support of a first-in-human clinical trial, the pharmacology program for M28z1XXPD1DNR CAR T cells consists of a series of orthogonal in vitro specificity, cytotoxicity, accumulation, and cytokine secretion studies and in vivo tumor efficacy and survival studies in mice, the results of which suggest an effective dose for translation to clinical use. Table 4 provides an integrated summary of the nonclinical pharmacology and toxicology assays performed along with their key findings.

TABLE 4
Integrated summary of pharmacology
and toxicology assays and their key findings.

| Experiment | Purpose | Key findings |
|---|---|---|
| Mesothelin | Define distribution of | Mesothelin expressed in solid |
| expression in | cell-surface mesothelin | tumors |
| human tumors | in human tumors | No mesothelin expression in |
| | | normal lung and breast tissue |
| Transduction | Determine viral | Viral supernatant concentration |
| | supernatant | is directly proportional to CAR |
| | transduction efficiency | and PD1DNR transduction |
| | | efficiency |
| Vector copy | Determine number of | Positive correlation between |
| number | vector insertions in T- | CAR expression and VCN |
| (VCN) | cell genome | CAR transduction optimized to |
| | | 35%-70% to keep VCN ≤ 5 |
| PD1DNR | Distinctly quantify | PD1 extracellular domain |
| quantification | endogenous PD1 and | upregulated at protein level |
| | PD1DNR levels | (>2-fold) and mRNA level |
| | | (>100-fold) for |
| | | mycM28z1XXPD1DNR |
| | | CAR T cells |
| Myc-tag | Investigate impact of | M28z1XXPD1DNR CAR T cells |
| interference | myc-tag on CAR | with and without myc-tag both |
| | function | exhibit identical antitumor |
| | | efficacy in vitro |
| Cytotoxicity | Determine antigen- | mycM28z1XXPD1DNR CAR T |
| | specific cytotoxicity | cells mediate antigen-specific, |
| | | HLA-independent tumor lysis |
| Accumulation | Investigate CAR T-cell | mycM28z1XXPD1DNR CAR T |
| | accumulation | cells found to proliferate and |
| | | accumulate up to 622-fold over |
| | | six antigen stimulations |
| Repeated | Investigate | Initial antigen stimulation: |
| antigen | cytotoxicity under | Similar cytotoxicity between |
| stimulation | continuous antigen | mycM28z1XXPD1DNR and |
| | exposure | mycM28z CAR T cells |
| | | Repeated antigen stimulation: |
| | | Cytotoxicity retained for |
| | | mycM28z1XXPD1DNR but |
| | | decreased for mycM28z CAR T |
| | | cells |
| Cytokine | Quantify cytokine | mycM28z1XXPD1DNR CAR T |
| secretion | secretion upon antigen | cells secrete effector cytokines |
| | stimulation | (IL-2, IFN-γ, TNF-α) upon |
| | | antigen stimulation |
| | | Effector cytokine secretion |
| | | decreased upon repeated antigen |
| | | stimulation |
| Antitumor | Investigate antitumor | Efficacy and tumor eradication |
| efficacy | efficacy and survival | observed at a single intrapleural |
| in vivo | in vivo | dose of 3 × 10⁴ |
| | | mycM28z1XXPD1DNR CAR T |
| | | cells/mouse |
| | | Mice treated with |
| | | mycM28z1XXPD1DNR (dose |
| | | 5 × 10⁴ and 1 × 10⁵) CAR T cells |
| | | remained tumor-free until |
| | | termination of study (day 68; |
| | | median survival not reached vs. |
| | | 50 days for mice treated with |
| | | 1 × 10⁵ mycM28z CAR T cells) |
| | | Weight gain and no toxicities |
| | | observed in mice treated with |
| | | mycM28z1XXPD1DNR CAR T |
| | | cells |
| Immuno- | Detect CAR T cells in | Regionally administered |
| fluorescence | primary tumor of | mycM28z1XXPD1DNR CAR T |
| staining of | intrapleurally treated | cells found infiltrating tumor and |
| ex vivo tumor | mice | enriched in peritumoral areas |
| Repeated | Investigate functional | Single intrapleural dose of 1 × 10⁵ |
| antigen | persistence and long- | mycM28z1XXPD1DNR CAR T |
| challenge | term antitumor | cells demonstrated superior |
| in vivo | efficacy | functional persistence and |
| | | enhanced long-term antitumor |
| | | activity by resisting establishment |
| | | of rechallenged intraperitoneal |
| | | tumor |
| | | mycM28z CAR T cells became |
| | | dysfunctional upon antigen |
| | | rechallenge |
| Antitumor | Validate antitumor | Cryopreserved clinical-grade |
| efficacy of | efficacy of | M28z1XXPD1DNR CAR T |
| clinical-grade | cryopreserved T cells | demonstrated high viability after |
| CAR T cells | transduced with vector | thawing, exhibited antitumor |
| in vivo | stock for clinical trial | efficacy, and prolonged survival |
| | | in vivo |
| | | No toxicities observed |
| Toxicity | Investigate toxicity in | No mortality or morbidity |
| | vivo | No significant clinical signs |
| | | No significant difference in body |
| | | weight compared to nontumor |
| | | controls |
| | | Histopathology: No microscopic |
| | | findings related to acute or |
| | | delayed toxicity |
| | | Hematology: Female mice |
| | | sacrificed on study day 15 had a |
| | | high average % monocyte value; |
| | | no correlation with any |
| | | microscopic findings. |
| | | Clinical chemistry: Male mice |
| | | sacrifice on study day 14 had a |
| | | low average total protein value; |
| | | no correlation with any micro- |
| | | scopic findings. |
| | | Single orthotopic administration |
| | | of 1 × 10⁵ mycM28z1XXPD1DNR |
| | | CAR T cells in a mesothelioma |
| | | xenograft model is well tolerated. |

To facilitate the detection of CART cells in preclinical studies, CART cells comprising a myc-tag at the N-terminus of the mesothelin-specific scFv (mycM28z1XXPD1DNR) were generated. To evaluate consistency between mycM28z1XXPD1DNR and M28z1XXPD1DNR CAR T cells, the transduction efficiencies of the viral supernatants were compared and a consistent, concentration-dependent expression of vector components between the two constructs was observed. In addition, both CAR and PD1DNR were expressed proportionally within each transduced cell due to the presence of P2A self-cleaving peptide, which efficiently mediates bicistronic transgene expression. Upon comparison of the percentage of transduced cells expressing CAR to the number of vector copy insertions in the T-cell genome, a positive linear association between the dilution of viral supernatant used for transduction and the resulting vector copy number (VCN) was observed. From these observations, it was determined that a range of 35%-70% of T cells expressing CAR ensures optimal transduction efficiency, which (1) maintains a low VCN:cell ratio and (2) lowers the risk of insertional mutagenesis, which is usually higher with a high VCN:cell ratio.

To confirm the expression of PD1DNR and distinguish it from its endogenous counterpart, flow cytometry and qPCR assays were performed to measure and compare the cell-surface protein and intracellular mRNA expression of PD1, respectively, in T cells transduced with mycM28z1XXPD1DNR and mycM28z. At the cell-surface protein level, compared with mycM28z CAR T cells, mycM28z1XXPD1DNR CAR T cells exhibited a 2-fold increase in the percent of cells stained positive for PD1 and a 3-fold increase in the median fluorescence intensity (MFI) displayed by PD1-positive cells. At the mRNA level, relative to un-transduced T cells, mycM28z CAR T cells showed a 4-fold increase in both PD1 extracellular and intracellular domains, whereas mycM28z1XXPD1DNR CAR T cells exhibited a 157-fold increase in PD1 extracellular domain and only a 2-fold increase in PD1 intracellular domain. That expression of PD1 extracellular domain higher by orders of magnitude indicates high expression of PD1DNR, which serves to combat checkpoint inhibition.

Finally, to rule out any potential interference of the myc-tag (used in preclinical studies) with CAR function, the antitumor efficacy of mycM28z1XXPD1DNR and M28z1XXPD1DNR CAR T cells was compared using an impedance-based cytotoxicity assay that revealed no difference in the kinetics and overall killing of mesothelin-positive tumor cells across 3 different donors, confirming that the myc-tag does not interfere with CAR function.

Next, the specificity, cytotoxicity, accumulation, and cytokine secretion of mycM28z1XXPD1DNR CAR T cells were analyzed. In a ⁵¹Cr cytotoxicity assay, mycM28z1XXPD1DNR CAR T cells exhibited antigen-specific and human leukocyte antigen (HLA)-independent cytotoxicity against mesothelin-positive tumor cells with both constitutive expression and overexpression of PD-L1. Nonspecific cytotoxicity against mesothelin-negative tumor cells was not observed. mycM28z1XXPD1DNR CAR T cells did not express any cytotoxicity against PD-L1-overexpressing targets in the absence of mesothelin antigen expression. Upon conducting repeated antigen stimulation assay (antigen stress test), an up to 622-fold expansion in both mycM28z1XXPD1DNR and mycM28z CAR T cells over a period of 6 antigen stimulations was noticed. At the timepoints tested, the constructs demonstrated similar cytotoxicity following the first antigen stimulation and retained similar cytotoxicity up to the fourth antigen stimulation. When the effector-to-target (E:T) ratio was decreased further to increase antigen stress, mycM28z1XXPD1DNR CAR T cells retained cytotoxicity better than mycM28z CAR T cells upon the seventh antigen stimulation. However, over the course of the assay, secretion of effector cytokines (IL-2, IFN-γ, and TNF-α) was gradually reduced for both mycM28z1XXPD1DNR and mycM28z CAR T cells, indicating the safety profile of both constructs.

MPM tumor cells co-transduced with firefly luciferase (ffLuc) were administered intrapleurally to establish tumors representing an orthotopic cancer model of MPM. To monitor tumor growth non-invasively, tumor-bearing mice were injected intraperitoneally with a 150 mg/kg dose of luciferin and were visualized in an IVIS Spectrum imaging system (PerkinElmer, Waltham, Mass.) after 15 min using a protocol optimized for quantitatively monitoring pleural tumor regression or progression that was previously published by our laboratory (Servais et al., Curr Protoc Pharmacol. 2011; Chapter 14:Unit14 21).

In an initial experiment conducted to study antitumor activity of CAR T cells in vivo, NSG mice bearing orthotopic tumors were treated with a single intrapleural dose of 3×10⁴ mycM28z1XXPD1DNR CAR T cells and compared with mice treated with a single intrapleural dose of control CAR T cells specific for prostate-specific membrane antigen (P28z). Fifteen days later, mice treated with mycM28z1XXPD1DNR CAR T cells showed a significant reduction in tumor burden (p=0.0002), whereas mice treated with P28z CAR T cells started to become moribund due to high tumor burden.

In a second in vivo study, mice with orthotopic tumors were distributed into 3 groups, each receiving a single intrapleural dose of 1×10⁵ or 5×10⁴mycM28z1XXPD1DNR CAR T cells or 1×10⁵ mycM28z CAR T cells. Serial tumor imaging showed a decrease in tumor burden as early as 5 days after CAR T-cell administration, with complete tumor eradication, determined by a decrease in the bioluminescence imaging (BLI) signal to baseline level, at approximately day 19 for mice treated with 1×10⁵ mycM28z1XXPD1DNR CAR T cells and approximately day 26 for mice treated with 1×10⁵ mycM28z CART cells. Mice treated with either dose of mycM28z1XXPD1DNR CAR T cells remained tumor-free until termination of the study (68 days). Median survival was 50 days for mice treated with 1×10⁵ mycM28z CAR T cells; the median survival was not reached for mice treated with either dose of mycM28z1XXPD1DNR CAR T cells (>68 days; p=0.0085-0.0427). Ex vivo immunofluorescence imaging of pleural tumors revealed that mycM28z1XXPD1DNR CAR T cells surround the tumor periphery in a high density and invade the tumor parenchyma by 3 days after regional delivery. These results were reproduced by use of T cells from multiple donors with varying percentages of CD4 and CD8 T cells transduced with mycM28z1XXPD1DNR CAR. To investigate the functional persistence of mycM28z1XXPD1DNR CAR T cells, mice with orthotopic MPM that received a single intrapleural dose of either 1×10⁵ mycM28z1XXPD1DNR or mycM28z CAR T cells were rechallenged with escalating doses (2×10⁶ to 11×10⁶ cells/dose) of mesothelin-positive tumor cells administered intraperitoneally (repeated administration of cells is more feasible in the peritoneal cavity than in the pleural cavity) every 4-8 days up to 10 times. The BLI signal for mice treated with mycM28z1XXPD1DNR CAR T cells peaked shortly after each tumor rechallenge and returned to baseline level at all of the rechallenge time points. In contrast, mice treated with mycM28z CAR T cells showed the same trend for up to 5 tumor rechallenges, but failed to control tumor reestablishment following administration of higher tumor doses at the late tumor rechallenge time points (6 to 10), leading to tumor relapse and a moribund state. mycM28z1XXPD1DNR CAR T cells resisted intraperitoneal tumor establishment for 10 repeated challenges, even >126 days after a single intrapleural dose of 1×10⁵ CART cells, without any apparent signs of toxicity. In an environment of high antigen stress, mycM28z1XXPD1DNR CAR T cells exhibited superior functional persistence and enhanced antitumor efficacy, compared with mycM28z CAR T cells in vivo. To confirm that this enhanced efficacy was not due to graft-versus-host disease, which is commonly seen in NSG mice treated with CAR T cells at this time point, nonantigen-expressing targets were administered, resulting in an increase in tumor BLI with no antitumor response, confirming that the observed antitumor efficacy was antigen-specific.

To validate the antitumor efficacy of cryopreserved T cells transduced with the M28z1XXPD1DNR CAR-encoding viral supernatant produced to be used in the clinical trial, M28z1XXPD1DNR CAR T cells generated by the MSK Cell Therapy and Cell Engineering Facility (CTCEF) were thawed (viability: 88% after thawing) and injected intrapleurally into mice with orthotopic MPM at a dose of 6×10⁴ and 2×10⁵ CART cells/mouse. Tumor regression and eradication was observed for both doses with 100% of the mice surviving until the end of the observation period (day 70), whereas tumor progressed in untreated mice, causing death by day 19. Cryopreserved CAR T cells demonstrated high viability after thawing and were efficacious without any signs of toxicity.

In the conduct of the above efficacy experiments, no toxicities were observed in mice, and weights remained stable throughout.

Section 3 of this Example (entitled “Nonclinical toxicology”) describes a study conducted in mice to specifically evaluate the potential toxicity of mycM28z1XXPD1DNR CAR T cells in an orthotopic mouse model of MPM. Mortality, morbidity, weights, clinical signs, hematology and clinical chemistry, gross necropsy, and histopathologic evaluations were assessed in 96 (48 male and 48 female) NSG mice, bearing 8-days-old orthotopic mesothelioma that were randomly assigned to control and treatment groups. A dose of 1×10⁵ CART cells/mouse or control vehicle (5×10⁶ CAR T cells/kg) were administered once via orthotopic injection. On day 2 and day 14 after CAR T-cell or vehicle administration (interim and final sacrifice, respectively), mice were sedated for necropsy and assessment of hematology and clinical chemistry parameters. Day 14 was chosen as the time point for final sacrifice as the tumor had either regressed significantly or been eradicated at this time point (as evidenced by BLI or necropsy from prior experiments). Performing sacrifice and necropsy at this time point allows examination of any on-target, off-tumor effects (the scFv used in our CAR reacts to mouse mesothelin) (Feng et al., Mol Cancer Ther. 2009) on normal tissue—specifically pleura, peritoneum, and pericardium—with low levels of expression of mesothelin, following peak CAR T-cell expansion in the absence of tumor burden with high antigen expression.

No mortality or morbidity was observed in animals in this study, with the exception of 2 animals from the control vehicle-treated group that underwent selected sacrifice due to morbidity and labored breathing 20-22 days after tumor administration. Previous work in our laboratory showed that control vehicle-treated animals may become moribund due to tumor burden at approximately 20-22 days after tumor administration (Servais et al., Clin Cancer Res. 2012; Servais et al., Curr Protoc Pharmacol. 2011; Chapter 14:Unit14 21; Adusumilli et al., Sci Transl Med. 2014; 6(261):261ra151; Cherkassky et al., J Clin Invest. 2016; 126(8):3130-3144; Servais et al., PLoS One. 2011; 6(10):e26722). Therefore, these sacrifices were unscheduled but not unexpected. No mortality or morbidity was traced to the CAR T cells. Animals that received control vehicle showed a progressive decrease in body weight during the study period and a significant difference in weight, compared with nontumor controls and mice treated with mycM28z1XXPD1DNR CAR T cells. This was attributed to the increasing tumor burden of the control vehicle-treated animals. No significant clinical signs were observed for mice treated with mycM28z1XXPD1DNR CAR T cells. One test article-treated mouse was observed to have slight scabbing, which was attributed to irritation caused by the surgical clips, as no other animals were affected and animal activity was normal. Mice appeared normal throughout the monitoring period.

Female mice treated with mycM28z1XXPD1DNR CAR T cells that underwent final sacrifice 14 days after CAR T-cell administration had a high average percent monocyte value (average, 18.44%, n=5) (p≤0.0001), compared with tumor control vehicle mice (average, 3.34%, n=5). The reference range established for percent monocytes is 0.9%-18%. However, this did not correlate with any microscopic findings. No other significant or abnormal results were observed for the hematology parameters assessed. Any differences between test article-treated groups and the corresponding vehicle-treated groups were within normal reference ranges or were not biologically relevant or statistically significant.

Male mice treated with mycM28z1XXPD1DNR CAR T cells that underwent final sacrifice 14 days after CAR T-cell administration had a low average total protein value (average, 3.83 g/dL, n=5) (p=0.0022), compared with tumor control vehicle mice (average, 4.68 g/dL, n=4). The reference range established for total protein is 4.1-6.4 g/dL. However, this did not correlate with any microscopic findings. No other adverse effects on clinical chemistry parameters were observed with test article administration. Any differences between test article-treated groups and the corresponding vehicle-treated groups were within normal reference ranges or were not biologically relevant or statistically significant.

Histopathologic review showed that there were no microscopic findings at the interim and final sacrifice days related to acute or delayed toxicity from mycM28z1XXPD1DNR CAR T-cell administration. Microscopic findings for animals in the CAR T-cell interim sacrifice groups included the presence of mixed cellular infiltration within the xenograft tumors. This was considered to be related to test article administration but not to any test article toxicity. Any other observed findings were determined to occur sporadically, at a similar incidence as in controls, or were common in the species/strain utilized.

mycM28z1XXPD1DNR CAR T cells were found in the tumor and spleen 8 days after intrapleural administration, and BLI revealed that, at the 2-week time point, the tumor burden was significantly decreased in CAR T cell-treated mice, confirming both successful administration and the pharmacologic activity of the test article. Mouse plasma cytokine levels obtained at the same time point showed slightly higher levels of IL-4 in mice treated with CAR T cells than in mice treated with vehicle control. Levels of IL-1β, IL-6, KC/GRO, and TNF-α were generally low and were not significantly different between mice receiving CAR T cells and mice receiving vehicle control. IFN-γ, IL-12p70, IL-1β, IL-2, and IL-5 were not detectable (below the limit of quantitation).

In conclusion, the data indicate that M28z1XXPD1DNR CAR T cells are well-tolerated. The dose of 1×10⁵ cells/mouse is 5-fold higher than the starting dose for patients (1×10⁶ cells/kg on a body weight basis), which corresponds to 5×10⁶ cells/kg. M28z1XXPD1DNR CART cells possess the same antigen-targeting moiety as M28z CAR T cells, for which patient-safety data (n=50) are already available. In our clinical trial (IND16354), we did not observe any dose-limiting toxicities and no on-target, off-tumor toxicity with up to 6×10⁷ intrapleurally administered M28z CAR T cells/kg in combination with anti-PD1 checkpoint blockade antibody given 3 weeks after CAR T-cell administration. Four patients received a second dose of intrapleural M28z CAR T cells following multiple doses of anti-PD1 agent (washout period of 4 weeks) with no observed toxicity. Together, all of the preclinical and clinical data available reasonably indicate that the proposed starting dose and route of administration of M28z1XXPD1DNR CART cells do not pose unacceptable risk to our patients.

### 2. Non-Clinical Pharmacology

### A. Methods

CAR vectors. The vectors used in the nonclinical studies are summarized in Table 5.

TABLE 5
Summary of vectors used in nonclinical studies.

| Vector | Protein expression |
|---|---|
| mycM28z | Mesothelin-specific scFv (myc tag), |
| | CD28, CD3ζ |
| mycM28z1XXPD1DNR | Mesothelin-specific scFv (myc tag), |
| | CD28, CD3ζ(1XX), P2A, PD1DNR |
| M28z1XXPD1DNR | Mesothelin-specific scFv, CD28, |
| (research-grade) | CD3ζ(1XX), P2A, PD1DNR |
| M28z1XXPD1DNR | Mesothelin-specific scFv, CD28, |
| (clinical-grade*) | CD3ζ(1XX), P2A, PD1DNR |
| GFP-ffLuc (transduced | GFP, firefly luciferase |
| into tumor cells) | |
| P28z (negative control) | Prostate-specific membrane antigen |
| | (PSMA) scFv, CD28, CD3ζ |

*Clinical grade: CAR T cells manufactured by the MSK Cell Therapy and Cell Engineering Facility using viral supernatant produced for the clinical trial.

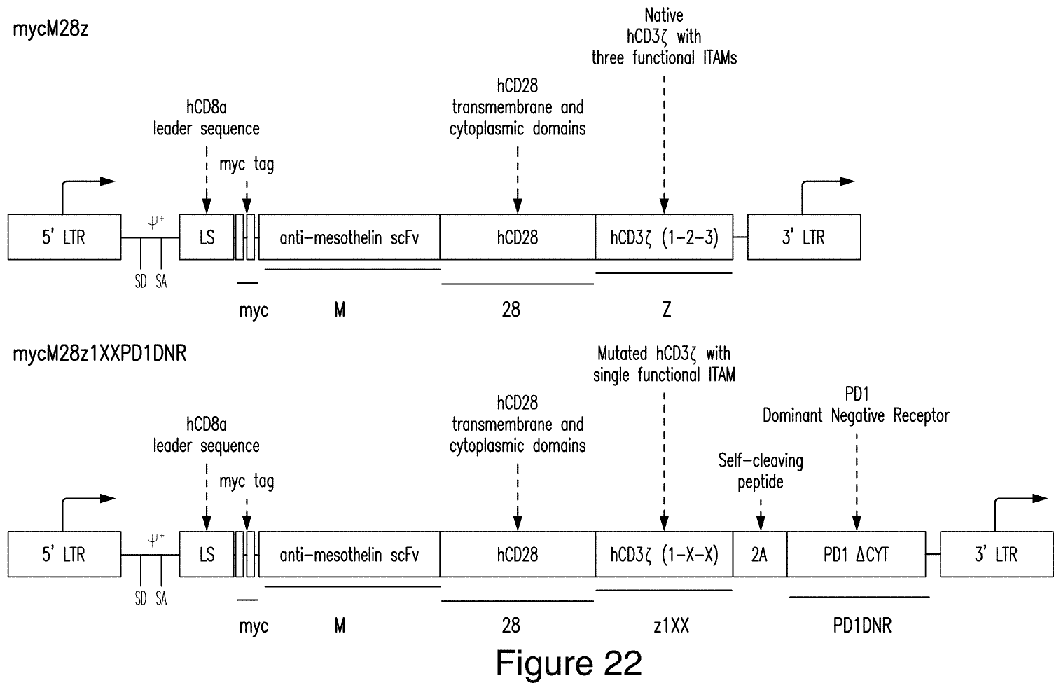

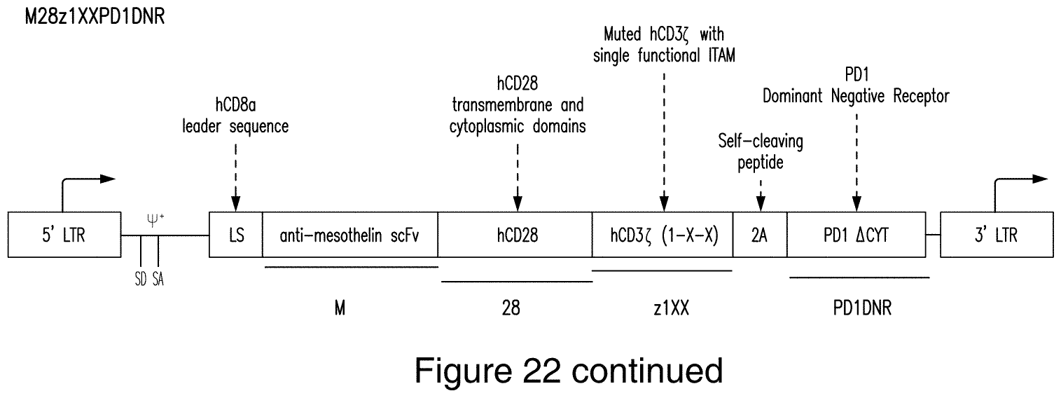

Mesothelin-targeted CAR constructs contain the mesothelin-specific scFv (clone m912) (Feng et al., Molt Cancer Ther. 2009) fused to a CD28 costimulatory domain and a CD3ζ signaling domain (M28z). The CD3 chain was mutated in two of its three ITAMs, resulting in a single functional ITAM (termed 1XX) (Feucht et al., Nat Med. 2019; 25(1):82-88). The CAR is fused to PD1DNR through a P2A site derived from porcine teschovirus-1. PD1DNR is composed of the PD1 signaling peptide and PD1 extracellular domain fused to the CD8 transmembrane and hinge domains (Cherkassky et al., J Clin Invest. 2016; 126(8):3130-3144). This decoy receptor is depleted of the PD1 signaling domain, thereby providing T-cell-intrinsic checkpoint blockade. To facilitate detection of CAR, a myc-tag (amino acid sequence EQKLISEEDL ×2) was fused to the N-terminus of the scFv in the constructs mycM28z and mycM28z1XXPD1DNR. To avoid any potential risk of immunogenicity in humans, the clinical-grade construct M28z1XXPD1DNR does not contain a myc-tag. In addition, the protein expression is codon-optimized to avoid any immunogenicity in the construction of the CAR and PD1DNR. The detailed structure of the constructs used in nonclinical studies is depicted in FIG. 22.

The expression of the CAR constructs is under the control of the Moloney murine leukemia virus long terminal repeat (LTR) of the retroviral SFG vector (Riviere et al., Proc Natl Acad Sci USA. 1995; 92(15):6733-6737). Expression of both CAR and PD1DNR is driven by the retroviral LTR.

All CAR vectors were transfected into 293T H29 packaging cell lines, and the viral supernatant produced by these cells was used to transduce and generate stable 293T RD114 cell lines.

CAR T cells. Human primary T lymphocytes were isolated from the blood of healthy volunteer donors under an institutional review board-approved protocol. Phytohemagglutinin-activated peripheral blood mononuclear cells (PBMCs) were isolated by low-density centrifugation on Lymphocyte Separation Medium (Corning, New York, N.Y.). Two days after isolation, PBMCs were transduced with viral supernatant containing mycM28z, mycM28z1XXPD1DNR, or M28z1XXPD1DNR vectors via spinoculation at 1800 g for 60 min at 24° C. on 6-well culture plates coated with 15 μg/mL RetroNectin (Takara, Shiga, Japan). Following the day of spinoculation, transduced PBMCs were maintained in RPMI-1640 supplemented with 10% fetal bovine serum (FBS), 2 mM L-glutamine, 100 units/mL penicillin, 100 μg/mL streptomycin, and 20 units/mL IL-2. Transduction efficiencies were determined by flow cytometry analysis of myc-tag expression on the scFv of the tagged CARs or by staining with a F(ab′)₂ fragment-specific anti-human IgG antibody to detect the expression of the scFv of the untagged CAR of M28z1XXPD1DNR. CAR T cells were tested for viability >70%, T-cell purity >95% by anti-human CD3 staining, transduction efficiency of 35%-70% by flow cytometry, and CD4/CD8 expression. The characteristics of T cells used in nonclinical studies are summarized in Table 6.

TABLE 6
Characteristics of T cells used in nonclimcal studies.

| PBMC | | % CD3 + | % CD3 + CAR | % CD3 + |
|---|---|---|---|---|
| donor | % CD3 | CAR+ | CD4+ | CAR CD8+ |
| H16 | 97-98 | 43-62 | 48-57 | 33-45 |
| H18 | 97-99 | 38-57 | 42-45 | 45-52 |
| H19 | 98 | 37-64 | 41-46 | 47-51 |
| C | 97-98 | 65-66 | 37-38 | 55-57 |
| C1 | 97 | 64-70 | 46 | 48-49 |
| H1 | 97 | 56-60 | 38 | Not |
| | | | | determined |

Tumor cells. Cells from the MSTO-211H human pleural mesothelioma cell line (ATCC CRL-2081) were genetically modified and used for in vitro and in vivo studies (Table 7).

TABLE 7
Summary of tumor cells used in nonclinical studies.

| Tumor cell | Protein expression |
|---|---|
| MSTOG | GFP, ffLuc |
| MGM | GFP, ffLuc, mesothelin |
| MGM-PDL1 | GFP, ffLuc, mesothelin, PD-L1 |

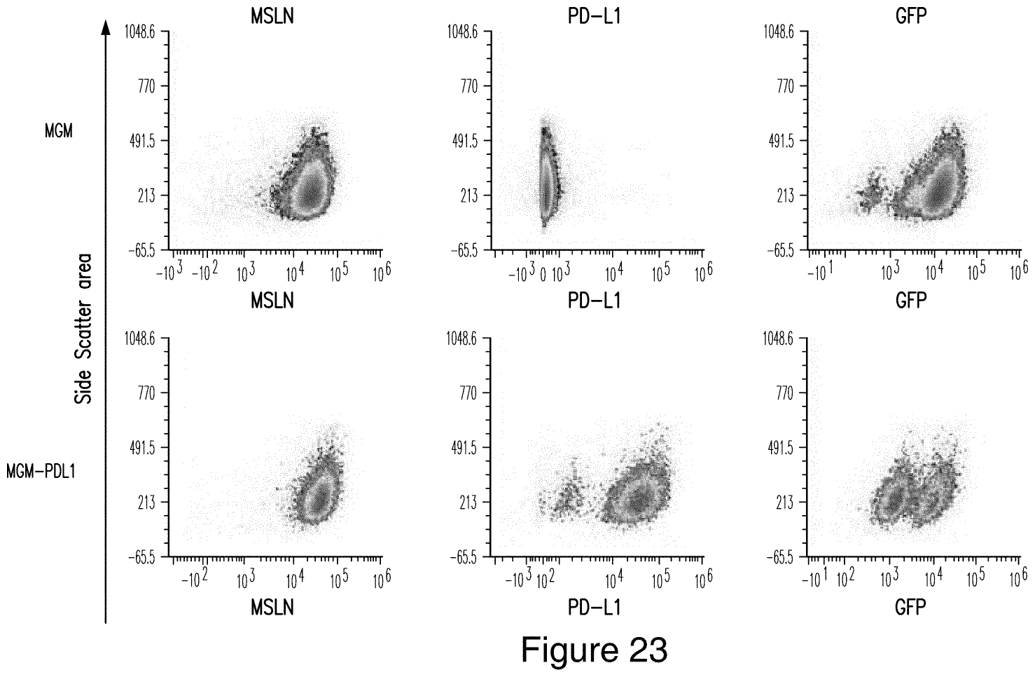

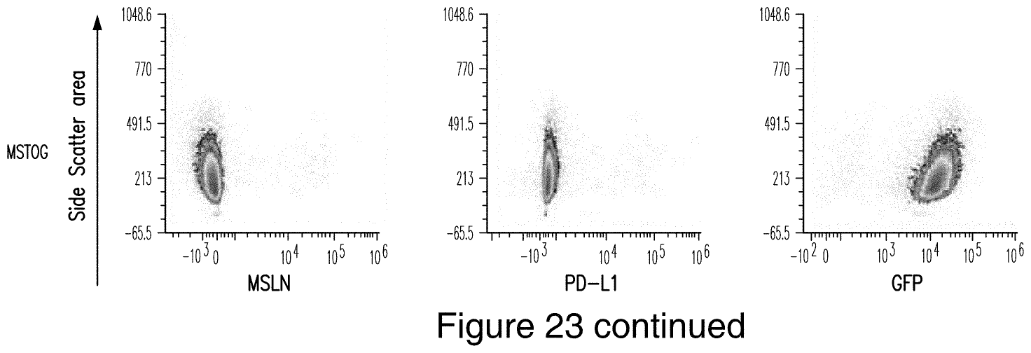

MSTO-211H is a biphasic MPM cancer cell line that lacks expression of endogenous CD80/86 costimulatory ligands. MSTO-211H cells were retrovirally transduced to express GFP and the ffLuc protein, termed MSTOG, allowing noninvasive in vivo BLI using SFG retroviral vectors constructed at MSK. Media containing filtered virus was added to cells permeabilized using 8 ug/mL Polybrene (Sigma-Aldrich, St. Louis, Mo.). Cells were re-infected with freshly collected virus 24 h later. These cells were transduced with the human mesothelin-variant 1 (isolated from a human ovarian cancer cell line [OVCAR-3]) subcloned into an SFG retroviral vector to generate mesothelin+ MSTO-211H cells, termed MGM. Similarly, MGM cells were transduced with PD-L1 (OriGene cDNA subcloned into SFG vector), resulting in MGM-PDL1. Tumor cells were maintained in RPMI-1640 media with 10% FBS, 2 mM L-glutamine, 100 units/mL penicillin, and 100 ug/mL streptomycin in a 5% CO₂ humidified incubator at 37° C. A linear correlation between the number of luciferase-expressing tumor cells and BLI photon counts in vitro (Pearson r=0.999, p<0.0001, data not shown) was observed. The relative expression levels of transduced proteins is depicted in FIG. 23.

Flow cytometry. Flow cytometry was performed using the Attune NxT Flow Cytometer (ThermoScientific, Waltham, Mass.) or BD LSRFortessa (BD Biosciences, San Jose, Calif.). Human mesothelin cell-surface expression on tumor cells was detected using a phycoerythrin-conjugated anti-human mesothelin rat IgG2a (R&D Systems, Minneapolis, Minn.). Human PD-L1 cell-surface expression on tumor cells was detected using a phycoerythrin-cyanine 7-conjugated anti-human PD-L1 mouse IgG1 (BD Biosciences). Human T cells were analyzed for their cell-surface expression of human CD3 using an allophycocyanin-cyanine 7-conjugated anti-human CD3 mouse IgG2a or phycoerythrin-cyanine 7-conjugated anti-human CD3 mouse IgG1 antibody (BioLegend, San Diego, Calif.) and either human CD4 or human CD8 using a fluorescein isothiocyanate-conjugated anti-human CD4 mouse IgG1 (BioLegend) or an Alexa Fluor 488-conjugated anti-human CD8 mouse IgG1 (BioLegend), respectively. Cell-surface expression of CAR was quantified using a phycoerythrin-conjugated anti-myc-tag antibody (Cell Signaling Technology, Danvers, Mass.) or an Alexa Fluor 647-conjugated F(ab′)₂ fragment-specific goat anti-human F(ab′)₂ fragment (Jackson ImmunoResearch, West Grove, Pa.). Cell-surface expression of PD1 on CAR T cells was analyzed with a Brilliant Violet 711-conjugated anti-human PD1 mouse IgG1 (BioLegend). For ex vivo detection of human T cells, processed mouse tissue was stained with a phycoerythrin-cyanine 7-conjugated anti-human CD3 mouse IgG1 antibody and with an allophycocyanin-cyanine 7-conjugated anti-human CD45 mouse IgG1 antibody (BioLegend). Discrimination of live cells from dead cells was performed by staining cells with either 4′,6-diamidino-2-phenylindole (DAPI, ThermoFisher Scientific, Waltham, Mass.) or eFluor 506 (ThermoFisher Scientific). Data analysis was performed using FCS Express (De Novo Software, Pasadena, Calif.) and FlowJo (BD Biosciences) software. Table 8 summarizes the antibodies used for flow cytometry.

TABLE 8
Flow cytometry antibodies for nonclinical pharmacology studies.

| | | | Antibody | Fluoro- | Manu- |
|---|---|---|---|---|---|
| Marker | Clone(s) | Species | format | phores | facturer |
| CD3 | HIT3a | Mouse | IgG2a | APC-CY7 | BioLegend |
| | UCHT1 | Mouse | IgG1 | PE-CY7 | BioLegend |
| CD45 | 2D1 | Mouse | IgG1 | APC-CY7 | BioLegend |
| CD4 | RPA-T4 | Mouse | IgG1 | FITC | BioLegend |
| CD8 | RPA-T8 | Mouse | IgG1 | AF700 | BioLegend |
| PD1 | EH12.2H7 | Mouse | IgG1 | BV711 | BioLegend |
| Myc- | 1B11 | Mouse | IgG2a | PE | Cell |
| tag | | | | | Signaling |
| Anti- | Polyclonal | Goat | F(ab′)₂ | AF647 | Jackson |
| F(ab′)₂ | | | | | Immuno- |
| | | | | | Research |
| Meso- | 420411 | Rat | IgG2a | PE | R&D |
| thelin | | | | | Systems |
| PD-L1 | MIH1 | Mouse | IgG1 | PE-CY7 | BD |
| | | | | | Biosciences |

Determination of VCN. Total genomic DNA from CAR T cells was isolated using the Miniprep Kit (Qiagen, Hilden, Germany). TaqMan PCR primers and probes were used to detect SFG and the housekeeping gene albumin (ALB). Human SFG probe and primer sequences:
Probe sequence: 5′-VIC-AGGACCTTACACAGTCCTGCTGAC-TAMRA-3′ [SEQ ID NO: 126]
Forward primer sequence: 5′-AGAACCTAGAACCTCGCTGGA-3′ [SEQ ID NO: 127]
Reverse primer sequence: 5′-CTGCGATGCCGTCTACTTTG-3′ [SEQ ID NO: 128]

Human-ALB probe and primer sequences:
Probe sequence: 5′-VIC-TGCTGAAACATTCACCTTCCATGCAGA-TAMRA-3′ [SEQ ID NO: 129]
Forward primer sequence: 5′-TGAAACATACGTTCCCAAAGAGTTT-3′ [SEQ ID NO: 130]
Reverse primer sequence: 5′-CTCTCCTTCTCAGAAAGTGTGCATAT-3′ [SEQ ID NO: 131]

The amplification reaction (25 μL) contained 5 μL (150 μg) of genomic DNA and 12.5 μL of TaqMan Fast Advanced Master Mix (ThermoFisher Scientific), 0.8 μL of primers (forward and reverse), 0.2 μL of TaqMan probe, and 5.7 uL of distilled water. qPCR conditions were as follows: 50° C. (2 min), 95° C. (20 min), followed by 42 cycles of 95° C. (15 sec) and 60° C. (1 min) using a QuantStudio 7-Flex Real-Time PCR System (ThermoFisher Scientific). All PCR measures were performed in triplicate. VCN per cell was calculated as the ratio of (mean quantity of SFG/mean quantity of ALB)*2. Mean quantities were extrapolated from SFG and ALB standard curves.

Determination of PD1 mRNA expression. Total RNA from CAR T cells was isolated using the Miniprep Kit (Qiagen) and subjected to reverse-transcriptional reaction using the High-Capacity cDNA Reverse Transcription Kit (ThermoFisher Scientific). The SYBR Green assay was used to detect extracellular and intracellular domains of human PDCD1. Human glyceraldehyde 3-phosphate dehydrogenase (GAPDH) was used for normalization. The following primers were used.

GAPDH primer sequences:

Forward primer sequence:
[SEQ ID NO: 132]
5′-GAAGGTGAAGGTCGGAGT-3′
Reverse primer sequence:
[SEQ ID NO: 133]
5′-CATGGGTGGAATCATATTGGAA-3′

PD1 extracellular domain primer sequences (Yoon et al., Science. 2015; 349(6247):1261669):

Forward primer sequence:
[SEQ ID NO: 134]
5′-CCAGGATGGTTCTTAGACTCCC-3′
Reverse primer sequence:
[SEQ ID NO: 135]
5′-TTTAGCACGAAGCTCTCCGAT-3′

PD1 intracellular domain primer sequences (Hsu et al., J Immunol. 2016; 197(5):1884-1892:

Forward primer sequence:
[SEQ ID NO: 136]
5′-ACGAGGGACAATAGGAGCCA-3′
Reverse primer sequence:
[SEQ ID NO: 137]
5′-GGCATACTCCGTCTGCTCAG-3′

cDNA was diluted 5 times for subsequent qPCR assay. The amplification reaction (20 μL) contained cDNA from 200 ng of total RNA and 10 μL of QuantiTect SYBR Green PCR Mix (Qiagen), 4 μL of primers (forward and reverse, each 200 nM), and distilled water. qPCR conditions were as follows: 95° C. (15 min), 95° C. (20 min), followed by 45 cycles of 94° C. (15 sec), 60° C. (30 sec), 72° C. (30 sec), and 50° C. (20 sec for data collection) using a QuantStudio 7 Flex Real-Time PCR system (ThermoFisher Scientific). All PCR measures were performed in triplicate. All primers were synthesized by Integrated DNA Technologies (Coralville, Iowa), and amplification efficacy (E) values were calculated. Relative expression of target genes was normalized to the reference group as a ratio according to the Pfaffl formula (Pfaff, Nucleic Acids Res. 2001; 29(9):e45):

Relative ratio=(E_{target})^{ΔCt target (control-sample)}/(E_{ref})^{ΔCt ref (control-sample)}

Target=PD1 extracellular/intracellular domain; Ref=GAPDH; control=un-transduced T cells.

⁵¹Cr cytotoxicity assay. The cytotoxicity of mycM28z1XXPD1DNR CART cells, compared with mycM28z T cells, was determined by standard 51Cr-release assay. In 96-well round-bottom plates, 5×10⁵ to 1×10⁶ total T cells in 200 μL of RPMI with 10% FBS, 2 mM L-glutamine, 100 units/mL penicillin, and 100 ug/mL streptomycin were serially diluted 1:2 in 100 μL of media. Target cells were incubated with 75 μCi of ⁵¹Cr per 1×10⁶ cells for 2 h and were resuspended at a final concentration of 5×10³ cells/100 μL. After 3 washes with media, 100 μL of the target cells were added to the T cells in triplicate and incubated for 4-18 h in a 5% CO₂ humidified incubator at 37° C. Supernatants were collected, plated on 96-well Lumina plates (PerkinElmer), and measured on a PerkinElmer TopCount. Spontaneous 51^{Cr} release was evaluated in target cells incubated with medium alone, and maximal 51^{Cr} release was determined with target cells incubated in 100 μL of 0.2% Triton X-100. The percentage of specific lysis was calculated as follows: [(experimental counts per minute (cpm)−spontaneous release cpm)/(total cpm−spontaneous release cpm)]×100. Data are reported as the mean of triplicate measurements+/−the standard error of the mean and were analyzed using Microsoft Excel (Microsoft, Redmond, Wash.) or GraphPad Prism (GraphPad Software, La Jolla, Calif.).

Impedance assay. CAR T cell-induced killing of target cells in vitro was assessed in real time using the xCELLigence Real Time Cell Analysis instrument (ACEA Biosciences, San Diego, Calif.). First, 50 μL of RPMI with 10% FBS, 2 mM L-glutamine, 100 units/mL penicillin, and 100 ug/mL streptomycin as culture medium for target and effector cells was added in a 96-well microtiter plate coated with gold microelectrodes (ACEA Biosciences) to measure the background impedance. Second, 10,000 target cells in 100 μL of medium per well were plated, and target cell adherence was monitored for 24-34 h before CAR T cells were added in 50 μL of medium in triplicate at E:T ratios of 1:1 to 1:3. To assess changes in impedance as a result of CAR T cell-induced killing and detachment of target cells, data were recorded every 15 min in a 5% CO₂ humidified incubator at 37° C. for up to 4 days after addition of effector cells.

Repeated antigen stimulation. To investigate the antitumor efficacy of CAR T cells upon repeated antigen stimulation in vitro, 3.3×10⁵ to 1×10⁶ T cells transduced with mycM28z1XXPD1DNR or mycM28z as control were cocultured with 3.3×10⁵ irradiated target cells (E:T ratio, 1:1 to 3:1) in 1 mL of RPMI with 10% FBS, 2 mM L-glutamine, 100 units/mL penicillin, and 100 ug/mL streptomycin in 24-well cell-culture plates. After 48 h of coculture, T cells were pooled, counted, analyzed for their expression of CAR by flow cytometry, and replated at the same E:T ratio with irradiated target cells for up to 6 rounds of repeated antigen exposure. After 1, 3, and 6 rounds of antigen exposure, the cytotoxicity of CAR T cells was assessed using ⁵¹Cr-release and impedance-based assays.

Accumulation. Accumulation was assessed by coculturing 3.3×10⁵ T cells transduced with mycM28z1XXPD1DNR or mycM28z as control with 3.3×10⁵ irradiated target cells (E:T ratio, 1:1) in 1 mL of RPMI with 10% FBS, 2 mM L-glutamine, 100 units/mL penicillin, and 100 ug/mL streptomycin in 24-well cell-culture plates. After 48 h of coculture, T cells were pooled, counted, analyzed for their expression of CAR by flow cytometry, and replated at the same E:T ratio with irradiated target cells for up to 6 rounds of repeated antigen exposure. The number of CAR T cells after each antigen stimulation cycle was used to determine the accumulation of CAR T cells over time by absolute T-cell count.

Cytokine quantification. Cytokine-release assays were performed by coculturing 3.3×10⁵ T cells transduced with mycM28z1XXPD1DNR or mycM28z as control with 3.3×10³ target cells (E:T ratio, 1:1) in RPMI with 10% FBS, 2 mM L-glutamine, 100 units/mL penicillin, and 100 ug/mL streptomycin in 24-well cell-culture plates. After 48 h of coculture, T cells were pooled, counted, analyzed for their expression of CAR by flow cytometry, and replated at the same E:T ratio with irradiated target cells for up to 6 rounds of repeated antigen exposure. For cytokine quantification, supernatants were collected 24 h after coculture for repeated antigen stimulations 1, 3, and 6 and were centrifuged at 800 g for 10 min at room temperature to remove cells and debris. Cytokine levels were determined in duplicate using the Human Cytokine Magnetic 30-plex Panel (Invitrogen, Carlsbad, Calif.) and the MAGPIX system (Luminex, Austin, Tex.), in accordance with the manufacturers' instructions.

Orthotopic mouse model. Orthotopic tumor models are considered more clinically relevant and better at predicting drug efficacy than standard subcutaneous models. Due to the fact that tumor cells are implanted directly into the organ of origin, these tumors reflect the original situation (e.g., microenvironment) much better than conventional subcutaneous xenograft tumor models. The combination of luciferase gene-transfected tumor cells together with orthotopic implantation of these cells allows noninvasive visualization of tumor growth, tumor distribution, and growth of metastases.

Female and male NOD/SCID gamma mice at 6-10 weeks of age (The Jackson Laboratory, Bar Harbor, Me.) were used to generate the orthotopic model. All procedures were performed under approved Institutional Animal Care and Use Committee (IACUC) protocols. Mice were anesthetized using inhaled isoflurane and oxygen. To establish orthotopic MPM tumors, direct intrapleural injection of mesothelin-expressing cells (8×10⁵ tumor cells) in 200 μL of serum-free media was performed via a right thoracic incision. Tumor was established in >95% of mice following inoculation at 8-12 days post-injection. Mice were sacrificed when moribund, in accordance with IACUC guidelines.

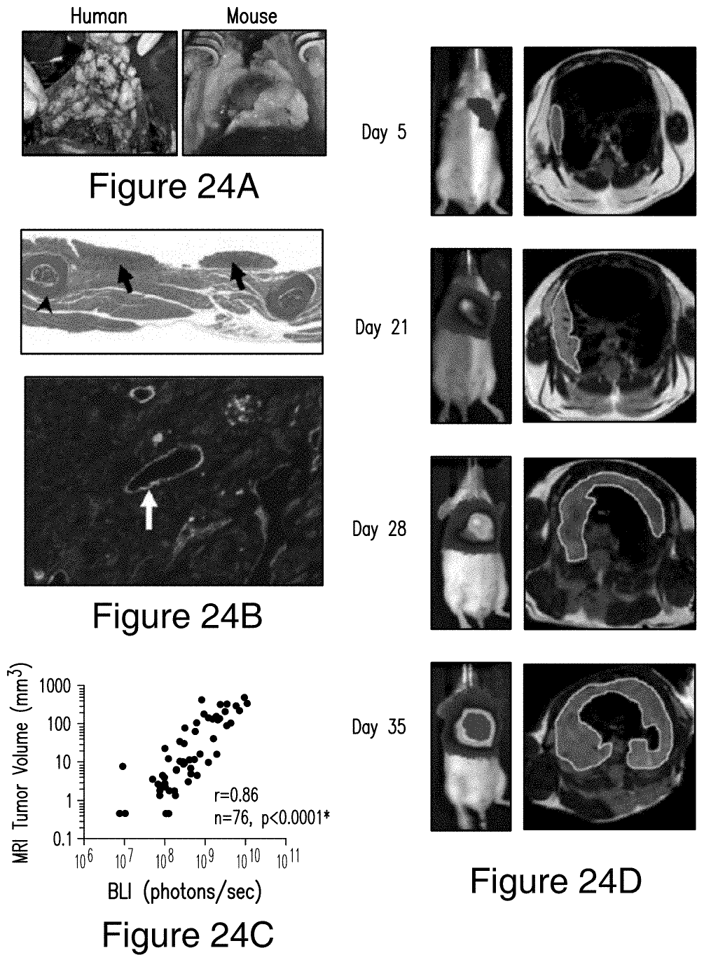

This model recapitulates the human tumor microenvironment in that it reflects the gross appearance and histopathologic profile of MPM (see FIG. 24A). In addition, the extensive lympho-vascularization of the MPM tumors in our mouse model (see FIG. 24B) is characteristic of human MPM.

BLI is a sensitive modality in vivo that is capable of detecting as few as 1×10³tumor cells in the pleural space. Standardization and sensitivity are based on our own experiments (Kachala et al., Clin Cancer Res. 2014; 20(4):1020-1028; Servais et al., Clin Cancer Res. 2012; Servais et al., Curr Protoc Pharmacol. 2011; Chapter 14:Unit14 21; Servais et al., PLoS One. 2011; 6(10):e26722; Servais et al., J Mol Med (Berl). 2011; 89(8):753-769). A strong correlation was observed between BLI tumor signal and pleural tumor volume determined by magnetic resonance imaging (MRI), a gold standard for tumor volume assessment (r=0.86, p<0.0001, adjusted for within-mouse clustering; FIGS. 24C and 24D). Our findings of quantitative BLI are attributable to the fact that tumor grows along the chest wall as a thickening of the pleural rind, minimizing tumor depth (FIG. 24A). Thus, BLI in the orthotopic pleural cancer model can provide an accurate quantitative evaluation of tumor burden, thereby providing a comparable standard for noninvasive serial evaluation of biomarker performance in the live mouse. Using immunohistochemical and flow cytometry analysis, we observed that mesothelin expression is sustained in the orthotopic MPM model even at advanced stages of disease (data not shown).

Mouse tissue processing. Mice were euthanized with CO₂, and pleural tumor and spleen were collected in a 50 mL conical tube with ice-cold RPMI-1640. For processing, the tissue was ground through a 40 μm cell strainer and centrifuged at 450 g for 5 min at 4° C. If the cell pellet appeared bloody, it was resuspended in 2 mL of ACK lysis buffer (Lonza, Basel, Switzerland) and incubated for 5 min at room temperature. After an additional centrifugation step at 450 g for 5 min at 4° C., the cell pellet was resuspended in PBS with 5% bovine serum albumin for washing and antibody staining for immediate use in flow cytometry.

Immunofluorescence. Female NSG mice with established MGM pleural tumor were injected with 5×10⁵ mycM28z1XXPD1DNR CART cells, mycM28z CART cells, or untransduced T cells. Three days after injection, mice were sacrificed, and pleural tumors were isolated, fixed in 4% paraformaldehyde overnight at room temperature, and processed for paraffin embedding using a Leica ASP6025 tissue processor (Leica Biosystems, Wetzlar, Germany). Freshly cut 5 μm paraffin sections were stained for sequential double immunofluorescence on a Leica Bond RX (Leica Biosystems) with 1.25 ug/mL CD45 mouse monoclonal antibody clone 2B11+PD7/26 (Dako) for 1 h with 10 min of 1:200 Tyramide Alexa Fluor 594 detection (Life Technologies, Carlsbad, Calif.) on Leica Bond Protocol F, followed by 0.03 ug/mL Mesothelin Rabbit Monoclonal Clone D9R5G (Cell Signaling) for 1 h with 10 min of 1:200 Tyramide Alexa Fluor 488 detection (Life Technologies) on Leica Bond Protocol F. The sections were pretreated with Leica Bond ER2 Buffer (Leica Biosystems) for 20 min at 100° C. before each staining. After staining, the sections were mounted with Mowiol for digital scanning with a Vectra 3.0 multispectral microscope (Perkin Elmer) using a 20× objective.

Tumor histology and immunostaining. Histopathologic evaluation of tumors was performed after hematoxylin and eosin staining of paraffin-embedded, 4% paraformaldehyde-fixed tissue samples. Immunohistochemical analysis for human mesothelin was performed with a mouse anti-human mesothelin IgG (1:100; Vector Labs, Burlingame, Calif.) using the Ventana platform. Grading of mesothelin was performed by a pathologist who was blinded to the clinical data, as follows: 0 (absent stain), 1 (weak expression), 2 (moderate expression), and 3 (strong expression). The distribution of mesothelin-positive tumor cells versus all tumor cells found in a single core was graded as 0 (absent), 1 (1%-50%), and 2 (51%-100%).

MRI. MRI was performed using a Bruker 4.7T USR scanner (Bruker Biospin, Ettlingen, Germany) equipped with a 400 mT/m gradient coil and a 32-mm ID custom built birdcage resonator. Thoracic axial MRI images were acquired using a RARE fast spin-echo sequence [repetition time (TR)=1.7 sec, echo time (TE)=40 msec, and 12 averages], triggered by animal respiration, to reduce respiration-induced motion artifacts. The slice thickness was 0.7 mm, and the in-plane image resolution was 117×156 mm. Tumor volumes (mm³) were measured by tracing tumor boundaries in each slice using Bruker ParaVision Xtip software (Bruker Biospin) and then calculated from the areas of tumor regions in each slice.

BLI. BLI was used to noninvasively image tumor burden. Mice were injected intraperitoneally with D-Luciferin at a dose of 150 mg/kg. Tumor bioluminescence was measured after 15 min, with mice in the dorsal and ventral position, using an IVIS Spectrum in vivo imaging system (PerkinElmer). The average total flux of dorsal and ventral was reported as the BLI signal in photons per second.

Quantification of cytokines in mouse plasma samples. Cytokine levels in plasma in a subset of mice from the toxicology study were quantified using the 10-plex V-Plex Mouse Proinflammatory Panel 1 assay (Meso Scale Diagnostics, Rockville, Md.). The following cytokines were analyzed: IFN-γ, IL-1β, IL-2, IL-4, IL-5, IL-6, IL-1β, IL-12p70, KC/GRO, and TNF-α. Where available, 60 μL of plasma was diluted 2-fold and plated into duplicate wells. Results (n=2 per sample) were reported as picograms per milliliter of plasma. The cytokine quantification was performed by the Immune Monitoring Facility at MSK.

### B. Human Tissue Mesothelin and In Vitro CAR T-Cell Studies

B.1. Mesothelin Expression in Tumor and Normal Tissues

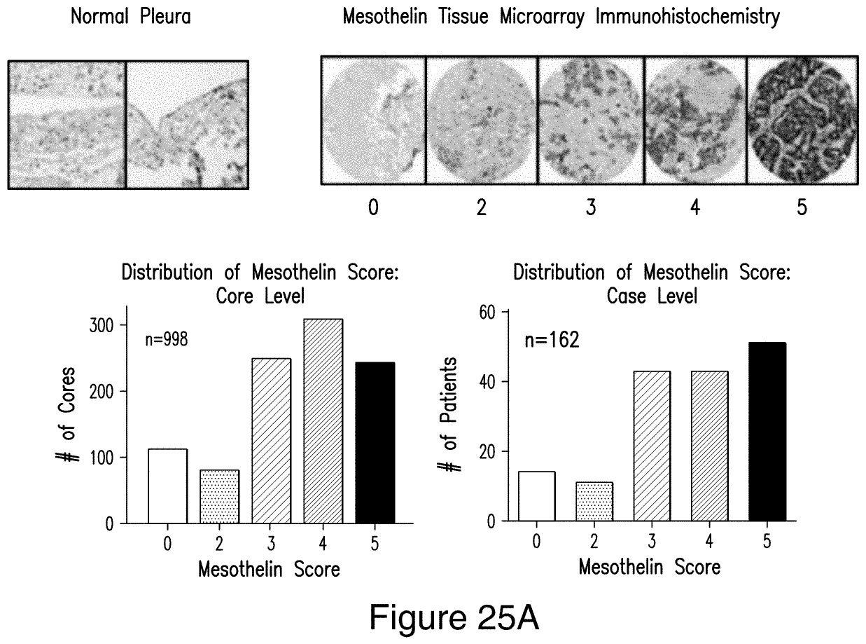

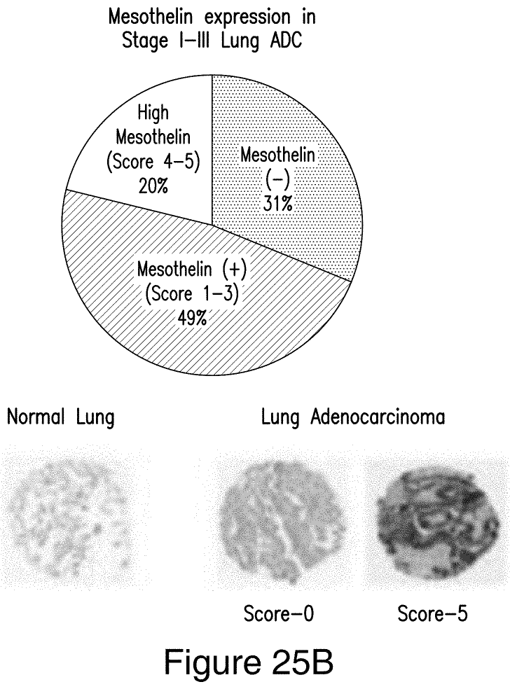

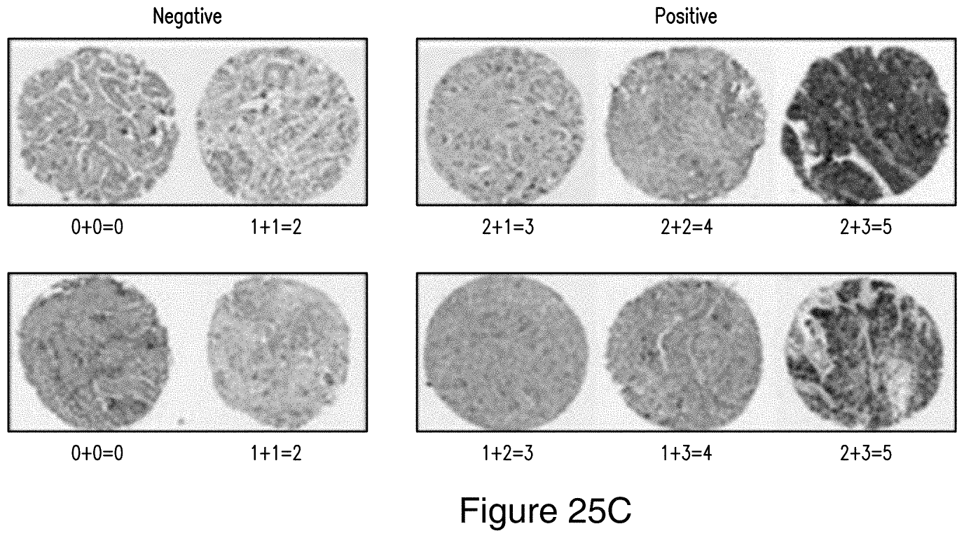

Published studies have observed very low mesothelin expression in normal pleura, peritoneum, and pericardium. Extensive studies in MPM, lung adenocarcinoma, and triple-negative breast cancer human tissues, along with normal tissue controls, were conducted using mesothelin immunohistochemical analysis (Kachala et al., Clin Cancer Res. 2014; 20(4):1020-1028; Servais et al., Clin Cancer Res. 2012; Rizk et al., Cancer Epidemiol Biomarkers Prey. 2012; 21(3):482-486). As shown in FIGS. 25A-25C, mesothelin was overexpressed in human MPM and lung carcinoma, compared with normal tissues (see FIG. 25A). Mesothelin was overexpressed in 69% of lung adenocarcinomas, with no expression in normal lungs (see FIG. 25B). Similarly, mesothelin was not expressed in normal breast tissue but is overexpressed in triple-negative breast cancers (see FIG. 25C).

B.2. Transduction of Human T Cells with M28z1XXPD1DNR

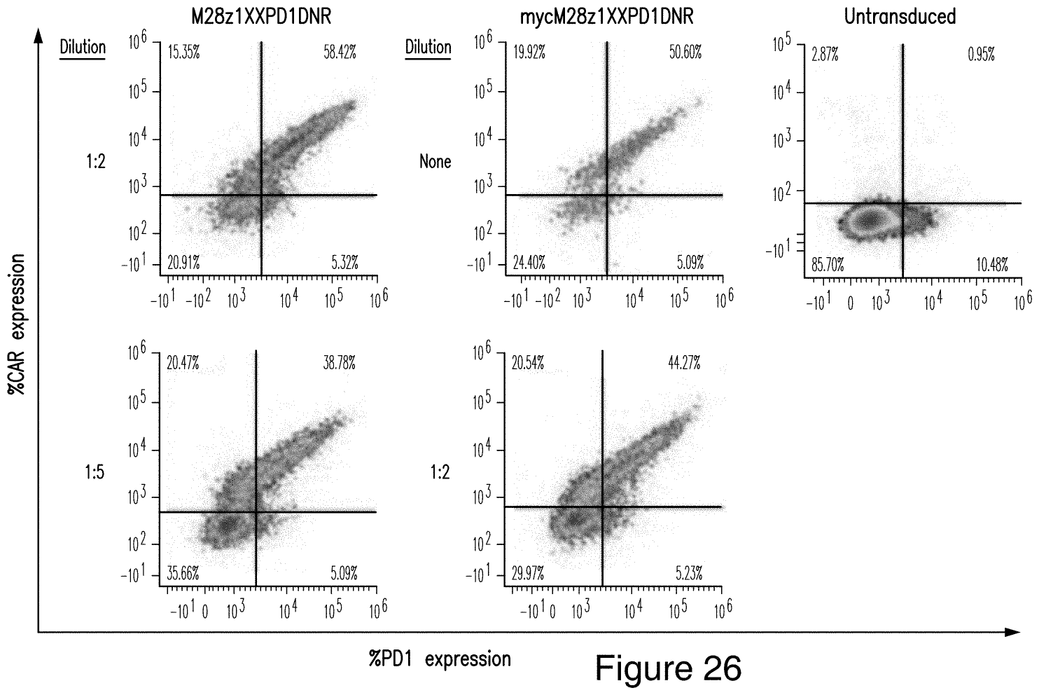

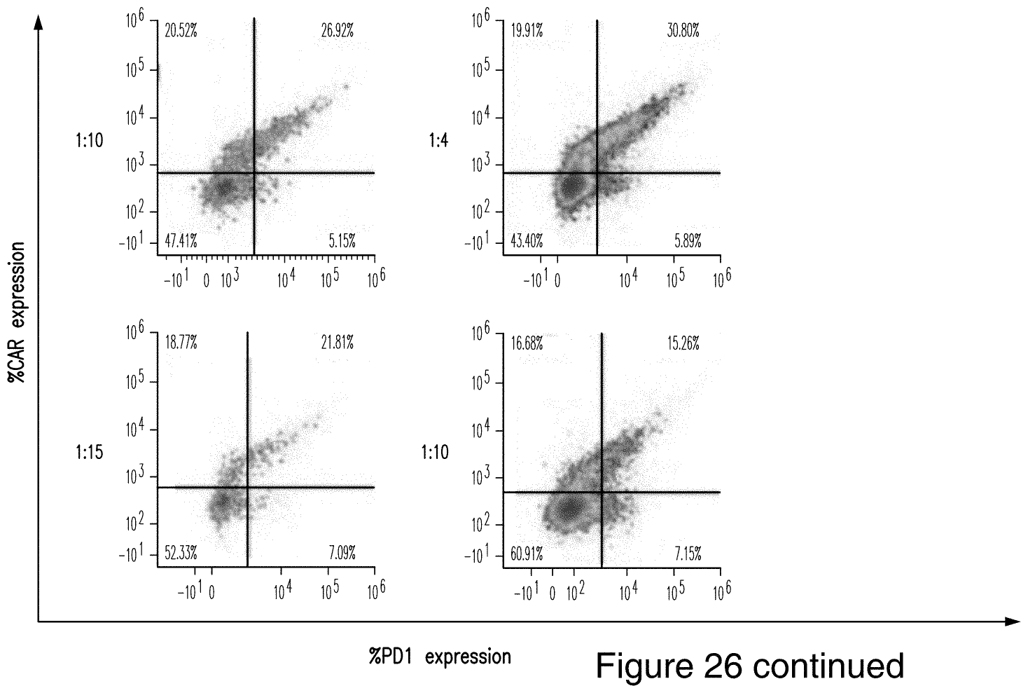

Viral supernatant encoding M28z1XXPD1DNR or mycM28z1XXPD1DNR obtained from stable 293T RD114 cell lines was titrated to assess the transduction efficacy of human T cells by flow cytometry. T cells were successfully transduced with CAR and PD1DNR, with donor-dependent surface expression levels ranging from 32% to 73% CAR and 22% to 64% PD1 (including PD1DNR and endogenous PD1 expression) for the tested dilutions of viral supernatant (see FIG. 26). CAR and PD1DNR were expressed proportionally due to the bicistronic transgene expression mediated by the P2A self-cleaving peptide.

B.3. Vector Copy Number (VCN)

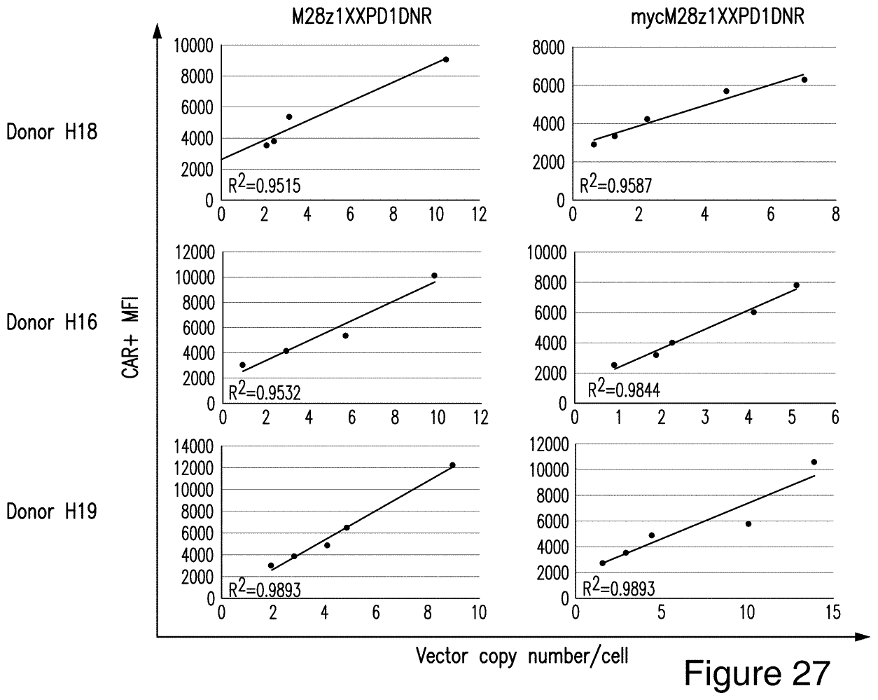

Following successful transduction of human T cells with M28z1XXPD1DNR, next the relationship between CAR expression and the integration of vector copies in the T-cell genome was analyzed. A close correlation between the MFI of CAR-positive cells and the VCN was found across multiple donors and dilutions of viral supernatant encoding M28z1XXPD1DNR or mycM28z1XXPD1DNR, yielding R² values in the range from 0.89 to 0.99 (see FIG. 27). Transduction efficiency and VCN were found to be donor-dependent, but high VCNs (>5/cell) were observed in particular for transduction efficiencies >70%, and low VCNs (<1/cell) were observed in particular for transduction efficiencies <35% (data not shown). To avoid a high risk of insertional mutagenesis and to ensure efficient transduction of T cells, we titrated the viral supernatants for all constructs to yield transduction efficiencies in the 35%-70% range for all subsequent in vitro and in vivo experiments.

B.4. M28z1XXPD1DNR CAR T Cells Overexpress the PD1 Extracellular Domain

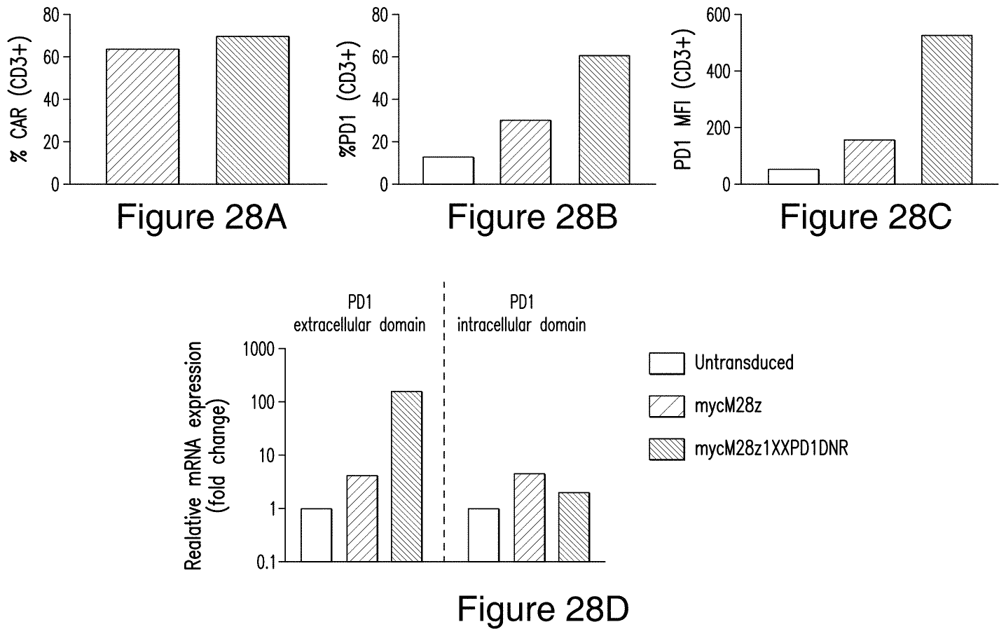

Human T cells transduced with M28z1XXPD1DNR express PD1DNR, a decoy receptor depleted of the intracellular PD1 signaling domain. The relative PD1 protein surface and mRNA expression in mycM28z1XXPD1DNR CAR T cells and mycM28z CAR T cells were investigated by flow cytometry and qPCR, respectively. At comparable CAR surface expression (64%-70%, see FIG. 28A), cell-surface PD1 staining led to detection of higher PD1 levels, both in percent positive cells (2-fold) and intensity (3.4-fold), on mycM28z1XXPD1DNR CAR T cells, compared with mycM28z CAR T cells (see FIGS. 28B and 28C). Detection of PD1 cell-surface expression was limited to the extracellular domain, which was present in PD1DNR as well as endogenous PD1. Therefore, although higher surface expression of the PD1 extracellular domain was caused by and indicative of expression of PD1DNR, discrimination between PD1DNR and endogenous PD1 was not possible at the cell-surface level.

To further explore differences in expression of PD1DNR and endogenous PD1, relative mRNA expression by qPCR was investigated. To distinguish between the expression of PD1DNR and that of endogenous PD1, primers specific for the PD1 extracellular and intracellular domains were designed and mRNA expression of both the PD1 extracellular and intracellular domains of mycM28z1XXPD1DNR and mycM28z CAR T cells relative to un-transduced T cells was measured. mycM28z CAR T cells did not express PD1DNR and hence express only endogenous PD1 at an equimolar ratio of extracellular to intracellular domain. While expression of the intracellular domain was exclusive to endogenous PD1, the extracellular domain was expressed by both PD1DNR and endogenous PD1. It was found that mycM28z CART cells expressed 4-fold higher levels of PD1 extracellular and intracellular domains, compared with un-transduced T cells. However, mycM28z1XXPD1DNR CAR T cells exhibited a 157-fold upregulation of the PD1 extracellular domain; un-transduced T cells exhibited only a 2-fold upregulation of the PD1 intracellular domain (see FIG. 28D).

In summary, these data confirm that PD1DNR is overexpressed both at the mRNA level and protein level, compared with endogenous PD1, in mycM28z1XXPD1DNR CAR T cells. This finding provides the basis for T cell-intrinsic checkpoint blockade.

B.5. Functional Comparison of M28z1XXPD1DNR CAR T Cells With and Without Myc-Tag

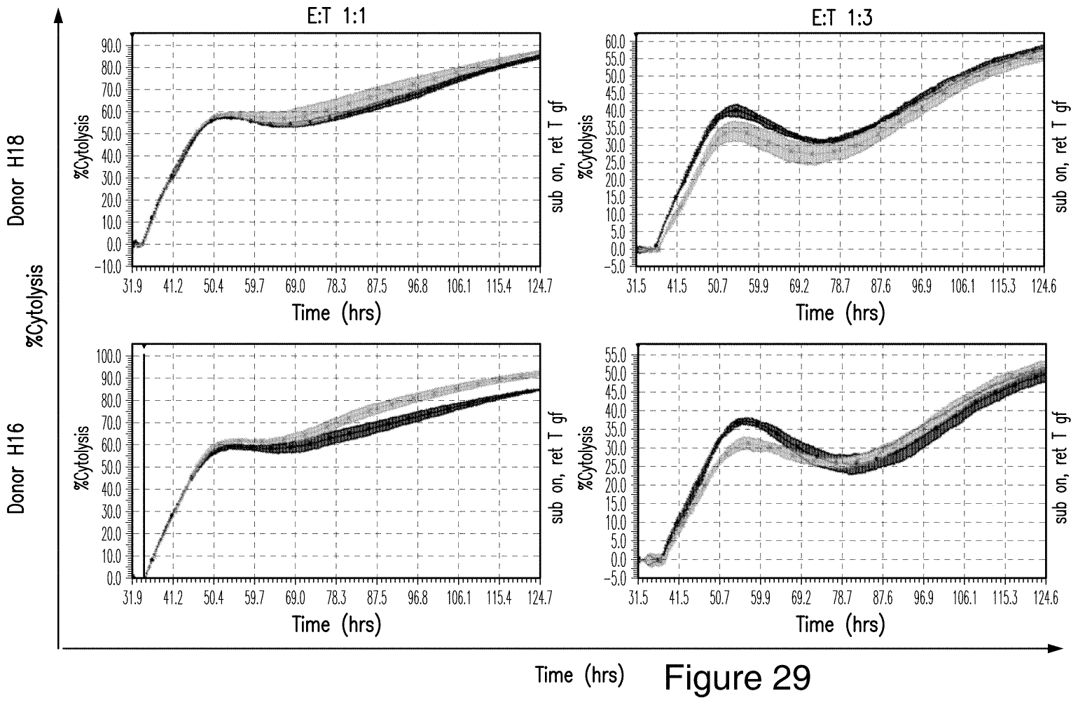

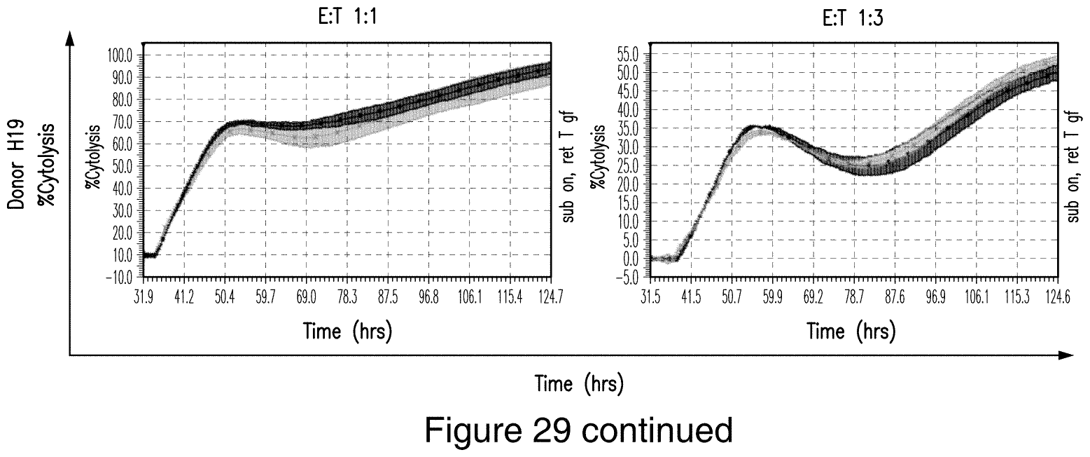

To facilitate detection of CAR expression, M28z1XXPD1DNR CART cells were generated with a myc-tag fused to the N-terminus of the anti-mesothelin scFv, resulting in mycM28z1XXPD1DNR. To rule out any potential interference of the myc-tag with CAR function, a head-to-head comparison of antitumor efficacy between M28z1XXPD1DNR (without tag) and mycM28z1XXPD1DNR CAR T cells was performed. Human T cells prepared from 3 independent donors transduced with both M28z1XXPD1DNR and mycM28z1XXPD1DNR at similar transduction levels (37%-63%, as determined by flow cytometry of cells stained with an anti-human F(ab′)₂ fragment-specific goat F(ab′)₂ fragment) exhibited no difference in kinetics and overall killing of MGM target cells at different E:T ratios (see FIG. 29).

B.6. mycM28z1XXPDIDNR CAR T Cells Mediate Antigen-Specific, HLA-Independent Tumor Lysis

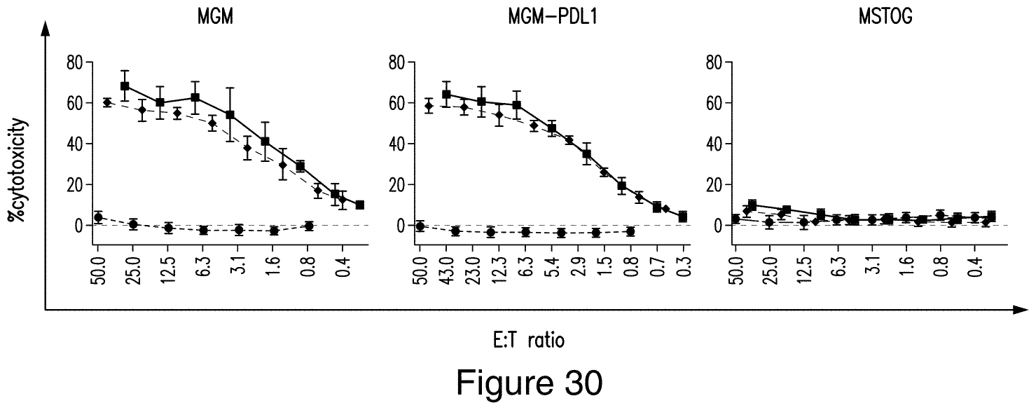

The cytotoxic activity of mycM28z1XXPD1DNR versus mycM28z CAR T cells was determined against a panel of tumor cell lines, including human mesothelioma cells (MSTO-211H) with (GM) and without (G) mesothelin expression as well as with constitutive PD-L1 expression, by ⁵¹Cr-release assay. mycM28z1XXPD1DNR CAR T cells efficiently killed MGM and MGM-PDL1 targets, similarly to mycM28z CAR T cells, upon 18 h coculture with mesothelin-expressing tumor cells at multiple E:T ratios, as shown in the cytotoxicity assay results below (see FIG. 30). No unspecific killing by either mycM28z1XXPD1DNR or mycM28z CAR T cells towards MSTOG tumor cells negative for mesothelin was observed. Untransduced T cells did not exhibit any killing of either target. These results confirm that M28z1XXPD1DNR CART cells kill target cells in a mesothelin-specific and HLA-independent manner.

B. 7. mycM28z1XXPDIDNR CAR T Cells Accumulate

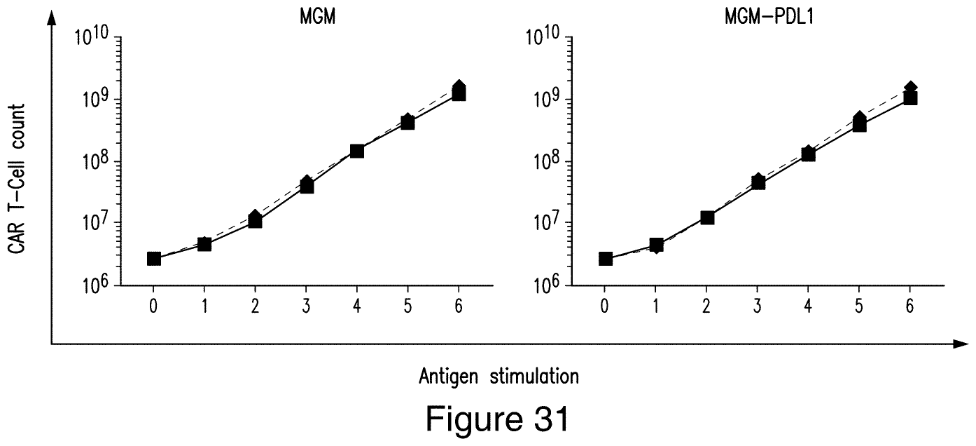

To investigate whether mycM28z1XXPD1DNR CAR T cells sustain T-cell accumulation on repeated antigen stimulation by mesothelin-expressing tumor cells with inducible PD-L1 expression (MGM) or constitutive PD-L1 expression (MGM-PDL1), the expansion of mycM28z1XXPD1DNR CAR T cells was quantified and compared with that of mycM28z CAR T cells. Over the course of 6 repeated antigen stimulations, mycM28z1XXPD1DNR CAR T cells expanded up to 622-fold, similarly to mycM28z CAR T cells (see FIG. 31).

B.8. Antitumor Efficacy of mycM28z1XXPDIDNR CAR T Cells Upon Repeated Antigen Stimulation

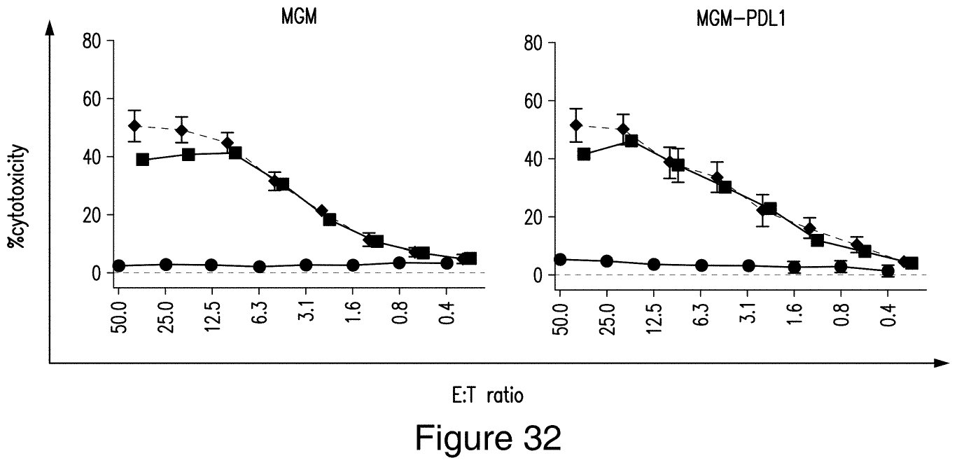

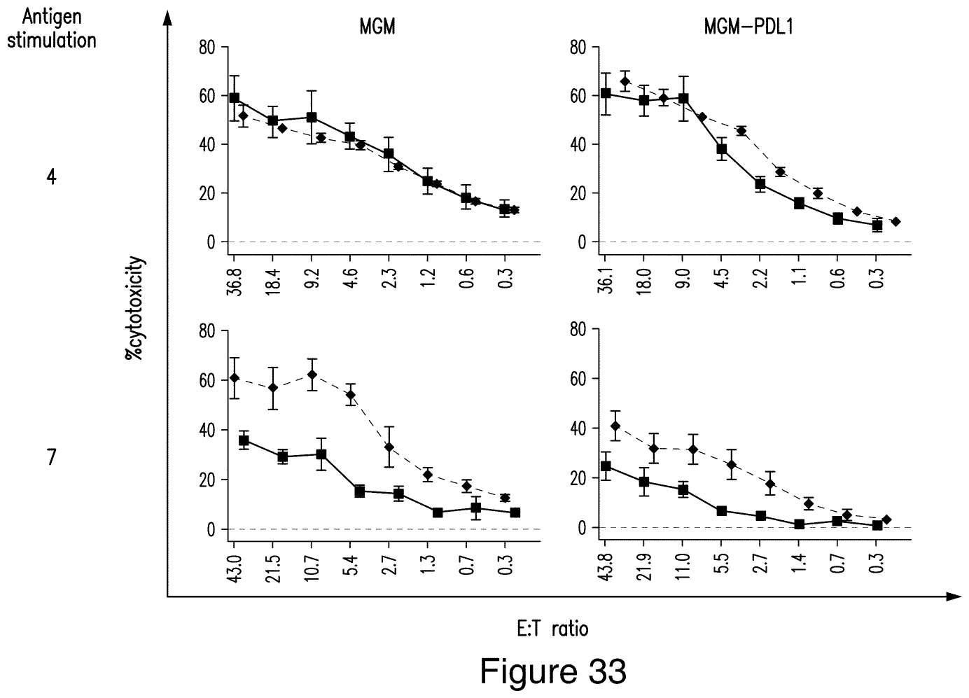

Upon the first antigen exposure, mycM28z1XXPD1DNR CAR T cells exhibited E:T ratio-dependent cytotoxicity toward MGM and MGM-PDL1 target cells but did not show differences in cytotoxicity, compared with mycM28z CAR T cells (see FIG. 32). To investigate the cytotoxic ability of mycM28z1XXPD1DNR and mycM28z CAR T cells during continuous antigen exposure (or high antigen stress), CAR T cells were repeatedly cocultured with irradiated MGM or MGM-PDL1 target cells every 48 h at an E:T ratio of 3:1 for 4 cycles. At the end of the third cycle, a sample of CAR T cells was subjected to a fourth antigen stimulation in a ⁵¹Cr cytotoxicity assay. mycM28z1XXPD1DNR and mycM28z CAR T cells demonstrated comparable cytotoxicity during the fourth antigen stimulation (see FIG. 33).

To further increase antigen stress, mimicking high tumor burden in solid tumors, CAR T cells were then co-cultured with target cells at an E:T ratio of 1:1 in the fifth and sixth cycles of antigen stimulation. At the end of the sixth cycle, another sample of CAR T cells was collected and subjected to the seventh cycle of antigen stimulation in a ⁵¹Cr cytotoxicity assay. Cytotoxicity upon the seventh antigen stimulation was substantially reduced for mycM28z CAR T cells against both target cell lines. In contrast, mycM28z1XXPD1DNR CAR T cells retained cytotoxicity on target cells. mycM28z1XXPD1DNR CAR T cells, compared with mycM28z CAR T cells, exhibited superior tumor cell kill on target cells, with high constitutive PD-L1 expression (see FIG. 33), although cytotoxicity was reduced compared with the initial and fourth antigen stimulations. Collectively, these data indicate that mycM28z CAR T cells reach an exhaustive state upon chronic antigen exposure, whereas mycM28z1XXPD1DNR CAR T cells are capable of maintaining antitumor activity even in environments of high antigen stress. The observed effects were dependent on biologic parameters such as donor, E:T ratio, and coculture time between tumor and CAR T cells.

B.9. Target-Stimulated Cytokine Release by mycM28z1XXPDIDNR CAR T Cells

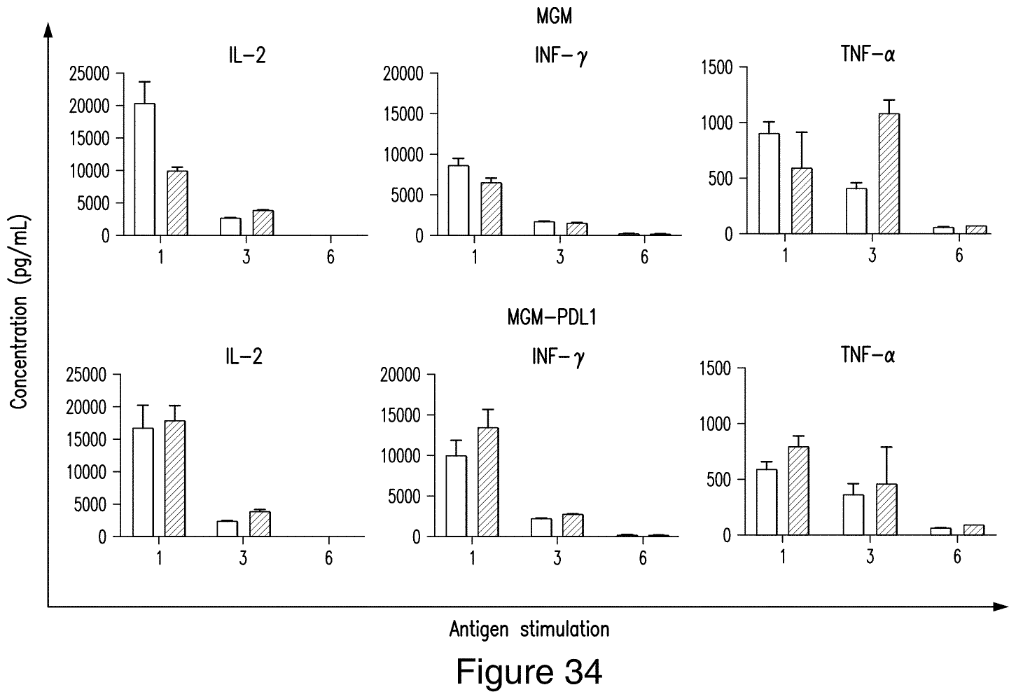

Secretion of effector cytokines, upon repeated antigen stimulation, by mycM28z1XXPD1DNR and mycM28z CAR T cells was assessed by Luminex assay. Both mycM28z1XXPD1DNR and mycM28z CAR T cells secreted high levels of IL-2, IFN-γ, and TNF-α after the first antigen stimulation. However, effector cytokine secretion decreased with both CAR T cells upon repeated antigen stimulation (see FIG. 34).

### C. In Vivo Studies

C.1. Antitumor Efficacy of mycM28z1XXPDIDNR CAR T Cells Visualized by ffLuc Tumor Cells

Using an orthotopic model, a series of in vivo experiments in mice were conducted to investigate the specificity and potency of mycM28z1XXPD1DNR CAR T cells by measuring tumor burden and animal survival. Serial BLI was used to confirm establishment of tumor, to equalize tumor burden across intervention groups before initiation of T-cell therapy, and subsequently to measure response to therapy.

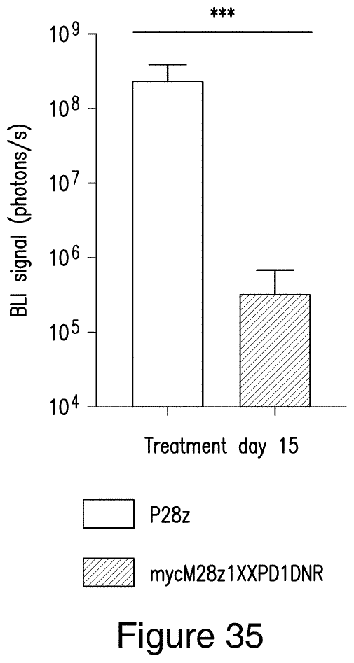

To investigate the antitumor efficacy of mycM28z1XXPD1DNR CAR T cells, a single low dose of 3×10⁴ CART cells was administered intrapleurally into female NSG mice 13 days after inoculation with orthotopic MGM tumor. The low dose was purposefully chosen to mimic the high tumor antigen burden faced by CAR T cells. Compared with control mice that received P28z CAR T cells, mice treated with a single dose of mycM28z1XXPD1DNR CAR T cells showed substantial reduction in tumor burden 15 days after T-cell administration (p=0.0002) (see FIG. 35). By day 15 after treatment, mice administered P28z CAR T cells started to become moribund, as expected, from high tumor burden, whereas no signs of toxicity were observed in mice that received mycM28z1XXPD1DNR CAR T cells.

Having observed efficacy and tumor eradication at a single dose of 3×10⁴ mycM28z1XXPD1DNR CART cells/mouse (translated to 1.2×10⁶ to 1.5×10⁶ cells/kg; mouse weight, 20-25 g), the inventors chose to increase the dose level by a factor of 3-4 to study toxicity in mice (reported in Section 3 of this Example (entitled “Nonclinical toxicology”)).

In a subsequent experiment, female NSG mice with established MGM-PDL1 pleural tumor were treated 11 days after tumor inoculation with a single intrapleural administration of 1×10⁵ or 5×10⁴mycM28z1XXPD1DNR CART cells per mouse (E:T ratio, 1:1000 to 1:2000; 2.5×10⁶ to 5×10⁶ cells/kg; average mouse weight, 20 g) or 1×10⁵ mycM28z CAR T cells/mouse as control. E:T ratios were estimated from tumor burden quantification, as described by us previously (Servais et al., Curr Protoc Pharmacol. 2011; Chapter 14:Unit14 21; Servais et al., PLoS One. 2011; 6(10):e26722).

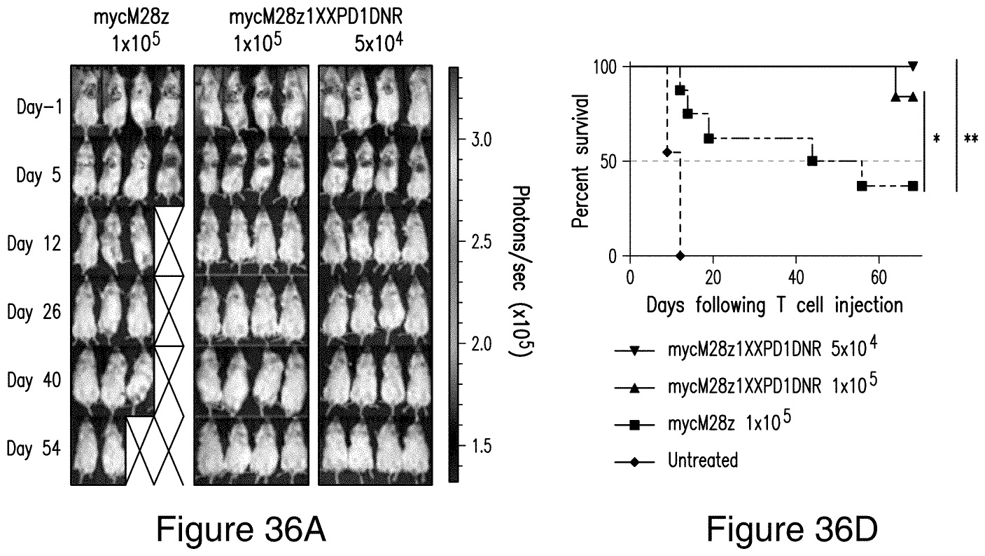

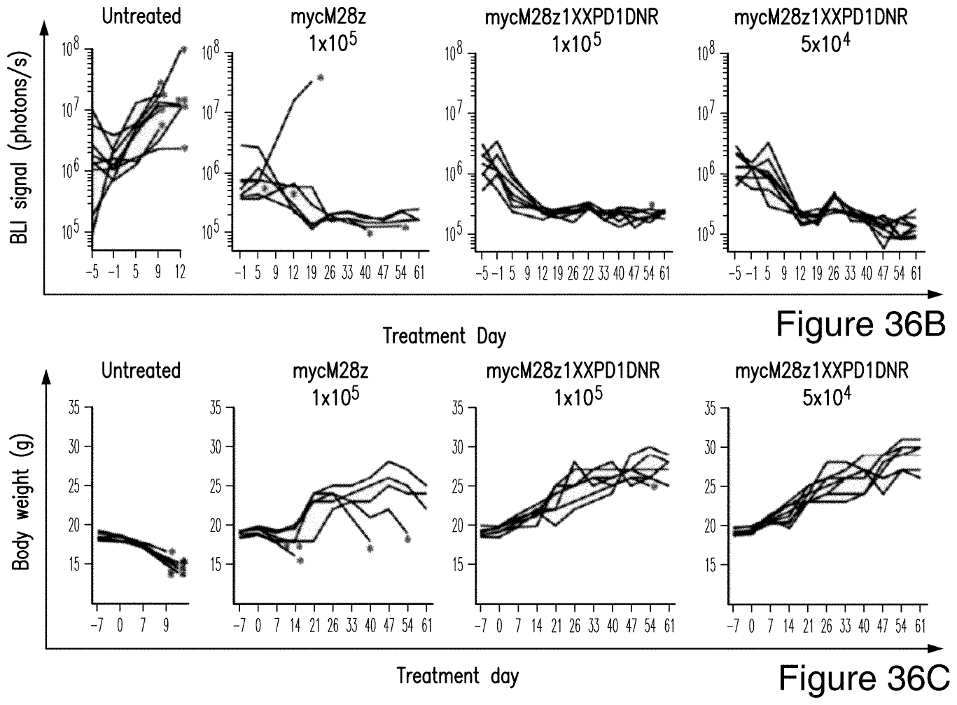

Serial tumor imaging with D-Luciferin (luciferin for ffluc) showed a decrease in tumor burden by BLI as early as 5 days after CAR T-cell administration, with complete tumor eradication (background BLI signal) at approximately day 19 for mice treated with 1×10⁵ mycM28z1XXPD1DNR CART cells and at approximately day 26 for mice treated with 1×10⁵ mycM28z CAR T cells (see FIGS. 36A and 36B). Untreated mice became moribund 9-12 days after the start of treatment (see FIG. 36B). Tumor eradication was maintained by mycM28z1XXPD1DNR CART cells (dose, 1×10⁵ and 5×10⁴) until termination of the study at day 68. However, in mice treated with mycM28z CAR T cells that were sacrificed before day 68 due to moribund status and loss of weight, necropsy showed presence of tumor. Mice treated with either dose of mycM28z1XXPD1DNR CAR T cells gained weight in a linear fashion throughout the time of the study, whereas mice treated with mycM28z CAR T cells lost weight toward the end of the study (see FIG. 36C). Median survival was 12 days for untreated mice and 50 days for mice treated with 1×10⁵ mycM28z CAR T cells; median survival was not reached for mice treated with either dose of mycM28z1XXPD1DNR CAR T cells (see FIG. 36D). Survival was significantly prolonged for mice treated with mycM28z1XXPD1DNR CAR T cells compared to mice treated with mycM28z CART cells (p=0.0085 for 5×10⁴mycM28z1XXPD1DNR and p=0.0427 for 1×10⁵ mycM28z1XXPD1DNR versus 1×10⁵ mycM28z CAR T cells). No apparent clinical signs of toxicity were observed in mice treated with mycM28z1XXPD1DNR CAR T cells.

C.2. mycM28z1XXPD1DNR CAR T-Cell Detection in Primary Tumor

In a subgroup of mice, after interpleural injection of 5×10⁵mycM28z1XXPD1DNR CAR T cells, mycM28z CAR T cells, or un-transduced T cells into MGM pleural tumor-bearing NSG mice, pleural tumor was isolated for 3 days post-injection and analyzed for human T-cell infiltration by human CD45 and human tumor mesothelin staining using immunofluorescence.

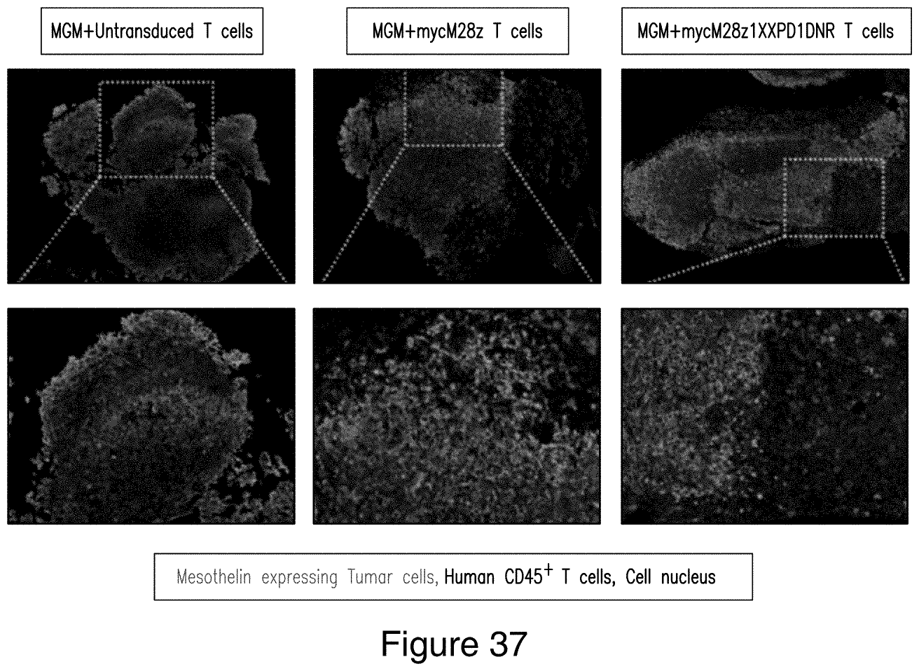

Among mice treated with un-transduced T cells, only a few un-transduced T cells were found in the tumor periphery and parenchyma, whereas mycM28z1XXPD1DNR CAR T cells and mycM28z CAR T cells were found in a higher density in the peritumoral area and at the interface between the tumor and the peritumoral area (see FIG. 37). These data suggest that regionally (intrapleurally) administered mesothelin-targeted T cells enrich peritumorally and infiltrate from the tumor periphery into the tumor.

C. 3. Antitumor Activity of mycM28z1XXPDIDNR CAR T Cells Upon Tumor Rechallenge In Vivo

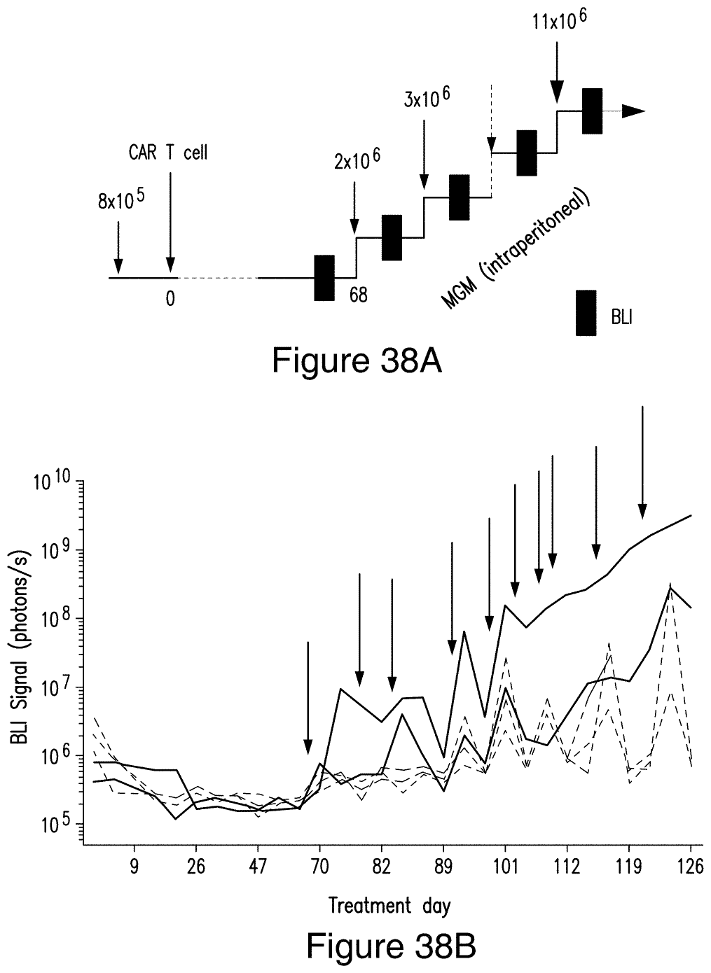

To investigate the functional persistence of mycM28z1XXPD1DNR CAR T cells, mice with pleural MGM-PDL1 tumor (n=5) treated with a single intrapleural dose of 1×10⁵ mycM28z1XXPD1DNR or mycM28z CAR T cells were rechallenged with escalating doses of MGM tumor cells intraperitoneally. Rechallenge started 68 days after intrapleural T-cell administration and following eradication of pleural tumor with a starting rechallenge dose of 2×10⁶ tumor cells. The tumor dose was escalated to 11×10⁶ tumor cells over a total of 10 rechallenges every 4-8 days (see FIG. 38A). Tumor rechallenge was associated with an increase in BLI signal shortly after each tumor administration, followed by a decrease in BLI, indicating T cell-mediated tumor regression (see FIG. 38B). BLI peak signals increased following subsequent escalating doses of tumor cells, and mice treated with mycM28z CAR T cells showed substantially higher increases in BLI signal, compared with mice treated with the same dose of mycM28z1XXPD1DNR CAR T cells. In particular, the BLI signal for mice treated with mycM28z1XXPD1DNR CAR T cells peaked shortly after each tumor rechallenge but returned to baseline BLI signal even at the highest rechallenge dose (see FIG. 38B). In contrast, mice treated with mycM28z CAR T cells showed a decrease in BLI signal following the initial increase in BLI for up to 5 tumor rechallenges, but they eventually lost their ability to control tumor reestablishment following higher tumor doses, leading to a continuous increase in BLI signal and tumor burden and a moribund state. mycM28z1XXPD1DNR CAR T cells resisted intraperitoneal tumor establishment for 10 repeated challenges, even >126 days after a single intrapleural dose of 1×10⁵CAR T cells, without any apparent clinical signs of toxicity. These data indicate that mycM28z1XXPD1DNR CAR T cells are capable of controlling tumor not only locally in the pleural space but also at distant locations within the body. In vivo, mycM28z1XXPD1DNR CAR T cells remained functional upon chronic antigen exposure via repeated antigen challenge and persisted long-term, whereas mycM28z CAR T cells became dysfunctional upon repeated antigen rechallenge, indicating superior functional persistence and enhanced long-term antitumor activity of mycM28z1XXPD1DNR CAR T cells. We have confirmed that this enhanced efficacy is not due to graft-versus-host disease. We administered nonantigen-expressing targets and noticed an increase in tumor BLI, with no antitumor response, confirming that the antitumor efficacy is antigen-specific.

C.4. Antitumor Efficacy of Clinical-Grade M28z1XXPDIDNR CAR T Cells

The purpose of this study was to validate the antitumor efficacy of cryopreserved M28z1XXPD1DNR CAR T cells that were generated by the MSK Cell Therapy and Cell Engineering Facility (CTCEF). Before intrapleural injection, cryopreserved aliquots of

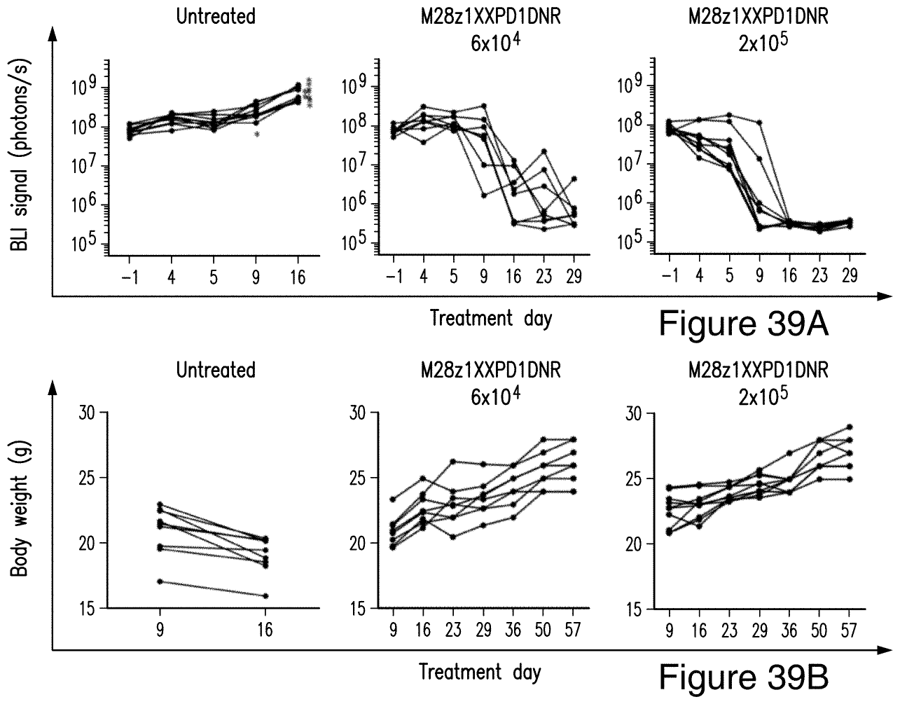

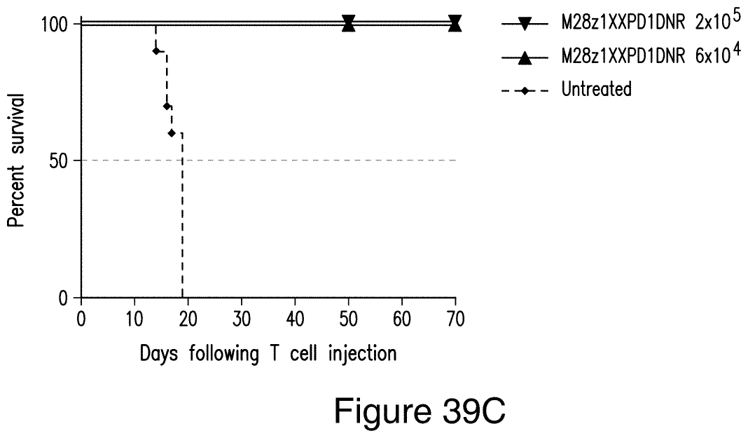

M28z1XXPD1DNR CAR T cells were thawed in RPMI-1640 with 10% FBS, washed twice in RPMI-1640 without FBS and resuspended in RPMI-1640 without FBS as a vehicle for injection. The viability of CART cells after thawing and before injection was determined to be 88% by acridine orange/propidium iodide staining. CAR T cells were intrapleurally injected into female NSG mice bearing pleural MGM tumors at a dose of 6×10⁴ or 2×10⁵ viable CAR T cells/mouse (n=8-10). Serial tumor BLI revealed tumor eradication by day 16 for mice treated with 2×10⁵ CAR T cells (see FIG. 39A). Mice treated with 6×10⁴ CAR T cells showed tumor regression and eradication by day 29 (see FIG. 39A). No weight loss was observed for either treatment group whereas untreated tumor-bearing mice lost weight and died within 19 days after start of treatment (see FIGS. 39B and 39C). No toxicities were observed for the CAR T cell-treated mice and 100% of the treated mice were alive at the end of the observation period (day 70, see FIG. 39C). This study confirmed that cryopreserved M28z1XXPD1DNR CAR T cells manufactured using the vector stocks produced for the proposed clinical trial demonstrated high viability after thawing, exhibited antitumor efficacy in vivo, and were well tolerated in mice.

### 3. Nonclinical Toxicology

### A. Summary of Nonclinical Safety Studies

M28z1XXPD1DNR CAR T cell binding and activity are specific to human mesothelin, and thus there is no ideal pharmacologically relevant species in which to conduct nonclinical safety studies. Additionally, variability in the expression pattern of target antigens and differences in the clearing mechanisms and immunogenicity of human polypeptides, such as the CAR in immunocompetent mice, hinder the usefulness of animals to predict the toxicity of CAR T cells in humans. Because M28z1XXPD1DNR CAR T cells have a relevant pharmacodynamic effect (cytokines, accumulation, tumor regression) in an orthotopic, immune-deficient mouse model expressing human mesothelin, we conducted a safety study in this xenogeneic model. This was a Good Laboratory Practice (GLP) study conducted at MSK by the Antitumor Assessment Core Facility. The design, methods, and results of this study are discussed in this section.

The dose chosen of the CART cells for this study was 1×10⁵ mycM28z1XXPD1DNR CAR T cells. This dose was chosen because it is 3-4× higher than the minimum effective dose tested (3×10⁴mycM28z1XXPD1DNR CART cells) for treatment of orthotopic mesothelioma tumors in our preclinical mouse model. Importantly, the selected dose of the CAR T cells (1×10⁵ cells/mouse or 5×10⁶ cells/kg) is 5× higher than the starting dose proposed in the current study (1×10⁶ cells/kg). In inventors' published studies, mesothelin-targeted CAR with PD1DNR (Cherkassky et al., J Clin Invest. 2016; 126(8):3130-3144. at a dose of 40,000 to 50,000 CART cells, was shown to eradicate high pleural tumor burden in mice with orthotopic pleural mesothelioma.

The CAR T cells were delivered once via intrapleural injection into NSG mice harboring mesothelioma xenografts in the pleural cavity. Unlike other agents, such as antibodies, for which intrapleural administration is initially limited to the pleural cavity, it has been shown that, in both mice and humans, intrapleurally administered CAR T cells circulate systemically within a day or two and are not limited to the pleural cavity (Adusumilli et al., Sci Transl Med. 2014; 6(261):261ra151; Cherkassky et al., J Clin Invest. 2016; 126(8):3130-3144; Adusumilli et al., Cancer Res. 2019; 79(13)). In particular, the inventors have observed in both mice and humans that intrapleurally administered CAR T cells are antigen-activated and proliferate 5-10-fold higher than the initially administered dose within a short period (Adusumilli et al., Sci Transl Med. 2014; 6(261):261ra151; Cherkassky et al., J Clin Invest. 2016; 126(8):3130-3144), so the actual number of CART cells tested in this study is 5-10-fold higher than the initially administered dose within a short period.

The following considerations are important in assessing the human safety of M28z1XXPD1DNR CAR T cells.

1. The previous human experience with CAR T cells indicates that exaggerated pharmacology is the main cause of adverse events, which are both monitorable and manageable. There have been no reports of adverse events linked to on-target, off-tumor toxicity of mesothelin-targeted CART cells in our phase I clinical study (Adusumilli et al., Cancer Res. 2019; 79(13)).

2. In in vitro studies, it was observed that the pharmacologic activity of mycM28z1XXPD1DNR CAR T cells correlated with the expression of mesothelin on the surface of the target cell.

3. In the inventors' orthotopic model of MPM, specific cytotoxicity and increased survival were observed after a single intrapleural injection of mycM28z1XXPD1DNR CAR T cells in mice bearing MPM tumors. Without treatment with mycM28z1XXPD1DNR CAR T cells, these mice would have died within 20-22 days of tumor implant. Within 7 days, tumor burden was substantially reduced, and animals remained cured beyond 68 days; in contrast, untreated controls had a median survival of 12 days, and mycM28z-treated controls had a median survival of 50 days.

Mortality and morbidity, weight, clinical signs, hematology and clinical chemistry, gross necropsy, and histopathologic data were assessed in 96 (48 male and 48 female) NSG mice (The Jackson Laboratory) with 8-day-old orthotopic MGM tumors randomly assigned to control and treatments groups. A dose of 1×10⁵ cells/mouse (approximately 5×10⁶ cells/kg) in RPMI-1640 was selected, which corresponds to at least 3-4× the approximate minimum effective dose determined in in vitro and in vivo proof-of-principle experiments in this model (3×10⁴ cells/mouse). The dose and necropsy regimen were chosen rationally: (a) although a higher initial test dose may allow investigation of any immediate untoward effects of dose administration (no such effects were seen in the ongoing clinical trials), the biology and pharmacology do not reflect the functional persistence and proliferation of mycM28z1XXPD1DNR CAR T cells (i.e., in the solid-tumor microenvironment, it is important to allow a relatively higher number of CAR T cells present in the tumor/body for a prolonged period of time than in flash kinetics of high dose-induced tumor eradication), and (b) day 14 was chosen because the tumor has either regressed significantly or been eradicated at this time point (as evidenced by BLI or necropsy from prior experiments) (Adusumilli et al., Sci Transl Med. 2014; 6(261):261ra151; Cherkassky et al., J Clin Invest. 2016; 126(8):3130-3144). Sacrifice and necropsy at this time point allow examination of any on-target, off-tumor effects (the scFv used in our CAR reacts to mouse mesothelin) (Feng et al., Mol Cancer Ther. 2009) on normal tissue, specifically pleura, peritoneum, and pericardium, with low levels of expression of mesothelin, following peak CAR T-cell expansion in the absence of tumor burden with high mesothelin expression.

mycM28z1XXPD1DNR CAR T cells or control vehicle were administered once via orthotopic injection on study day 1 (male mice) or study day 2 (female mice). All animals were observed for mortality and morbidity twice per week before study day −8, followed by daily monitoring (weekdays) until study day 1. After dosing, animals were monitored daily until the end of the study (study day 15). Body weights were recorded twice per week before study day −8, followed by daily monitoring (weekdays) until study day 1. After dosing, body weights were recorded daily until the end of the study (study day 15). Clinical signs were recorded twice per week before study day 1, followed by daily monitoring from study day 1 to 15. On study day 2 (interim sacrifice of male mice), study day 3 (interim sacrifice of female mice), study day 14 (final sacrifice of male mice), and study day 15 (final sacrifice of female mice), mice were sedated with isoflurane, and blood was collected for hematology and clinical chemistry. For gross and complete necropsies, tissues were collected and fixed in formalin. No tissues were discarded during necropsy. At the time of necropsy, gross examinations of each animal were performed by members of the Antitumor Assessment Core Facility. Any macroscopic lesions or other abnormal findings were recorded using standard terminology and provided to the pathologist for correlation with microscopic findings. For histopathologic analysis, all tissues of necropsied animals were preserved in formalin. After at least 24 h in fixative, the tissues were processed and embedded in paraffin. Paraffin blocks were then sectioned at 4 μm. The resulting unstained slides were then stained with hematoxylin and eosin. Slides were then shipped to HSRL. for examination by a board-certified pathologist. Lesions were recorded using morphologic diagnoses following standardized nomenclature. On study days 9 (male mice) and 10 (female mice), plasma was collected from a subgroup of mice, and cytokine assessment was performed by the Immune Monitoring Core Facility at MSK (non-GLP). In addition, spleen and tumor were isolated from a subgroup of mice for identification of mycM28z1XXPD1DNR CAR T cells via flow cytometry (non-GLP). On study days −1/1, 7/8, and 14/15, a subgroup of animals were imaged using luciferin (dorsal/ventral) to assess tumor burden and test article efficacy.

No mortality or morbidity was observed for animals in this study, with the exception of 2 control animals in the imaging cohort that underwent elected sacrifice due to morbidity and labored breathing on study days 12 and 14 (day 20-22 after tumor administration). Previous work in the inventors' laboratory has shown that control animals may become moribund due to tumor burden at approximately 20-22 days after tumor administration (Servais et al., Clin Cancer Res. 2012; Servais et al., Curr Protoc Pharmacol. 2011; Chapter 14:Unit14 21; Adusumilli et al., Sci Transl Med. 2014; 6(261):261ra151; Cherkassky et al., J Clin Invest. 2016; 126(8):3130-3144; Servais et al., PLoS One. 2011; 6(10):e26722). Therefore, these sacrifices were unscheduled, but not unexpected. No mortality or morbidity was traced to the test article.

Animals that received control vehicle showed a progressive decrease in body weight during the study and a significant difference in weight compared with nontumor controls and mice treated with mycM28z1XXPD1DNR CAR T cells. This was attributed to the increasing tumor burden of the control animals.

No significant clinical signs were observed for mice treated with mycM28z1XXPD1DNR CAR T cells. One test article-treated mouse was observed to have slight scabbing, which was attributed to irritation caused by the surgical clips, as no other animals were affected and animal activity was normal. Mice appeared normal throughout the monitoring period.

Female mice treated with mycM28z1XXPD1DNR CAR T cells in group 12 (final sacrifice on study day 15) had a high average percent monocyte value (average, 18.44%, n=5) (p≤0.0001), compared with group 10 (tumor control vehicle) (average, 3.34%, n=5). The reference range established for percent monocytes is 0.9%-18%. However, this did not correlate with any microscopic findings. No other significant or abnormal results were observed for the hematology parameters assessed. Any differences between test article-treated groups and the corresponding vehicle-treated groups were within normal reference ranges or were not biologically relevant or statistically significant.

Male mice treated with mycM28z1XXPD1DNR CAR T cells in group 11 (final sacrifice on study day 14) had a low average total protein value (average, 3.83 g/dL, n=5) (p=0.0022), compared with group 9 (tumor control vehicle) (average, 4.68 g/dL, n=4). The reference range established for total protein is 4.1-6.4 g/dL. However, this did not correlate with any microscopic findings. No other adverse effects on clinical chemistry parameters were observed with test article administration. Any differences between test article-treated groups and the corresponding vehicle-treated groups were within normal reference ranges or were not biologically relevant or statistically significant.

At the time of necropsy, tumor tissue was not collected but was observed in many animals in the interim sacrifice, as anticipated. At the time of final sacrifice, tumor burden was evident in control vehicle animals, but for most cases minor to no tumor foci were observed in the test article-treated animals. Gross observations at the time of necropsy for individual animals included spotted liver, small spleen, blue seminal vesicle, dark green gallbladder, blue gallbladder, oily kidney, bubble in liver, and white spot on spleen. None of these findings correlated to microscopic findings. A small spleen is expected in the NSG mouse phenotype due to lymphocyte depletion.

Histopathologic review determined that there were no microscopic findings at the interim (study day 2/3) and final (study day 14/15) sacrifice days related to acute or delayed toxicity from test article administration. Microscopic findings for animals in groups 5 and 6 (test article; interim sacrifice) included the presence of mixed cellular infiltration within the xenograft tumors in the lungs. This was considered to be related to test article administration but not related to any test article toxicity. This finding was confirmed in a separate study by immunofluorescence staining of mycM28z1XXPD1DNR CAR T cells in the primary tumor of intrapleurally treated mice (see Section 2.C.2 of this Example). Any other observed findings were determined to occur sporadically, at a similar incidence as in controls, or were common in the species/strain utilized.

In vivo BLI on study day −1/1 indicated successful tumor administration for all animals in each group. Following control vehicle administration, tumor burden for animals in the male and female mice groups increased at each measured time point, with 2 animals requiring early imaging due to morbidity. Following test article administration, tumor burden for animals in the male and female mice groups demonstrated an increase 1 week after injection; however, at the 2-week time point, the burden was substantially decreased. Human T cells were detected in spleen tissue and tumor 8 days after intrapleural administration in mycM28z1XXPD1DNR CAR T cell-treated mice but not in vehicle-treated mice. Mouse plasma cytokine levels obtained at the same time point showed slightly elevated levels of IL-4 in mice treated with CAR T cells, compared with those treated with vehicle control. Levels of IL-1β, IL-6, KC/GRO, and TNF-α were generally low and were not significantly different between mice receiving CAR T cells and those receiving vehicle control. IFN-γ, IL-12p70, IL-1β, IL-2, and IL-5 were not detectable (below the limit of quantitation).

The purpose of this study was to assess the acute and delayed toxicity of mycM28z1XXPD1DNR CAR T cells in NSG mice following a single orthotopic injection. Throughout the study, body weight, clinical signs, hematology, clinical chemistry, and histopathologic data were collected and analyzed. The results of this study indicate that single orthotopic administration of 1×10⁵ mycM28z1XXPD1DNR CART cells in a mesothelioma xenograft model is well-tolerated.

### B. Methods

Test system. Experiments were conducted with 6-8-week-old male and female NSG mice from The Jackson Laboratory. Mice in the tumor groups received 800,000 MGM cells/mouse via intrapleural injection on study day 1 (male mice) and study day 2 (female mice). These mice are expected to develop symptoms of pleural disease approximately 5-20 days after injection.

Test article. The test article (mycM28z1XXPD1DNR CAR T cells) was prepared by the inventors at MSK. PBMCs from a healthy donor were thawed on Nov. 5, 2019 and transduced with retroviral particles encoding for mycM28z1XXPD1DNR on Nov. 7, 2019. Prior to injection, transduced PBMCs were maintained in RPMI-1640 media with 10% FBS, 100 units/mL penicillin, 100 μg/mL streptomycin, and 20 units/mL IL-2. On injection days Nov. 13, 2019 (male mice) and Nov. 14, 2019 (female mice), transduced cells were pooled, analyzed by flow cytometry for CD3 and CAR expression, washed, and resuspended in vehicle (RPMI-1640 without FBS and without phenol red) and stored on ice until injection.

mycM28z1XXPD1DNR CAR T cells were resuspended in vehicle at a final concentration of 5×10⁵ viable CART cells/mL. Cell viability was measured. The prepared solution was then transferred to the Animal Facility on ice for immediate use. The test article, ready for use and stored on ice, was considered stable under these conditions throughout the study period. Formulation of the CAR T cells was performed in the inventors' Laboratory within a laminar flow hood at room temperature under aseptic conditions. The viability and CAR expression was determined pre and post-dose.

Vehicle. The vehicle used in preparation of the CAR T cells formulations and for administration to the control group was sterile RPMI-1640 media without FBS and without phenol red (GIBCO, cat #32404, lot number 2099376). Vehicle was transferred to the Animal Facility on ice for immediate use. Vehicle, ready for use and stored on ice, was considered stable under these conditions throughout the study period, as supported by the certificate of analysis by the manufacturer. Preparation of the vehicle was performed in the inventors' Laboratory within a laminar flow hood at room temperature under aseptic conditions.

Study design. All cohorts were randomized into study groups before study day 1. Upon arrival, animals were randomly selected from the animal crates and placed in their respective treatment groups as outlined in the Table 9 below. Mice were identified by using ear punches.

Mice in the tumor groups received 8×10⁵ MGM cells via orthotopic injection in the pleura 8 days before test article administration. mycM28z1XXPD1DNR CAR T cells, or a control vehicle, were administered similarly via orthotopic injection once on study day 1 (male mice) or study day 2 (female mice). Tumor cells, vehicle, and test article were administered at an injection volume of 200 μL/mouse.

TABLE 9
Group assignment.

| | Mice/ | | Mice | Blood/Tissue | |
|---|---|---|---|---|---|
| Group* | Group | Sex | ID# | Collection | Necropsy |
| Interim Sacrifice | | | | | |
| (24 h) Day 2/3 | | | | | |
| 1. No Tumor - | 3 | M | 1-3 | CBC & Chem | Gross |
| Control Vehicle | | | | | |
| 2. No Tumor - | 3 | F | 4-6 | CBC & Chem | Gross |
| Control Vehicle | | | | | |
| 3. Tumor - | 10 | M | 7-16 | CBC & Chem | Complete |
| Control Vehicle | | | | | |
| 4. Tumor - | 10 | F | 17-26 | CBC & Chem | Complete |
| Control Vehicle | | | | | |
| 5. Tumor - Test | 10 | M | 27-36 | CBC & Chem | Complete |
| Article | | | | | |
| 6. Tumor - Test | 10 | F | 37-46 | CBC & Chem | Complete |
| Article | | | | | |
| Final Sacrifice | | | | | |
| Day 14/15 | | | | | |
| 7. No Tumor - | 3 | M | 47-49 | CBC & Chem | Gross |
| Control Vehicle | | | | | |
| 8. No Tumor - | 3 | F | 50-52 | CBC & Chem | Gross |
| Control Vehicle | | | | | |
| 9. Tumor - | 5 | M | 53-57 | CBC & Chem | Complete |
| Control Vehicle | | | | | |
| 10. Tumor - | 5 | F | 58-62 | CBC & Chem | Complete |
| Control Vehicle | | | | | |
| 11. Tumor - | 5 | M | 63-67 | CBC & Chem | Complete |
| Test Article | | | | | |
| 12. Tumor - | 5 | F | 68-72 | CBC & Chem | Complete |
| Test Article | | | | | |
| TK/Cytokines | | | | | |
| 13. Tumor - | 3 | M | 73-75 | Tumor, Spleen, | None |
| Control Vehicle | | | | 200 uL EDTA | |
| 14. Tumor - | 3 | F | 76-78 | Tumor, Spleen, | None |
| Control Vehicle | | | | 200 uL EDTA | |
| 15. Tumor - | 3 | M | 79-81 | Tumor, Spleen, | None |
| Test Article | | | | 200 uL EDTA | |
| 16. Tumor - | 3 | F | 82-84 | Tumor, Spleen, | None |
| Test Article | | | | 200 uL EDTA | |
| Imaging | | | | | |
| 17. Tumor - | 3 | M | 85-87 | None | None |
| Control Vehicle | | | | | |
| 18. Tumor - | 3 | F | 88-90 | None | None |
| Control Vehicle | | | | | |
| 19. Tumor - | 3 | M | 91-93 | None | None |
| Test Article | | | | | |
| 20. Tumor - | 3 | F | 94-96 | None | None |
| Test Article | | | | | |

*Explanation of groups:
No Tumor - Control Vehicle: Non tumor mice + RPMI1640
Tumor - Control Vehicle: Mice with MGM tumor + RPMI1640
Tumor - Test Article: Mice with MGM tumor + 1 × 10⁵ mycM28z1XXPD1DNR CAR T cells

Clinical signs and body weights were collected throughout the study to assess morbidity, and acute and delayed toxicity were assessed 1 and 14 days after dosing, respectively. Hematology, clinical chemistry, and histopathologic data were collected and assessed at the acute and delayed time points to determine test article tolerability.

Parameters Evaluated.

Mortality and morbidity. All animals were observed for mortality and morbidity twice per week before study day −8, followed by daily monitoring (weekdays) until study day 1. After dosing, animals were monitored daily until the end of the study (study day 15).

Body weight. Body weights were recorded twice per week before study day −8, followed by daily monitoring (weekdays) until study day 1. After dosing, body weights were recorded daily until the end of the study (study day 15).

Clinical signs. Clinical signs were recorded twice per week before study day 1, followed by daily monitoring from study day 1 to 15.

Hematology. On study day 2 (male groups 1, 3, 5), study day 3 (female groups 2, 4, 6), study day 14 (male groups 7, 9, 11), and study day 15 (female groups 8, 10, 12), mice were sedated with isoflurane, and whole blood was collected in an EDTA tube for the following measurements as shown in Table 10:

TABLE 10
Hematology parameters.

| | |
|---|---|
| Neutrophils | White blood cell count |
| Lymphocytes | Red blood cell count |
| Monocytes | Hemoglobin concentration |
| Eosinophils | Hematocrit |
| Basophils | Mean corpuscular volume |
| % Neutrophils | Mean corpuscular hemoglobin |
| % Lymphocytes | Mean corpuscular hemoglobin concentration |
| % Monocytes | Red blood cell distribution width |
| % Eosinophils | Platelet count |
| % Basophils | Mean platelet volume |

Clinical chemistry. On study day 2 (male groups 1, 3, 5), study day 3 (female groups 2, 4, 6), study day 14 (male groups 7, 9, 11), and study day 15 (female groups 8, 10, 12), mice were sedated with isoflurane. Whole blood was collected in a serum separator tube. Serum was separated and analyzed for the following measurements as shown in Table 11:

TABLE 11
Clinical chemistry parameters.

| | |
|---|---|
| Blood urea nitrogen | Cholesterol |
| Creatinine | Alanine aminotransferase |
| Phosphorus | Aspartate aminotransferase |
| Calcium | Alkaline phosphatase |
| Total protein | Total bilirubin |
| Albumin | Sodium |
| Globulin | Potassium |
| Albumin/globulin ratio | Chloride |
| Glucose | Sodium/potassium ratio |

Necropsy. On study day 2 (male groups 1, 3, 5), study day 3 (female groups 2, 4, 6), study day 14 (male groups 7, 9, 11), and study day 15 (female groups 8, 10, 12), mice were euthanized via CO₂ inhalation. Gross necropsies were performed on animals in groups 1, 2, 7, and 8, and complete necropsies were performed on animals in groups 3-6 and 9-12. Tissues were collected and fixed in formalin. No tissues were discarded during necropsy.

Gross pathology observations. At the time of necropsy, gross examinations of each animal were performed by members of the Antitumor Assessment Core Facility. Any macroscopic lesions or other abnormal findings were recorded using standard terminology and provided to the pathologist for correlation with microscopic findings.

Histopathology. All tissues of necropsied animals were preserved in formalin. After at least 24 h in fixative, the tissues listed in Table 12 were processed and embedded in paraffin (tissues denoted with an asterisk were decalcified before embedding). Paraffin blocks were then sectioned at 4 μm. The resulting unstained slides were then stained with hematoxylin and eosin. Slides were then shipped to HSRL for examination by a board-certified pathologist. Lesions were recorded using morphologic diagnoses following standardized nomenclature.

TABLE 12
Tissues examined microscopically.

| | |
|---|---|
| Adrenals | Pancreas |
| Bone (marrow)* | Rectum |
| Brain (brainstem, cerebellum, | Salivary glands |
| and cerebrum) | Skin |
| Esophagus | Spleen |
| Eyes | Sternum* |
| Gallbladder | Stifle joint* |
| Heart | Stomach |
| Inguinal lymph node | Testes, epididymis, seminal |
| Intestines (duodenum, jejunum, | vesicles, and prostate (males) |
| ileum, cecum, colon) | Thymus |
| Kidneys | Thyroid |
| Liver | Tongue |
| Lungs | Trachea |
| Mesenteric lymph node | Urinary bladder |
| Oral-nasal cavity (nasopharynx)* | Vertebral column* |
| Ovaries, uterine horns, uterus, | |
| cervix, and vagina (females) | |

Cytokine analysis (non-GLP). In study days 9 (male mice) and 10 (female mice), blood was collected from mice in groups 13, 15 (males), 14, and 16 (females) in EDTA tubes. Plasma was separated and frozen. Cytokine assessment was performed by the Immune Monitoring Core Facility at MSK (non-GLP).

Toxicokinetics: Identification of CART cells via flow cytometry (non-GLP). On study days 9 (male mice) and 10 (female mice), blood was collected from mice in groups 13, 15 (males), 14, and 16 (females). Gross necropsy was performed on all animals, at which time tumor tissue and spleens were placed in RPMI media. Samples were immediately provided to the inventors for identification of mycM28z1XXPD1DNR CAR T cells via flow cytometry (non-GLP).

Bioluminescence imaging. On study days −1/1, 7/8, and 14/15, animals in groups 17-20 were imaged using luciferin (dorsal/ventral) to assess tumor burden and test article efficacy.

Statistical methods. Group means and standard deviations were calculated for body weights, hematology, and clinical chemistry parameters. For hematology and clinical chemistries, at each time point, the percent difference between the mean of the test article-treated groups and corresponding vehicle-treated groups was calculated. The statistical significance of these differences was analyzed using an unpaired t test and considered statistically significant at p<0.05. Statistical analyses were performed using Ascentos Version 1.3.4, a preclinical laboratory information systems software by PDS Life Sciences (Mt. Arlington, N.J.). Review of clinical data was performed by assessing individual values for potential outliers using the 1.5× IQR test and parameter reference ranges established in the laboratory. Any values determined to be outliers were removed from statistical analysis.

### C. Results

C.1. Mortality and Morbidity

Previous work by the inventors indicated that control animals may become moribund due to tumor burden approximately 20-22 days after tumor administration. Two control animals in the imaging cohort (86 [group 17] and 88 [group 19]) underwent elected sacrifice on study days 12 and 14 due to morbidity and labored breathing. Gross necropsy of each animal following sacrifice indicated significant tumor burden in the thoracic cavity, surrounding the lungs and heart. Both animals were treated with control vehicle and demonstrated morbidity within the expected window. Therefore, while these sacrifices were unscheduled, they were not unexpected. No other mortality or morbidity was observed for any animals in this study.

C.2. Body Weights

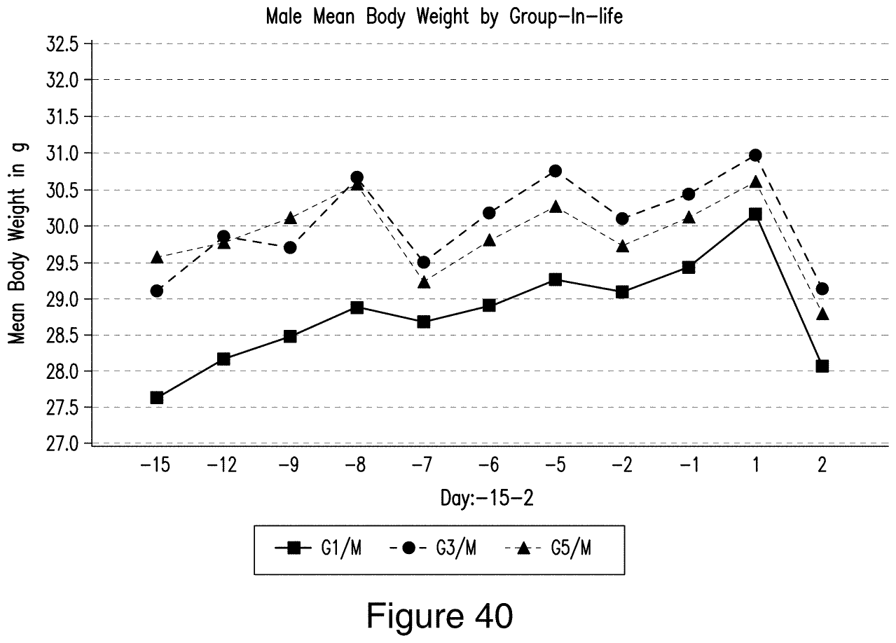

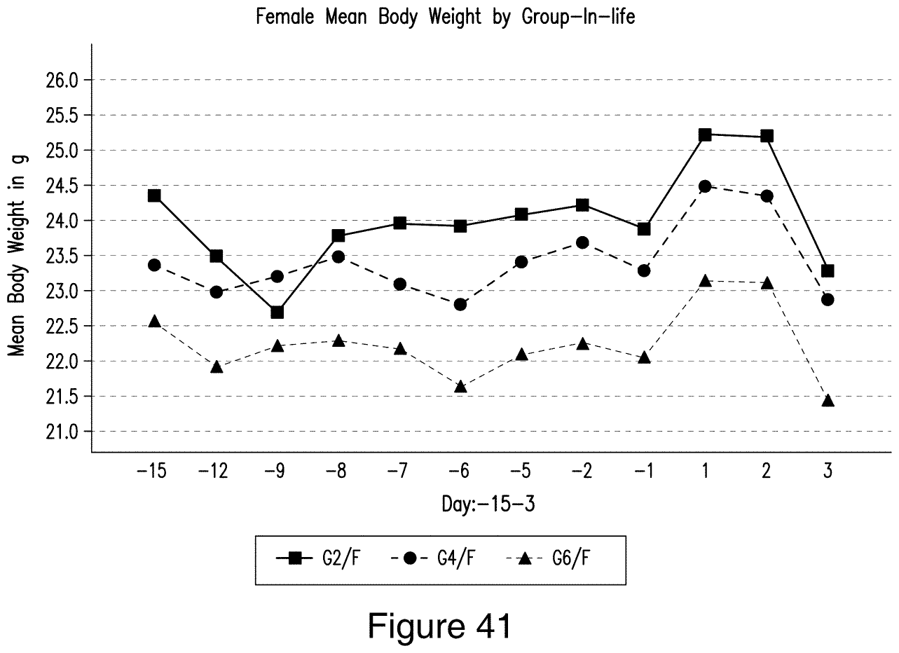

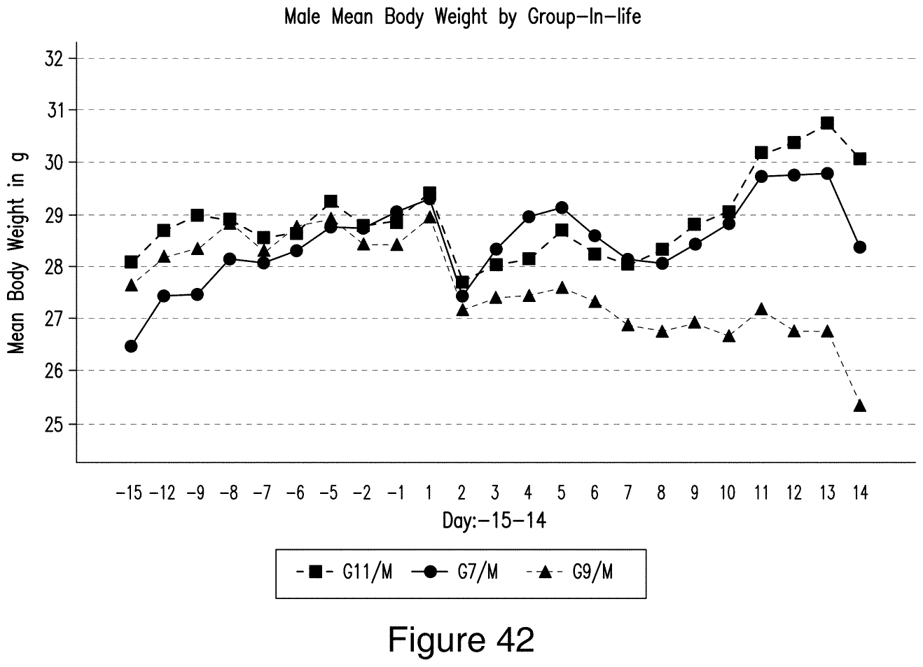

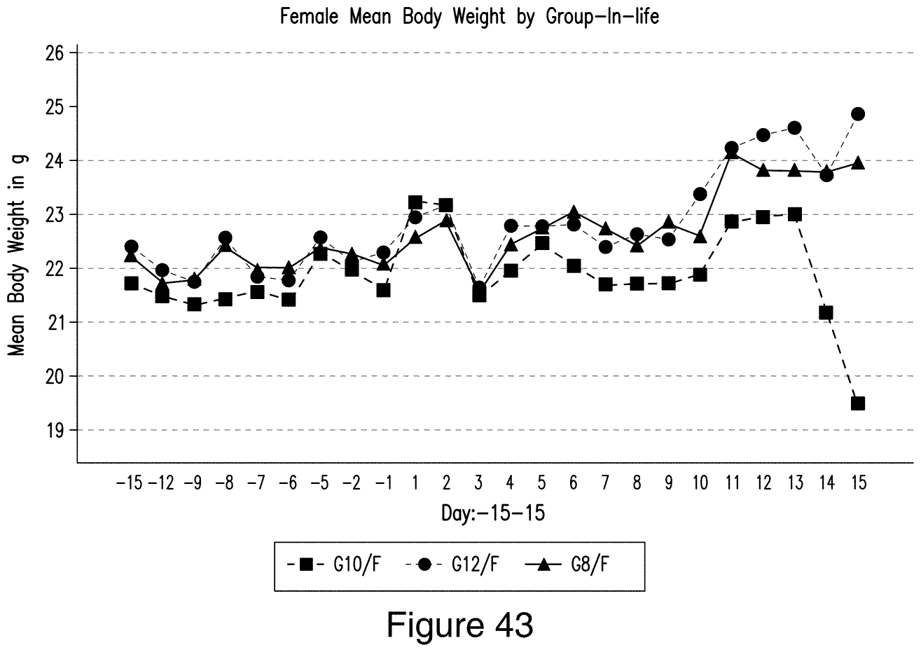

A summary of average body weight by group is shown in FIGS. 40-43. Animals in group 9 (control vehicle) showed a progressive decrease in body weight during the study, and a significant difference in weight, compared with the nontumor controls (group 7) and test article animals (group 11), on study day 14. This is attributed to the increasing tumor burden of the control animals (see FIG. 42). Similarly, animals in group 10 (control vehicle) demonstrated a significant difference in body weight, compared with group 8 (nontumor control vehicle) and group 12 (test article), beginning on study day 13 and continue through the end of the study (see FIG. 43). This is also attributed to tumor burden. All animals showed a drop in body weight the day following surgery (day 2 for male mice and day 3 for female mice) which was attributed to the surgery. The nontumor control and mice treated with mycM28z1XXPD1DNR CAR T cells regained their initial weight before surgery within 2-3 days after surgery and progressively gained weight during the remainder of the study whereas mice treated with vehicle showed little to no recovery in body weight after surgery and progressively lost weight with the concurrent increase in tumor burden (see FIGS. 42 and 43).

C.3. Clinical Signs

Animals in group 17 (control vehicle) demonstrated signs of hair loss (85, 87) and scabbing (87), which were attributed to an aggressor animal inside the cage. Once the animals were separated, signs lessened, indicating that observations were due to animals fighting. Animal 86 was observed to have labored breathing and reduced activity on study day 12, which was attributed to tumor burden and resulted in an elected sacrifice. Animal 93 (group 19, test article) was observed to have slight scabbing; however, this was attributed to irritation caused by the surgical clips, as no other animal was affected in the cage and animal activity was normal. Animal 88 (group 18, control vehicle) was observed to have reddened eyes 2 days before elected sacrifice. This clinical sign may have been an early indication of animal discomfort before the elected sacrifice due to tumor burden.

No other significant clinical signs were observed during this study—mice appeared normal throughout the monitoring period.

C.4. Hematology

Group 12 (female mice, test article) had a high average percent monocyte value (average, 18.44%, n=5) (p=≤0.0001), compared with group 10 (tumor control vehicle) (average, 3.34%, n=5). The reference range established for percent monocytes is 0.9%-18%. However, this did not correlate with any microscopic findings.

No other significant or abnormal results were observed for the hematology parameters assessed. Any differences between test article-treated groups and the corresponding vehicle-treated groups were within normal reference ranges or were not biologically relevant or statistically significant.

C.5. Clinical Chemistry

Group 11 (male mice, test article) had a low average total protein value (average, 3.83 g/dL, n=5) (p=0.0022), compared with group 9 (tumor control vehicle) (average, 4.68 g/dL, n=4). The reference range established for total protein is 4.1-6.4 g/dL. However, this did not correlate with any microscopic findings.

No other adverse effects on clinical chemistry parameters were observed with test article administration. Any differences between test article-treated groups and the corresponding vehicle-treated groups were within normal reference ranges or were not biologically relevant or statistically significant.

C. 6. Gross Pathology Observations

At the time of necropsy, tumor tissue was not collected but was observed in many animals at the interim sacrifice, as anticipated. At the time of final sacrifice, tumor burden was evident in control vehicle animals, but for most cases minor to no tumor foci were observed in the test article-treated animals. Additional findings at the time of necropsy are detailed in Table 13 below.

TABLE 13
Macroscopic observations with correlations to microscopic findings.

| | | | Test |
|---|---|---|---|
| Gross | Animal(s) Observed | | Article |
| Observation | (Group Number) | Correlated Finding | Related? |
| Spotted liver | 11 (G3-Control Vehicle) | Not correlated to | No |
| | 32 (G5-Test Article) | microscopic findings | |
| | 59 (G10-Control Vehicle) | | |
| | 60 (G10-Control Vehicle) | | |
| | 71 (G12-Test Article) | | |
| Small spleen | 11 (G3-Control Vehicle) | Lymphocyte depletion | No |
| | 16 (G3-Control Vehicle) | expected component of | |
| | 25 (G4-Control Vehicle) | NSG mouse phenotype | |
| | 30 (G5-Test Article) | | |
| | 34 (G5-Test Article) | | |
| | 40 (G6-Test Article) | | |
| | 45 (G6-Test Article) | | |
| | 56 (G9-Control Vehicle) | | |
| | 58 (G10-Control Vehicle) | | |
| | 59 (G10-Control Vehicle) | | |
| | 60 (G10-Control Vehicle) | | |
| | 65 (G11-Test Article) | | |
| | 71 (G12-Test Article) | | |
| Blue seminal | 57 (G9-Control Vehicle) | Not correlated to | No |
| vesicle | | microscopic findings | |
| Dark green | 107 (G9-Control Vehicle) | Not correlated to | No |
| gallbladder | | microscopic findings | |
| Blue | 62 (G10-Control Vehicle) | Not correlated to | No |
| gallbladder | | microscopic findings | |
| Oily kidney, | 21 (G4-Control Vehicle) | Not correlated to | No |
| spotted | 71 (G12-Test Article) | microscopic findings | |
| Bubble in | 40 (G6-Test Article) | Not correlated to | No |
| liver | | microscopic findings | |
| White spot | 69 (G12-Test Article) | Moderate metaplasia | No |
| on spleen | | considered spontaneous | |
| | | finding | |

No other gross observations were noted during necropsy.

C.7. Histopathology

Histopathologic review determined that there were no microscopic findings at the interim (day 2) and final (day 14) sacrifice days related to acute or delayed toxicity from test article administration.

Microscopic findings for animals in groups 5 and 6 (test article) included the presence of mixed cellular infiltration within the xenograft tumors in the lungs. This was considered to be related to test article administration but not related to any test article toxicity. This finding was confirmed in a separate study by immunofluorescence staining of mycM28z1XXPD1DNR CAR T cells in the primary tumor of intrapleurally treated mice (see Section 2.C.2 of this Example).

Any other observed findings were determined to occur sporadically, at a similar incidence as in controls, or were common in the species/strain utilized.

C.8. Cytokine Analysis

Cytokine levels in plasma collected from groups 13-16 (8 days after test article/vehicle administration) were measured to examine the toxicity of mycM28z1XXPD1DNR CAR T cells versus vehicle control on peripheral cytokine expression in treated mice. The following cytokines were analyzed: IFN-γ, IL-2, IL-4, IL-5, IL-6, IL-1β, IL-12p70, KC/GRO, and TNF-α.

Results from this analysis demonstrated undetectable levels of IFN-γ, IL-12p70, IL-1β, IL-2, and IL-5 (below the limit of quantitation) for all animals whether they received CAR T cells or vehicle control. IL-4 was detectable at low levels and was the only cytokine that showed slightly higher levels in mice receiving CART cells than in those receiving vehicle control. Levels of all other cytokines that were detected above the limit of quantitation, such as IL-1β, IL-6, KC/GRO, and TNF-α, were generally low and were not significantly different between mice receiving CAR T cells and those receiving vehicle control.

C.9. Identification of mycM28z1XXPD1DNR CAR T Cells in Tumor and Spleen

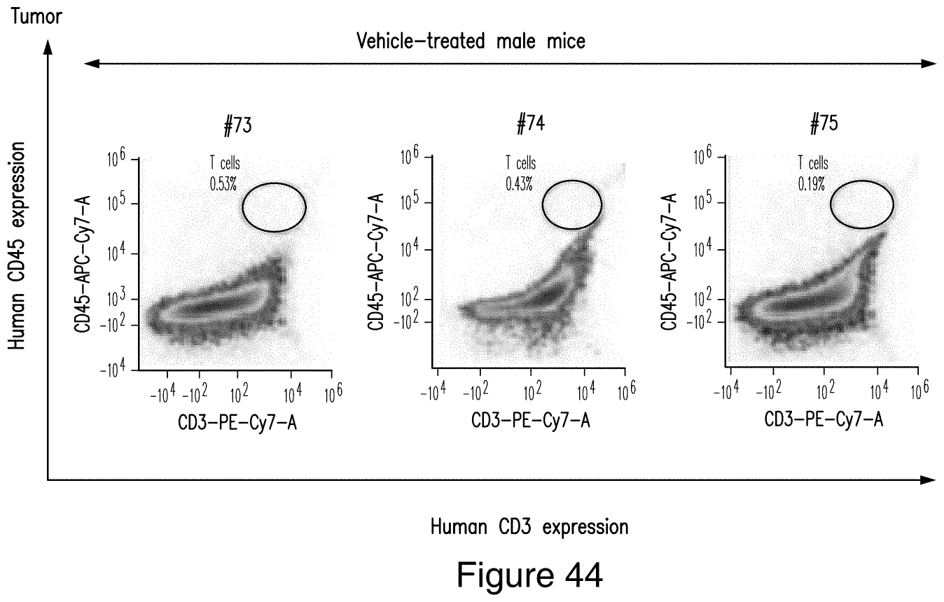

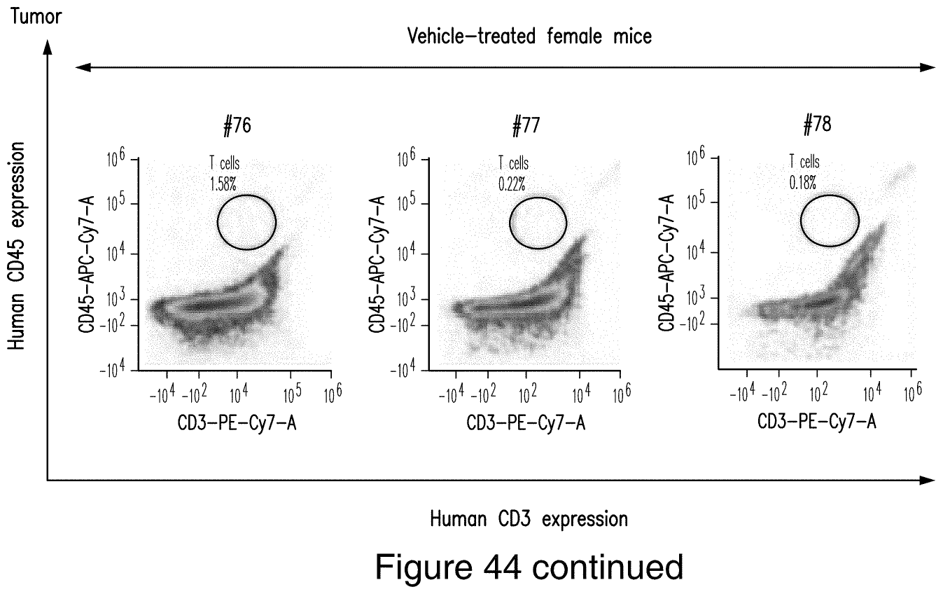

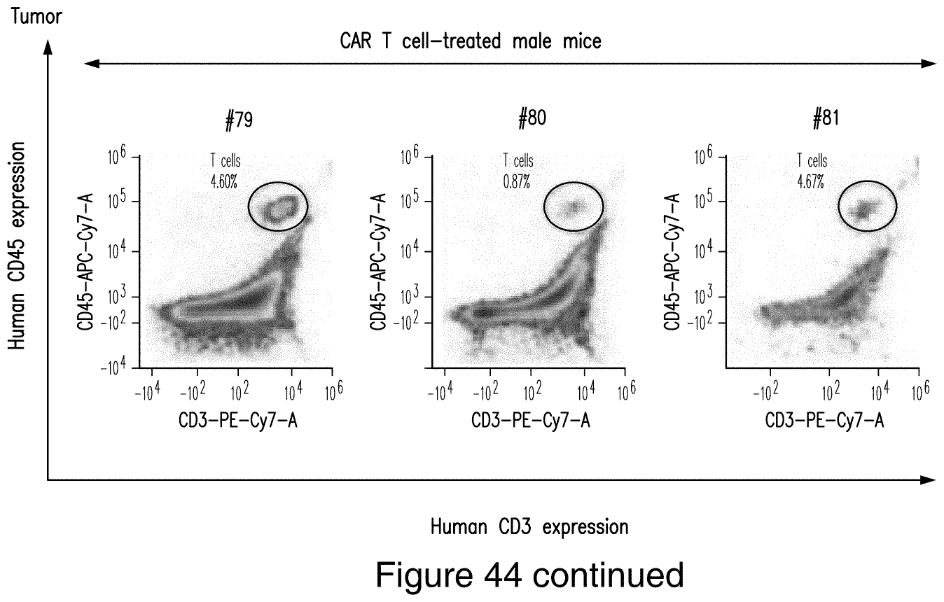

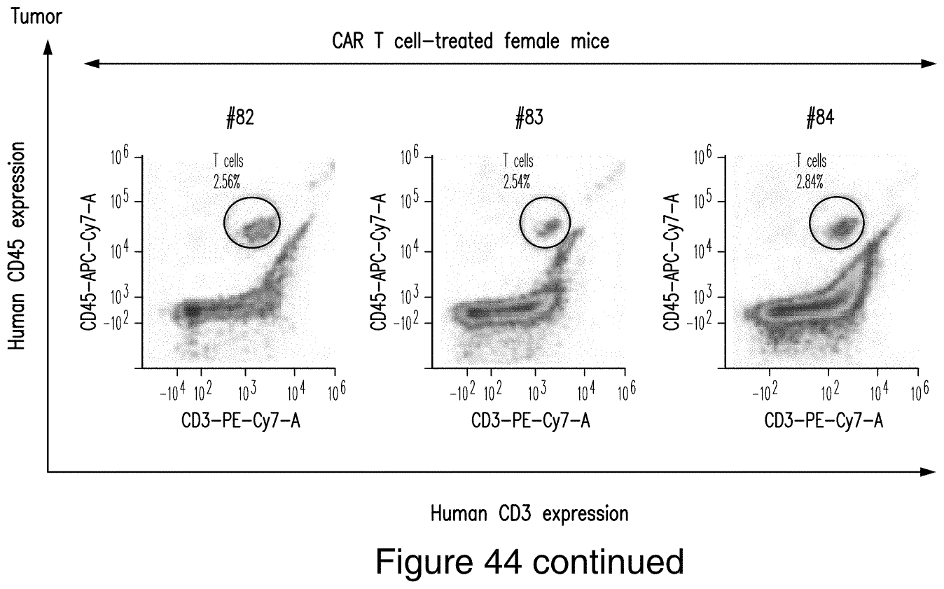

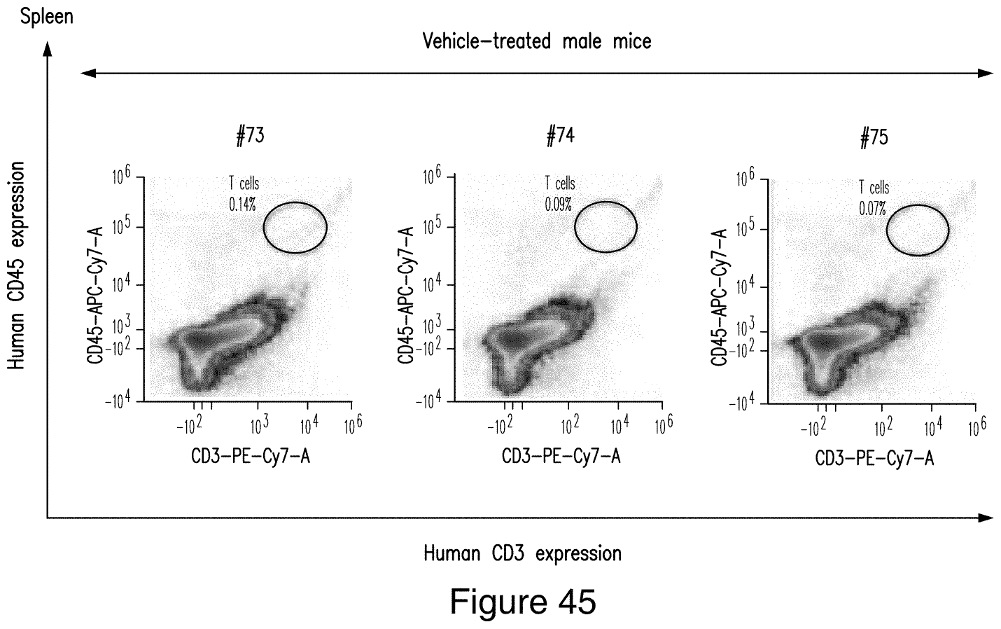

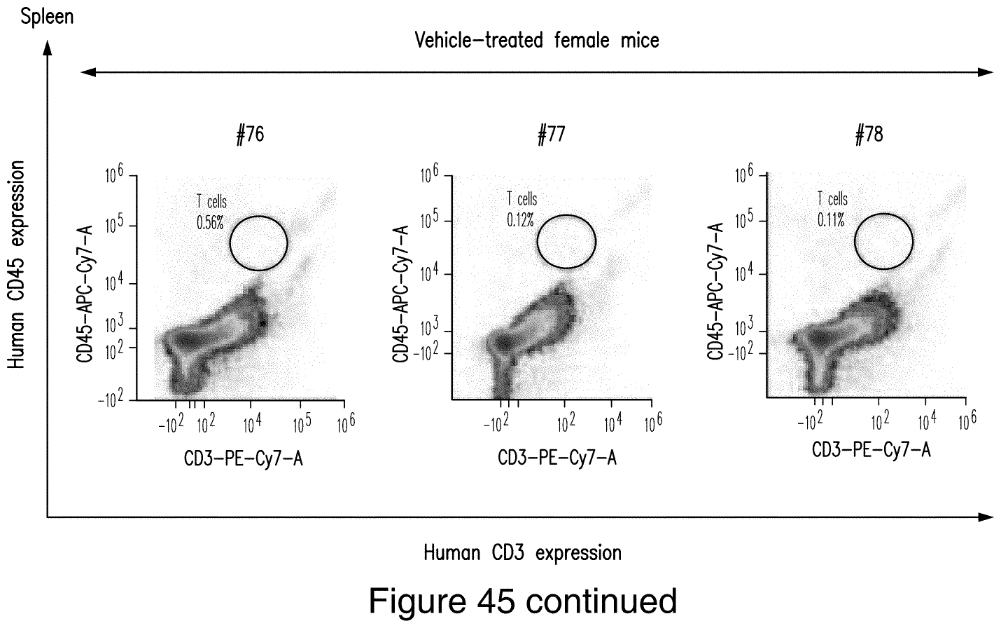

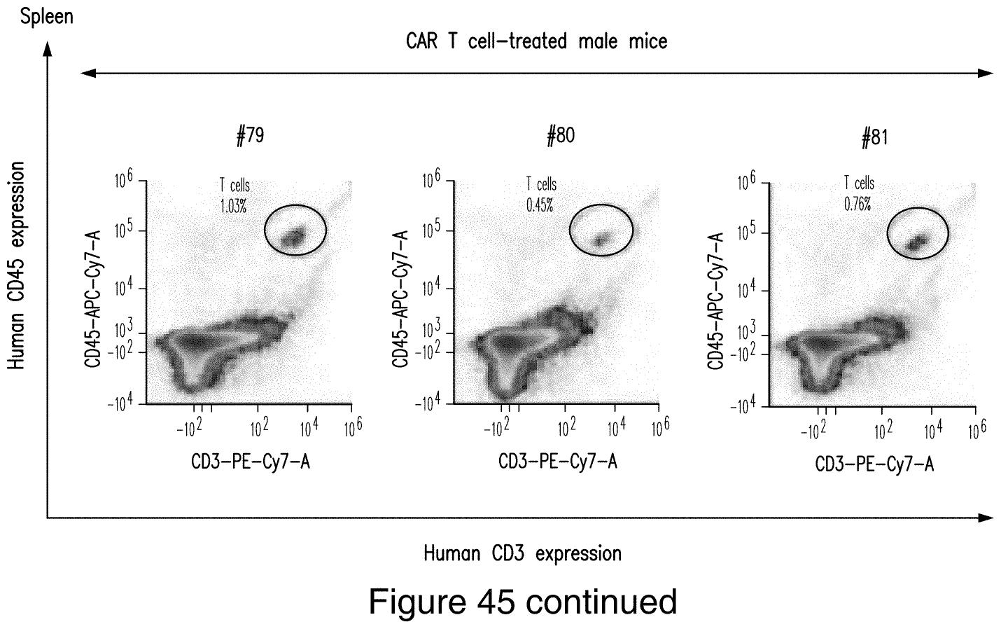

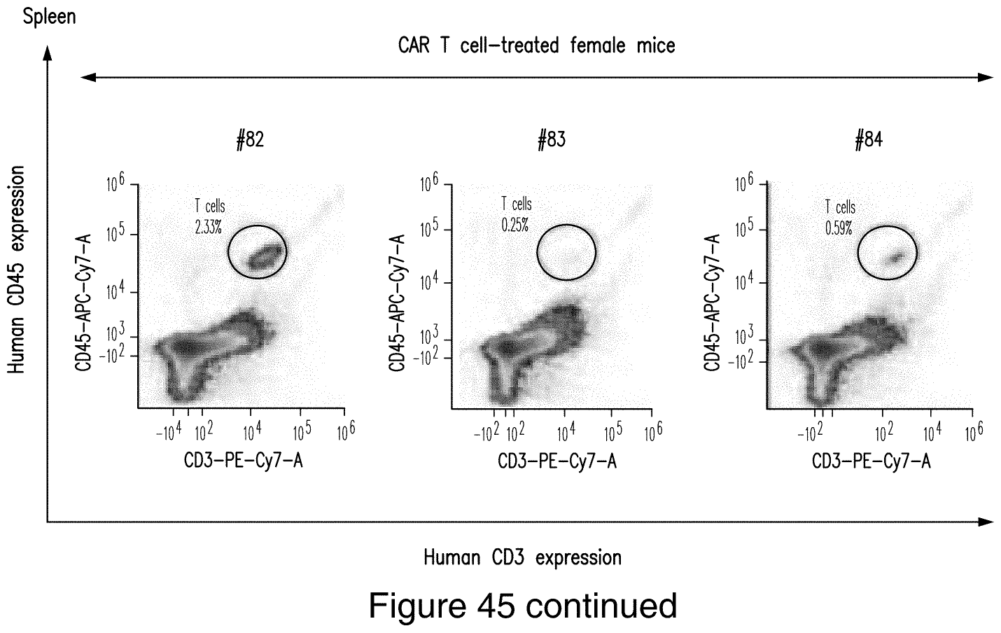

To confirm injection of mycM28z1XXPD1DNR CAR T cells, groups 13-16 were sacrificed 8 days after test/vehicle administration. Spleen and tumor were isolated, processed, and stained for human T cells (CD45+ CD3+) by flow cytometry. Human T cells were detected in tumor and spleen tissue from all mycM28z1XXPD1DNR CAR T cell-treated mice but not in those from vehicle-treated mice (see FIGS. 44 and 45).

C.10. Bioluminescence Imaging

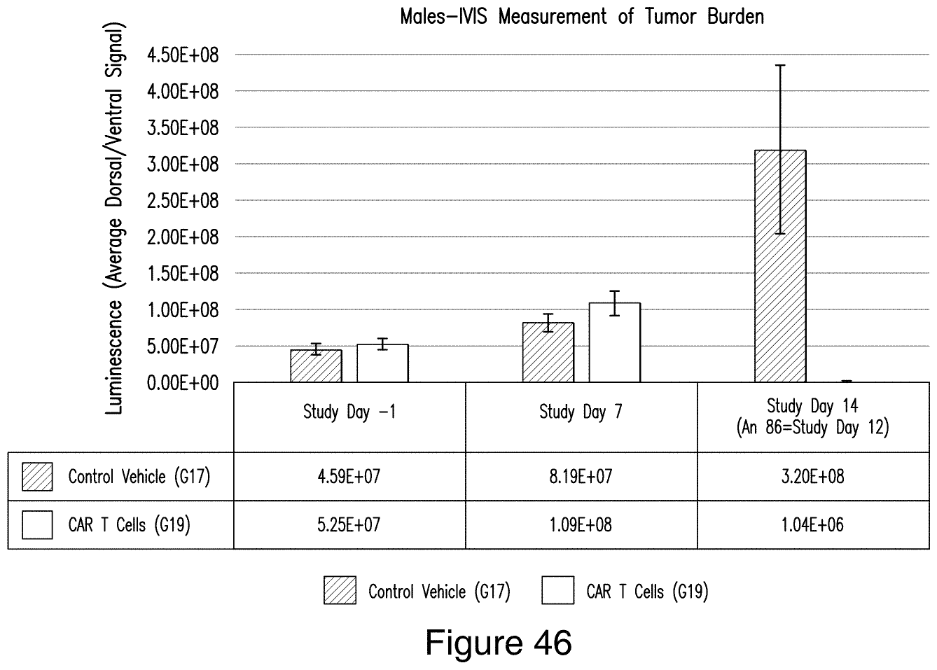

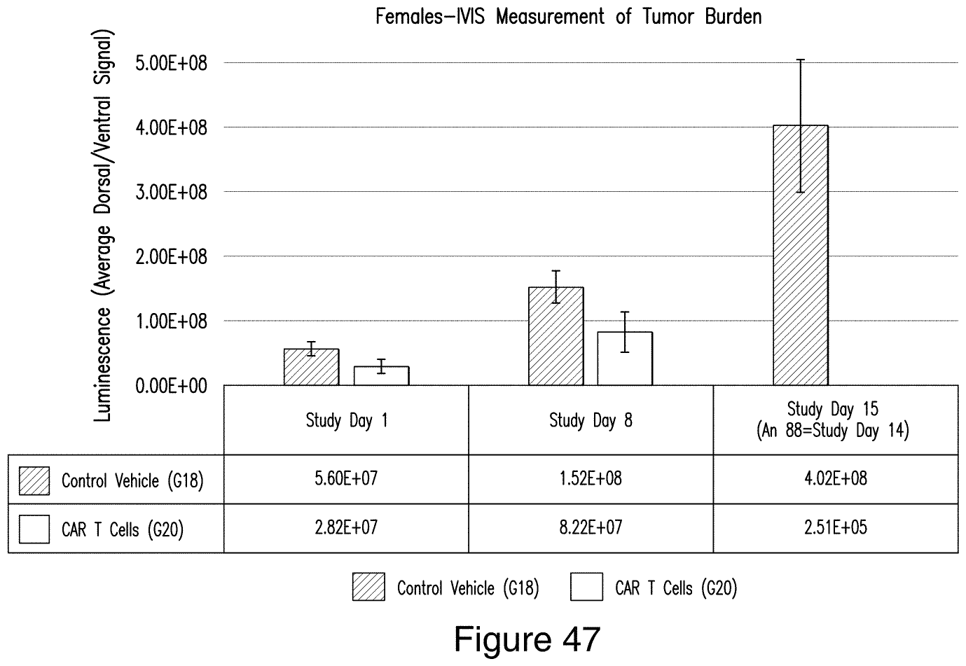

Whole-body optical BLI (IVIS) was performed on mice in groups 17-20 to provide a qualitative assessment of MGM tumor burden. Images from study day −1/1 indicate successful tumor administration for all animals in each group. Following control vehicle administration, the tumor burden for animals in groups 17 (male mice) and 18 (female mice) increased at each measured time point, with 2 animals requiring early imaging due to morbidity. Following test article administration, the tumor burden for animals in groups 19 (male mice) and 20 (female mice) demonstrated an increase 1 week after injection; however, at the 2-week time point, the burden was significantly decreased. The imaging results for male and female mice are shown in FIGS. 46 and 47, respectively.

### D. Discussion

Extensive pharmacology studies were conducted to determine the antitumor efficacy of M28z1XXPD1DNR CAR T cells in vitro and in vivo. It was shown that mycM28z1XXPD1DNR CAR T cells kill target cells in a mesothelin-dependent and HLA-independent manner, accumulate, and secrete effector cytokines when antigen-stimulated. A sophisticated antigen stress test using repeated antigen stimulation revealed that mycM28z1XXPD1DNR CAR T cells were able to retain cytotoxicity longer than mycM28z CAR T cells over the course of the assay. In vivo, repeated antigen challenge revealed superior functional persistence of mycM28z1XXPD1DNR CAR T cells, compared with mycM28z CAR T cells, leading to consecutive tumor regression over a series of 10 tumor rechallenges with escalating tumor doses for mycM28z1XXPD1DNR CAR T cell-treated mice but eventual tumor progression and relapse for mycM28z CAR T cells-treated mice.

Animal pharmacology studies are more relevant to study the antitumor efficacy of CAR T cells, as they cover important aspects of pharmacology and pharmacokinetics that cannot be studied in in vitro assays. Factors such as route of administration, trafficking to the tumor site, homing to lymphoid organs, systemic circulation, in vivo persistence, and, despite the limitations of the immunocompromised mouse strain used, interaction with the tumor immune microenvironment ultimately modulate antitumor efficacy. The inventors have developed a translationally relevant orthotopic mouse model of pleural mesothelioma that resembles human disease (Servais et al., Clin Cancer Res. 2012). The inventors have shown that regional (intrapleural) administration of T cells is vastly superior against not only pleural disease but also disseminated tumor sites (Adusumilli et al., Sci Transl Med. 2014; 6(261):261ra151). More importantly, results observed in ongoing clinical trials (IND 16354) mirror our original observations in the orthotopic MPM mouse model, attesting to the validity of the model for clinical relevance (Adusumilli et al., Cancer Res. 2019; 79(13)). In this study, complete tumor eradication was observed with a single low dose of 3×10⁴ mycM28z1XXPD1DNR CART cells delivered regionally and no signs of on-target, off-tumor toxicity were observed. A toxicity study of a single orthotopic dose of 1×10⁵ mycM28z1XXPD1DNR CAR T cells in the mesothelioma xenograft mouse model with a 2-week recovery period confirmed that mycM28z1XXPD1DNR CAR T cells were well-tolerated. No significant findings were found for mortality, clinical observations, changes in body weight, and gross/macroscopic examination. Microscopic examination revealed mixed cellular infiltration within xenograft tumors that was not observed for vehicle-treated mice but is indicative of the pharmacology of mycM28z1XXPD1DNR CAR T cells. Male treated mice had a low average total protein value, and female treated mice exhibited a high average percent monocyte value at the 2-week time point, but neither of these correlated with any microscopic findings.

CAR T cells, including mesothelin-targeted CAR T cells, have been evaluated in humans and have been reported to be safe, with defined, manageable side effects such as fevers, chills, myalgias, hypersensitivities, and anaphylaxis due to inflammation caused by T cells. The risk of replication-competent retroviruses will be minimized by extensive testing of the product. Furthermore, patient T cells will be tested every 12 weeks for up to 2 years or until disease progression. Patients will be followed for survival for up to 15 years following CAR T-cell administration.

The combined evaluation of these data has been used to establish a safe starting dose in humans and to support the dose-escalation scheme.

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A polypeptide composition comprising:
i) a chimeric antigen receptor (CAR) comprising:
(a) an extracellular antigen-binding domain comprising: a heavy chain variable region that comprises a CDR1 consisting of the amino acid sequence set forth in SEQ ID NO:76, a CDR2 consisting of the amino acid sequence set forth in SEQ ID NO:77, and a CDR3 consisting of the amino acid sequence set forth in SEQ ID NO:78; and a light chain variable region that comprises a CDR1 consisting of the amino acid sequence set forth in SEQ ID NO:79, a CDR2 consisting of the amino acid sequence set forth in SEQ ID NO:80, and a CDR3 consisting of the amino acid sequence set forth in SEQ ID NO:81,
(b) an intracellular signaling domain comprising a modified CD3ζ polypeptide comprising an ITAM2 variant and an ITAM3 variant, wherein each of the ITAM2 variant and the ITAM3 variant comprises two loss-of-function mutations; and
ii) a dominant negative form of programmed death 1 (PD-1 DN) comprising:
(a) at least a portion of an extracellular domain of programmed death 1 (PD-1) comprising a ligand binding region, and
(b) a first transmembrane domain.

2. The polypeptide composition of claim 1, wherein the extracellular antigen-binding domain of the CAR specifically binds to human mesothelin with an EC50 value of from about 1 nM to about 25 nM.

3. The polypeptide composition of claim 1, wherein the extracellular antigen-binding domain of the CAR comprises a single-chain variable fragment (scFv), a Fab that is optionally crosslinked, or a F(ab)₂.

4. The polypeptide composition of claim 3, wherein the extracellular antigen-binding domain of the CAR comprises a human scFv.

5. The polypeptide composition of claim 1, wherein the extracellular antigen-binding domain of the CAR recognizes human mesothelin with a mesothelin expression level of about 1,000 or more mesothelin binding sites/cell.

6. The polypeptide composition of claim 1, wherein the heavy chain variable region comprises an amino acid sequence that is at least about 80% homologous or identical to the amino acid sequence set forth in SEQ ID NO:82, and/or the light chain variable region comprising an amino acid sequence that is at least about 80% homologous or identical to the amino acid sequence set forth in SEQ ID NO:83.

7. The polypeptide composition of claim 1, wherein the heavy chain variable region comprises the amino acid sequence set forth in SEQ ID NO:82, and/or the light chain variable region comprises the amino acid sequence set forth in SEQ ID NO: 83.

8. The polypeptide composition of claim 1, wherein the heavy chain variable region comprises the amino acid sequence set forth in SEQ ID NO:82, and the light chain variable region comprises the amino acid sequence set forth in SEQ ID NO: 83.

9. The polypeptide composition of claim 1, wherein the extracellular antigen-binding domain of the CAR comprises a linker between the heavy chain variable region and the light chain variable region, and/or a leader is covalently joined to a N-terminus of the extracellular antigen-binding domain.

10. The polypeptide composition of claim 9, wherein the leader comprises a CD8 polypeptide.

11. The polypeptide composition of claim 10, wherein the CD8 polypeptide consists of the amino acid sequence set forth in SEQ ID NO: 71.

12. The polypeptide composition of claim 1, wherein the at least a portion of an extracellular domain of PD-1 comprises amino acids 21 to 165 of SEQ ID NO: 48.

13. The polypeptide composition of claim 1, wherein the first transmembrane domain of the PD-1 DN comprises a CD8 polypeptide, a CD28 polypeptide, a CD3ζ polypeptide, a CD4 polypeptide, a 4-1BB polypeptide, an OX40 polypeptide, a CD166 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40/My88 peptide, a NKGD2 peptide, or a combination thereof.

14. The polypeptide composition of claim 13, wherein the first transmembrane domain of the PD-1 DN comprises a CD8 polypeptide.

15. The polypeptide composition of claim 14, wherein the CD8 polypeptide comprised in the first transmembrane domain of the PD-1 DN comprises amino acids 137 to 207 of SEQ ID NO: 86.

16. The polypeptide composition of claim 1, wherein the PD-1 DN comprises amino acids 21 to 165 of SEQ ID NO: 48 and amino acids 137 to 207 of SEQ ID NO: 86.

17. The polypeptide composition of claim 1, wherein each of the two loss-of-function mutations is at a tyrosine amino acid residue.

18. The polypeptide composition of claim 1, wherein the ITAM2 variant comprises or consists of the amino acid sequence set forth in SEQ ID NO: 29, and/or the ITAM3 variant comprises or consists of the amino acid sequence set forth in SEQ ID NO: 33.

19. The polypeptide composition of claim 1, wherein the modified CD3ζ polypeptide comprises a native ITAM1.

20. The polypeptide composition of claim 19, wherein the native ITAM1 comprises or consists of the amino acid sequence set forth in SEQ ID NO: 23.

21. The polypeptide composition of claim 1, wherein the modified CD3ζ polypeptide comprises or consists of the amino acid sequence set forth in SEQ ID NO: 35.

22. The polypeptide composition of claim 1, wherein the PD-1 DN lacks an intracellular domain.

23. The polypeptide composition of claim 1, wherein the CAR further comprises a second transmembrane domain.

24. The polypeptide composition of claim 23, wherein the second transmembrane domain of the CAR comprises a CD8 polypeptide, a CD28 polypeptide, a CD3ζ polypeptide, a CD4 polypeptide, a 4-1BB polypeptide, an OX40 polypeptide, a CD166 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40/My88 peptide, a NKGD2 peptide, or a combination thereof.

25. The polypeptide composition of claim 24, wherein the second transmembrane domain of the CAR comprises a CD28 polypeptide

26. The polypeptide composition of claim 1, wherein the intracellular signaling domain of the CAR further comprises a co-stimulatory signaling region.

27. The polypeptide composition of claim 26, wherein the co-stimulatory signaling region comprises a CD28 polypeptide, a 4-1BB polypeptide, an OX40 polypeptide, an ICOS polypeptide, a DAP-10 polypeptide, a CD27 polypeptide, a CD40/My88 polypeptide, a NKGD2 polypeptide, or a combination thereof.

28. The polypeptide composition of claim 27, wherein the co-stimulatory signaling region comprises a CD28 polypeptide.

29. The polypeptide composition of claim 1, wherein the CAR comprises the amino acid sequence set forth in SEQ ID NO: 56.

30. An immunoresponsive cell comprising a polypeptide composition of claim 1.

31. The immunoresponsive cell of claim 30, wherein the PD-1 DN and/or the CAR is recombinantly expressed, and/or expressed from a vector.

32. The immunoresponsive cell of claim 30, wherein the immunoresponsive cell is selected from the group consisting of a T cell, a Natural Killer (NK) cell, and a pluripotent stem cell from which a lymphoid cell may be differentiated.

33. The immunoresponsive cell of claim 32, wherein the immunoresponsive cell is a T cell.

34. The immunoresponsive cell of claim 33, wherein the T cell is selected from the group consisting of a cytotoxic T lymphocyte (CTL), a regulatory T cell, and a Natural Killer T (NKT) cell.

35. The immunoresponsive cell of claim 32, wherein the pluripotent stem cell is an embryonic stem cell or an induced pluripotent stem cell.

36. The immunoresponsive cell of claim 30, wherein the immunoresponsive cell is autologous or allogenic.

37. A pharmaceutical composition comprising an effective amount of an immunoresponsive cell of claim 30 and a pharmaceutically acceptable excipient.

38. The pharmaceutical composition of claim 37, comprising between about 10⁴ and 10⁶, at least about 10⁵, or about 10⁵ of the immunoresponsive cells.

39. The pharmaceutical composition of claim 37, which is for preventing and/or treating a neoplasm in a subject, treating a subject having a relapse of a neoplasm, reducing tumor burden in a subject, increasing or lengthening survival of a subject having a neoplasm, preventing and/or treating an inflammatory disease in a subject, and/or preventing graft rejection in a subject who is a recipient of an organ transplant.

40. A nucleic acid composition comprising a polynucleotide encoding the polypeptide composition of claim 1.

41. A vector comprising the nucleic acid composition of claim 40.

42. A method for producing an immunoresponsive cell, the method comprising introducing into an immunoresponsive cell a nucleic acid composition of claim 40.

43. A kit comprising a polypeptide composition of claim 1.

44. A method of preventing and/or treating a neoplasm in a subject, reducing tumor burden in a subject, treating a subject having a relapse of a neoplasm, increasing or lengthening survival of a subject having a neoplasm, increasing immune-activating cytokine production in response to a cancer cell or a pathogen in a subject, preventing and/or treating an inflammatory disease in a subject, and/or preventing graft rejection in a subject who is a recipient of an organ transplant, the method comprising administering to the subject an effective amount of the immunoresponsive cells of claim 30.
